SEQUENCE LISTING (465 sequences)

SEQ ID NO: 1 - protein, 382 aa, Artificial
  synthetic modified protease
VRSKKLWISLLFALALIFTMAFGSTSSAQAAGKSNGEKKYIVGFKQTMSTMSAAKKKDVISEKGGKVQKQFKYVDAASATLNEKAVKELKKDPSVAYVEEDHVAHAYAQSVPYGVSQIKAPALHSQGYTGSNVKVAVIDSGIDSSHPDLKVAGGASMVPSETNPFQDNNSHGTHVAGTVAALNNSIGVLGVAPSASLYAVKVLGADGSGQYSWIINGIEWAIANNMDVINMSLGGPSGSAALKAAVDKAVASGVVVVAAAGNEGTSGSSSTVGYPGKYPSVIAVGAVDSSNQRASFSSVGPELDVMAPGVSIQSTLPGNKYGALNGTSMASPHVAGAAALILSKHPNWTNTQVRSSLENTTTKLGDSFYYGKGLINVQAAAQ

SEQ ID NO: 2 - DNA, 1149 bp, Artificial
  synthetic polynucleotide encoding SEQ ID NO1
gtgagaagcaaaaaattgtggatcagtttgctgtttgctttagcgttaatctttacgatggcgttcggcagcacatccagcgcgcaggcggcagggaaatcaaacggggaaaagaaatatattgtcgggtttaaacagacaatgagcacgatgagcgccgctaagaagaaagatgtcatttctgaaaaaggcgggaaagtgcaaaagcaattcaaatatgtagacgcagcttcagctacattaaacgaaaaagctgtaaaagaattgaaaaaagacccgagcgtcgcttacgttgaagaagatcacgtagcacacgcgtacgcgcagtccgtgccttacggcgtatcacaaattaaagcccctgctctgcactctcaaggctacactggatcaaatgttaaagtagcggttatcgacagcggtatcgattcttctcatcctgatttaaaggtagcaggcggagccagcatggttccttctgaaacaaatcctttccaagacaacaactctcacggaactcacgttgccggcacagttgcggctcttaataactcaatcggtgtattaggcgttgcgccaagcgcatcactttacgctgtaaaagttctcggtgctgacggttccggccaatacagctggatcattaacggaatcgagtgggcgatcgcaaacaatatggacgttattaacatgagcctcggcggaccttctggttctgctgctttaaaagcggcagttgataaagccgttgcatccggcgtcgtagtcgttgcggcagccggtaacgaaggcacttccggcagctcaagcacagtgggctaccctggtaaatacccttctgtcattgcagtaggcgctgttgacagcagcaaccaaagagcatctttctcaagcgtaggacctgagcttgatgtcatggcacctggcgtatctatccaaagcacgcttcctggaaacaaatacggcgcgttgaacggtacatcaatggcatctccgcacgttgccggagcggctgctttgattctttctaagcacccgaactggacaaacactcaagtccgcagcagtttagaaaacaccactacaaaacttggtgattctttctactatggaaaagggctgatcaacgtacaggcggcagctcagtaa

SEQ ID NO: 3 - protein, 30 aa, Artificial
  synthetic modified protease
VRSKKLWISLLFALALIFTMAFGSTSSAQA

SEQ ID NO: 4 - DNA, 90 bp, Artificial
  synthetic polynucleotide encoding SEQ ID NO3
gtgagaagcaaaaaattgtggatcagtttgctgtttgctttagcgttaatctttacgatggcgttcggcagcacatccagcgcgcaggcg

SEQ ID NO: 5 - protein, 77 aa, Artificial
  synthetic modified protease
AGKSNGEKKYIVGFKQTMSTMSAAKKKDVISEKGGKVQKQFKYVDAASATLNEKAVKELKKDPSVAYVEEDHVAHAY

SEQ ID NO: 6 - DNA, 231 bp, Artificial
  synthetic polynucleotide encoding SEQ ID NO5
gcagggaaatcaaacggggaaaagaaatatattgtcgggtttaaacagacaatgagcacgatgagcgccgctaagaagaaagatgtcatttctgaaaaaggcgggaaagtgcaaaagcaattcaaatatgtagacgcagcttcagctacattaaacgaaaaagctgtaaaagaattgaaaaaagacccgagcgtcgcttacgttgaagaagatcacgtagcacacgcgtac

SEQ ID NO: 7 - protein, 107 aa, Artificial
  synthetic modified protease
VRSKKLWISLLFALALIFTMAFGSTSSAQAAGKSNGEKKYIVGFKQTMSTMSAAKKKDVISEKGGKVQKQFKYVDAASATLNEKAVKELKKDPSVAYVEEDHVAHAY

SEQ ID NO: 8 - DNA, 321 bp, Artificial
  synthetic polynucleotide encoding SEQ ID NO7
gtgagaagcaaaaaattgtggatcagtttgctgtttgctttagcgttaatctttacgatggcgttcggcagcacatccagcgcgcaggcggcagggaaatcaaacggggaaaagaaatatattgtcgggtttaaacagacaatgagcacgatgagcgccgctaagaagaaagatgtcatttctgaaaaaggcgggaaagtgcaaaagcaattcaaatatgtagacgcagcttcagctacattaaacgaaaaagctgtaaaagaattgaaaaaagacccgagcgtcgcttacgttgaagaagatcacgtagcacacgcgtac

SEQ ID NO: 9 - protein, 275 aa, Artificial
  synthetic modified protease
AQSVPYGVSQIKAPALHSQGYTGSNVKVAVIDSGIDSSHPDLKVAGGASMVPSETNPFQDNNSHGTHVAGTVAALNNSIGVLGVAPSASLYAVKVLGADGSGQYSWIINGIEWAIANNMDVINMSLGGPSGSAALKAAVDKAVASGVVVVAAAGNEGTSGSSSTVGYPGKYPSVIAVGAVDSSNQRASFSSVGPELDVMAPGVSIQSTLPGNKYGALNGTSMASPHVAGAAALILSKHPNWTNTQVRSSLENTTTKLGDSFYYGKGLINVQAAAQ

SEQ ID NO: 10 - DNA, 828 bp, Artificial
  synthetic polynucleotide encoding SEQ ID NO9
gcgcagtccgtgccttacggcgtatcacaaattaaagcccctgctctgcactctcaaggctacactggatcaaatgttaaagtagcggttatcgacagcggtatcgattcttctcatcctgatttaaaggtagcaggcggagccagcatggttccttctgaaacaaatcctttccaagacaacaactctcacggaactcacgttgccggcacagttgcggctcttaataactcaatcggtgtattaggcgttgcgccaagcgcatcactttacgctgtaaaagttctcggtgctgacggttccggccaatacagctggatcattaacggaatcgagtgggcgatcgcaaacaatatggacgttattaacatgagcctcggcggaccttctggttctgctgctttaaaagcggcagttgataaagccgttgcatccggcgtcgtagtcgttgcggcagccggtaacgaaggcacttccggcagctcaagcacagtgggctaccctggtaaatacccttctgtcattgcagtaggcgctgttgacagcagcaaccaaagagcatctttctcaagcgtaggacctgagcttgatgtcatggcacctggcgtatctatccaaagcacgcttcctggaaacaaatacggcgcgttgaacggtacatcaatggcatctccgcacgttgccggagcggctgctttgattctttctaagcacccgaactggacaaacactcaagtccgcagcagtttagaaaacaccactacaaaacttggtgattctttctactatggaaaagggctgatcaacgtacaggcggcagctcagtaa

SEQ ID NO: 11 - protein, 107 aa, Bacillus amyloliquefaciens
MRGKKVWISLLFALALIFTMAFGSTSSAQAAGKSNGEKKYIVGFKQTMSTMSAAKKKDVISEKGGKVQKQFKYVDAASATLNEKAVKELKKDPSVAYVEEDHVAHAY

SEQ ID NO: 12 - protein, 107 aa, Bacillus amyloliquefaciens
MRGKKVWISLLFALALIFTMAFGSTTSAQAAGKSNGEKKYIVGFKQTMSTMSAAKKKDVISEKGGKVQKQFKYVDAASATLNEKAVKELKKDPSVAYVEEDHVAQAY

SEQ ID NO: 13 - protein, 107 aa, Bacillus amyloliquefaciens
MRGKKVWISLLFALALIFTMAFGSTSPAQAAGKSNGEKKYIVGFKQTMSTMSAAKKKDVISEKGGKVQKQFKYVDAASATLNEKAVKELKKDPSVAYVEEDHVAQAY

SEQ ID NO: 14 - protein, 107 aa, Bacillus sp.; identical to SEQ ID NO: 13

SEQ ID NO: 15 - protein, 107 aa, Bacillus amyloliquefaciens
MRGKKVWISLLFALALIFTMAFGSTSPAQAAGKSNGEKKYIVGFKQTMSTMSAAKKKDVISEKGGKVQKQFKYVDAASATLNEKAVKELKKDPSVAYVEEDHVAKAY

SEQ ID NO: 16 - protein, 107 aa, Geobacillus stearothermophilus; identical to SEQ ID NO: 13

SEQ ID NO: 17 - protein, 107 aa, Bacillus sp.; identical to SEQ ID NO: 12

SEQ ID NO: 18 - protein, 107 aa, Bacillus sp.; identical to SEQ ID NO: 13

SEQ ID NO: 19 - protein, 107 aa, Bacillus subtilis
MRGKKVWISLLFALALIFTMAFGSTSPAHAAGKSNGEKKYIVGFKQTMSTMSAAKKKDVIFEKGGKVQKQFKYVDAASATLNEKAVKELKKDPSVAYVEEDHVAQAY

SEQ ID NO: 20 - protein, 107 aa, Bacillus subtilis; identical to SEQ ID NO: 13

SEQ ID NO: 21 - protein, 106 aa, Bacillus subtilis
MRSKKLWISLLFALTLIFTMAFSNMSAQAAGKSSTEKKYIVGFKQTMSAMSSAKKKDVISEKGGKVQKQFKYVNAAAATLDEKAVKELKKDPSVAYVEEDHIAHEY

SEQ ID NO: 22 - protein, 106 aa, Bacillus subtilis; identical to SEQ ID NO: 21

SEQ ID NO: 23 - protein, 106 aa, Bacillus subtilis; identical to SEQ ID NO: 21

SEQ ID NO: 24 - protein, 106 aa, Bacillus subtilis
MRSKKLWISLLFALTLIFTMAFSNMSAQAAGKSSTEKKYIVGFKQTMSAMSSAKKKDVISEKGGKVQKQFKYVNAAAATLDAKAVKELKQDPSVAYVEEDHIAHQY

SEQ ID NO: 25 - protein, 99 aa, Bacillus subtilis
MSLLFALTLIFTMAFSNMSAQAAGKSSTEKKYIVGFKQTMSAMSSAKKKDVISEKGGKVQKQFKYVNAAAATLDEKAVKELKKDPSVAYVEEDHIAHEY

SEQ ID NO: 26 - protein, 106 aa, Bacillus subtilis; identical to SEQ ID NO: 21

SEQ ID NO: 27 - protein, 106 aa, Bacillus subtilis; identical to SEQ ID NO: 21

SEQ ID NO: 28 - protein, 106 aa, Bacillus subtilis; identical to SEQ ID NO: 21

SEQ ID NO: 29 - protein, 106 aa, Geobacillus stearothermophilus
MRSKKLWISLLFALTLIFTMAFSNMSVQAAGKSSTEKKYIVGFKQTMSAMSSAKKKDVISEKGGKVQKQFKYVNAAAATLDEKAVKELKKDPSVAYVEEDHIAHEY

SEQ ID NO: 30 - protein, 106 aa, Bacillus subtilis; identical to SEQ ID NO: 21

SEQ ID NO: 31 - protein, 106 aa, Bacillus subtilis; identical to SEQ ID NO: 21

SEQ ID NO: 32 - protein, 106 aa, Bacillus subtilis; identical to SEQ ID NO: 21

SEQ ID NO: 33 - protein, 106 aa, Bacillus subtilis; identical to SEQ ID NO: 21

SEQ ID NO: 34 - protein, 106 aa, Bacillus subtilis; identical to SEQ ID NO: 21

SEQ ID NO: 35 - protein, 106 aa, Bacillus subtilis
MRSKKLWISLLFALTLIFTMAFSNMSAQAAGKSSTEKKYIVGFKQTMSAMSSAKKKDVISEKGGKIQKQFKYVNAATATLNEKAVKELKQDPSVAYVEEDHIAHEY

SEQ ID NO: 36 - protein, 106 aa, Bacillus subtilis; identical to SEQ ID NO: 29

SEQ ID NO: 37 - protein, 106 aa, Bacillus sp.; identical to SEQ ID NO: 21

SEQ ID NO: 38 - protein, 106 aa, Bacillus subtilis
MRSKKLWISLLFALTLIFTMAFNNMSAQAAGKSSTEKKYIVGFKQTMSAMSSAKKKDVISEKGGKVQKQFKYVNAAAATLDEKAVKELKKDPSVAYVEEDHIAHEY

SEQ ID NO: 39 - protein, 106 aa, Bacillus subtilis
MRSKKLWISLLFAFTLIFTMAFSNMSAQAAGKNSEEKKYIVGFKQTMGAMSTAKKKDVISEKGGKVQKQFKYVNAAAATLDDKAVKELKKDPSVAYVEEDHVAHEY

SEQ ID NO: 40 - protein, 106 aa, Bacillus subtilis
MRGKKVWISLLFALTLIFTMAFSNMSAQAAGKSSTEKKYIVGFKQTMSAMSSAKKKDVISEKGGKVQKQFKYVNAATATLDEKAVKELKQDPSVAYVEEDHIAHEY

SEQ ID NO: 41 - protein, 106 aa, Bacillus subtilis
MRSKKLWISLLFALTLIFTMAFSNMSAQAAGKSSTEKKYIVGFKQTMSAMSSAKKKDVISEKGGKVQKQFKYVNAAAATLDEKAVKELKQDPSVAYVEEDHIALEY

SEQ ID NO: 42 - protein, 104 aa, Bacillus licheniformis
MKKKSLWLSVLTALLLVLSTVFSSPASAAQPAKDVEKDYIVGFKSSVKTAAVKKDVIKENGGKVDKQFKIINAAKATLDQEEVKALKKDPSVAYVEEDHIAHAM

SEQ ID NO: 43 - protein, 108 aa, Bacillus pumilus
MCVKKKNVMTSVLLAVPLLFSAGFGGTMANAETVSKTDSEKSYIVGFKASATTNSSKKQAVIQNGGKLEKQYRLINAAQVKMSEQAAKKLEHDPSIAYVEEDHKAEAY

SEQ ID NO: 44 - protein, 105 aa, Bacillus licheniformis
MMRKKSFWLGMLTALMLVFTMAFSDSASAAQPAKNVEKDYIVGFKSGVKTASVKKDIIKESGGKVDKQFRIINAAKAKLDKEALKEVKNDPDVAYVEEDHVAHAL

SEQ ID NO: 45 - protein, 108 aa, Bacillus pumilus; identical to SEQ ID NO: 43

SEQ ID NO: 46 - protein, 105 aa, Bacillus licheniformis; identical to SEQ ID NO: 44

SEQ ID NO: 47 - protein, 105 aa, Bacillus mojavensis
MMRKKSFWLGMLTAFMLVFTMAFGDSASAAQPAKNVEKDYIVGFKSGVKTASVKKDIIKESGGKVDKQFRIINAAKAKLDKEALKEVKNDPDVAYVEEDHVAHAL

SEQ ID NO: 48 - protein, 106 aa, Bacillus pumilus
MKKKNVMTSVLLAVPLLFSAGFGGSMANAETVSKSDSEKSYIVGFKASATTNSSKKQAVTQNGGKLEKQYRLINAAQVKMSEQAAKKLEHDPSIAYVEEDHKAEAY

SEQ ID NO: 49 - protein, 108 aa, Bacillus pumilus
MCVKKKNVMTSVLLAVPLLFSAGFGGSMANAETASKSESEKSYIVGFKASATTNSSKKQAVTQNGGKLEKQYRLINAAQVKMSEQAAKKLEHDPSIAYVEEDHKAEAY

SEQ ID NO: 50 - protein, 108 aa, Bacillus pumilus; identical to SEQ ID NO: 49

SEQ ID NO: 51 - protein, 108 aa, Bacillus pumilus
MCVKKKNVMTSVLLAVPLLFSAGFGGSIANAETASKSESEKSYIVGFKASATTNSSKKQAVTQNGGKLEKQYRLINAAQVKMYEQAAKKLEHDPSIAYVEEDHKAEAY

SEQ ID NO: 52 - protein, 105 aa, Bacillus licheniformis; identical to SEQ ID NO: 44

SEQ ID NO: 53 - protein, 105 aa, Bacillus licheniformis
MMRKKSFWLGMLTALMLVFTMAFSDSASAAQPAKNVEKDYIVGFKSGVKTASVKKDIIKESGGKVDKQFRIINAAKAKLDKEALEEVKNDPDVAYVEEDHVAHAL

SEQ ID NO: 54 - protein, 105 aa, Bacillus licheniformis
MMRKKSFWLGMLTAFMLVFTMAFSDSASAAQPAKNVEKDYIVGFKSGVKTASVKKDIIKESGGKVDKQFRIINAAKAKLDKEALKEVKNDPDVAYVEEDHVAHAL

SEQ ID NO: 55 - protein, 108 aa, Bacillus pumilus
MCVKEKNVMTSVLLAVPLLFSAGFGGSMANAETVSKTDSEKSYIVGFKASATTNSSKKQAVIQNGGKLEKQYRLINAAQVKMSEQAAKKLEHDPSIAYVEEDHKAEAY

SEQ ID NO: 56 - protein, 105 aa, Bacillus licheniformis; identical to SEQ ID NO: 54

SEQ ID NO: 57 - protein, 106 aa, Bacillus licheniformis
MKKKNVMTSVLLAVPLLFSAGFGGSMANAETVSKSASEKSYIVGFKASATTNSSKKQAVTQNGGKLEKQYRLINAAQVKMSEQAAKKLEHDPSIAYVEEDHKAEAY

SEQ ID NO: 58 - protein, 105 aa, Bacillus licheniformis; identical to SEQ ID NO: 54

SEQ ID NO: 59 - protein, 105 aa, Bacillus licheniformis
MMRKKSFWLGMLTAFMLVFTMAFSDSASAAQPAKNVEKNYIVGFKSGVKTASVKKDIIKESGGKVDKQFRIINAAKAKLDKEALKEVKNDPDVAYVEEDHVAHAL

SEQ ID NO: 60 - protein, 105 aa, Bacillus licheniformis; identical to SEQ ID NO: 54

SEQ ID NO: 61 - protein, 105 aa, Bacillus licheniformis; identical to SEQ ID NO: 54

SEQ ID NO: 62 - protein, 105 aa, Bacillus licheniformis; identical to SEQ ID NO: 54

SEQ ID NO: 63 - protein, 108 aa, Bacillus pumilus
MCVKKKNVMTSVLLAVPLLFSAGFGGSMANAETVSKTDSEKSYIVGFKASATTNSSKKQAVIQNGGKLEKQYRLINAAQVKMSEQAAKKLEHDPSIAYVEEDHKAEAY

SEQ ID NO: 64 - protein, 105 aa, Bacillus licheniformis
MMRKKSFWFGMLTAFMLVFTMEFSDSASAAQPGKNVEKDYFVGFKSGVKTASVKKDIIKESGGKVDKQFRIINAAKATLDKEALKEVKNDPDVAYVEEDHVAHAL

SEQ ID NO: 65 - protein, 105 aa, Bacillus licheniformis
MMRKKSFWLGMLTALMLVFTMAFSDSASAAQPGKNVEKDYIVGFKSGVKTASVKKDIIKESGGKVDKQFRIINAGKAKLDKEALKEVKNDPDVAYVEEDHVAHVL

SEQ ID NO: 66 - protein, 105 aa, Bacillus licheniformis
MMRKKSFWLGMLTAFMLVFTMAFSDSASAAQPAKNVEKDYIVGFKSGVKTASVKKDIIKESGGKVDKQFRIINAAKAKLDKEALKEVKNDPDVAYVEEDHVGHGL

SEQ ID NO: 67 - protein, 275 aa, Bacillus amyloliquefaciens [sequence omitted]

SEQ ID NO: 68 - protein, 275 aa, Bacillus amyloliquefaciens
AQSVPYGVSQIKAPALHSQGFTGSNVKVAVIDSGIDSSHPDLKVAGGASMVPSETNPFQDNNSHGTHVAGTVAALNNSVGVLGVAPSASLYAVKVLGADGSGQYSWIINGIEWAIANNMDVINMSLGGPSGSAALKAAVDKAVASGVVVVAAAGNEGTSGGSSTVGYPGKYPSVIAVGAVNSSNQRASFSSVGSELDVMAPGVSIQSTLPGNKYGAYNGTSMASPHVAGAAALILSKHPNWTNTQVRSSLENTTTKLGDAFYYGKGLINVQAAAQ

SEQ ID NO: 69 - protein, 275 aa, Bacillus amyloliquefaciens
AQSVPYGVSQIKAPALHSQGFTGSNVKVAVIDSGIDSSHPDLKVAGGASMVPSETNPFQDNNSHGTHVAGTVAALNNSVGVLGVAPSASLYAVKVLGADGSGQYSWIINGIEWAIANNMDVINMSLGGPSGSAALKAAVDKAVASGIVVVAAAGNEGTSGSSSTVGYPGKYPSVIAVGAVNSSNQRASFSSVGSELDVMAPGVSIQSTLPGNKYGAYNGTSMASPHVAGAAALILSKHPNWTNTQVRSSLENTTTKLGDAFYYGKGLINVQAAAQ

SEQ ID NO: 70 - protein, 275 aa, Bacillus sp.; identical to SEQ ID NO: 68

SEQ ID NO: 71 - protein, 275 aa, Bacillus amyloliquefaciens
AQSVPYGVSQIKAPALHSQGFTGSNVKVAVIDSGIDSSHPDLKVAGGASMVPSETNPFQDYNSHGTHVAGTVAALNNSVGVLGVAPSASLYAVKVLGADGSGQYSWIINGIEWAIANNMDVINMSLGGPSGSAALKAAVDKAVASGVVVVAAAGNEGTSGGSSTVGYPGKYPSVIAVGAVNSSNQRASFSSVGSELDVMAPGVSIQSTLPGNKYGAYNGTSMASPHVAGAAALILSKHPNWTNTQVRSSLENTTTKLGDAFYYGKGLINVQAAAQ

SEQ ID NO: 72 - protein, 275 aa, Geobacillus sterothermophilus
AQSVPYGVSQIKAPALHSQGFTGSNVKVAVIDSGIDSSHPDLKVAGGASMVPSETNPFQDNNSHGTHVAGTVAALNNSVGVLGVAPSASLYAVKVLGADGSGQYSWIINGIEWAIAYNMDVINMSLGGPSGSAALKAAVDKAVASGIVVVAAAGNEGTSGSSSTVGYPGKYPSVIAVGAVNSSNQRASFSSVGSELDVMAPGVSIQSTLPGNKYGAYNGTSMASPHVAGAAALILSKHPNWTNTQVRSSLENTTTKLGDAFYYGKGLINVQAAAQ

SEQ ID NO: 73 - protein, 275 aa, Bacillus sp.
AQSVPYGVSQIKAPALHSQGFTGSNVKVAVIDSGIDSSHPDLKVAGGASMVPSETNPFQDNNSHGTHVAGTVAALNNSVGVLGVAPSASLYAVKVLGADGSGQYSWIINGIEWAIANNMDVINMSLGGPSGSAALKAAVDKAVASGVVVVAAAGNEGTSGGSSTVGYPGKYPSVIAVGAVNSSNQRASFSSVGSELDVMAPGVSIQSTLPGNKYGAYNGTSMASPHVAGAAALILSKHPNWTNTQVRSSLENTATKLGDAFYYGKGLINVQAAAQ

SEQ ID NO: 74 - protein, 275 aa, Bacillus sp.
AQSVPYGVSQIKAPALHSQGFTGSNVKVAVIDSGIDSSHPDLKVAGGASMVPSETNPFQDNNSHGTHVAGTVAALNNSVRVLGGAPSASLYAVKVLGADGSGQYSWIINGIEWAIANNMDVINMSLGGPSGSAALKAAVDKAVASGIVVVAAAGNEGTSGSSSTVGYPGKYPSVIAVGAVNSSNQRASFSSVGSELDVMAPGVSIQSTLPGNKYGAYNGTSMASPHVAGAAALILSKHPNWTNTQVRSSLENTTTKLGDAFYYGKGLINVQAAAQ

SEQ ID NO: 75 - protein, 275 aa, Bacillus subtilis
AQSVPYGVSQIKAPALHSQGFTGSNVKVAVIDSGIDSSHPDLKVAGGASMVPSETNPFQDNNSHGTHVAGTVAALNNSVGVLGVAPSASLYAVKVLGADGSGQYSWIINGIEWAIANNMDVINMSLGGPSGSAALKAAVDKAVASGIVVVAAAGNEGTSGGSSTVGYPGKYPSVIAVGAVNSSNQRASFSSVGSELDVMAPGVSIQSTLPGNKYGAYNGTSMASPHVAGAAALILSKHPNWTNTQVRSSLENTTTKLGDAFYYGKGLINVQAAAQ

SEQ ID NO: 76 - protein, 275 aa, Bacillus subtilis
AQSVPYGVSQIKAPALHSQGFTGSNVKVAVIDSGIDSSHPDLKVAGGASMVPSETNPFQDNNSHGTHVAGTVAALNNSVFVLGVAPSASLYAVKVLGADGSGQYSWIINGIEWAIANNMDVINMSLGGPSGSAALKAAVDKAVASGVVVVAAAGNEGTSGGSSTVGYPGKYPSVIAVGAVNSSNQRASFSSVGSELDVMAPGVSIQSTLPGNKYGAYNGTSMASPHVAGAAALILFKHPNWTNTQVRSSLENTTTKLGDAFYYGKGLINVQAAAH

SEQ ID NO: 77 - protein, 275 aa, Bacillus subtilis
AQSVPYGISQIKAPALHSQGYTGSNVKVAVIDSGIDSSHPDLNVRGGASFVPSETNPYQDGSSHGTHVAGTIAALNNSIGVLGVAPSASLYAVKVLDSTGSGQYSWIINGIEWAISNNMDVINMSLGGPSGSTALKTVVDKAVSSGIVVAAAAGNEGSSGSSSTVGYPAKYPSTIAVGAVNSSNQRASFSSAGSELDVMAPGVSIQSTLPGGTYGAYNGTSMATPHVAGAAALILSKHPTWTNAQVRDRLESTATYLGNSFYYGKGLINVQAAAQ

SEQ ID NO: 78 - protein, 275 aa, Bacillus subtilis
AQSVPYGISQIKAPALHSQGYTGSNVKVAVIDSGIDSSHPDLNVRGGASFVPSETNPYQDGSSHGTHVAGTIAALNNSIGVLGVAPSASLYAVKVLDSTGSGQYSWIINGIEWAISNNMDVINMSLGGPTGSTALKTVVDKAVSSGIVVAAAAGNEGSSGSTSTVGYPAKYPSTIAVGAVNSSNQRASFSSVGSELDVMAPGVSIQSTLPGGTYGAYNGTSMATPHVAGAAALILSKHPTWTNAQVRDRLESTATYLGNSFYYGKGLINVQAAAQ

SEQ ID NO: 79 - protein, 275 aa, Bacillus subtilis
AQSVPYGISQIKAPALHSQGYTGSNVKVAVIDSGIDSSHPDLNVRGGASFVPSETNPYQDGSSHGTHVAGTIAALNNSIGVLGVSPSASLYAVKVLDSTGSGQYSWIINGIEWAISNNMDVINMSLGGPSGSTALKTVVDKAVSSGIVVAAAAGNEGSSGSSSTVGYPAKYPSTIAVGAVNSSNQRASFSSAGSELDVMAPGVSIQSTLPGGTYGAYNGTSMATPHVAGAAALILSKHPTWTNAQVRDRLESTATYLGNSFYYGKGLINVQAAAQ

SEQ ID NO: 80 - protein, 275 aa, ABacillus subtilis
AQSVPYGISQIKAPALHSQGYTGSNVKVAVIDSGIDSSHPDLNVRGGASFVPSETNPYQDGSSHGTHVAGTVAALNNSIGVLGVAPNASLYAVKVLDSTGNGQYSWIINGIEWAISNKMDVINMSLGGPSGSTALKSVVDRAVASGIVVVAAAGNEGTSGSSSTIGYPAKYPSTIAVGAVNSSNQRGSFSSVGPELDVMAPGVSIQSTLPGGTYGAYNGTSMATPHVAGAAALILSKHPTWTNAQVRDRLESTTTYLGNSFYYGKGLINVQAAAQ

SEQ ID NO: 81 - protein, 275 aa, Bacillus subtilis; identical to SEQ ID NO: 77

SEQ ID NO: 82 - protein, 275 aa, Bacillus subtilis
AQSVPYGISQIKAPALHSQGYTGSNVKVAVIDSGIDSSHPDLNVRGGASFVPSETNPYQDGSSHGTHVAGTIAALNNSIGVLGVAPSASLYAVKVLDSTGSGQYSWIINGIEWAISNNMDVINMSLGGPSGSTALKTVVDKAASSGIVVAAAAGNEGSSGSSSTVGYPAKYPSTIAVGAVNSSNQRASFSSAGSELDVMAPGVSIQSTLPGGTYGAYNGTSMATPHVAGAAALILSKHPTWTNAQVRDRLESTATYLGNSFYYGKGLINVQAAAQ

SEQ ID NO: 83 - protein, 275 aa, Bacillus subtilis
AQSVPYGISQIKAPALHSQGYTGSNVKVAVIDSGIDSSHPDLNVRGGASFVPSETNPYQDGSSHGTHVAGTIAALNNSIGVLGVAPSASLYAVKVLDSTGSGQYSWIINGIEWAISNNMDVINMSLGGPTGSTALKTVVDKAVSSGIVVAAAAGNEGSSGSSSTVGYPAKYPSTIAVGAVNSSNQRASFSSAGSELDVMAPGVSIQSTLPGGTYGAYNGTSMATPHVAGAAALILSKHPTWTNAQVRDRLESTATYLGNSFYYGKGLINVQAAAQ

SEQ ID NO: 84 - protein, 275 aa, Bacillus subtilis
AQSVPYGISQIKAPALHSQGYTGSNVKVAVIDSGIDSSHPDLNVRGGASFVPSETNPYQDGSSHGTHVAGTIAALNNSIGVLGVAPSASLYAVKVLDSTGSGQYSWIINGIEWAISNNMDVINMSLGGPTGSTALKTVVDKAVSSGIVVAAAAGNEGSSGSTSTVGYPAKYPSTIAVGAVNSSNQRASFSSVGSELDVMAPGVSIQSTLPGGTYGAYNGTSMATPHVAGAAALILSKHPTWTNAQVRDRLESTATYLGSSFYYGKGLINVQAAAQ

SEQ ID NO: 85 - protein, 275 aa, Geobacillus stearothermophilus; identical to SEQ ID NO: 79

SEQ ID NO: 86 - protein, 275 aa, Bacillus subtilis
AQSVPYGISQIKAPALHSQGYTGSNVKVAVIDSGIDSSHPDLNVRGGASFVPSETNPYQDGSSHGTHVAGTIAALNNSIGVLGVAPSASLYAVKVLDSTGSGQYSWIINGIEWAISNNMDVINMSLGGPTGSTALKTVVDKAVSSGIVVAAAAGNEGSSGSTSTVGYPAKYPSTIAVGAVNSSNQRASFSSAGSELDVMAPGVSIQSTLPGGTYGAYNGTSMATPHVAGAAALILSKHPTWTNAQVRDRLESTATYLGNSFYYGKGLINVQAAAQ

SEQ ID NO: 87 - protein, 275 aa, Bacillus subtilis
AQSVPYGISQIKAPALHSQGYTGSNVKVAVIDSGIDSSHPDLNVRGGASFVPSETNPYQDGSSHGTHVAGTIAALNNSIGVLGVAPSASLYAVKVLDSTGSGQYSWIINGIEWAISNNMDVINMSLGGPTGSTALKTVVDKAVSSGIVVAAAAGNEGSSGSTSTVGYPAKYPSTIAVGAVNSSNQRASFSSAGSELDVMAPGVSIQSTLPGGTYGAYNGTSMATPHVAGAAALILSKHPTWTNAQVRDRLESTATYLGSSFYYGKGLINVQAAAQ

SEQ ID NO: 88 - protein, 275 aa, Bacillus subtilis
AQSVPYGISQIKAPALHSQGYTGSNVKVAVIDSGIDSSHPDLNVRGGASFVPSETNPYQDGSSHGTHVAGTIAALNNSIGVLGVAPSASLYAVKVLDSTGSGQYSWIINGIEWAISNNMDVINMSLGGPTGSTALKTVVDKAVSSGIVVAAAAGNEGSSGSSSTVGYPAKYPSTIAVGAVNSSNQRASFSSAGSEFDVMAPGVSIQSTLPGGTYGAYNGTSMATPHVAGAAALILSKHPTWTNAQVRDRLESTATYLGNSFYYGKGLINVQAAAQ

SEQ ID NO: 89 - protein, 275 aa, Bacillus subtilis
AQSVPYGISQIKAPALHSQGYTGSNVKVAVIDSGIDSSHPDLNVRGGASFVPSETNPYQDGSSHGTHVAGYIAALNNSIGVLGVSPSASLYAVKVLDSTGSGQYSWIINGIEWAISNNMDVINMSLGGPSGSTALKTVVDKAVSSGIVVAAAAGNEGSSGSSSTVGYPAKYPSTIAVGAVNSSNQRASFSSAGSELDVMAPGVSIQSTLPGGTYGAYNGTSMATPHVAGAAALILSKHPTWTNAQVRDRLESTATYLGNSFYYGKGLINVQAAAQ

SEQ ID NO: 90 - protein, 275 aa, Bacillus subtilis
AQSVPYGISQIKAPALHSQGYTGSNVKVAVIDSGIDSSHPDLNVRGGASFVPSETNPYQDGSSHGTHVAGTIAALNNSIGVLGVSPSASLYAVKVLDSTGSGQYSWIINGIEWAISNNMDVINMSLGGPTGSTALKTVVDKAVSSGIVVAAAAGNEGSSGSTSTVGYPAKYPSTIAVGAVNSSNQRASFSSAGSELDVMAPGVSIQSTLPGGTYGAYNGTSMATPHVAGAAALILSKHPTWTNAQVRDRLESTATYLGNSFYYGKGLINVQAAAQ

SEQ ID NO: 91 - protein, 275 aa, Bacillus subtilis
AQSVPYGISQIKAPALHSQGYTGSNVKVAVIDSGIDSSHPDLNVRGGASFVPSETNPYQDGSSHGTHVAGTIAALNNSIGVLGVAPNASLYAVKVLDSTGSGQYSWIINGIEWAISNNMDVINMSLGGPSGSTALKTVVDKAVSNGIVVAAAAGNEGSSGSTSTVGYPAKYPSTIAVGAVNSSNQRASFSSAGPELDVMAPGVSIQSTLPGGTYGAYNGTSMATPHVAGAAALILSKHPTWTNAQVRDRLESTATYLGNSFYYGKGLINVQAAAQ

SEQ ID NO: 92 - protein, 275 aa, Bacillus subtilis; identical to SEQ ID NO: 90

SEQ ID NO: 93 - protein, 275 aa, Bacillus sp.
AQSVPYGISQIKAPALHSQGYTGSNVKVAVIDSGIDSSHPDLNVRGGASFVPSETNPYQDGSSHGTHVAGTIAALNNSIGVLGVAPSASLYAVKVLDSTGSGQYSWIINGIEWAISNNMDIINMSLGGPTGSTALKTVVDKAVSSGIVVAAAAGNEGSSGSTSTVGYPAKYPSTIAVGAVNSSNQRASFSSAGSELDVMAPGVSIQSTLPGGTYGAYNGTSMATPHVAGATALILSKHPTWTNAQVRDRLESTATYLGSSFYYGKGLINVQAAAQ

SEQ ID NO: 94 - protein, 275 aa, Bacillus subtilis
AQSVPYGISQIKAPALHSQGYTGSNVKVAVIDSGIDSSHPHLNVRGGASFVPSETNPYQDGSSHGTHVAGTIAALNNSICVLGVAPSASLYAVKVLDSTGSGQYSWIINGIEWAISNNMDVINMSLGGPTGSTALKTVVDKAVSSGIVVAAAAGNEGSSGSSSTVGYPAKYPSTIAVGAVNSSNQRASFSSAGSELDVMAPGVSIQSTLPGGTYGAYNGTSMATPHVAGAAALILSKHPTWTNAQVRDRLESTATYLGNSFYYGKGLINVQAAAQ

SEQ ID NO: 95 - protein, 275 aa, Bacillus subtilis
AQSVPYGISQIKAPALHSQGYTGSNVKVAVIDSGIDSSHPDLNVRGGASFVPSETNPYQDGSSHGTHVAGTVAALNNTIGVLGVAPSASLYAVKVLDSTGSGQYSWIINGIEWAISNNMDVINMSLGGPTGSTALKTVVDKAVASGIVVVAAAGNEGSSGSTSTVGYPAKYPSTIAVGAVNSSNQRASFSSAGSELDVMAPGVSIQSTLPGGTYGSYNGTSMATPHVAGAAALILSKHPTWSNAQVRDRLESTATNLGSSFYYGKGLINVQAAAQ

SEQ ID NO: 96 - protein, 275 aa, Bacillus subtilis
AQSVPYGISQIKAPALHSQGYTGSNVKVAVIDSGIDSSHPDLNVKGGASFVPSETNPYQDGSSHGTHVAGTIAALNNTIGVLGVAPNASLYAVKVLDSTGSGQYSWIINGIEWAISNNMDVINMSLGGPSGSTALKTVVDKAVSSGIVVAAAAGNEGSSGSTSTVGYPAKYPSTIAVGAVNSSNQRASFSSAGSELDVMAPGVSIQSTLPGGTYGAYNGTSMATPHVAGAAALILSKHPTWTNAQVRDRLESTATYLGSSFYYGKGLINVQAAAQ

SEQ ID NO: 97 - protein, 275 aa, Bacillus subtilis
AQSVPYGISQIKAPALHSQGYTGSNVKVAVIDSGIDSSHPDLNVRGGASFVPSETNPYQGRSSHGTHVAGTISAFNNSIGVLGVAPNASLYAVKVLDSTGSGQYSWIINGIEWAISNNMDVINMSLGGPSGSTALKTVVDKAVSSGIVVAAAAGNEGSSGSTSTVGYPAKYPSTIAVGAVNSSTQRASFSSAGSELDVMAPGVSIQSTLPGGTYGAYNGTSMATPHVAGAAALILSKHPTWTNAQVRDRLESTATYLGNSFYYGKGLINVQAAAQ

SEQ ID NO: 98 - protein, 274 aa, Bacillus licheniformis
AQTVPYGIPLIKADKVQAQGYKGANVKVGIIDTGIASSHTDLKVVGGASFVSGESYNTDGNGHGTHVAGTVAALDNTTGVLGVAPNVSLYAIKVLNSSGSGTYSAIVSGIEWATQNGLDVINMSLGGPSGSTALKQAVDKAYASGIVVVAAAGNSGSSGSQNTIGYPAKYDSVIAVGAVDSNKNRASFSSVGSELEVMAPGVSVYSTYPSNTYTSLNGTSMASPHVAGAAALILSKYPTLSASQVRNRLSSTATNLGDSFYYGKGLINVEAAAQ

SEQ ID NO: 99 - protein, 275 aa, Bacillus pumilus
AQTVPYGIPQIKAPAVHAQGYKGANVKVAVLDTGIHAAHPDLNVAGGASFVPSEPNATQDFQSHGTHVAGTIAALDNTIGVLGVAPNASLYAVKVLDRNGDGQYSWIISGIEWAVANNMDVINMSLGGPSGSTALKNAVDTANNRGVVVVAAAGNSGSSGSRSTVGYPAKYDSTIAVANVNSSNVRNSSSSAGPELDVSAPGTSILSTVPSSGYTSYTGTSMASPHVAGAAALILSKNPNLTNSQVRQRLENTATPLGDSFYYGKGLINVQAASN

SEQ ID NO: 100 - protein, 274 aa, Bacillus licheniformis
AQTVPYGVPLIKADKVQAQGFKGANVKVAVLDTGIQASHPDLNVVGGASFVAGEAYNTDGNGHGTHVAGTVAALDNTTGVLGVAPSVSLYAVKVLNSSGSGSYSGIVSGIEWATTNGMDVINMSLGGASGSTAMKQAVDNAYARGVVVVAAAGNSGSSGNTNTIGYPAKYDSVIAVGAVDSNSNRASFSSVGAELEVMAPGAGVYSTYPTNTYATLNGTSMASPHVAGAAALILSKHPNLSASQVRNRLSSTATYLGSSFYYGKGLINVEAAAQ

SEQ ID NO: 101 - protein, 275 aa, Bacillus pumilus
AQTVPYGIPQIKAPAVHAQGYKGANVKVAVLDTGIHAAHPDLNVAGGASFVPSEPNATQDFQSHGTHVAGTIAALDNTIGVLGVAPNASLYAVKVLDRNGDGQYSWIISGIEWAVANNMDVINMSLGGPSGSTALKNAVDTANNRGVVVVAAAGNSGSSGSRSTVGYPAKYDSTIAVANVNSNNVRNSSSSAGPELDVSAPGTSILSTVPSSGYTSYTGTSMASPHVAGAAALILSKNPNLTNSQVRQRLENTATPLGDSFYYGKGLINVQAASN

SEQ ID NO: 102 - protein, 274 aa, Bacillus licheniformis
AQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGSYSGIVSGIEWATTNGMDVINMSLGGASGSTAMKQAVDNAYARGVVVVAAAGNSGSSGNTNTIGYPAKYDSVIAVGAVDSNSNRASFSSVGAELEVMAPGAGVYSTYPTNTYATLNGTSMASPHVAGAAALILSKHPNLSASQVRNRLSSTATYLGSSFYYGKGLINVEAAAQ

SEQ ID NO: 103 - protein, 274 aa, Bacillus mojavensis; identical to SEQ ID NO: 102

SEQ ID NO: 104 - protein, 275 aa, Bacillus pumilus
AQTVPYGIPQIKAPAVHAQGYKGANVKVAVLDTGIHAAHPDLNVAGGASFVPSEPNATQDFQSHGTHVAGTIAALDNTIGVLGVAPSASLYAVKVLDRYGDGQYSWIISGIEWAVANNMDVINMSLGGPNGSTALKNAVDTANNRGVVVVAAAGNSGSTGSTSTVGYPAKYDSTIAVANVNSSNVRNSSSSAGPELDVSAPGTSILSTVPSSGYTSYTGTSMASPHVAGAAALILSKYPNLSTTQVRQRLENTATPLGNSFYYGKGLINVQAASN

SEQ ID NO: 105 - protein, 275 aa, Bacillus pumilus
AQTVPYGIPQIKAPAVHAQGYKGANVKVAVLDTGIHAAHPDLNVAGGASFVPSEPNATQDFQSHGTHVAGTIAALDNTIGVLGVAPSASLYAVKVLDRYGDGQYSWIISGIEWAVANNMDVINMSLGGPNGSTALKNAVDTANNRGVVVVAAAGNSGSTGSTSTVGYPAKYDSTIAVANVNSNNVRNSSSSAGPELDVSAPGTSILSTVPSSGYTSYTGTSMASPHVAGAAALILSKYPNLSTSQVRQRLENTATPLGNSFYYGKGLINVQAASN

SEQ ID NO: 106 - protein, 275 aa, Bacillus pumilus
AQTVPYGIPQIKAPAVHAQGYKGANVKVAVLDTGIHAAHPDLNVAGGASFVPSEPNATQDFQSHGTHVAGTIAALDNTIGVLGVAPSASLYAVKVLDRYGDGQYSWIISGIEWAVANNMDVINMSLGGPNGSTALKKAVDTANNRGVVVVAAAGNSGSTGSTSTVGYPAKYDSTIAVANVNSNNVRNSSSSAGPELDVSAPGTSILSTVPSSGYTSYTGTSMASPHVAGAAALILSKYPNLSTSQVRQRLENTATPLGNSFYYGKGLINVQAASN

SEQ ID NO: 107 - protein, 275 aa, Bacillus pumilus; identical to SEQ ID NO: 105

SEQ ID NO: 108 - protein, 269 aa, Bacillus licheniformis
AQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGSYSGIVSGIEWATTNGMDVINMSLGGASGSTAMKQAVDNAYARGVVVVAAAGNSGSSGNTNTIGYPAKYDSVIAVGAVDSNSNRASFSSVGAELEVMAPGAGVYSTYPTNTYATLNGTSMASPHVAGAAALILSKHPNLSASQVRNRLSSTATYLGSSFYYGKGLINV

SEQ ID NO: 109 - protein, 274 aa, Bacillus licheniformis
AQTVPYGIPLIKADKVQAQGYKGANVKVAVLDTGIQASHPDLNVVGGASFVAGEAYNTDGNGHGTHVAGTVAALDNTTGVLGVAPNVSLYAVKVLNSSGSGSYSGIVSGIEWATTNGMDVINMSLGGPSGSTAMKQAVDNAYARGVVVVAAAGNSGSSGNTNTIGYPAKYDSVIAVGAVDSNSNRASFSSVGAELEVMAPGAGVYSTYPTSTYATLNGTSMASPHVAGAAALILSKHPNLSASQVRNRLSSTATYLGSSFYYGKGLINVEAAAQ

SEQ ID NO: 110 - protein, 274 aa, Bacillus licheniformis
AQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGTYSGIVSGIEWATTNGMDVINMSLGGPSGSTAMKQAVDNAYARGVVVVAAAGNSGSSGNTNTIGYPAKYDSVIAVGAVDSNSNRASFSSVGAELEVMAPGAGVYSTYPTSTYATLNGTSMASPHVAGAAALILSKHPNLSASQVRNRLSSTATYLGSSFYYGKGLINVEAAAQ

SEQ ID NO: 111 - protein, 275 aa, Bacillus pumilus
AQTVPYGIPQIKAPAVHAQGYKGANVKVAVLDTGIHAAHPDLNAAGGASFVPSEPNATQDFQSHGTHVAGTIAALDNTIGVLGVAPSASLYAVKALDRNGDGQYSWIISGIEWAVANNMDVINMSLGGASGSTALKNAVDTANSRGVVAVAAAGNSGSSGSRSTVGYPAKYESTIAVANVNSNNVRNSSSSAGPELDVSAPGTSILSTVPSSGYTSYTGTSMASPHVAGAAALILSKNPNLTNSQVRQRLENTATPLGDSFYYGKGLINVQAASN

SEQ ID NO: 112 - protein, 274 aa, Bacillus licheniformis
AQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGSYSGIVSGIEWATTNGMDVINMSLGGASGSTAMKQAVDNAYAKGVVVVAAAGNSGSSGNTNTIGYPAKYDSVIAVGAVDSNSNRASFSSVGAELEVMAPGAGVYSTYPTNTYATLNGTSMASPHVAGAAALILSKHPNLSASQVRNRLSSTATYLGSSFYYGKGLINVEAAAQ

SEQ ID NO: 113 - protein, 275 aa, Bacillus intermedius
AQTVPYGIPQIKAPAVHAQGYKGANVKVAVLDTGIHAAHPDLNVAGGASFVPSEPNATQDFQSHGTHVAGTIAALDNTIGVLGVAPSASLYAVKVLDRNGDGQYSWIISGIEWAVANNMDVINMSLGGPNGSTALKNAVDTANNRGVVVVAAAGNSGSTGSTSTVGYPAKYDSTIAVANVNSSNVRNSSSSAGPELDVSAPGTSILSTVPSSGYTSYTGTSMASPHVAGAAALILSKNPNLSNSQVRQRLENTATPLGNSFYYGKGLINAQAASN

SEQ ID NO: 114 - protein, 274 aa, Bacillus licheniformis; identical to SEQ ID NO: 102

SEQ ID NO: 115 - protein, 269 aa, Bacillus licheniformis; identical to SEQ ID NO: 108

SEQ ID NO: 116 - protein, 269 aa, Bacillus licheniformis
AQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGSYSGIVSGIEWATTNGMDVINMSLGGASGSTAMKQAVDNAYAKGVVVVAAAGNSGSSGNTNTIGYPAKYDSVIAVGAVDSNSNRASFSSVGAELEVMAPGAGVYSTYPTNTYATLNGTSMASPHVAGAAALILSKHPNLSASQVRNRLSSTATYLGSSFYYGKGLINV

SEQ ID NO: 117 - protein, 274 aa, Bacillus licheniformis
AQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGSYSGIVSGIEWATTNGMDVINMSLGGASGSTAMKQAVDNAYARGVVVVAAAGNSGSSGNTNTIGYPAKYDSVIAVGAVDSNSNRASFSSVGAELEVMAPGAGVYSTYPTNTYATLNGTSMASPHVAGAAALILSKHPNLSASQVRNRLSSTATYLGSSFYYGKGLINVEGAAQ

SEQ ID NO: 118 - protein, 274 aa, Bacillus licheniformis
AQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGSYSGIVSGIEWATTNGMDVINMSLGGASGSTAMKQAVDNAYARGVVVVAAAGNSGSSGNTNTIGYPAKYDSVIAVGAVDSNSNRASFSSVGAELEVMAPGAGVYSTYPTNTYATLNGTSMVSPHVAGAAALILSKHPNLSASQVRNRLSSTATYLGSSFYYGKGLINVEAAAQ

SEQ ID NO: 119 - protein, 275 aa, Bacillus pumilus
AQTVPYGIPQIKAPAVHAQGYKGANVKVAVLDTGIHAAHPDLNVAGGASFVPSEPNATQDFQSHGTHVAGTIAALDNTIGVLGVAPSASLYAVKVLDRNGDGQYSWIISGIEWAVANNMDVINMSLGGPNGSTALKNAVDTANNRGVVVVAAAGNSGSFGSTSTVGYPAKYDSTIAVANVNGNNVRNSSSSAGPELDVSAPGTSILSTVPSSGYTSYTGTSMASPHVAGAAALILSKYPNLSTSQVRQRLENTATPLGDSFYYGKGLINVQAASN

SEQ ID NO: 120 - protein, 274 aa, Bacillus licheniformis
GQTVPYGIPLIKADKVQAQGFKGANVKVAVLDTGIQASHPDLNVVGGASFVAGEAYNTDGNGHGTHVAGTVAALDNTTGVLGVAPSVSLFAVKVLNSSGSGSYSGIVSGIEWATTNGMDVINMSLGGPSGSTAMKQAVDNAYSKGVVPVAAAGNSGSSGYTNTIGYPAKYDSVIAVGAVDSNSNRASFSSVGAELEVMAPGAGVYSTYPTNTYATLNGTSMASPHVAGAAALILSKHPNLSASQVRNRLSSTATYLGSSFYYGKGLINVEAAAQ

SEQ ID NO: 121 - protein, 274 aa, Bacillus licheniformis
GQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGSYSAIVSGIEWATTTGMDVINMSLGGASVSTAMKQAVDHAYARGAVVVSSAGNSGSSGNTNTIGYPAKYDSVIAVGAVDSNSNRASFSSVGAELEVMAPGAGVYSTYPTNTYATLNGTSMASPHVAGAAALILSKHPNLSASQVRTRLSRTATYLGSSFSYGRGLINVEAAAQ

SEQ ID NO: 122 - protein, 274 aa, Bacillus licheniformis
GQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGSYSGIVSGIEWVTTNGMDVINMSLGGASGSTAMKQAVDNAYARGVVVVAAAGNSGSSGNTNTIGYPAKCDSVIPVGGEDSNSNRSSFSSVGAELEVMAPVSGVYSTYPTNTYTTLNGTSMASPHVAGTSALILSKHPNLSASQVRNRLSRTATYLGSSFYYGKGLINVEAAAQ

SEQ ID NO: 123 - DNA, 22 bp, Artificial
  synthetic primer
ttattgtctcatgagcggatac

SEQ ID NO: 124 - DNA, 36 bp, Artificial
  synthetic primer
aactcttcavnntctttaccctctccttttaaaaaa

SEQ ID NO: 125 - DNA, 36 bp, Artificial
  synthetic primer
aactcttcavnncactctttaccctctccttttaaa

SEQ ID NO: 126 - DNA, 36 bp, Artificial
  synthetic primer
aactcttcavnntctcactctttaccctctcctttt

SEQ ID NO: 127 - DNA, 36 bp, Artificial
  synthetic primer
aactcttcavnngcttctcactctttaccctctcct

SEQ ID NO: 128 - DNA, 36 bp, Artificial
  synthetic primer
aactcttcavnntttgcttctcactctttaccctct

SEQ ID NO: 129 - DNA, 37 bp, Artificial
  synthetic primer
aactcttcavnnttttttgcttctcactctttaccct

SEQ ID NO: 130 - DNA, 36 bp, Artificial
  synthetic primer
aactcttcavnncaattttttgcttctcactcttta

SEQ ID NO: 131 - DNA, 36 bp, Artificial
  synthetic primer
aactcttcavnnccacaattttttgcttctcactct

SEQ ID NO: 132 - DNA, 36 bp, Artificial
  synthetic primer
aactcttcavnngatccacaattttttgcttctcac

SEQ ID NO: 133 - DNA, 36 bp, Artificial
  synthetic primer
aactcttcavnnactgatccacaattttttgcttct

SEQ ID NO: 134 - DNA, 36 bp, Artificial
  synthetic primer
aactcttcavnncaaactgatccacaattttttgct

SEQ ID NO: 135 - DNA, 36 bp, Artificial
  synthetic primer
aactcttcavnncagcaaactgatccacaatttttt

SEQ ID NO: 136 - DNA, 36 bp, Artificial
  synthetic primer
aactcttcavnnaaacagcaaactgatccacaattt

SEQ ID NO: 137 - DNA, 36 bp, Artificial
  synthetic primer
aactcttcavnnagcaaacagcaaactgatccacaa

SEQ ID NO: 138 - DNA, 36 bp, Artificial
  synthetic primer
aactcttcavnntaaagcaaacagcaaactgatcca

SEQ ID NO: 139 - DNA, 36 bp, Artificial
  synthetic primer
aactcttcavnncgctaaagcaaacagcaaactgat

SEQ ID NO: 140 - DNA, 36 bp, Artificial
  synthetic primer
aactcttcavnntaacgctaaagcaaacagcaaact

SEQ ID NO: 141 - DNA, 36 bp, Artificial
  synthetic primer
aactcttcavnngattaacgctaaagcaaacagcaa

SEQ ID NO: 142 - DNA, 36 bp, Artificial
  synthetic primer
aactcttcavnnaaagattaacgctaaagcaaacag

SEQ ID NO: 143 - DNA, 36 bp, Artificial
  synthetic primer
aactcttcavnncgtaaagattaacgctaaagcaaa

SEQ ID NO: 144 - DNA, 35 bp, Artificial
  synthetic primer
aactcttcavnncatcgtaaagattaacgctaaag

SEQ ID NO: 145 - DNA, 36 bp, Artificial
  synthetic primer
aactcttcavnncgccatcgtaaagattaacgctaa

SEQ ID NO: 146 - DNA, 34 bp, Artificial
  synthetic primer
aactcttcavnngaacgccatcgtaaagattaac

SEQ ID NO: 147 - DNA, 36 bp, Artificial
  synthetic primer
aactcttcavnngccgaacgccatcgtaaagattaa

SEQ ID NO: 148 - DNA, 36 bp, Artificial
  synthetic primer
aactcttcavnngctgccgaacgccatcgtaaagat

SEQ ID NO: 149 - DNA, 36 bp, Artificial
  synthetic primer
aactcttcavnntgtgctgccgaacgccatcgtaaa

SEQ ID NO: 150 - DNA, 36 bp, Artificial
  synthetic primer
aactcttcavnnggatgtgctgccgaacgccatcgt

SEQ ID NO: 151 - DNA, 36 bp, Artificial
  synthetic primer
aactcttcavnngctggatgtgctgccgaacgccat

SEQ ID NO: 152 - DNA, 34 bp, Artificial
  synthetic primer
aactcttcavnncgcgctggatgtgctgccgaac

SEQ ID NO: 153 - DNA, 36 bp, Artificial
  synthetic primer
aactcttcavnnctgcgcgctggatgtgctgccgaa

SEQ ID NO: 154 - DNA, 34 bp, Artificial
  synthetic primer
aactcttcavnncgcctgcgcgctggatgtgctg

SEQ ID NO: 155 - DNA, 36 bp, Artificial
  synthetic primer
aactcttcavnntgccgcctgcgcgctggatgtgct

SEQ ID NO: 156 - DNA, 36 bp, Artificial
  synthetic primer
aactcttcavnnccctgccgcctgcgcgctggatgt

SEQ ID NO: 157 - DNA, 36 bp, Artificial
  synthetic primer
aactcttcavnntttccctgccgcctgcgcgctgga

SEQ ID NO: 158 - DNA, 36 bp, Artificial
  synthetic primer
aactcttcavnntgatttccctgccgcctgcgcgct

SEQ ID NO: 159 - DNA, 33 bp, Artificial
  synthetic primer
aactcttcavnngtttgatttccctgccgcctg

SEQ ID NO: 160 - DNA, 36 bp, Artificial
  synthetic primer
aactcttcavnncccgtttgatttccctgccgcctg

SEQ ID NO: 161 - DNA, 32 bp, Artificial
  synthetic primer
aactcttcavnnttccccgtttgatttccctg

SEQ ID NO: 162 - DNA, 35 bp, Artificial
  synthetic primer
aactcttcavnncttttccccgtttgatttccctg

SEQ ID NO: 163 - DNA, 34 bp, Artificial
  synthetic primer
aactcttcavnntttcttttccccgtttgatttc

SEQ ID NO: 164 - DNA, 36 bp, Artificial
  synthetic primer
aactcttcavnnatatttcttttccccgtttgattt

SEQ ID NO: 165 - DNA, 36 bp, Artificial
  synthetic primer
aactcttcavnnaatatatttcttttccccgtttga

SEQ ID NO: 166 - DNA, 36 bp, Artificial
  synthetic primer
aactcttcavnngacaatatatttcttttccccgtt

SEQ ID NO: 167 - DNA, 33 bp, Artificial
  synthetic primer
aactcttcavnncccgacaatatatttcttttc

SEQ ID NO: 168 - DNA, 36 bp, Artificial
  synthetic primer
aactcttcavnnaaacccgacaatatatttcttttc

SEQ ID NO: 169 - DNA, 36 bp, Artificial
  synthetic primer
aactcttcavnntttaaacccgacaatatatttctt

SEQ ID NO: 170 - DNA, 36 bp, Artificial
  synthetic primer
aactcttcavnnctgtttaaacccgacaatatattt

SEQ ID NO: 171 - DNA, 36 bp, Artificial
  synthetic primer
aactcttcavnntgtctgtttaaacccgacaatata

SEQ ID NO: 172 - DNA, 36 bp, Artificial
  synthetic primer
aactcttcavnncattgtctgtttaaacccgacaat

SEQ ID NO: 173 - DNA, 36 bp, Artificial
  synthetic primer
aactcttcavnngctcattgtctgtttaaacccgac

SEQ ID NO: 174 - DNA, 34 bp, Artificial
  synthetic primer
aactcttcavnncgtgctcattgtctgtttaaac

SEQ ID NO: 175 - DNA, 36 bp, Artificial
  synthetic primer
aactcttcavnncatcgtgctcattgtctgtttaaa

SEQ ID NO: 176 - DNA, 36 bp, Artificial
  synthetic primer
aactcttcavnngctcatcgtgctcattgtctgttt

SEQ ID NO: 177 - DNA, 36 bp, Artificial
  synthetic primer
aactcttcavnnggcgctcatcgtgctcattgtctg

SEQ ID NO: 178 - DNA, 36 bp, Artificial
  synthetic primer
aactcttcavnnagcggcgctcatcgtgctcattgt

SEQ ID NO: 179 - DNA, 36 bp, Artificial
  synthetic primer
aactcttcavnncttagcggcgctcatcgtgctcat

SEQ ID NO: 180 - DNA, 36 bp, Artificial
  synthetic primer
aactcttcavnncttcttagcggcgctcatcgtgct

SEQ ID NO: 181 - DNA, 36 bp, Artificial
  synthetic primer
aactcttcavnntttcttcttagcggcgctcatcgt

SEQ ID NO: 182 - DNA, 36 bp, Artificial
  synthetic primer
aactcttcavnnatctttcttcttagcggcgctcat

SEQ ID NO: 183 - DNA, 36 bp, Artificial
  synthetic primer
aactcttcavnngacatctttcttcttagcggcgct

SEQ ID NO: 184 - DNA, 33 bp, Artificial
  synthetic primer
aactcttcavnnaatgacatctttcttcttagc

SEQ ID NO: 185 - DNA, 36 bp, Artificial
  synthetic primer
aactcttcavnnagaaatgacatctttcttcttagc

SEQ ID NO: 186 - DNA, 36 bp, Artificial
  synthetic primer
aactcttcavnnttcagaaatgacatctttcttctt

SEQ ID NO: 187 - DNA, 36 bp, Artificial
  synthetic primer
aactcttcavnntttttcagaaatgacatctttctt

SEQ ID NO: 188 - DNA, 36 bp, Artificial
  synthetic primer
aactcttcavnngcctttttcagaaatgacatcttt

SEQ ID NO: 189 - DNA, 36 bp, Artificial
  synthetic primer
aactcttcavnncccgcctttttcagaaatgacatc

SEQ ID NO: 190 - DNA, 36 bp, Artificial
  synthetic primer
aactcttcavnntttcccgcctttttcagaaatgac

SEQ ID NO: 191 - DNA, 36 bp, Artificial
  synthetic primer
aactcttcavnncactttcccgcctttttcagaaat

SEQ ID NO: 192 - DNA, 36 bp, Artificial
  synthetic primer
aactcttcavnnttgcactttcccgcctttttcaga

SEQ ID NO: 193 - DNA, 36 bp, Artificial
  synthetic primer
aactcttcavnncttttgcactttcccgcctttttc

SEQ ID NO: 194 - DNA, 36 bp, Artificial
  synthetic primer
aactcttcavnnttgcttttgcactttcccgccttt

SEQ ID NO: 195 - DNA, 32 bp, Artificial
  synthetic primer
aactcttcavnngaattgcttttgcactttcc

SEQ ID NO: 196 - DNA, 34 bp, Artificial
  synthetic primer
aactcttcavnntttgaattgcttttgcactttc

SEQ ID NO: 197 - DNA, 36 bp, Artificial
  synthetic primer
aactcttcavnnatatttgaattgcttttgcacttt

SEQ ID NO: 198 - DNA, 36 bp, Artificial
  synthetic primer
aactcttcavnntacatatttgaattgcttttgcac

SEQ ID NO: 199 - DNA, 36 bp, Artificial
  synthetic primer
aactcttcavnngtctacatatttgaattgcttttg

SEQ ID NO: 200 - DNA, 36 bp, Artificial
  synthetic primer
aactcttcavnntgcgtctacatatttgaattgctt

SEQ ID NO: 201 - DNA, 36 bp, Artificial
  synthetic primer
aactcttcavnnagctgcgtctacatatttgaattg

SEQ ID NO: 202 - DNA, 36 bp, Artificial
  synthetic primer
aactcttcavnntgaagctgcgtctacatatttgaa

SEQ ID NO: 203 - DNA, 36 bp, Artificial
  synthetic primer
aactcttcavnnagctgaagctgcgtctacatattt

SEQ ID NO: 204 - DNA, 36 bp, Artificial
  synthetic primer
aactcttcavnntgtagctgaagctgcgtctacata

SEQ ID NO: 205 - DNA, 36 bp, Artificial
  synthetic primer
aactcttcavnntaatgtagctgaagctgcgtctac

SEQ ID NO: 206 - DNA, 36 bp, Artificial
  synthetic primer
aactcttcavnngtttaatgtagctgaagctgcgtc

SEQ ID NO: 207 - DNA, 36 bp, Artificial
  synthetic primer
aactcttcavnnttcgtttaatgtagctgaagctgc

SEQ ID NO: 208 - DNA, 35 bp, Artificial
  synthetic primer
aactcttcavnntttttcgtttaatgtagctgaag

SEQ ID NO: 209 - DNA, 36 bp, Artificial
  synthetic primer
aactcttcavnnagctttttcgtttaatgtagctga

SEQ ID NO: 210 - DNA, 35 bp, Artificial
  synthetic primer
aactcttcavnntacagctttttcgtttaatgtag

SEQ ID NO: 211 - DNA, 36 bp, Artificial
  synthetic primer
aactcttcavnnttttacagctttttcgtttaatgt

SEQ ID NO: 212 - DNA, 36 bp, Artificial
  synthetic primer
aactcttcavnnttcttttacagctttttcgtttaa

SEQ ID NO: 213 - DNA, 36 bp, Artificial
  synthetic primer
aactcttcavnncaattcttttacagctttttcgtt

SEQ ID NO: 214 - DNA, 36 bp, Artificial
  synthetic primer
aactcttcavnntttcaattcttttacagctttttc

SEQ ID NO: 215 - DNA, 36 bp, Artificial
  synthetic primer
aactcttcavnnttttttcaattcttttacagcttt

SEQ ID NO: 216 - DNA, 35 bp, Artificial
  synthetic primer
aactcttcavnngtcttttttcaattcttttacag

SEQ ID NO: 217 - DNA, 36 bp, Artificial
  synthetic primer
aactcttcavnncgggtcttttttcaattcttttac

SEQ ID NO: 218 - DNA, 36 bp, Artificial
  synthetic primer
aactcttcavnngctcgggtcttttttcaattcttt

SEQ ID NO: 219 - DNA, 36 bp, Artificial
  synthetic primer
aactcttcavnngacgctcgggtcttttttcaattc

SEQ ID NO: 220 - DNA, 36 bp, Artificial
  synthetic primer
aactcttcavnnagcgacgctcgggtcttttttcaa

SEQ ID NO: 221 - DNA, 36 bp, Artificial
  synthetic primer
aactcttcavnngtaagcgacgctcgggtctttttt

SEQ ID NO: 222 - DNA, 36 bp, Artificial
  synthetic primer
aactcttcavnnaacgtaagcgacgctcgggtcttt

SEQ ID NO: 223 - DNA, 36 bp, Artificial
  synthetic primer
aactcttcavnnttcaacgtaagcgacgctcgggtc

SEQ ID NO: 224 - DNA, 34 bp, Artificial
  synthetic primer
aactcttcavnnttcttcaacgtaagcgacgctc

SEQ ID NO: 225 - DNA, 36 bp, Artificial
  synthetic primer
aactcttcavnnatcttcttcaacgtaagcgacgct

SEQ ID NO: 226 - DNA, 36 bp, Artificial
  synthetic primer
aactcttcavnngtgatcttcttcaacgtaagcgac

SEQ ID NO: 227 - DNA, 35 bp, Artificial
  synthetic primer
aactcttcavnntacgtgatcttcttcaacgtaag

SEQ ID NO: 228 - DNA, 22 bp, Artificial
  synthetic primer
tgtcgataaccgctactttaac

SEQ ID NO: 229 - DNA, 36 bp, Artificial
  synthetic primer
aactcttcannbagaagcaaaaaattgtggatcagt

SEQ ID NO: 230 - DNA, 36 bp, Artificial
  synthetic primer
aactcttcannbagcaaaaaattgtggatcagtttg

SEQ ID NO: 231 - DNA, 36 bp, Artificial
  synthetic primer
aactcttcannbaaaaaattgtggatcagtttgctg

SEQ ID NO: 232 - DNA, 36 bp, Artificial
  synthetic primer
aactcttcannbaaattgtggatcagtttgctgttt

SEQ ID NO: 233 - DNA, 36 bp, Artificial
  synthetic primer
aactcttcannbttgtggatcagtttgctgtttgct

SEQ ID NO: 234 - DNA, 36 bp, Artificial
  synthetic primer
aactcttcannbtggatcagtttgctgtttgcttta

SEQ ID NO: 235 - DNA, 34 bp, Artificial
  synthetic primer
aactcttcannbatcagtttgctgtttgctttag

SEQ ID NO: 236 - DNA, 36 bp, Artificial
  synthetic primer
aactcttcannbagtttgctgtttgctttagcgtta

SEQ ID NO: 237 - DNA, 36 bp, Artificial
  synthetic primer
aactcttcannbttgctgtttgctttagcgttaatc

SEQ ID NO: 238 - DNA, 36 bp, Artificial
  synthetic primer
aactcttcannbctgtttgctttagcgttaatcttt

SEQ ID NO: 239 - DNA, 35 bp, Artificial
  synthetic primer
aactcttcannbtttgctttagcgttaatctttac

SEQ ID NO: 240 - DNA, 36 bp, Artificial
  synthetic primer
aactcttcannbgctttagcgttaatctttacgatg

SEQ ID NO: 241 - DNA, 34 bp, Artificial
  synthetic primer
aactcttcannbttagcgttaatctttacgatgg

SEQ ID NO: 242 - DNA, 36 bp, Artificial
  synthetic primer
aactcttcannbgcgttaatctttacgatggcgttc

SEQ ID NO: 243 - DNA, 34 bp, Artificial
  synthetic primer
aactcttcannbttaatctttacgatggcgttcg

SEQ ID NO: 244 - DNA, 35 bp, Artificial
  synthetic primer
aactcttcannbatctttacgatggcgttcggcag

SEQ ID NO: 245 - DNA, 36 bp, Artificial
  synthetic primer
aactcttcannbtttacgatggcgttcggcagcaca

SEQ ID NO: 246 - DNA, 35 bp, Artificial
  synthetic primer
aactcttcannbacgatggcgttcggcagcacatc

SEQ ID NO: 247 - DNA, 35 bp, Artificial
  synthetic primer
aactcttcannbatggcgttcggcagcacatccag

SEQ ID NO: 248 - DNA, 33 bp, Artificial
  synthetic primer
aactcttcannbgcgttcggcagcacatccagc

SEQ ID NO: 249 - DNA, 36 bp, Artificial
  synthetic primer
aactcttcannbttcggcagcacatccagcgcgcag

SEQ ID NO: 250 - DNA, 33 bp, Artificial
  synthetic primer
aactcttcannbggcagcacatccagcgcgcag

SEQ ID NO: 251 - DNA, 36 bp, Artificial
  synthetic primer
aactcttcannbagcacatccagcgcgcaggcggca

SEQ ID NO: 252 - DNA, 34 bp, Artificial
  synthetic primer
aactcttcannbacatccagcgcgcaggcggcag

SEQ ID NO: 253 - DNA, 36 bp, Artificial
  synthetic primer
aactcttcannbtccagcgcgcaggcggcagggaaa

SEQ ID NO: 254 - DNA, 36 bp, Artificial
  synthetic primer
aactcttcannbagcgcgcaggcggcagggaaatca

SEQ ID NO: 255 - DNA, 36 bp, Artificial
  synthetic primer
aactcttcannbgcgcaggcggcagggaaatcaaac

SEQ ID NO: 256 - DNA, 33 bp, Artificial
  synthetic primer
aactcttcannbcaggcggcagggaaatcaaac

SEQ ID NO: 257 - DNA, 36 bp, Artificial
  synthetic primer
aactcttcannbgcggcagggaaatcaaacggggaa

SEQ ID NO: 258 - DNA, 36 bp, Artificial
  synthetic primer
aactcttcannbgcagggaaatcaaacggggaaaag

SEQ ID NO: 259 - DNA, 36 bp, Artificial
  synthetic primer
aactcttcannbgggaaatcaaacggggaaaagaaa

SEQ ID NO: 260 - DNA, 36 bp, Artificial
  synthetic primer
aactcttcannbaaatcaaacggggaaaagaaatat

SEQ ID NO: 261 - DNA, 36 bp, Artificial
  synthetic primer
aactcttcannbtcaaacggggaaaagaaatatatt

SEQ ID NO: 262 - DNA, 36 bp, Artificial
  synthetic primer
aactcttcannbaacggggaaaagaaatatattgtc

SEQ ID NO: 263 - DNA, 33 bp, Artificial
  synthetic primer
aactcttcannbggggaaaagaaatatattgtc

SEQ ID NO: 264 - DNA, 36 bp, Artificial
  synthetic primer
aactcttcannbgaaaagaaatatattgtcgggttt

SEQ ID NO: 265 - DNA, 36 bp, Artificial
  synthetic primer
aactcttcannbaagaaatatattgtcgggtttaaa

SEQ ID NO: 266 - DNA, 36 bp, Artificial
  synthetic primer
aactcttcannbaaatatattgtcgggtttaaacag

SEQ ID NO: 267 - DNA, 36 bp, Artificial
  synthetic primer
aactcttcannbtatattgtcgggtttaaacagaca

SEQ ID NO: 268 - DNA, 36 bp, Artificial
  synthetic primer
aactcttcannbattgtcgggtttaaacagacaatg

SEQ ID NO: 269 - DNA, 35 bp, Artificial
  synthetic primer
aactcttcannbgtcgggtttaaacagacaatgag

SEQ ID NO: 270 - DNA, 35 bp, Artificial
  synthetic primer
aactcttcannbgggtttaaacagacaatgagcac

SEQ ID NO: 271 - DNA, 36 bp, Artificial
  synthetic primer
aactcttcannbtttaaacagacaatgagcacgatg

SEQ ID NO: 272 - DNA, 35 bp, Artificial
  synthetic primer
aactcttcannbaaacagacaatgagcacgatgag

SEQ ID NO: 273 - DNA, 32 bp, Artificial
  synthetic primer
aactcttcannbcagacaatgagcacgatgag

SEQ ID NO: 274 - DNA, 36 bp, Artificial
  synthetic primer
aactcttcannbacaatgagcacgatgagcgccgct

SEQ ID NO: 275 - DNA, 36 bp, Artificial
  synthetic primer
aactcttcannbatgagcacgatgagcgccgctaag

SEQ ID NO: 276 - DNA, 36 bp, Artificial
  synthetic primer
aactcttcannbagcacgatgagcgccgctaagaag

SEQ ID NO: 277 - DNA, 36 bp, Artificial
  synthetic primer
aactcttcannbacgatgagcgccgctaagaagaaa

SEQ ID NO: 278 - DNA, 36 bp, Artificial
  synthetic primer
aactcttcannbatgagcgccgctaagaagaaagat

SEQ ID NO: 279 - DNA, 36 bp, Artificial
  synthetic primer
aactcttcannbagcgccgctaagaagaaagatgtc

SEQ ID NO: 280 - DNA, 36 bp, Artificial
  synthetic primer
aactcttcannbgccgctaagaagaaagatgtcatt

SEQ ID NO: 281 - DNA, 36 bp, Artificial
  synthetic primer
aactcttcannbgctaagaagaaagatgtcatttct

SEQ ID NO: 282 - DNA, 36 bp, Artificial
  synthetic primer
aactcttcannbaagaagaaagatgtcatttctgaa

SEQ ID NO: 283 - DNA, 36 bp, Artificial
  synthetic primer
aactcttcannbaagaaagatgtcatttctgaaaaa

SEQ ID NO: 284 - DNA, 34 bp, Artificial
  synthetic primer
aactcttcannbaaagatgtcatttctgaaaaag

SEQ ID NO: 285 - DNA, 32 bp, Artificial
  synthetic primer
aactcttcannbgatgtcatttctgaaaaagg

SEQ ID NO: 286 - DNA, 36 bp, Artificial
  synthetic primer
aactcttcannbgtcatttctgaaaaaggcgggaaa

SEQ ID NO: 287 - DNA, 36 bp, Artificial
  synthetic primer
aactcttcannbatttctgaaaaaggcgggaaagtg

SEQ ID NO: 288 - DNA, 36 bp, Artificial
  synthetic primer
aactcttcannbtctgaaaaaggcgggaaagtgcaa

SEQ ID NO: 289 - DNA, 36 bp, Artificial
  synthetic primer
aactcttcannbgaaaaaggcgggaaagtgcaaaag

SEQ ID NO: 290 - DNA, 36 bp, Artificial
  synthetic primer
aactcttcannbaaaggcgggaaagtgcaaaagcaa

SEQ ID NO: 291 - DNA, 36 bp, Artificial
  synthetic primer
aactcttcannbggcgggaaagtgcaaaagcaattc

SEQ ID NO: 292 - DNA, 36 bp, Artificial
  synthetic primer
aactcttcannbgggaaagtgcaaaagcaattcaaa

SEQ ID NO: 293 - DNA, 36 bp, Artificial
  synthetic primer
aactcttcannbaaagtgcaaaagcaattcaaatat

SEQ ID NO: 294 - DNA, 36 bp, Artificial
  synthetic primer
aactcttcannbgtgcaaaagcaattcaaatatgta

SEQ ID NO: 295 - DNA, 36 bp, Artificial
  synthetic primer
aactcttcannbcaaaagcaattcaaatatgtagac

SEQ ID NO: 296 - DNA, 36 bp, Artificial
  synthetic primer
aactcttcannbaagcaattcaaatatgtagacgca

SEQ ID NO: 297 - DNA, 36 bp, Artificial
  synthetic primer
aactcttcannbcaattcaaatatgtagacgcagct

SEQ ID NO: 298 - DNA, 36 bp, Artificial
  synthetic primer
aactcttcannbttcaaatatgtagacgcagcttca

SEQ ID NO: 299 - DNA, 36 bp, Artificial
  synthetic primer
aactcttcannbaaatatgtagacgcagcttcagct

SEQ ID NO: 300 - DNA, 36 bp, Artificial
  synthetic primer
aactcttcannbtatgtagacgcagcttcagctaca

SEQ ID NO: 301 - DNA, 36 bp, Artificial
  synthetic primer
aactcttcannbgtagacgcagcttcagctacatta

SEQ ID NO: 302 - DNA, 36 bp, Artificial
  synthetic primer
aactcttcannbgacgcagcttcagctacattaaac

SEQ ID NO: 303 - DNA, 36 bp, Artificial
  synthetic primer
aactcttcannbgcagcttcagctacattaaacgaa

SEQ ID NO: 304 - DNA, 36 bp, Artificial
  synthetic primer
aactcttcannbgcttcagctacattaaacgaaaaa

SEQ ID NO: 305 - DNA, 36 bp, Artificial
  synthetic primer
aactcttcannbtcagctacattaaacgaaaaagct

SEQ ID NO: 306 - DNA, 36 bp, Artificial
  synthetic primer
aactcttcannbgctacattaaacgaaaaagctgta

SEQ ID NO: 307 - DNA, 36 bp, Artificial
  synthetic primer
aactcttcannbacattaaacgaaaaagctgtaaaa

SEQ ID NO: 308 - DNA, 36 bp, Artificial
  synthetic primer
aactcttcannbttaaacgaaaaagctgtaaaagaa

SEQ ID NO: 309 - DNA, 36 bp, Artificial
  synthetic primer
aactcttcannbaacgaaaaagctgtaaaagaattg

SEQ ID NO: 310 - DNA, 36 bp, Artificial
  synthetic primer
aactcttcannbgaaaaagctgtaaaagaattgaaa

SEQ ID NO: 311 - DNA, 36 bp, Artificial
  synthetic primer
aactcttcannbaaagctgtaaaagaattgaaaaaa

SEQ ID NO: 312 - DNA, 36 bp, Artificial
  synthetic primer
aactcttcannbgctgtaaaagaattgaaaaaagac

SEQ ID NO: 313 - DNA, 36 bp, Artificial
  synthetic primer
aactcttcannbgtaaaagaattgaaaaaagacccg

SEQ ID NO: 314 - DNA, 35 bp, Artificial
  synthetic primer
aactcttcannbaaagaattgaaaaaagacccgag

SEQ ID NO: 315 - DNA, 36 bp, Artificial
  synthetic primer
aactcttcannbgaattgaaaaaagacccgagcgtc

SEQ ID NO: 316 - DNA, 36 bp, Artificial
  synthetic primer
aactcttcannbttgaaaaaagacccgagcgtcgct

SEQ ID NO: 317 - DNA, 36 bp, Artificial
  synthetic primer
aactcttcannbaaaaaagacccgagcgtcgcttac

SEQ ID NO: 318 - DNA, 36 bp, Artificial
  synthetic primer
aactcttcannbaaagacccgagcgtcgcttacgtt

SEQ ID NO: 319 - DNA, 36 bp, Artificial
  synthetic primer
aactcttcannbgacccgagcgtcgcttacgttgaa

SEQ ID NO: 320 - DNA, 36 bp, Artificial
  synthetic primer
aactcttcannbccgagcgtcgcttacgttgaagaa

SEQ ID NO: 321 - DNA, 36 bp, Artificial
  synthetic primer
aactcttcannbagcgtcgcttacgttgaagaagat

SEQ ID NO: 322 - DNA, 36 bp, Artificial
  synthetic primer
aactcttcannbgtcgcttacgttgaagaagatcac

SEQ ID NO: 323 - DNA, 36 bp, Artificial
  synthetic primer
aactcttcannbgcttacgttgaagaagatcacgta

SEQ ID NO: 324 - DNA, 36 bp, Artificial
  synthetic primer
aactcttcannbtacgttgaagaagatcacgtagca

SEQ ID NO: 325 - DNA, 36 bp, Artificial
  synthetic primer
aactcttcannbgttgaagaagatcacgtagcacac

SEQ ID NO: 326 - DNA, 33 bp, Artificial
  synthetic primer
aactcttcannbgaagaagatcacgtagcacac

SEQ ID NO: 327 - DNA, 36 bp, Artificial
  synthetic primer
aactcttcannbgaagatcacgtagcacacgcgtac

SEQ ID NO: 328 - DNA, 33 bp, Artificial
  synthetic primer
aactcttcannbgatcacgtagcacacgcgtac

SEQ ID NO: 329 - DNA, 36 bp, Artificial
  synthetic primer
aactcttcannbcacgtagcacacgcgtacgcgcag

SEQ ID NO: 330 - DNA, 35 bp, Artificial
  synthetic primer
aactcttcannbgtagcacacgcgtacgcgcagtc

SEQ ID NO: 331 - DNA, 35 bp, Artificial
  synthetic primer
aactcttcannbgcacacgcgtacgcgcagtccgt

SEQ ID NO: 332 - DNA, 33 bp, Artificial
  synthetic primer
aactcttcannbcacgcgtacgcgcagtccgtg

SEQ ID NO: 333 - DNA, 117 bp, Artificial
  synthetic aprE short promoter
gaattcatctcaaaaaaatgggtctactaaaatattattccatctattacaataaattcacagaatagtcttttaagtaagtctactctgaatttttttaaaaggagagggtaaaga

SEQ ID NO: 334 - DNA, 1329 bp, Artificial
  synthetic expression cassette
gaattcatctcaaaaaaatgggtctactaaaatattattccatctattacaataaattcacagaatagtcttttaagtaagtctactctgaatttttttaaaaggagagggtaaagagtgagaagcaaaaaattgtggatcagtttgctgtttgctttagcgttaatctttacgatggcgttcggcagcacatccagcgcgcaggcggcagggaaatcaaacggggaaaagaaatatattgtcgggtttaaacagacaatgagcacgatgagcgccgctaagaagaaagatgtcatttctgaaaaaggcgggaaagtgcaaaagcaattcaaatatgtagacgcagcttcagctacattaaacgaaaaagctgtaaaagaattgaaaaaagacccgagcgtcgcttacgttgaagaagatcacgtagcacacgcgtacgcgcagtccgtgccttacggcgtatcacaaattaaagcccctgctctgcactctcaaggctacactggatcaaatgttaaagtagcggttatcgacagcggtatcgattcttctcatcctgatttaaaggtagcaggcggagccagcatggttccttctgaaacaaatcctttccaagacaacaactctcacggaactcacgttgccggcacagttgcggctcttaataactcaatcggtgtattaggcgttgcgccaagcgcatcactttacgctgtaaaagttctcggtgctgacggttccggccaatacagctggatcattaacggaatcgagtgggcgatcgcaaacaatatggacgttattaacatgagcctcggcggaccttctggttctgctgctttaaaagcggcagttgataaagccgttgcatccggcgtcgtagtcgttgcggcagccggtaacgaaggcacttccggcagctcaagcacagtgggctaccctggtaaatacccttctgtcattgcagtaggcgctgttgacagcagcaaccaaagagcatctttctcaagcgtaggacctgagcttgatgtcatggcacctggcgtatctatccaaagcacgcttcctggaaacaaatacggcgcgttgaacggtacatcaatggcatctccgcacgttgccggagcggctgctttgattctttctaagcacccgaactggacaaacactcaagtccgcagcagtttagaaaacaccactacaaaacttggtgattctttctactatggaaaagggctgatcaacgtacaggcggcagctcagtaaactcgagataaaaaaccggccttggccccgccggttttttattatttttcttcctccggatcc

SEQ ID NO: 335 - protein, 106 aa, Artificial
  synthetic pre-pro polypeptide sequence
VRSKKLWISLLFALALIFTMAFGSTSSAQAAGKSNGEKKYIVGFKHMSTMSAAKKKDVISEKGGKVQKQFKYVDAASATLNEKAVKELKKDPSVAYVEEDHVAHAY

SEQ ID NO: 336 - DNA, 318 bp, Artificial
  synthetic polynucleotide encoding SEQ ID NO335
gtgagaagcaaaaaattgtggatcagtttgctgtttgctttagcgttaatctttacgatggcgttcggcagcacatccagcgcgcaggcggcagggaaatcaaacggggaaaagaaatatattgtcgggtttaaacatatgagcacgatgagcgccgctaagaagaaagatgtcatttctgaaaaaggcgggaaagtgcaaaagcaattcaaatatgtagacgcagcttcagctacattaaacgaaaaagctgtaaaagaattgaaaaaagacccgagcgtcgcttacgttgaagaagatcacgtagcacacgcgtac

SEQ ID NO: 337 - protein, 107 aa, Artificial
  synthetic pre-pro polypeptide sequence
VRSKKLWISLLFALALIFTMAFGSTSSAQAAGKSNGEKKYIVGFKQTMCTMSAAKKKDVISEKGGKVQKQFKYVDAASATLNEKAVKELKKDPSVAYVEEDHVAHAY

SEQ ID NO: 338 - DNA, 321 bp, Artificial
  synthetic polynucleotide encoding SEQ ID NO337
gtgagaagcaaaaaattgtggatcagtttgctgtttgctttagcgttaatctttacgatggcgttcggcagcacatccagcgcgcaggcggcagggaaatcaaacggggaaaagaaatatattgtcgggtttaaacagacaatgtgcacgatgagcgccgctaagaagaaagatgtcatttctgaaaaaggcgggaaagtgcaaaagcaattcaaatatgtagacgcagcttcagctacattaaacgaaaaagctgtaaaagaattgaaaaaagacccgagcgtcgcttacgttgaagaagatcacgtagcacacgcgtac

SEQ ID NO: 339 - protein, 107 aa, Artificial
  synthetic pre-pro polypeptide sequence
VRSKKLWISLLFALALIFTMAFGSTSSAQAAGKSNGEKKYIVGFKQTMSTMSAAKKKDVISEKGGKVQKGFKYVDAASATLNEKAVKELKKDPSVAYVEEDHVAHAY

SEQ ID NO: 340 - DNA, 321 bp, Artificial
  synthetic polynucleotide encoding SEQ ID NO339
gtgagaagcaaaaaattgtggatcagtttgctgtttgctttagcgttaatctttacgatggcgttcggcagcacatccagcgcgcaggcggcagggaaatcaaacggggaaaagaaatatattgtcgggtttaaacagacaatgagcacgatgagcgccgctaagaagaaagatgtcatttctgaaaaaggcgggaaagtgcaaaagggattcaaatatgtagacgcagcttcagctacattaaacgaaaaagctgtaaaagaattgaaaaaagacccgagcgtcgcttacgttgaagaagatcacgtagcacacgcgtac

SEQ ID NO: 341 - protein, 107 aa, Artificial
  synthetic pre-pro polypeptide sequence
VRSKKLWISLLFALALIFTMAFGSTSSAQAAGKSNGEKKYIVGFKQTMSTMSAAKKKDVISEKGGKVQKLFKYVDAASATLNEKAVKELKKDPSVAYVEEDHVAHAY

SEQ ID NO: 342 - DNA, 321 bp, Artificial
  synthetic polynucleotide encoding SEQ ID NO341
gtgagaagcaaaaaattgtggatcagtttgctgtttgctttagcgttaatctttacgatggcgttcggcagcacatccagcgcgcaggcggcagggaaatcaaacggggaaaagaaatatattgtcgggtttaaacagacaatgagcacgatgagcgccgctaagaagaaagatgtcatttctgaaaaaggcgggaaagtgcaaaagttgttcaaatatgtagacgcagcttcagctacattaaacgaaaaagctgtaaaagaattgaaaaaagacccgagcgtcgcttacgttgaagaagatcacgtagcacacgcgtac

SEQ ID NO: 343 - protein, 107 aa, Artificial
  synthetic pre-pro polypeptide sequence
VRSKKLWISLLFALALIFTMAFGSTSSAQAAGKSNGEKKYIVGFKQTMSTMHAAKKKDVISEKGGKVQKQFKYVDAASATLNEKAVKELKKDPSVAYVEEDHVAHAY

SEQ ID NO: 344 - DNA, 321 bp, Artificial
  synthetic polynucleotide encoding SEQ ID NO343
gtgagaagcaaaaaattgtggatcagtttgctgtttgctttagcgttaatctttacgatggcgttcggcagcacatccagcgcgcaggcggcagggaaatcaaacggggaaaagaaatatattgtcgggtttaaacagacaatgagcacgatgcatgccgctaagaagaaagatgtcatttctgaaaaaggcgggaaagtgcaaaagcaattcaaatatgtagacgcagcttcagctacattaaacgaaaaagctgtaaaagaattgaaaaaagacccgagcgtcgcttacgttgaagaagatcacgtagcacacgcgtac

SEQ ID NO: 345 - protein, 106 aa, Artificial
  synthetic pre-pro polypeptide sequence
VRSKKLWISLLFALALIFTMAFGSTSSAQAAGKSNGEKKYIVGFKQTMSTMSAAKKDVISEKGGKVQKQFKYVDAASATLNEKAVKELKKDPSVAYVEEDHVAHAY

SEQ ID NO: 346 - DNA, 318 bp, Artificial
  synthetic polynucleotide encoding SEQ ID NO345
gtgagaagcaaaaaattgtggatcagtttgctgtttgctttagcgttaatctttacgatggcgttcggcagcacatccagcgcgcaggcggcagggaaatcaaacggggaaaagaaatatattgtcgggtttaaacagacaatgagcacgatgagcgccgcgaagaaagatgtcatttctgaaaaaggcgggaaagtgcaaaagcaattcaaatatgtagacgcagcttcagctacattaaacgaaaaagctgtaaaagaattgaaaaaagacccgagcgtcgcttacgttgaagaagatcacgtagcacacgcgtac

SEQ ID NO: 347 - protein, 106 aa, Artificial
  synthetic primer
VRSKKLWISLLFALALIFTMAFGSTSSAQAAGKSNGKKYIVGFKQTMSTMSAAKKKDVISEKGGKVQKQFKYVDAASATLNEKAVKELKKDPSVAYVEEDHVAHAY

SEQ ID NO: 348 - DNA, 318 bp, Artificial
  synthetic polynucleotide encoding SEQ ID NO347
gtgagaagcaaaaaattgtggatcagtttgctgtttgctttagcgttaatctttacgatggcgttcggcagcacatccagcgcgcaggcggcagggaaatcaaacgggaagaaatatattgtcgggtttaaacagacaatgagcacgatgagcgccgctaagaagaaagatgtcatttctgaaaaaggcgggaaagtgcaaaagcaattcaaatatgtagacgcagcttcagctacattaaacgaaaaagctgtaaaagaattgaaaaaagacccgagcgtcgcttacgttgaagaagatcacgtagcacacgcgtac

SEQ ID NO: 349 - protein, 107 aa, Artificial
  synthetic pre-pro polypeptide sequence
VRSKKLWISLLFALALIFTMAFGSTSSAQAAGKSNGEKKYIVGFKQTMSTMSAAKKKDVISEKGGKVQKMFKYVDAASATLNEKAVKELKKDPSVAYVEEDHVAHAY

SEQ ID NO: 350 - DNA, 321 bp, Artificial
  synthetic polynucleotide encoding SEQ ID NO349
gtgagaagcaaaaaattgtggatcagtttgctgtttgctttagcgttaatctttacgatggcgttcggcagcacatccagcgcgcaggcggcagggaaatcaaacggggaaaagaaatatattgtcgggtttaaacagacaatgagcacgatgagcgccgctaagaagaaagatgtcatttctgaaaaaggcgggaaagtgcaaaagatgttcaaatatgtagacgcagcttcagctacattaaacgaaaaagctgtaaaagaattgaaaaaagacccgagcgtcgcttacgttgaagaagatcacgtagcacacgcgtac

SEQ ID NO: 351 - protein, 108 aa, Artificial
  synthetic pre-pro polypeptide sequence
VRSKKLWISLLFALALIFTMAFGSTSSAQAAGKSNGGEKKYIVGFKQTMSTMSAAKKKDVISEKGGKVQKQFKYVDAASATLNEKAVKELKKDPSVAYVEEDHVAHAY

SEQ ID NO: 352 - DNA, 324 bp, Artificial
  synthetic polynucleotide encoding SEQ ID NO351
gtgagaagcaaaaaattgtggatcagtttgctgtttgctttagcgttaatctttacgatggcgttcggcagcacatccagcgcgcaggcggcagggaaatcaaacgggggggaaaagaaatatattgtcgggtttaaacagacaatgagcacgatgagcgccgctaagaagaaagatgtcatttctgaaaaaggcgggaaagtgcaaaagcaattcaaatatgtagacgcagcttcagctacattaaacgaaaaagctgtaaaggaattgaaaaaagacccgagcgtcgcttacgttgaagaagatcacgtagcacacgcgtac

SEQ ID NO: 353 - protein, 107 aa, Artificial
  synthetic pre-pro polypeptide sequence
VRSKKLWISLLFALALIFTMAFGSTSSAQAAGKSNGEKKYIVGFKQTMSTMSAAKKKDVIFEKGGKVQKQFKYVDAASATLNEKAVKELKKDPSVAYVEEDHVAHAY

SEQ ID NO: 354 - DNA, 321 bp, Artificial
  synthetic polynucleotide encoding SEQ ID NO353
gtgagaagcaaaaaattgtggatcagtttgctgtttgctttagcgttaatctttacgatggcgttcggcagcacatccagcgcgcaggcggcagggaaatcaaacggggaaaagaaatatattgtcgggtttaaacagacaatgagcacgatgagcgccgctaagaagaaagatgtcattttcgaaaaaggcgggaaagtgcaaaagcaattcaaatatgtagacgcagcttcagctacattaaacgaaaaagctgtaaaagaattgaaaaaagacccgagcgtcgcttacgttgaagaagatcacgtagcacacgcgtac

SEQ ID NO: 355 - protein, 107 aa, Artificial; identical to SEQ ID NO: 339
  synthetic pre-pro polypeptide sequence

SEQ ID NO: 356 - DNA, 321 bp, Artificial
  synthetic polynucleotide encoding SEQ ID NO355
gtgagaagcaaaaaattgtggatcagtttgctgtttgctttagcgttaatctttacgatggcgttcggcagcacatccagcgcccaggcggcagggaaatcaaacggggaaaagaaatatattgtcgggtttaaacagacaatgagcacgatgagcgccgctaagaagaaagatgtcatttctgaaaaaggcgggaaagtgcaaaaggggttcaaatatgtagacgcagcttcagctacattaaacgaaaaagctgtaaaagaattgaaaaaagacccgagcgtcgcttacgttgaagaagatcacgtagcacacgcgtac

SEQ ID NO: 357 - protein, 107 aa, Artificial
  synthetic pre-pro polypeptide sequence
VRSKKLWISLLFALALIFTMAFGSTSSAQAAGKSNGGKKYIVGFKQTMSTMSAAKKKDVISEKGGKVQKQFKYVDAASATLNEKAVKELKKDPSVAYVEEDHVAHAY

SEQ ID NO: 358 - DNA, 321 bp, Artificial
  synthetic polynucleotide encoding SEQ ID NO357
gtgagaagcaaaaaattgtggatcagtttgctgtttgctttagcgttaatctttacgatggcgttcggcagcacatccagcgcgcaggcggcagggaaatcaaacgggggtaagaaatatattgtcgggtttaaacagacaatgagcacgatgagcgccgctaagaagaaagatgtcatttctgaaaaaggcgggaaagtgcaaaagcaattcaaatatgtagacgcagcttcagctacattaaacgaaaaagctgtaaaagaattgaaaaaagacccgagcgtcgcttacgttgaagaagatcacgtagcacacgcgtac

SEQ ID NO: 359 - protein, 107 aa, Artificial
  synthetic pre-pro polypeptide sequence
VRSKKLWISLLFALALIFTMAFGSTSSAQAAGKSNGEKKYIVGFKQTMSTMSAAKKKDVISVKGGKVQKQFKYVDAASATLNEKAVKELKKDPSVAYVEEDHVAHAY

SEQ ID NO: 360 - DNA, 321 bp, Artificial
  synthetic polynucleotide encoding SEQ ID NO359
gtgagaagcaaaaaattgtggatcagtttgctgtttgctttagcgttaatctttacgatggcgttcggcagcacatccagcgcgcaggcggcagggaaatcaaacggggaaaagaaatatattgtcgggtttaaacagacaatgagcacgatgagcgccgctaagaagaaagatgtcatttctgtcaaaggcgggaaagtgcaaaagcaattcaaatatgtagacgcagcttcagctacattaaacgaaaaagctgtaaaagaattgaaaaaagacccgagcgtcgcttacgttgaagaagatcacgtagcacacgcgtac

SEQ ID NO: 361 - protein, 108 aa, Artificial
  synthetic pre-pro polypeptide sequence
VRTSKKLWISLLFALALIFTMAFGSTSSAQAAGKSNGEKKYIVGFKQTMSTMSAAKKKDVISEKGGKVQKQFKYVDAASATLNEKAVKELKKDPSVAYVEEDHVAHAY

SEQ ID NO: 362 - DNA, 324 bp, Artificial
  synthetic polynucleotide encoding SEQ ID NO361
gtgagaacgagcaaaaaattgtggatcagtttgctgtttgctttagcgttaatctttacgatggcgttcggcagcacatccagcgcgcaggcggcagggaaatcaaacggggaaaagaaatatattgtcgggtttaaacagacaatgagcacgatgagcgccgctaagaagaaagatgtcatttctgaaaaaggcgggaaagtgcaaaagcaattcaaatatgtagacgcagcttcagctacattaaacgaaaaagctgtaaaagaattgaaaaaagacccgagcgtcgcttacgttgaagaagatcacgtagcacacgcgtac

SEQ ID NO: 363 - protein, 108 aa, Artificial
  synthetic pre-pro polypeptide sequence
VRSKKLWISLLFALALIFTMAFGSTSSAQAAGKSNGEKKYIVGFKQTMSTMSAAKKKDAVISEKGGKVQKQFKYVDAASATLNEKAVKELKKDPSVAYVEEDHVAHAY

SEQ ID NO: 364 - DNA, 324 bp, Artificial
  synthetic polynucleotide encoding SEQ ID NO363
gtgagaagcaaaaaattgtggatcagtttgctgtttgctttagcgttaatctttacgatggcgttcggcagcacatccagcgcgcaggcggcagggaaatcaaacggggaaaagaaatatattgtcgggtttaaacagacaatgagcacgatgagcgccgctaagaagaaagatgccgtcatttctgaaaaaggcgggaaagtgcaaaagcaattcaaatatgtagacgcagcttcagctacattaaacgaaaaagctgtaaaagaattgaaaaaagacccgagcgtcgcttacgttgaagaagatcacgtagcacacgcgtac

SEQ ID NO: 365 - protein, 107 aa, Artificial
  synthetic pre-pro polypeptide sequence
VRSKKLWISLLFALALIFTMAFGSTSSAQAAGKSNGEKKYIVGFKQTMSTMSAAKKKDVISEKGGKVQKQFKYVDAAVATLNEKAVKELKKDPSVAYVEEDHVAHAY

SEQ ID NO: 366 - DNA, 321 bp, Artificial
  synthetic polynucleotide encoding SEQ ID NO365
gtgagaagcaaaaaattgtggatcagtttgctgtttgctttagcgttaatctttacgatggcgttcggcagcacatccagcgcgcaggcggcagggaaatcaaacggggaaaagaaatatattgtcgggtttaaacagacaatgagcacgatgagcgccgctaagaagaaagatgtcatttctgaaaaaggcgggaaagtgcaaaagcaattcaaatatgtagacgcagctgtcgctacattaaacgaaaaagctgtaaaagaattgaaaaaagacccgagcgtcgcttacgttgaagaagatcacgtagcacacgcgtac

SEQ ID NO: 367 - protein, 105 aa, Artificial
  synthetic pre-pro polypeptide sequence
VRSKKLWISLLFALALIFTMAFGSTSSAQAAGKSNGEKKYIVGFKQTMSTMSAAKKDVISEKGGKVQKQFKYVDAASATLNEKAVKELKKDPSVAYVEEDHVAHA

SEQ ID NO: 368 - DNA, 315 bp, Artificial
  synthetic polynucleotide encoding SEQ ID NO367
gtgagaagcaaaaaattgtggatcagtttgctgtttgctttagcgttaatctttacgatggcgttcggcagcacatccagcgcgcaggcggcagggaaatcaaacggggaaaagaaatatattgtcgggtttaaacagacaatgagcacgatgagcgccgcgaagaaagatgtcatttctgaaaaaggcgggaaagtgcaaaagcaattcaaatatgtagacgcagcttcagctacattaaacgaaaaagctgtaaaagaattgaaaaaagacccgagcgtcgcttacgttgaagaagatcacgtagcacacgcg

SEQ ID NO: 369 - protein, 107 aa, Artificial
  synthetic pre-pro polypeptide sequence
VRSKKLWISLLFALALIFTMAFGSTSSAQAAGKSNGEKKYIVGFKQTMSTMSAAKKKDVISEKGGKVQKQFKYVDAASATLNEKAVKELKADPSVAYVEEDHVAHAY

SEQ ID NO: 370 - DNA, 321 bp, Artificial
  synthetic polynucleotide encoding SEQ ID NO369
gtgagaagcaaaaaattgtggatcagtttgctgtttgctttagcgttaatctttacgatggcgttcggcagcacatccagcgcgcaggcggcagggaaatcaaacggggaaaagaaatatattgtcgggtttaaacagacaatgagcacgatgagcgccgctaagaagaaagatgtcatttctgaaaaaggcgggaaagtgcaaaagcaattcaaatatgtagacgcagcttcagctacattaaacgaaaaagctgtaaaagaattgaaagcggacccgagcgtcgcttacgttgaagaagatcacgtagcacacgcgtac

SEQ ID NO: 371 - protein, 107 aa, Artificial
  synthetic pre-pro polypeptide sequence
VRSKKLWISLLFALALIFTMAFGSTSSAQAAGKSNGEKKYIVGFKQTMSTMSAAKKKDVISEKGGKVQKEFKYVDAASATLNEKAVKELKKDPSVAYVEEDHVAHAY

SEQ ID NO: 372 - DNA, 321 bp, Artificial
  synthetic polynucleotide encoding SEQ ID NO371
gtgagaagcaaaaaattgtggatcagtttgctgtttgctttagcgttaatctttacgatggcgttcggcagcacatccagcgcgcaggcggcagggaaatcaaacggggaaaagaaatatattgtcgggtttaaacagacaatgagcacgatgagcgccgctaagaagaaagatgtcatttctgaaaaaggcgggaaagtgcaaaaggagttcaaatatgtagacgcagcttcagctacattaaacgaaaaagctgtaaaagaattgaaaaaagacccgagcgtcgcttacgttgaagaagatcacgtagcacacgcgtac

SEQ ID NO: 373 - protein, 107 aa, Artificial
  synthetic pre-pro polypeptide sequence
VRSKKLWISLLFALALIFTMAFGSTSSAQAAGKSNGEKKYIVGFKQTMATMSAAKKKDVISEKGGKVQKQFKYVDAASATLNEKAVKELKKDPSVAYVEEDHVAHAY

SEQ ID NO: 374 - DNA, 321 bp, Artificial
  synthetic polynucleotide encoding SEQ ID NO373
gtgagaagcaaaaaattgtggatcagtttgctgtttgctttagcgttaatctttacgatggcgttcggcagcacatccagcgcgcaggcggcagggaaatcaaacggggaaaagaaatatattgtcgggtttaaacagacaatggccacgatgagcgccgctaagaagaaagatgtcatttctgaaaaaggcgggaaagtgcaaaagcaattcaaatatgtagacgcagcttcagctacattaaacgaaaaagctgtaaaagaattgaaaaaagacccgagcgtcgcttacgttgaagaagatcacgtagcacacgcgtac

SEQ ID NO: 375 - protein, 107 aa, Artificial
  synthetic pre-pro polypeptide sequence
VRSKKLWISLLFALALIFTMAFGTTSSAQAAGKSNGEKKYIVGFKQTMSTMSAAKKKDVISEKGGKVQKQFKYVDAASATLNEKAVKELKKDPSVAYVEEDHVAHAY

SEQ ID NO: 376 - DNA, 321 bp, Artificial
  synthetic polynucleotide encoding SEQ ID NO375
gtgagaagcaaaaaattgtggatcagtttgctgtttgctttagcgttaatctttacgatggcgttcggcaccacatccagcgcgcaggcggcagggaaatcaaacggggaaaagaaatatattgtcgggtttaaacagacaatgagcacgatgagcgccgctaagaagaaagatgtcatttctgaaaaaggcgggaaagtgcaaaagcaattcaaatatgtagacgcagcttcagctacattaaacgaaaaagctgtaaaagaattgaaaaaagacccgagcgtcgcttacgttgaagaagatcacgtagcacacgcgtac

SEQ ID NO: 377 - protein, 107 aa, Artificial
  synthetic pre-pro polypeptide sequence
VRSKKLWISLLFALALIFTMAFGSTSSAQAAGKSNGEKKYIVGFKQTMSTMSAAKKKDVISEKGGKVQKQFKYVDAAMATLNEKAVKELKKDPSVAYVEEDHVAHAY

SEQ ID NO: 378 - DNA, 321 bp, Artificial
  synthetic polynucleotide encoding SEQ ID NO377
gtgagaagcaaaaaattgtggatcagtttgctgtttgctttagcgttaatctttacgatggcgttcggcagcacatccagcgcgcaggcggcagggaaatcaaacggggaaaagaaatatattgtcgggtttaaacagacaatgagcacgatgagcgccgctaagaagaaagatgtcatttctgaaaaaggcgggaaagtgcaaaagcaattcaaatatgtagacgcagctatggctacattaaacgaaaaagctgtaaaagaattgaaaaaagacccgagcgtcgcttacgttgaagaagatcacgtagcacacgcgtac

SEQ ID NO: 379 - protein, 107 aa, Artificial
  synthetic pre-pro polypeptide sequence
VRSKKLWISLLFALALIFTMAFGSTSSAQAAGKSNGEKKYIVGFKQTMSTMSAAKKKDVISEKGGKVQKQFKYVDAASATLNEKAVKELKKDSSVAYVEEDHVAHAY

SEQ ID NO: 380 - DNA, 321 bp, Artificial
  synthetic polynucleotide encoding SEQ ID NO379
gtgagaagcaaaaaattgtggatcagtttgctgtttgctttagcgttaatctttacgatggcgttcggcagcacatccagcgcgcaggcggcagggaaatcaaacggggaaaagaaatatattgtcgggtttaaacagacaatgagcacgatgagcgccgctaagaagaaagatgtcatttctgaaaaaggcgggaaagtgcaaaagcaattcaaatatgtagacgcagcttcagctacattaaacgaaaaagctgtaaaagaattgaaaaaagactcgagcgtcgcttacgttgaagaagatcacgtagcacacgcgtac

SEQ ID NO: 381 - protein, 109 aa, Artificial
  synthetic pre-pro polypeptide sequence
VRSKKLWISLLFALALIFTATMAFGSTSSAQAAGKSNGEKKYIVGFKQTMSTMSAAKKKDVISEKGGKVQKQFKYVDAASATLNEKAVKELKKDPSVAYVEEDHVAHAY

SEQ ID NO: 382 - DNA, 327 bp, Artificial
  synthetic polynucleotide encoding SEQ ID NO381
gtgagaagcaaaaaattgtggatcagtttgctgtttgctttagcgttaatctttacggccacgatggcgttcggcagcacatccagcgcgcaggcggcagggaaatcaaacggggaaaagaaatatattgtcgggtttaaacagacaatgagcacgatgagcgccgctaagaagaaagatgtcatttctgaaaaaggcgggaaagtgcaaaagcaattcaaatatgtagacgcagcttcagctacattaaacgaaaaagctgtaaaagaattgaaaaaagacccgagcgtcgcttacgttgaagaagatcacgtagcacacgcgtac

SEQ ID NO: 383 - protein, 106 aa, Artificial
  synthetic pre-pro polypeptide sequence
VRSKKLWISLLFALALIFTMAFGSTSSAQAAGKSNGEKKYIVGFKQMSTMSAAKKKDVISEKGGKVQKQFKYVDAASATLNEKAVKELKKDPSVAYVEEDHVAHAY

SEQ ID NO: 384 - DNA, 318 bp, Artificial
  synthetic polynucleotide encoding SEQ ID NO383
gtgagaagcaaaaaattgtggatcagtttgctgtttgctttagcgttaatctttacgatggcgttcggcagcacatccagcgcgcaggcggcagggaaatcaaacggggaaaagaaatatattgtcgggtttaaacagatgagcacgatgagcgccgctaagaagaaagatgtcatttctgaaaaaggcgggaaagtgcaaaagcaattcaaatatgtagacgcagcttcagctacattaaacgaaaaagctgtaaaagaattgaaaaaagacccgagcgtcgcttacgttgaagaagatcacgtagcacacgcgtac

SEQ ID NO: 385 - protein, 107 aa, Artificial; identical to SEQ ID NO: 339
  synthetic pre-pro polypeptide sequence

SEQ ID NO: 386 - DNA, 321 bp, Artificial
  synthetic polynucleotide encoding SEQ ID NO385
gtgagaagcaaaaaattgtggatcagtttgctgtttgctttagcgttaatctttacgatggcgttcggcagcacatccagcgcgcaggcggcagggaaatcaaacggggaaaagaaatatattgtcgggtttaaacagacaatgagcacgatgagcgccgctaagaagaaagatgtcatttctgaaaaaggcgggaaagtgcaaaaggggttcaaatatgtagacgcagcttcagctacattaaacgaaaaagctgtaaaagaattgaaaaaagacccgagcgtcgcttacgttgaagaagatcacgtagcacacgcgtac

SEQ ID NO: 387 - protein, 106 aa, Artificial
  synthetic pre-pro polypeptide sequence
VRSKKLWISLLFALALIFTMAFGSTSSAQAAGKSNGEKKYIVGFKQTITMSAAKKKDVISEKGGKVQKQFKYVDAASATLNEKAVKELKKDPSVAYVEEDHVAHAY

SEQ ID NO: 388 - DNA, 318 bp, Artificial
  synthetic polynucleotide encoding SEQ ID NO387
gtgagaagcaaaaaattgtggatcagtttgctgtttgctttagcgttaatctttacgatggcgttcggcagcacatccagcgcgcaggcggcagggaaatcaaacggggaaaagaaatatattgtcgggtttaaacagacaatcacgatgagcgccgctaagaagaaagatgtcatttctgaaaaaggcgggaaagtgcaaaagcaattcaaatatgtagacgcagcttcagctacattaaacgaaaaagctgtaaaagaattgaaaaaagacccgagcgtcgcttacgttgaagaagatcacgtagcacacgcgtac

SEQ ID NO: 389 - protein, 107 aa, Artificial; identical to SEQ ID NO: 343
  synthetic pre-pro polypeptide sequence

SEQ ID NO: 390 - DNA, 321 bp, Artificial; identical to SEQ ID NO: 344
  synthetic polynucleotide encoding SEQ ID NO389

SEQ ID NO: 391 - protein, 105 aa, Artificial
  synthetic pre-pro polypeptide sequence
VRSKKLWISLLFALALIFTMASTSSAQAAGKSNGEKKYIVGFKQTMSTMSAAKKKDVISEKGGKVQKQFKYVDAASATLNEKAVKELKKDPSVAYVEEDHVAHAY

SEQ ID NO: 392 - DNA, 315 bp, Artificial
  synthetic polynucleotide encoding SEQ ID NO391
gtgagaagcaaaaaattgtggatcagtttgctgtttgctttagcgttaatctttacgatggcgagcacatccagcgcgcaggcggcagggaaatcaaacggggaaaagaaatatattgtcgggtttaaacagacaatgagcacgatgagcgccgctaagaagaaagatgtcatttctgaaaaaggcgggaaagtgcaaaagcaattcaaatatgtagacgcagcttcagctacattaaacgaaaaagctgtaaaagaattgaaaaaagacccgagcgtcgcttacgttgaagaagatcacgtagcacacgcgtac

SEQ ID NO: 393 - protein, 107 aa, Artificial; identical to SEQ ID NO: 373
  synthetic pre-pro polypeptide sequence

SEQ ID NO: 394 - DNA, 321 bp, Artificial; identical to SEQ ID NO: 374
  synthetic polynucleotide encoding SEQ ID NO393

SEQ ID NO: 395 - protein, 106 aa, Artificial; identical to SEQ ID NO: 345
  synthetic pre-pro polypeptide sequence

SEQ ID NO: 396 - DNA, 318 bp, Artificial
  synthetic polynucleotide encoding SEQ ID NO395
gtgagaagcaaaaaattgtggatcagtttgctgtttgctttagcgttaatctttacgatggcgttcggcagcacatccagcgcgcaggcggcagggaaatcaaacggggaaaagaaatatattgtcgggtttaaacagacaatgagcacgatgagcgccgctaagaaggatgtcatttctgaaaaaggcgggaaagtgcaaaagcaattcaaatatgtagacgcagcttcagctacattaaacgaaaaagctgtaaaagaattgaaaaaagacccgagcgtcgcttacgttgaagaagatcacgtagcacacgcgtac

SEQ ID NO: 397 - protein, 108 aa, Artificial
  synthetic pre-pro polypeptide sequence
VRSKKLWISLLFALALIFTMAFGSTSSAQAAGGKSNGEKKYIVGFKQTMSTMSAAKKKDVISEKGGKVQKQFKYVDAASATLNEKAVKELKKDPSVAYVEEDHVAHAY

SEQ ID NO: 398 - DNA, 324 bp, Artificial
  synthetic polynucleotide encoding SEQ ID NO397
gtgagaagcaaaaaattgtggatcagtttgctgtttgctttagcgttaatctttacgatggcgttcggcagcacatccagcgcgcaggcggcaggtgggaaatcaaacggggaaaagaaatatattgtcgggtttaaacagacaatgagcacgatgagcgccgctaagaagaaagatgtcatttctgaaaaaggcgggaaagtgcaaaagcaattcaaatatgtagacgcagcttcagctacattaaacgaaaaagctgtaaaagaattgaaaaaagacccgagcgtcgcttacgttgaagaagatcacgtagcacacgcgtac

SEQ ID NO: 399 - protein, 107 aa, Artificial
  synthetic pre-pro polypeptide sequence
VRSKKLWISLLFALALIFTMAFGSTSSAQAAGKSNGEKKYIVGFKQTMSTMSAAKKKDVISEKGGKVQKQFDYVDAASATLNEKAVKELKKDPSVAYVEEDHVAHAY

SEQ ID NO: 400 - DNA, 321 bp, Artificial
  synthetic polynucleotide encoding SEQ ID NO399
gtgagaagcaaaaaattgtggatcagtttgctgtttgctttagcgttaatctttacgatggcgttcggcagcacatccagcgcgcaggcggcagggaaatcaaacggggaaaagaaatatattgtcgggtttaaacagacaatgagcacgatgagcgccgctaagaagaaagatgtcatttctgaaaaaggcgggaaagtgcaaaagcaattcgattatgtagacgcagcttcagctacattaaacgaaaaagctgtaaaagaattgaaaaaagacccgagcgtcgcttacgttgaagaagatcacgtagcacacgcgtac

SEQ ID NO: 401 - protein, 108 aa, Artificial
  synthetic pre-pro polypeptide sequence
VRSKKLWISLLFALALIFTMASFGSTSSAQAAGKSNGEKKYIVGFKQTMSTMSAAKKKDVISEKGGKVQKQFKYVDAASATLNEKAVKELKKDPSVAYVEEDHVAHAY

SEQ ID NO: 402 - DNA, 324 bp, Artificial
  synthetic polynucleotide encoding SEQ ID NO401
gtgagaagcaaaaaattgtggatcagtttgctgtttgctttagcgttaatctttacgatggcgagtttcggcagcacatccagcgcgcaggcggcagggaaatcaaacggggaaaagaaatatattgtcgggtttaaacagacaatgagcacgatgagcgccgctaagaagaaagatgtcatttctgaaaaaggcgggaaagtgcaaaagcaattcaaatatgtagacgcagcttcagctacattaaacgaaaaagctgtaaaagaattgaaaaaagacccgagcgtcgcttacgttgaagaagatcacgtagcacacgcgtac

SEQ ID NO: 403 - protein, 107 aa, Artificial
  synthetic pre-pro polypeptide sequence
VRSKKLWISLLFALALIFTMAFGSTSSAQAAGKSNGEKKYIVGFKQTMSTMSAAKKLDVISEKGGKVQKQFKYVDAASATLNEKAVKELKKDPSVAYVEEDHVAHAY

SEQ ID NO: 404 - DNA, 321 bp, Artificial
  synthetic polynucleotide encoding SEQ ID NO403
gtgagaagcaaaaaattgtggatcagtttgctgtttgctttagcgttaatctttacgatggcgttcggcagcacatccagcgcgcaggcggcagggaaatcaaacggggaaaagaaatatattgtcgggtttaaacagacaatgagcacgatgagcgccgctaagaagttggatgtcatttctgaaaaaggcgggaaagtgcaaaagcaattcaaatatgtagacgcagcttcagctacattaaacgaaaaagctgtaaaagaattgaaaaaagacccgagcgtcgcttacgttgaagaagatcacgtagcacacgcgtac

SEQ ID NO: 405 - protein, 108 aa, Artificial
  synthetic pre-pro polypeptide sequence
VRSKKLWISLLFALALIFTMAFGSTSSAQAAAGKSNGEKKYIVGFKQTMSTMSAAKKKDVISEKGGKVQKQFKYVDAASATLNEKAVKELKKDPSVAYVEEDHVAHAY

SEQ ID NO: 406 - DNA, 324 bp, Artificial
  synthetic polynucleotide encoding SEQ ID NO405
gtgagaagcaaaaaattgtggatcagtttgctgtttgctttagcgttaatctttacgatggcgttcggcagcacatccagcgcgcaggcggccgcagggaaatcaaacggggaaaagaaatatattgtcgggtttaaacagacaatgagcacgatgagcgccgctaagaagaaagatgtcatttctgaaaaaggcgggaaagtgcaaaagcaattcaaatatgtagacgcagcttcagctacattaaacgaaaaagctgtaaaagaattgaaaaaagacccgagcgtcgcttacgttgaagaagatcacgtagcacacgcgtac

SEQ ID NO: 407 - protein, 107 aa, Artificial
  synthetic pre-pro polypeptide sequence
VRSKKLWISLLFALALIFTMAFGGTSSAQAAGKSNGEKKYIVGFKQTMSTMSAAKKKDVISEKGGKVQKQFKYVDAASATLNEKAVKELKKDPSVAYVEEDHVAHAY

SEQ ID NO: 408 - DNA, 321 bp, Artificial
  synthetic polynucleotide encoding SEQ ID NO407
gtgagaagcaaaaaattgtggatcagtttgctgtttgctttagcgttaatctttacgatggcgttcggcggcacatccagcgcgcaggcggcagggaaatcaaacggggaaaagaaatatattgtcgggtttaaacagacaatgagcacgatgagcgccgctaagaagaaagatgtcatttctgaaaaaggcgggaaagtgcaaaagcaattcaaatatgtagacgcagcttcagctacattaaacgaaaaagctgtaaaagaattgaaaaaagacccgagcgtcgcttacgttgaagaagatcacgtagcacacgcgtac

SEQ ID NO: 409 - protein, 105 aa, Artificial
  synthetic pre-pro polypeptide sequence
VRSKKLWISLLFALALWMAFGSTSSAQAAGKSNGEKKYIVGFKQTMSTMSAAKKKDVISEKGGKVQKQFKYVDAASATLNEKAVKELKKDPSVAYVEEDHVAHAY

SEQ ID NO: 410 - DNA, 315 bp, Artificial
  synthetic polynucleotide encoding SEQ ID NO409
gtgagaagcaaaaaattgtggatcagtttgctgtttgctttagcgttatggatggcgttcggcagcacatcctctgcccaggcggcagggaaatcaaacggggaaaagaaatatattgtcgggtttaaacagacaatgagcacgatgagcgccgctaagaagaaagatgtcatttctgaaaaaggcgggaaagtgcaaaagcaattcaaatatgtagacgcagcttcagctacattaaacgaaaaagctgtaaaagaattgaaaaaagacccgagcgtcgcttacgttgaagaagatcacgtagcacacgcgtac

SEQ ID NO: 411 - DNA, 21 bp, Artificial
  synthetic primer
acccaactgatcttcagcatc

SEQ ID NO: 412 - DNA, 20 bp, Artificial
  synthetic primer
accgtcagcaccgagaactt

SEQ ID NO: 413 - DNA, 24 bp, Artificial
  synthetic primer
ataggaattcatctcaaaaaaatg

SEQ ID NO: 414 - DNA, 22 bp, Artificial
  synthetic primer
tgtcgataaccgctactttaac

SEQ ID NO: 415 - DNA, 37 bp, Artificial
  synthetic primer
aggcggcaggtgggaaatcaaacggggaaaagaaata

SEQ ID NO: 416 - DNA, 40 bp, Artificial
  synthetic primer
tttccccgtttgatttcccacctgccgcctgcgcgctgga

SEQ ID NO: 417 - DNA, 52 bp, Artificial
  synthetic primer
ttccatctattacaataaattcacagaatagtcttttaagtaagtctactct

SEQ ID NO: 418 - DNA, 26 bp, Artificial
  synthetic primer
ctgtgaatttattgtaatagatggaa

SEQ ID NO: 419 - DNA, 35 bp, Artificial
  synthetic primer
tttaaacagacaatcacgatgagcgccgctaagaa

SEQ ID NO: 420 - DNA, 40 bp, Artificial
  synthetic primer
agcggcgctcatcgtgattgtctgtttaaacccgacaata

SEQ ID NO: 421 - DNA, 40 bp, Artificial
  synthetic primer
tgtagacgcagctgtcgctacattaaacgaaaaagctgta

SEQ ID NO: 422 - DNA, 40 bp, Artificial
  synthetic primer
tcgtttaatgtagcgacagctgcgtctacatatttgaatt

SEQ ID NO: 423 - DNA, 38 bp, Artificial
  synthetic primer
cgatgagcgccgcgaagaaagatgtcatttctgaaaaa

SEQ ID NO: 424 - DNA, 37 bp, Artificial
  synthetic primer
gaaatgacatctttcttcgcggcgctcatcgtgctca

SEQ ID NO: 425 - DNA, 39 bp, Artificial
  synthetic primer
tgtaaaagaattgaaagcggacccgagcgtcgcttacgt

SEQ ID NO: 426 - DNA, 40 bp, Artificial
  synthetic primer
gacgctcgggtccgctttcaattcttttacagctttttcg

SEQ ID NO: 427 - DNA, 38 bp, Artificial
  synthetic primer
aatgagcacgatgcatgccgctaagaagaaagatgtca

SEQ ID NO: 428 - DNA, 40 bp, Artificial
  synthetic primer
ttcttcttagcggcatgcatcgtgctcattgtctgtttaa

SEQ ID NO: 429 - DNA, 35 bp, Artificial
  synthetic primer
aatctttacgatggcgagcacatccagcgcgcagg

SEQ ID NO: 430 - DNA, 37 bp, Artificial
  synthetic primer
cgcgctggatgtgctcgccatcgtaaagattaacgct

SEQ ID NO: 431 - DNA, 39 bp, Artificial
  synthetic primer
ggtttaaacagacaatggccacgatgagcgccgctaaga

SEQ ID NO: 432 - DNA, 40 bp, Artificial
  synthetic primer
gcggcgctcatcgtggccattgtctgtttaaacccgacaa

SEQ ID NO: 433 - DNA, 36 bp, Artificial
  synthetic primer
atggcgttcggcaccacatccagcgcgcaggcggca

SEQ ID NO: 434 - DNA, 38 bp, Artificial
  synthetic primer
ctgcgcgctggatgtggtgccgaacgccatcgtaaaga

SEQ ID NO: 435 - DNA, 39 bp, Artificial
  synthetic primer
gagaagcaaaaaattatggatcagtttgctgtttgcttt

SEQ ID NO: 436 - DNA, 40 bp, Artificial
  synthetic primer
cagcaaactgatccataattttttgcttctcactctttac

SEQ ID NO: 437 - DNA, 40 bp, Artificial
  synthetic primer
tgtagacgcagctatggctacattaaacgaaaaagctgta

SEQ ID NO: 438 - DNA, 40 bp, Artificial
  synthetic primer
tcgtttaatgtagccatagctgcgtctacatatttgaatt

SEQ ID NO: 439 - DNA, 39 bp, Artificial
  synthetic primer
aagaattgaaaaaagactcgagcgtcgcttacgttgaag

SEQ ID NO: 440 - DNA, 38 bp, Artificial
  synthetic primer
aagcgacgctcgagtcttttttcaattcttttacagct

SEQ ID NO: 441 - DNA, 40 bp, Artificial
  synthetic primer
gcgttaatctttacggccacgatggcgttcggcagcacat

SEQ ID NO: 442 - DNA, 40 bp, Artificial
  synthetic primer
gaacgccatcgtggccgtaaagattaacgctaaagcaaac

SEQ ID NO: 443 - DNA, 40 bp, Artificial
  synthetic primer
gtgcaaaagcaattcgattatgtagacgcagcttcagcta

SEQ ID NO: 444 - DNA, 40 bp, Artificial
  synthetic primer
tgcgtctacataatcgaattgcttttgcactttcccgcct

SEQ ID NO: 445 - DNA, 607 bp, Artificial
  synthetic long aprE promoter
aattctccattttcttctgctatcaaaataacagactcgtgattttccaaacgagctttcaaaaaagcctctgccccttgcaaatcggatgcctgtctataaaattcccgatattggttaaacagcggcgcaatggcggccgcatctgatgtctttgcttggcgaatgttcatcttatttcttcctccctctcaataattttttcattctatcccttttctgtaaagtttatttttcagaatacttttatcatcatgctttgaaaaaatatcacgataatatccattgttctcacggaagcacacgcaggtcatttgaacgaattttttcgacaggaatttgccgggactcaggagcatttaacctaaaaaagcatgacatttcagcataatgaacatttactcatgtctattttcgttcttttctgtatgaaaatagttatttcgagtctctacggaaatagcgagagatgatatacctaaatagagataaaatcatctcaaaaaaatgggtctactaaaatattattccatctattacaataaattcacagaatagtcttttaagtaagtctactctgaatttttttaaaaggagagggtaaaga

SEQ ID NO: 446 - DNA, 20 bp, Artificial
  synthetic primer
gcgcgcgtaatacgactcac

SEQ ID NO: 447 - DNA, 43 bp, Artificial
  synthetic primer
atttttttgagatgattttatctctatttaggtatatcatctc

SEQ ID NO: 448 - DNA, 42 bp, Artificial
  synthetic primer
taaatagagataaaatcatctcaaaaaaatgggtctactaaa

SEQ ID NO: 449 - DNA, 25 bp, Artificial
  synthetic primer
atgtatcaagataagaaagaacaag

SEQ ID NO: 450 - DNA, 22 bp, Artificial
  synthetic primer
gcaggaattctccattttcttc

SEQ ID NO: 451 - DNA, 26 bp, Artificial
  synthetic primer
tttattttataaactcattccctgat

SEQ ID NO: 452 - DNA, 2010 bp, Artificial
  synthetic pJH-FNA vector
aattctccattttcttctgctatcaaaataacagactcgtgattttccaaacgagctttcaaaaaagcctctgccccttgcaaatcggatgcctgtctataaaattcccgatattggttaaacagcggcgcaatggcggccgcatctgatgtctttgcttggcgaatgttcatcttatttcttcctccctctcaataattttttcattctatcccttttctgtaaagtttatttttcagaatacttttatcatcatgctttgaaaaaatatcacgataatatccattgttctcacggaagcacacgcaggtcatttgaacgaattttttcgacaggaatttgccgggactcaggagcatttaacctaaaaaagcatgacatttcagcataatgaacatttactcatgtctattttcgttcttttctgtatgaaaatagttatttcgagtctctacggaaatagcgagagatgatatacctaaatagagataaaatcatctcaaaaaaatgggtctactaaaatattattccatctattacaataaattcacagaatagtcttttaagtaagtctactctgaatttttttaaaaggagagggtaaagagtgagaagcaaaaaattgtggatcagtttgctgtttgctttagcgttaatctttacgatggcgttcggcagcacatcctctgcccaggcggcagggaaatcaaacggggaaaagaaatatattgtcgggtttaaacagacaatgagcacgatgagcgccgctaagaagaaagatgtcatttctgaaaaaggcgggaaagtgcaaaagcaattcaaatatgtagacgcagcttcagctacattaaacgaaaaagctgtaaaagaattgaaaaaagacccgagcgtcgcttacgttgaagaagatcacgtagcacatgcgtacgcgcagtccgtgccttacggcgtatcacaaattaaagcccctgctctgcactctcaaggctacactggatcaaatgttaaagtagcggttatcgacagcggtatcgattcttctcatcctgatttaaaggtagcaggcggagccagcatggttccttctgaaacaaatcctttccaagacaacaactctcacggaactcacgttgccggcacagttgcggctcttaataactcaatcggtgtattaggcgttgcgccaagcgcatcactttacgctgtaaaagttctcggtgctgacggttccggccaatacagctggatcattaacggaatcgagtgggcgatcgcaaacaatatggacgttattaacatgagcctcggcggaccttctggttctgctgctttaaaagcggcagttgataaagccgttgcatccggcgtcgtagtcgttgcggcagccggtaacgaaggcacttccggcagctcaagcacagtgggctaccctggtaaatacccttctgtcattgcagtaggcgctgttgacagcagcaaccaaagagcatctttctcaagcgtaggacctgagcttgatgtcatggcacctggcgtatctatccaaagcacgcttcctggaaacaaatacggcgcgttgaacggtacatcaatggcatctccgcacgttgccggagcggctgctttgattctttctaagcacccgaactggacaaacactcaagtccgcagcagtttagaaaacaccactacaaaacttggtgattctttctactatggaaaagggctgatcaacgtacaggcggcagctcagtaaaacataaaaaaccggccttggccccgccggttttttattatttttcttcctccgcatgttcaatccgctccataatcgacggatggctccctctgaaaattttaacgagaaacggcgggttgacccggctcagtcccgtaacggccaagtcctgaaacgtctcaatcgccgcttcccggtttccggtcagctcaatgccgtaacggtcggcggcgttttcctgataccgggagacggcattcgtaatcggatcc

SEQ ID NO: 453 - protein, 4 aa, Artificial
  synthetic motif
HGTH

SEQ ID NO: 454 - protein, 7 aa, Artificial
  synthetic motif
GTSMAXP

SEQ ID NO: 455 - protein, 6 aa, Artificial
  synthetic primer
GTSXXP

SEQ ID NO: 456 - protein, 4 aa, Artificial
  synthetic motif
HGTR

SEQ ID NO: 457 - protein, 4 aa, Artificial
  synthetic reagent
AAPF

SEQ ID NO: 458 - DNA, 13 bp, Artificial
  synthetic exemplary fragment
aaannbtgaagag

SEQ ID NO: 459 - DNA, 16 bp, Artificial
  synthetic exemplary fragment
aaatttnnbtgaagag

SEQ ID NO: 460 - DNA, 10 bp, Artificial
  synthetic exemplary fragment
ctcttcannb

SEQ ID NO: 461 - DNA, 13 bp, Artificial
  synthetic exemplary fragment
ctcttcannbccc

SEQ ID NO: 462 - DNA, 16 bp, Artificial
  synthetic exemplary fragment
ctcttcannbtttccc

SEQ ID NO: 463 - DNA, 12 bp, Artificial
  synthetic mutant sequence
aaannbtttccc

SEQ ID NO: 464 - DNA, 12 bp, Artificial
  synthetic mutant sequence
aaatttnnbccc

SEQ ID NO: 465 - DNA, 15 bp, Artificial
  synthetic mutant sequence
aaatttnnbtttccc

(19) United States
(12) Patent Application Publication
(10) Pub. No.: US 2011/0171718 A1
(43) Pub. Date: Jul. 14, 2011

(54) Proteases With Modified Pre-Pro Regions

(75) Inventors: Alexander Pisarchik, Belmont, CA (US); Brian F. Schmidt, Half Moon Bay, CA (US)

(73) Assignee: DANISCO US INC., Palo Alto, CA (US)

(21) Appl. No.: 12/761,253

(22) Filed: Apr. 15, 2010

Related U.S. Application Data
(60) Provisional application No. 61/230,247, filed on Jul. 31, 2009.

(51) Int. Cl. C12N 9/54; C07H 21/00; C12N 9/48; C12N 15/63; C12N 1/21

(52) U.S. Cl. 435/221; 536/23.2; 435/212; 435/320.1; 435/252.31

(57) ABSTRACT

The invention relates to modified polynucleotides encoding modified proteases, and methods for altering the production of proteases in microorganisms. In particular, the modified polynucleotides comprise one or more mutations that encode modified proteases having modifications of the pre-pro region that enhance the production of the active enzyme. The present invention further relates to methods for altering the production of proteases in microorganisms, such as Bacillus species.

### FIELD OF THE INVENTION

This invention relates to modified polynucleotides encoding modified proteases, and methods for altering the production of proteases in microorganisms. In particular, the modified polynucleotides comprise one or more mutations that encode modified proteases having modifications of the pre-pro region that enhance the production of the active enzyme. The present invention further relates to methods for altering the production of proteases in microorganisms, such as Bacillus species.

### BACKGROUND

Proteases of bacterial origin are important industrial enzymes that are responsible for the majority of all enzyme sales, and are utilized extensively in a variety of industries, including detergents, meat tenderization, cheese-making, dehairing, baking, brewery, the production of digestive aids, and the recovery of silver from photographic film. The use of these enzymes as detergent additives stimulated their commercial development and resulted in a considerable expansion of fundamental research into these enzymes (Germano et al. Enzyme Microb. Technol. 32:246-251 [2003]). In addition to detergent and food additives, proteases e.g. alkaline proteases have substantial utilization in other industrial sectors such as leather, textile, organic synthesis, and waste water treatment (Kalisz, Adv. Biochem. Eng. Biotechnol., 36:1-65 [1988]) and (Kumar and Takagi, Biotechnol. Adv., 17:561-594 [1999]).

Consequent to the high demand for these industrial enzymes, alkaline proteases with novel properties have continued to be the focus of research interest, which has led to newer protease preparations with improved catalytic efficiency and better stability towards temperature, oxidizing agents and changing usage conditions. However, the overall cost of enzyme production and downstream processing remains the major obstacle against the successful application of any technology in the enzyme industry. To this end, researchers and process engineers have used several methods to increase the yields of alkaline proteases with respect to their industrial requirements.

In spite of the implementation of various approaches for increasing protease yield, including screening for hyper-producing strains, cloning and over-expressing proteases, improving fed-batch and chemostat fermentations, and optimizing fermentation technologies, there remains a need for additional means for enhancing the production of proteases.

### SUMMARY OF THE INVENTION

This invention provides modified polynucleotides encoding modified proteases, and methods for altering the production of proteases in microorganisms. In particular, the modified polynucleotides comprise one or more mutations that encode modified proteases having modifications of the pre-pro region that enhance the production of the active enzyme. The present invention further relates to methods for altering the production of proteases in microorganisms, such as Bacillus species.

In one embodiment, the present invention provides an isolated modified polynucleotide that encodes a modified full-length protease, wherein the isolated modified polynucleotide comprises a first polynucleotide that encodes the pre-pro region of the full-length protease, and that is operably linked to a second polynucleotide that encodes the mature region of the full-length protease, wherein the first polynucleotide encodes the pre-pro region of SEQ ID NO:7, which is further mutated to comprise at least one mutation that enhances the production of the protease by a host cell. Preferably, the host cell is a Bacillus sp. host cell e.g. a Bacillus subtilis host cell. In some embodiments, the modified full-length protease is a serine protease that is derived from a wild-type or a variant parent serine protease e.g. a Bacillus subtilis, a Bacillus amyloliquefaciens, a Bacillus pumilis or a Bacillus licheniformis serine protease.

In another embodiment, the present invention provides an isolated modified polynucleotide that encodes a modified full-length protease, wherein the isolated modified polynucleotide comprises a first polynucleotide that encodes the pre-pro region of the full-length protease, and that is operably linked to a second polynucleotide that encodes the mature region of the full-length protease, wherein the first polynucleotide encodes the pre-pro region of SEQ ID NO:7, which is further mutated to comprise at least one mutation that enhances the production of the protease by a host cell, and the second polynucleotide encodes a protease that has at least about 65% identity to the mature protease of SEQ ID NO:9. Preferably, the second polynucleotide encodes the mature protease of SEQ ID NO:9. In some embodiments, the modified full-length protease is a serine protease that is derived from a wild-type or a variant parent serine protease e.g. a Bacillus subtilis, a Bacillus amyloliquefaciens, a Bacillus pumilis or a Bacillus licheniformis serine protease. Preferably, the host cell is a Bacillus sp. host cell e.g. a Bacillus subtilis host cell.

The present invention also provides an isolated modified polynucleotide that encodes a modified full-length protease, wherein the isolated modified polynucleotide comprises a first polynucleotide that encodes the pre-pro region of the full-length protease, and that is operably linked to a second polynucleotide that encodes the mature region of the full-length protease, wherein the first polynucleotide encodes the pre-pro region of SEQ ID NO:7, which is further mutated to comprise at least one mutation that enhances the production of the protease by a host cell. In some embodiments, the at least one mutation of the first polynucleotide encodes at least one amino acid substitution at one or more positions selected from positions 2, 3, 6, 7, 8, 10, 11, 12, 13, 14, 15, 16, 17, 19, 20, 21, 22, 23, 24, 25, 26, 27, 28, 29, 30, 31, 32, 33, 34, 35, 36, 37, 38, 39, 45, 46, 47, 48, 49, 50, 51, 52, 53, 54, 55, 57, 58, 59, 61, 62, 63, 64, 66, 67, 68, 69, 70, 72, 74, 75, 76, 77, 78, 80, 82, 83, 84, 87, 88, 89, 90, 91, 93, 96, 100, and 102, wherein the positions are numbered by correspondence with the amino acid sequence of the pre-pro polypeptide of the FNA protease set forth as SEQ ID NO:7. In other embodiments, the at least one mutation encodes at least one substitution selected from X2F, N, P, and Y; X3A, M, P, and R; X6K, and M; X7E; I8W; X10A, C, G, M, and T; X11A, F, and T; X12C, P, T; X13C, G, and S; X14F; X15G, M, T, and V; X16V; X17S; X19P, and S; X20V; X21S; X22E; X23F, Q, and W; X24G, T and V; X25A, D, and W; X26C, and H; X27A, F, H, P, T, V, and Y; X28V; X29E, I, R, S, and T; X30C; X31H, K, N, S, V, and W; X32C, F, M, N, P, S, and V; X33E, F, M, P, and S; X34D, H, P, and V; X35C, Q, and S; X36C, D, L, N, S, W, and Y; X37C, G, K, and Q; X38F, Q, S, and W; X39A, C, G, I, L, M, P, S, T, and V; X45G and S; X46S; X47E and F; X48G, I, T, W, and Y; X49A, C, E and I; X50D, and Y; X51A and H; X52A, H, I, and M; X53D, E, M, Q, and T; X54F, G, H, I, and S; X55D; X57E, N, and R; X58A, C, E, F, G, K, R, S, T, W; X59E; X61A, F, I, and R; X62A, F, G, H, N, S, T and V; X63A, C, E, F, G, N, Q, R, and T; G64D, M, Q, and S; X66E; X67G and L; X68C, D, and R; X69Y; X70E, G, K, L, M, P, S, and V; X72D and N; X74C and Y; X75G; X76V; X77E, V, and Y; X78M, Q and V; X80D, L, and N; X82C, D, P, Q, S, and T; X83G, and N; X84M; X87R; X88A, D, G, T, and V; X89V; X90D and Q; X91A; X92E and S; X93G, N, and S; X96G, N, and T; X100Q; and X102T, wherein the positions are numbered by correspondence with the amino acid sequence of the pre-pro polypeptide of the FNA protease set forth as SEQ ID NO:7. In some other embodiments, the at least one mutation encodes at least one substitution selected from R2F, N, P, and Y; S3A, M, P, and R; L6K, and M; W7E; I8W; L10A, C, G, M, and T; L11A, F, and T; F12C, P, T; A13C, G, and S; L14F; A15G, M, T, and V; L16V; I17S; T19P, and S; M20V; A21S; F22E; G23F, Q, and W; S24G, T and V; T25A, D, and W; S26C, and H; S27A, F, H, P, T, V, and Y; A28V; Q29E, I, R, S, and T; A30C; A31H, K, N, S, V, and W; G32C, F, M, N, P, S, and T; K33E, F, M, P, and S; S34D, H, P, and V; N35C, Q, and S; G36C, D, L, N, S, W, and Y; E37C, G, K, and Q; K38F, Q, S, and W; K39A, C, G, I, L, M, P, S, T, and V; K45G and S; Q46S; T47E and F; M48G, I, T, W, and Y; S49A, C, E and I; T50D, and Y; M51A and H; S52A, H, I, and M; A53D, E, M, Q, and T; A54F, G, H, I, and S; K55D; K57E, N, and R; D58A, C, E, F, G, K, R, S, T, W; V59E; S61A, F, I, and R; E62A, F, G, H, N, S, T and V; K63A, C, E, F, G, N, Q, R, and T; 64D, M, Q, and S; K66E; V67G and L; Q68C, D, and R; K69Y; Q70E, G, K, L, M, P, S, and V; K72D and N; V74C and Y; D75G; A76V; A77E, V, and Y; S78M, Q and V; T80D, L, and N; N82C, D, P, Q, S, and T; E83G, and N; K84M; K87R; E88A, D, G, T, and V; L89V; K90D and Q; K91A; D92E and S; P93G, N, and S; A96G, N, and T; E100Q; and H102T, wherein the positions are numbered by correspondence with the amino acid sequence of the pre-pro polypeptide of the FNA protease set forth as SEQ ID NO:7. The host cell is a Bacillus sp. host cell e.g. a Bacillus subtilis host cell. The modified full-length protease is a serine protease that is derived from a wild-type or a variant parent serine protease. In some embodiments, the wild-type or variant parent serine protease is a Bacillus subtilis, a Bacillus amyloliquefaciens, a Bacillus pumilis or a Bacillus licheniformis serine protease. In some embodiments, the second polynucleotide encodes a protease that has at least about 65% identity to the protease of SEQ ID NO:9. Preferably, the second polynucleotide encodes the mature protease of SEQ ID NO:9.

The present invention also provides an isolated modified polynucleotide that encodes a modified full-length protease, wherein the isolated modified polynucleotide comprises a first polynucleotide that encodes the pre-pro region of the full-length protease, and that is operably linked to a second polynucleotide that encodes the mature region of the full-length protease, wherein the first polynucleotide encodes the pre-pro region of SEQ ID NO:7, which is further mutated to comprise at least one mutation that enhances the production of the protease by a host cell. The at least one mutation of the first polynucleotide encodes a combination of mutations that encodes a combination of substitutions selected from X49A-X24T, X49A-X72D, X49A-X78M, X49A-X78V, X49A-X93S, X49C-X24T, X49C-X72D, X49C-X78M, X49C-X78V, X49C-X91A, X49C-X93S, X91A-x24T, X91A-X49A, X91A-X52H, X91A-X72D, X91A-X78M, X91A-X78V, X93S-X24T, X93S-X49C, X93S-X52H, X93S-X72D, X93S-X78M, and X93S-X78V, wherein the positions are numbered by correspondence with the amino acid sequence of the pre-pro polypeptide of the FNA protease set forth as SEQ ID NO:7. In other embodiments, the at least one mutation that is a combination of mutations that encodes a combination of substitutions is selected from S49A-S24T, S49A-K72D, S49A-S78M, S49A-S78V, S49A-P93S, S49C-S24T, S49C-K72D, S49C-S78M, S49C-S78V, S49C-K91A, S49C-P93S, K91A-S24T, K91A-S49A, K91A-S52H, K91A-K72D, K91A-S78M, K91A-S78V, P93S-S24T, P93S-S49C, P93S-S52H, P93S-K72D, P93S-S78M, and P93S-S78V, wherein the positions are numbered by correspondence with the amino acid sequence of the pre-pro polypeptide of the FNA protease set forth as SEQ ID NO:7. The host cell is a Bacillus sp. host cell e.g. a Bacillus subtilis host cell. The modified full-length protease is a serine protease that is derived from a wild-type or a variant parent serine protease. In some embodiments, the wild-type or variant parent serine protease is a Bacillus subtilis, a Bacillus amyloliquefaciens, a Bacillus pumilis or a Bacillus licheniformis serine protease. In some embodiments, the second polynucleotide encodes a protease that has at least about 65% identity to the protease of SEQ ID NO:9. Preferably, the second polynucleotide encodes the mature protease of SEQ ID NO:9.

The present invention also provides an isolated modified polynucleotide that encodes a modified full-length protease, wherein the isolated modified polynucleotide comprises a first polynucleotide that encodes the pre-pro region of the full-length protease, and that is operably linked to a second polynucleotide that encodes the mature region of the full-length protease, wherein the first polynucleotide encodes the pre-pro region of SEQ ID NO:7, which is further mutated to comprise at least one mutation that enhances the production of the protease by a host cell. The at least one mutation of the first polynucleotide of the first polynucleotide encodes at least one deletion selected from p.X18_X19del, p.X22_{—}23del, pX37del, pX49del, p.X47del, pX55del and p.X57del, wherein the positions are numbered by correspondence with the amino acid sequence of the pre-pro polypeptide of the FNA protease set forth as SEQ ID NO:7. In some embodiments, the at least one mutation encodes at least one deletion selected from p.I18_T19del, p.F22_G23del, p.E37del, p.T47del, p.S49del, p.K55del, and p.K57del, wherein the positions are numbered by correspondence with the amino acid sequence of the pre-pro polypeptide of the FNA protease set forth as SEQ ID NO:7. The host cell is a Bacillus sp. host cell e.g. a Bacillus subtilis host cell. The modified full-length protease is a serine protease that is derived from a wild-type or a variant parent serine protease. In some embodiments, the wild-type or variant parent serine protease is a Bacillus subtilis, a Bacillus amyloliquefaciens, a Bacillus pumilis or a Bacillus licheniformis serine protease. In some embodiments, the second polynucleotide encodes a protease that has at least about 65% identity to the protease of SEQ ID NO:9. Preferably, the second polynucleotide encodes the mature protease of SEQ ID NO:9.

The present invention also provides an isolated modified polynucleotide that encodes a modified full-length protease, wherein the isolated modified polynucleotide comprises a first polynucleotide that encodes the pre-pro region of the full-length protease, and that is operably linked to a second polynucleotide that encodes the mature region of the full-length protease, wherein the first polynucleotide encodes the pre-pro region of SEQ ID NO:7, which is further mutated to comprise at least one mutation that enhances the production of the protease by a host cell. The at least one mutation of the first polynucleotide of the first polynucleotide encodes at least one insertion selected from p.X2_X3insT, p.X30_X31insA, p.X19_X20insAT, p.X21_X22insS, p.X32_X33insG, p.X36_X37insG, and p.X58_X59insA, wherein the positions are numbered by correspondence with the amino acid sequence of the pre-pro polypeptide of the FNA protease set forth as SEQ ID NO:7. In some embodiments, the at least one mutation encodes at least one insertion selected from p.R2_S3insT, p.A30_A31insA, p.T19_M20insAT, p.A21_F22insS, p.G32_K33insG, p.G36_E37insG, and p.D58_V59insA, wherein the positions are numbered by correspondence with the amino acid sequence of the pre-pro polypeptide of the FNA protease set forth as SEQ ID NO:7. The host cell is a Bacillus sp. host cell e.g. a Bacillus subtilis host cell. The modified full-length protease is a serine protease that is derived from a wild-type or a variant parent serine protease. In some embodiments, the wild-type or variant parent serine protease is a Bacillus subtilis, a Bacillus amyloliquefaciens, a Bacillus pumilis or a Bacillus licheniformis serine protease. In some embodiments, the second polynucleotide encodes a protease that has at least about 65% identity to the protease of SEQ ID NO:9. Preferably, the second polynucleotide encodes the mature protease of SEQ ID NO:9.

The present invention also provides an isolated modified polynucleotide that encodes a modified full-length protease, wherein the isolated modified polynucleotide comprises a first polynucleotide that encodes the pre-pro region of the full-length protease, and that is operably linked to a second polynucleotide that encodes the mature region of the full-length protease, wherein the first polynucleotide encodes the pre-pro polypeptide of SEQ ID NO:7, which is further mutated to comprise at least two mutations that enhance the production of the protease by a host cell. The at least two mutations of the first polynucleotide encode at least one substitution and at least one deletion selected from X46H-p.X47del, X49A-p.X22_X23del, X49C-p.X22_X23del, X48I-p.X49del, X17W-p.X18_X19del, X78M-p.X22_X23del, X78V-p.X22_X23del, X78V-p.X57del, X91A-p.X22_X23del, X91A-X48I-pX49del, X91A-p.X57del, X93S-p.X22_X23del, and X93S-X48I-p.X49del, and wherein the positions are numbered by correspondence with the amino acid sequence of the pre-pro polypeptide of the FNA protease set forth as SEQ ID NO:7. In some embodiments, the at least one substitution and at least one deletion are selected from Q46H-p.T47del, S49A-p.F22_G23del, S49C-p.F22_G23del, M48I-p.S49del, I17W-p.I18_T19del, S78M-p.F22_G23del, S78V-p.F22_G23del, K91A-p.F22_G23del, K91A-M48I-pS49del, K91A-p.K57del, P93S-p.F22_G23del, and P93S-M48I-p.S49del, and wherein the positions are numbered by correspondence with the amino acid sequence of the pre-pro polypeptide of the FNA protease set forth as SEQ ID NO:7. The host cell is a Bacillus sp. host cell e.g. a Bacillus subtilis host cell. The modified full-length protease is a serine protease that is derived from a wild-type or a variant parent serine protease. In some embodiments, the wild-type or variant parent serine protease is a Bacillus subtilis, a Bacillus amyloliquefaciens, a Bacillus pumilis or a Bacillus licheniformis serine protease. In some embodiments, the second polynucleotide encodes a protease that has at least about 65% identity to the protease of SEQ ID NO:9. Preferably, the second polynucleotide encodes the mature protease of SEQ ID NO:9.

The present invention also provides an isolated modified polynucleotide that encodes a modified full-length protease, wherein the isolated modified polynucleotide comprises a first polynucleotide that encodes the pre-pro region of the full-length protease, and that is operably linked to a second polynucleotide that encodes the mature region of the full-length protease, wherein the first polynucleotide encodes the pre-pro polypeptide of SEQ ID NO:7, which is further mutated to comprise at least two mutations that enhance the production of the protease by a host cell. The at least two mutations of the first polynucleotide encode at least one substitution and at least one insertion are selected from X49A-p.X2_X3insT, X49A-p32X_X33insG, X49A-p.X19_X20insAT, X49C-p.X19_X20insAT, X49C-p.X32_X33insG, X52H-p.X19_X20insAT, X72D-p.X19_X20insAT, X78M-p.X19_X20insAT, X78V-p.X19_X20insAT, X91A-p.X19_X20insAT, X91A-p.X32_X33insG, X93S-p.X19_X20insAT, and X93S-p.X32_X33insG, and wherein the positions are numbered by correspondence with the amino acid sequence of the pre-pro polypeptide of the FNA protease set forth as SEQ ID NO:7. In some embodiments, the at least one substitution and at least one insertion are selected from S49A-p.R2_S3insT, S49A-p32G_K33insG, S49A-p.T19_M20insAT, S49C-p.T19_M20insAT, S49C-p.G32_K33insG, S49C-p.T19_M20insAT, S52H-p.T19_M20insAT, K72D-p.T19_M20insAT, S78M-p.T19_M20insAT, S78V-p.T19_M20insAT, K91A-p.T19_M20insAT, K91A-p.G32_K33insG, P93S-p.T19_M20insAT, and P93S-p.G32_K33insG, wherein the positions are numbered by correspondence with the amino acid sequence of the pre-pro polypeptide of the FNA protease set forth as SEQ ID NO:7. The host cell is a Bacillus sp. host cell e.g. a Bacillus subtilis host cell. The modified full-length protease is a serine protease that is derived from a wild-type or a variant parent serine protease. In some embodiments, the wild-type or variant parent serine protease is a Bacillus subtilis, a Bacillus amyloliquefaciens, a Bacillus pumilis or a Bacillus licheniformis serine protease. In some embodiments, the second polynucleotide encodes a protease that has at least about 65% identity to the protease of SEQ ID NO:9. Preferably, the second polynucleotide encodes the mature protease of SEQ ID NO:9.

The present invention also provides an isolated modified polynucleotide that encodes a modified full-length protease, wherein the isolated modified polynucleotide comprises a first polynucleotide that encodes the pre-pro region of the full-length protease, and that is operably linked to a second polynucleotide that encodes the mature region of the full-length protease, wherein the first polynucleotide encodes the pre-pro polypeptide of SEQ ID NO:7, which is further mutated to comprise at least two mutations that enhance the production of the protease by a host cell. The at least two mutations of the first polynucleotide encode at least one deletion and at least one insertion selected from p.X57del-p.X19_X20insAT, and p.X22_X23del-p.X2_X3insT, and wherein the positions are numbered by correspondence with the amino acid sequence of the pre-pro polypeptide of the FNA protease set forth as SEQ ID NO:7. In some embodiments, the at least one deletion and the at least one insertion are selected from pK57del-p.T19_M20insAT, and p.F22_G23del-p.R2_S3insT. Preferably, the first polynucleotide encodes the pre-pro polypeptide of SEQ ID NO:7, which is mutated to comprise at least two mutations that enhance the production of the protease by a host cell. The host cell is a Bacillus sp. host cell e.g. a Bacillus subtilis host cell. The modified full-length protease is a serine protease that is derived from a wild-type or a variant parent serine protease. In some embodiments, the wild-type or variant parent serine protease is a Bacillus subtilis, a Bacillus amyloliquefaciens, a Bacillus pumilis or a Bacillus licheniformis serine protease. In some embodiments, the second polynucleotide encodes a protease that has at least about 65% identity to the protease of SEQ ID NO:9. Preferably, the second polynucleotide encodes the mature protease of SEQ ID NO:9.

The present invention also provides an isolated modified polynucleotide that encodes a modified full-length protease, wherein the isolated modified polynucleotide comprises a first polynucleotide that encodes the pre-pro region of the full-length protease, and that is operably linked to a second polynucleotide that encodes the mature region of the full-length protease, wherein the first polynucleotide encodes the pre-pro polypeptide of SEQ ID NO:7, which is further mutated to comprise at least three mutations that enhance the production of the protease by a host cell. The at least three mutations of the first polynucleotide encode at least one deletion, one insertion and one substitution corresponding to p.X49del-p.X19_X20insAT-X48I, and wherein the positions are numbered by correspondence with the amino acid sequence of the pre-pro polypeptide of the FNA protease set forth as SEQ ID NO:7. In some embodiments, the at least three mutations encoding at least one deletion, one insertion and one substitution correspond to p.S49del-p.T19_M20insAT-M48I, wherein the positions are numbered by correspondence with the amino acid sequence of the pre-pro polypeptide of the FNA protease set forth as SEQ ID NO:7. The host cell is a Bacillus sp. host cell e.g. a Bacillus subtilis host cell. The modified full-length protease is a serine protease that is derived from a wild-type or a variant parent serine protease. In some embodiments, the wild-type or variant parent serine protease is a Bacillus subtilis, a Bacillus amyloliquefaciens, a Bacillus pumilis or a Bacillus licheniformis serine protease. In some embodiments, the second polynucleotide encodes a protease that has at least about 65% identity to the protease of SEQ ID NO:9. Preferably, the second polynucleotide encodes the mature protease of SEQ ID NO:9.

In another embodiment, the invention provides for polypeptides encoded by any one of the modified full-length polynucleotides described above.

In another embodiment, the invention provides an expression vector that comprises any one of the isolated modified polynucleotides described above. In some embodiments, the expression vector further comprises an AprE promoter. e.g SEQ ID NO:333 or SEQ ID NO:445.

In another embodiment, the invention provides a Bacillus sp. host cell e.g. Bacillus subtilis that comprises the expression vector of the invention, and capable of expressing any one of the modified polynucleotides provided above. Preferably, the expression vector is stably integrated into the genome of the host cell. In some embodiments, the host cell of the invention is a Bacillus sp. host cell. In some embodiments, the Bacillus sp. host cell is selected from B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, and B. thuringiensis. In some embodiments, the Bacillus sp. host cell is a B. subtilis host cell.

In another embodiment, the invention provides a method for producing a mature protease in a Bacillus sp. host cell that comprises (a) providing the expression vector comprising an isolated modified polynucleotide that encodes a modified full-length protease, which comprises a first polynucleotide that encodes the pre-pro region of the full-length protease, and that is operably linked to a second polynucleotide that encodes the mature region of the full-length protease, wherein the first polynucleotide encodes the pre-pro polypeptide of SEQ ID NO:7, which is further mutated to comprise at least one mutation that enhances the production of the mature protease by the host cell, wherein the at least one mutation is selected from X2F, N, P, and Y; X3A, M, P, and R; X6K, and M; X7E; I8W; X10A, C, G, M, and T; X11A, F, and T; X12C, P, T; X13C, G, and S; X14F; X15G, M, T, and V; X16V; X17S; X19P, and S; X20V; X21S; X22E; X23F, Q, and W; X24G, T and V; X25A, D, and W; X26C, and H; X27A, F, H, P, T, V, and Y; X28V; X29E, I, R, S, and T; X30C; X31H, K, N, S, V, and W; X32C, F, M, N, P, S, and V; X33E, F, M, P, and S; X34D, H, P, and V; X35C, Q, and S; X36C, D, L, N, S, W, and Y; X37C, G, K, and Q; X38F, Q, S, and W; X39A, C, G, I, L, M, P, S, T, and V; X45G and S; X46S; X47E and F; X48G, I, T, W, and Y; X49A, C, E and I; X50D, and Y; X51A and H; X52A, H, I, and M; X53D, E, M, Q, and T; X54F, G, H, I, and S; X55D; X57E, N, and R; X58A, C, E, F, G, K, R, S, T, W; X59E; X61A, F, I, and R; X62A, F, G, H, N, S, T and V; X63A, C, E, F, G, N, Q, R, and T; G64D, M, Q, and S; X66E; X67G and L; X68C, D, and R; X69Y; X70E, G, K, L, M, P, S, and V; X72D and N; X74C and Y; X75G; X76V; X77E, V, and Y; X78M, Q and V; X80D, L, and N; X82C, D, P, Q, S, and T; X83G, and N; X84M; X87R; X88A, D, G, T, and V; X89V; X90D and Q; X91A; X92E and S; X93G, N, and S; X96G, N, and T; X100Q; X102T; X49A-X24T, X49A-X72D, X49A-X78M, X49A-X78V, X49A-X93S, X49C-X24T, X49C-X72D, X49C-X78M, X49C-X78V, X49C-X91A, X49C-X93S, X91A-x24T, X91A-X49A, X91A-X52H, X91A-X72D, X91A-X78M, X91A-X78V, X93S-X24T, X93S-X49C, X93S-X52H, X93S-X72D, X93S-X78M, X93S-X78V, p.X18_X19del, p.X22_X23del, pX37del, pX49del, p.X47del, pX55del, p.X57del, p.X2_X3insT, p.X30_X31insA, p.X19_X20insAT, p.X21_X22insS, p.X32_X33insG, p.X36_X37insG, p.X58_X59insA, X46H-p.X47del, X49A-p.X22_X23del, X49C-p.X22_X23del, X48I-p.X49del, X17W-p.X18_X19del, X78M-p.X22_X23del, X78V-p.X22_X23del, X78V-p.X57del, X91A-p.X22_X23del, X91A-X48I-pX49del, X91A-p.X57del, X93S-p.X22_X23del, X93S-X48I-p.X49del, X49A-p.X2_X3insT, X49A-p32X_X33insG, X49A-p.X19_X20insAT, X49C-p.X19_X20insAT, X49C-p.X32_X33insG, X52H-p.X19_X20insAT, X72D-p.X19_X20insAT, X78M-p.X19_X20insAT, X78V-p.X19_X20insAT, X91A-p.X19_X20insAT, X91A-p.X32_X33insG, X93S-p.X19_X20insAT, X93S-p.X32_X33insG, p.X57del-p.X19_X20insAT, p.X22_X23del-p.X2_X3insT, p.X49del-p.X19_X20insAT-X48I, and p.X49del-p.X19_X20insAT-X48I, and wherein the positions are numbered by correspondence with the amino acid sequence of the pre-pro polypeptide of the FNA protease set forth as SEQ ID NO:7; (b) transforming the host cell with the expression vector, and (c) culturing the transformed host cell under suitable conditions to allow for the production of the mature protease. In some embodiments, the method further comprises recovering the mature protease. In some embodiments, the protease is an serine protease. In some embodiments, the Bacillus sp. host cell is a Bacillus subtilis host cell. In some embodiments, the modified polynucleotide encodes a full-length protease that comprises a mature region that is at least 65% identical to SEQ ID NO:9. Preferably, the second polynucleotide encodes the mature protease of SEQ ID NO:9. The host cell is a Bacillus sp. host cell e.g. a Bacillus subtilis host cell. The modified full-length protease is a serine protease that is derived from a wild-type or a variant parent serine protease. In some embodiments, the wild-type or variant parent serine protease is a Bacillus subtilis, a Bacillus amyloliquefaciens, a Bacillus pumilis or a Bacillus licheniformis serine protease.

In another embodiment, the invention provides a method for producing a mature protease in a Bacillus sp. host cell that comprises (a) providing an expression vector, which in turn comprises a first polynucleotide of SEQ ID NO:7 that is operably linked to a second polynucleotide that encodes the pro-pro region of SEQ ID NO:9, wherein the first polynucleotide is mutated to encode at least one mutation that enhances the production of the mature protease by the cell, wherein the at least one mutation is selected from R2F, N, P, and Y; S3A, M, P, and R; L6K, and M; W7E; I8W; L10A, C, G, M, and T; L11A, F, and T; F12C, P, T; A13C, G, and S; L14F; A15G, M, T, and V; L16V; I17S; T19P, and S; M20V; A21S; F22E; G23F, Q, and W; S24G, T and V; T25A, D, and W; S26C, and H; S27A, F, H, P, T, V, and Y; A28V; Q29E, I, R, S, and T; A30C; A31H, K, N, S, V, and W; G32C, F, M, N, P, S, and T; K33E, F, M, P, and S; S34D, H, P, and V; N35C, Q, and S; G36C, D, L, N, S, W, and Y; E37C, G, K, and Q; K38F, Q, S, and W; K39A, C, G, I, L, M, P, S, T, and V; K45G and S; Q46S; T47E and F; M48G, I, T, W, and Y; S49A, C, E and I; T50D, and Y; M51A and H; S52A, H, I, and M; A53D, E, M, Q, and T; A54F, G, H, I, and S; K55D; K57E, N, and R; D58A, C, E, F, G, K, R, S, T, W; V59E; S61A, F, I, and R; E62A, F, G, H, N, S, T and V; K63A, C, E, F, G, N, Q, R, and T; 64D, M, Q, and S; K66E; V67G and L; Q68C, D, and R; K69Y; Q70E, G, K, L, M, P, S, and V; K72D and N; V74C and Y; D75G; A76V; A77E, V, and Y; S78M, Q and V; T80D, L, and N; N82C, D, P, Q, S, and T; E83G, and N; K84M; K87R; E88A, D, G, T, and V; L89V; K90D and Q; K91A; D92E and S; P93G, N, and S; A96G, N, and T; E100Q; H102T, S49A-S24T, S49A-K72D, S49A-S78M, S49A-S78V, S49A-P93S, S49C-S24T, S49C-K72D, S49C-S78M, S49C-S78V, S49C-K91A, S49C-P93S, K91A-S24T, K91A-S49A, K91A-S52H, K91A-K72D, K91A-S78M, K91A-S78V, P93S-S24T, P93S-S49C, P93S-S52H, P93S-K72D, P93S-S78M, P93S-S78V, p.I18_T19del, p.F22_G23del, p.E37del, p.T47del, p.S49del, p.K55del, p.K57del, p.R2_S3insT, p.A30_A31insA, p.T19_M20insAT, p.A21_F22insS, p.G32_K33insG, p.G36_E37insG, p.D58_V59insA, Q46H-p.T47del, 549A-p.F22_G23del, S49C-p.F22_G23del, M48I-p.S49del, I17W-p.I18_T19del, S78M-p.F22_G23del, S78V-p.F22_G23del, K91A-p.F22_G23del, K91A-M48I-pS49del, K91A-p.K57del, P93S-p.F22_G23del, P93S-M481-p.S49del, S49A-p.R2_S3insT, S49A-p32G_K33insG, S49A-p.T19_M20insAT, S49C-p.T19_M20insAT, S49C-p.G32_K33insG, S49C-p.T19_M20insAT, S52H-p.T19_M20insAT, K72D-p.T19_M20insAT, S78M-p.T19_M20insAT, S78V-p.T19_M20insAT, K91A-p.T19_M20insAT, K91A-p.G32_K33insG, P93S-p.T19_M20insAT, P93S-p.G32_K33insG, pK57del-p.T19_M20insAT, p.F22_G23del-p.R2_S3insT, and p.S49del-p.T19_M20insAT-M48I; (b) transforming the Bacillus sp. host cell with the expression vector; and (c) culturing the transformed host cell under suitable conditions to allow for the production of the mature protease. In some embodiments, the method further comprises recovering the mature protease. In some embodiments, the protease is a serine protease, and wherein the positions are numbered by correspondence with the amino acid sequence of the pre-pro polypeptide of the FNA protease set forth as SEQ ID NO:7. In some embodiments, the Bacillus sp. host cell is a Bacillus subtilis host cell. In some embodiments, the at least one mutation increases the production of the mature protease.

### BRIEF DESCRIPTION OF THE DRAWINGS

FIG. 1 provides the amino acid sequence of the full-length FNA protease of SEQ ID NO:1. Amino acids 1-107 (SEQ ID NO:7), and amino acids 108-382 (SEQ ID NO:9) correspond to the pre-pro polypeptide and the mature portion of FNA (SEQ ID NO:1), respectively.

FIG. 2 shows an alignment of the amino acid sequence of the unmodified pre-pro region of FNA (SEQ ID NO:7) with that of unmodified pre-pro regions of proteases from various Bacillus sp.

FIG. 3 shows an alignment of the amino acid sequence of the mature region of FNA (SEQ ID NO:9) with that of mature regions of proteases from various Bacillus sp.

FIG. 4 shows a diagram illustrating the method used for creating in-frame deletions and insertions. Library quality: 33% had no insertions or deletions; 33% had insertions and 33% had deletions; there were no frame shift mutations.

FIG. 5 shows a diagram of plasmid pAC-FNAare, which was used for the expression of FNA protease in B. subtilis. The plasmid elements are as follows: pUB110=DNA fragment from plasmid pUB110 [McKenzie T., Hoshino T., Tanaka T., Sueoka N. (1986) The Nucleotide Sequence of pUB110: Some Salient Features in Relation to Replication and Its Regulation. Plasmid 15:93-103], pBR322=DNA fragment from plasmid pBR322 [Bolivar F, Rodriguez R L, Greene P J, Betlach M C, Heyneker H L, Boyer H W. (1977). Construction and characterization of new cloning vehicles. II. A multipurpose cloning system. Gene 2:95-113], pC194=DNA fragment from plasmid pC194 [Horinouchi S., Weisblum B. (1982) Nucleotide sequence and functional map of pC194, a plasmid that specifies inducible chloramphenicol resistance. J. Bacteriol 150:815-825].

FIG. 6 shows a diagram of integrating vector pJH-FNA (Ferrari et al. J. Bacteriol. 154:1513-1515 [1983]) used for expression of FNA protease in B. subtilis.

FIG. 7 shows a bar diagram depicting the percent relative activity of mature FNA (SEQ ID NO:9) processed from a modified full-length FNA protein having a mutated pre-pro polypeptide containing the amino acid substitution P93S, and the deletion p.F22_G23del (clone 684) relative to the production of the same mature FNA when processed from the unmodified full-length FNA precursor protein (unmodified; SEQ ID NO:1).

### DESCRIPTION OF THE INVENTION

This invention provides modified polynucleotides encoding modified proteases, and methods for altering the production of proteases in microorganisms. In particular, the modified polynucleotides comprise one or more mutations that encode modified proteases having modifications of the pre-pro region that enhance the production of the active enzyme. The present invention further relates to methods for altering the production of proteases in microorganisms, such as Bacillus species.

Unless defined otherwise herein, all technical and scientific terms used herein have the same meaning as commonly understood by one of ordinary skill in the art to which this invention pertains (e.g. Singleton and Sainsbury, Dictionary of Microbiology and Molecular Biology, 2d Ed., John Wiley and Sons, NY [1994]; and Hale and Markham, The Harper Collins Dictionary of Biology, Harper Perennial, NY [1991]). Although any methods and materials similar or equivalent to those described herein find use in the practice of the present invention, the preferred methods and materials are described herein. Accordingly, the terms defined immediately below are more fully described by reference to the Specification as a whole. Also, as used herein, the singular “a”, “an” and “the” includes the plural reference unless the context clearly indicates otherwise. Numeric ranges are inclusive of the numbers defining the range. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

It is intended that every maximum numerical limitation given throughout this specification include every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All patents, patent applications, articles and publications mentioned herein, both supra and infra, are hereby expressly incorporated herein by reference.

Furthermore, the headings provided herein are not limitations of the various aspects or embodiments of the invention which can be had by reference to the specification as a whole. Accordingly, the terms defined immediately below are more fully defined by reference to the specification as a whole. Nonetheless, in order to facilitate understanding of the invention, a number of terms are defined below.

### DEFINITIONS

As used herein, the terms “isolated” and “purified” refer to a nucleic acid or amino acid (or other component) that is removed from at least one component with which it is naturally associated.

The term “modified polynucleotide” herein refers to a polynucleotide sequence that has been altered to contain at least one mutation to encode a “modified” protein.

As used herein, the terms “protease” and “proteolytic activity” refer to a protein or peptide exhibiting the ability to hydrolyze peptides or substrates having peptide linkages. Many well known procedures exist for measuring proteolytic activity (Kalisz, “Microbial Proteinases,” In: Fiechter (ed.), Advances in Biochemical Engineering/Biotechnology, [1988]). For example, proteolytic activity may be ascertained by comparative assays which analyze the produced protease's ability to hydrolyze a commercial substrate. Exemplary substrates useful in such analysis of protease or proteolytic activity, include, but are not limited to di-methyl casein (Sigma C-9801), bovine collagen (Sigma C-9879), bovine elastin (Sigma E-1625), and bovine keratin (ICN Biomedical 902111). Colorimetric assays utilizing these substrates are well known in the art (See e.g., WO 99/34011; and U.S. Pat. No. 6,376,450, both of which are incorporated herein by reference. The AAPF assay (See e.g., Del Mar et al., Anal. Biochem., 99:316-320 [1979]) also finds use in determining the production of mature protease. This assay measures the rate at which p-nitroaniline is released as the enzyme hydrolyzes the soluble synthetic substrate, succinyl-alanine-alanine-proline-phenylalanine-p-nitroanilide (sAAPF-pNA). The rate of production of yellow color from the hydrolysis reaction is measured at 410 nm on a spectrophotometer and is proportional to the active enzyme concentration. In particular, the term “protease” herein refers to a “serine protease”.

As used herein, the terms “subtilisin” and “serine protease” are used interchangeably to refer to any member of the S8 serine protease family as described in MEROPS—The Peptidase Data base (Rawlings et al., MEROPS: the peptidase database, Nucleic Acids Res, 34 Database issue, D270-272, 2006, at the website merops.sangerac.uk/cgi-bin/merops.cgi?id=s08;action=.). The following information was derived from MEROPS—The Peptidase Data base as of Nov. 6, 2008 “Peptidase family S8 contains the serine endopeptidase serine protease and its homologues (Biochem J, 290:205-218, 1993). Family S8, also known as the subtilase family, is the second largest family of serine peptidases, and can be divided into two subfamilies, with subtilisin (S08.001) the type-example for subfamily S8A and kexin (S08.070) the type-example for subfamily S8B. Tripeptidyl-peptidase II (TPP-II; S08.090) was formerly considered to be the type-example of a third subfamily, but has since been determined to be misclassified. Members of family S8 have a catalytic triad in the order Asp, His and Ser in the sequence, which is a different order to that of families S1, S9 and S10. In subfamily S8A, the active site residues frequently occurs in the motifs Asp-Thr/Ser-Gly (which is similar to the sequence motif in families of aspartic endopeptidases in clan AA), His-Gly-Thr-His and Gly-Thr-Ser-Met-Ala-Xaa-Pro. In subfamily S8B, the catalytic residues frequently occur in the motifs Asp-Asp-Gly, His-Gly-Thr-Arg and Gly-Thr-Ser-Ala/Val-Ala/Ser-Pro. Most members of the S8 family are endopeptidases, and are active at neutral-mildly alkali pH. Many peptidases in the family are thermostable. Casein is often used as a protein substrate and a typical synthetic substrate is suc-AAPF. Most members of the family are nonspecific peptidases with a preference to cleave after hydrophobic residues. However, members of subfamily S8B, such as kexin (S08.070) and furin (S08.071), cleave after dibasic amino acids. Most members of the S8 family are inhibited by general serine peptidase inhibitors such as DFP and PMSF. Because many members of the family bind calcium for stability, inhibition can be seen with EDTA and EGTA, which are often thought to be specific inhibitors of metallopeptidases. Protein inhibitors include turkey ovomucoid third domain (I01.003), Streptomyces subtilisin inhibitor (I16.003), and members of family I13 such as eglin C (I13.001) and barley inhibitor Cl-1A (I13.005), many of which also inhibit chymotrypsin (S01.001). The subtilisin propeptide is itself inhibitory, and the homologous proteinase B inhibitor from Saccharomyces inhibits cerevisin (S08.052). The tertiary structures for several members of family S8 have now been determined. A typical S8 protein structure consists of three layers with a seven-stranded β sheet sandwiched between two layers of helices. Subtilisin (S08.001) is the type structure for clan SB (SB). Despite the different structure, the active sites of subtilisin and chymotrypsin (S01.001) can be superimposed, which suggests the similarity is the result of convergent rather than divergent evolution.

The terms “precursor protease” and “parent protease” herein refer to an unmodified full-length protease comprising a pre-pro region and a mature region of a full-length wild-type or variant parent protease. The precursor protease can be derived from naturally-occurring i.e. wild-type proteases, or from variant proteases. It is the pre-pro region of the wild-type or variant precursor protease that is modified to generate a modified protease. In this context, both “modified” and “precursor” proteases are full-length proteases comprising a signal peptide, a pro region and a mature region. The polynucleotides that encode the modified sequence are referred to as “modified polynucleotides”, and the polynucleotides that encode the precursor protease are referred to as “precursor polynucleotides”. “Precursor polypeptides” and “precursor polynucleotides” can be interchangeably referred to as “unmodified precursor polypeptides” or “unmodified precursor polynucleotides”, respectively.

“Naturally-occurring” or “wild-type” herein refer to a protease, or a polynucleotide encoding a protease having the unmodified amino acid sequence identical to that found in nature. Naturally occurring enzymes include native enzymes, those enzymes naturally expressed or found in the particular microorganism. A sequence that is wild-type or naturally-occurring refers to a sequence from which a variant is derived. The wild-type sequence may encode either a homologous or heterologous protein.

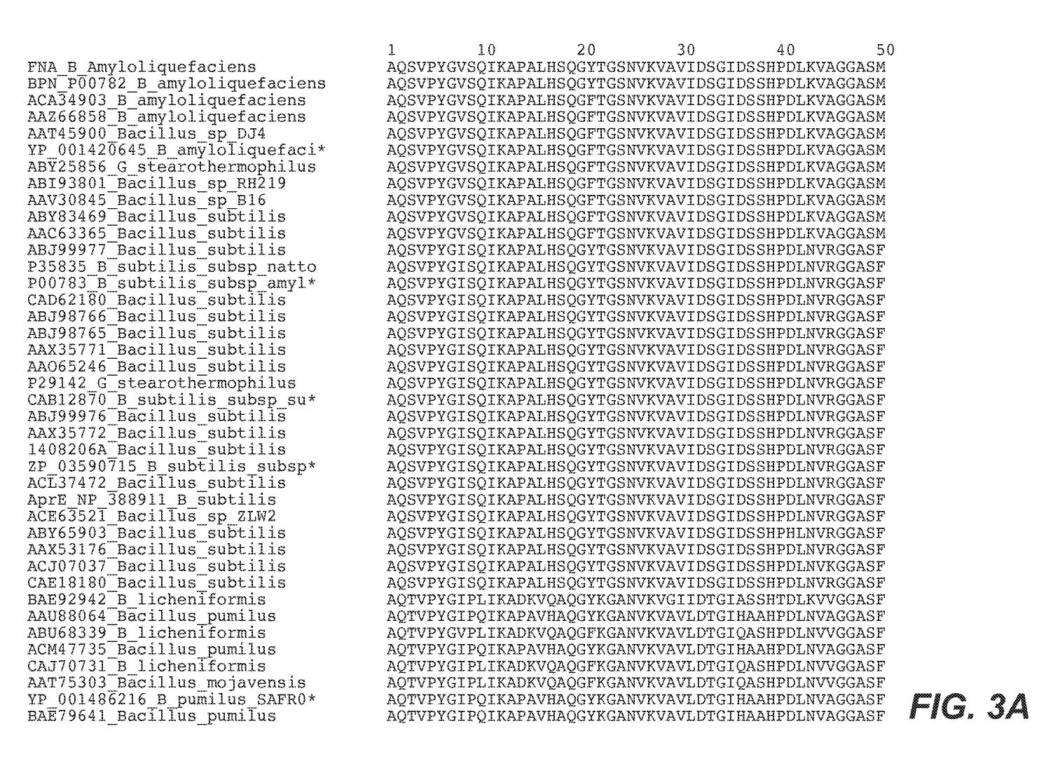

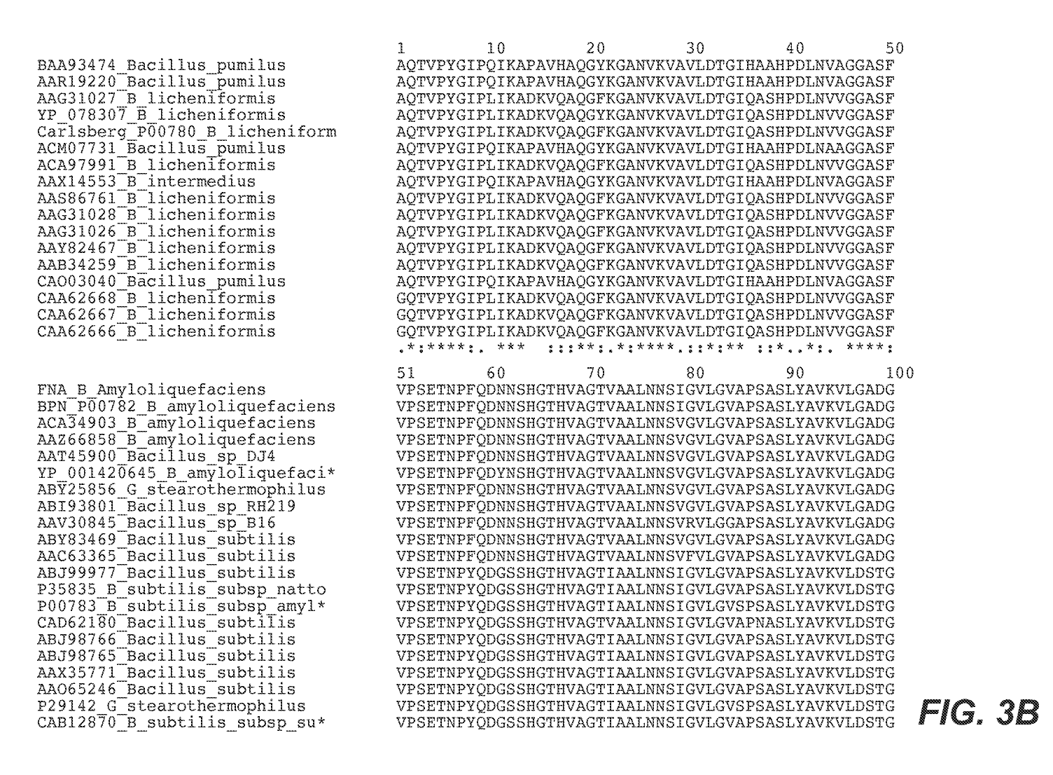

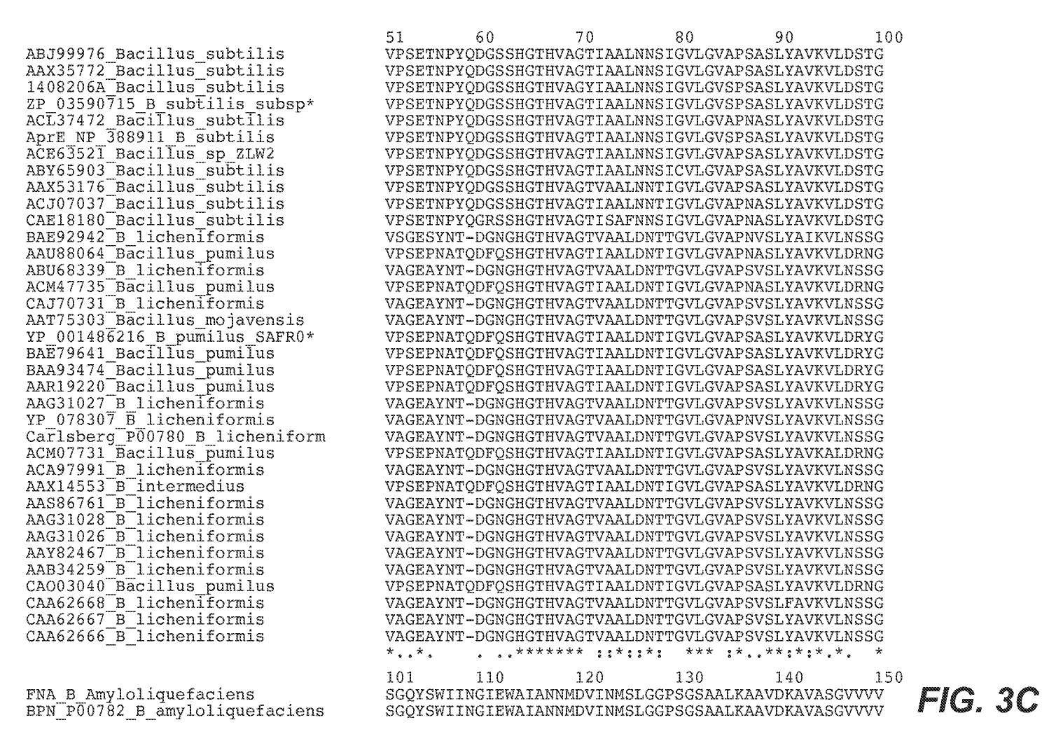

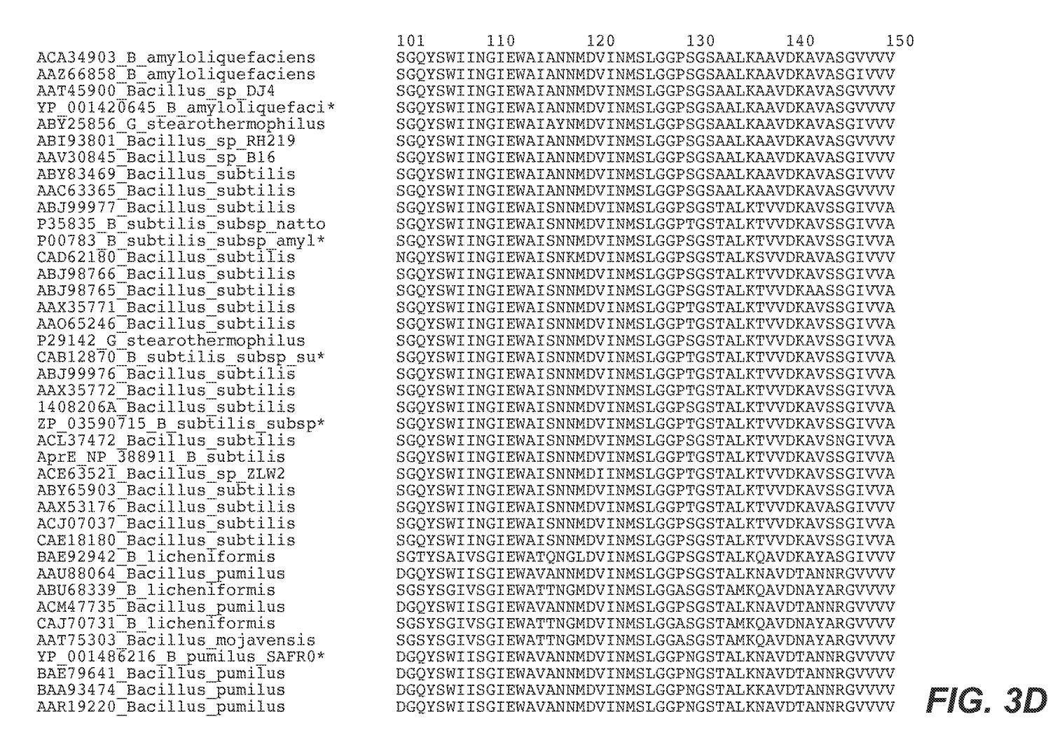

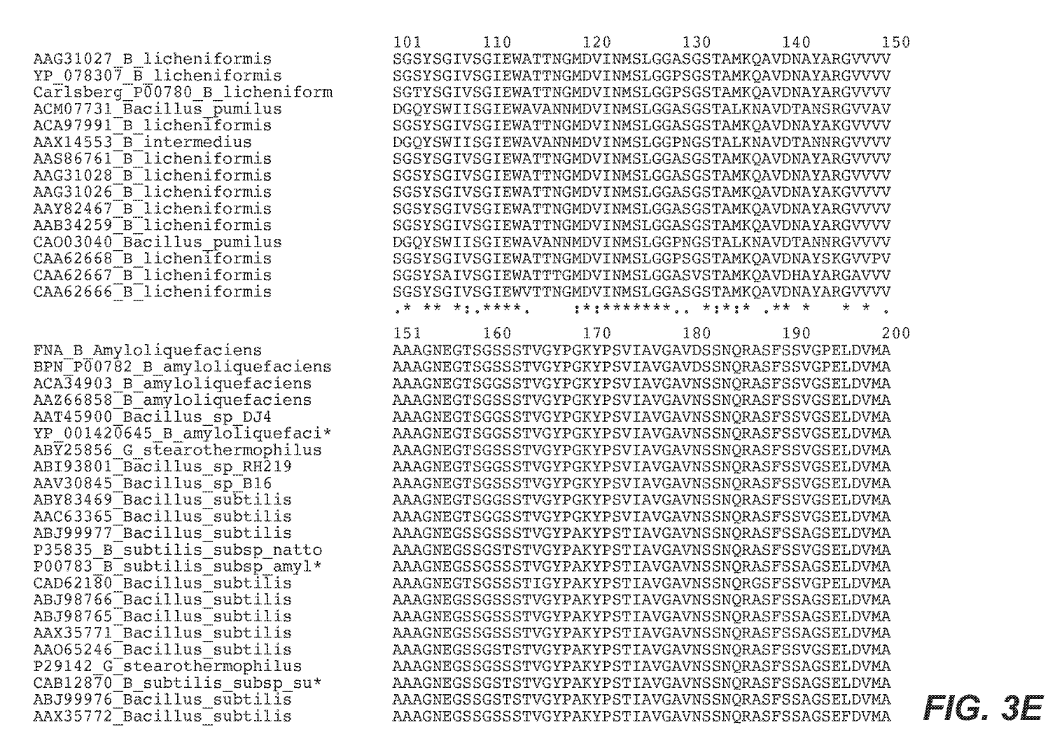

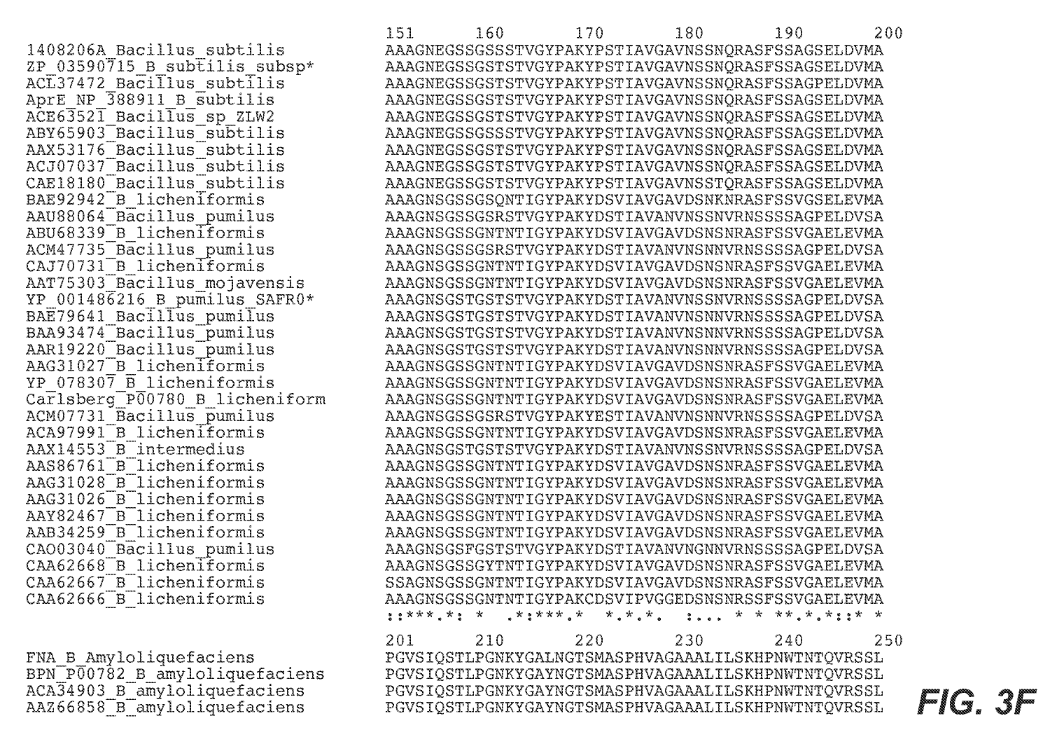

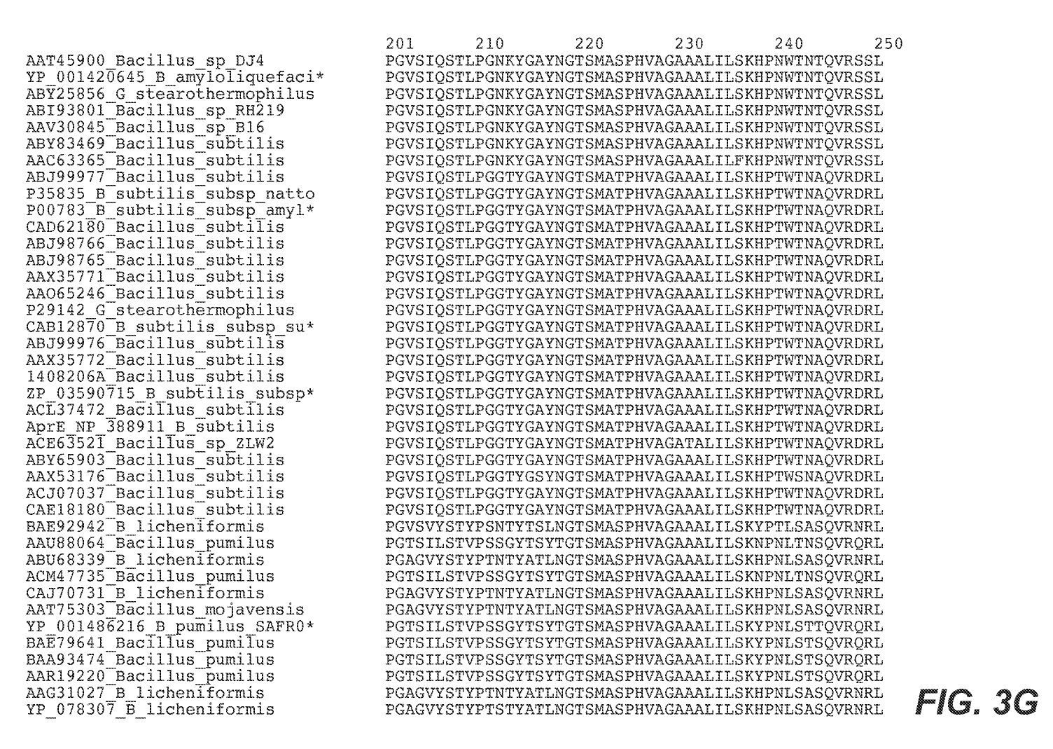

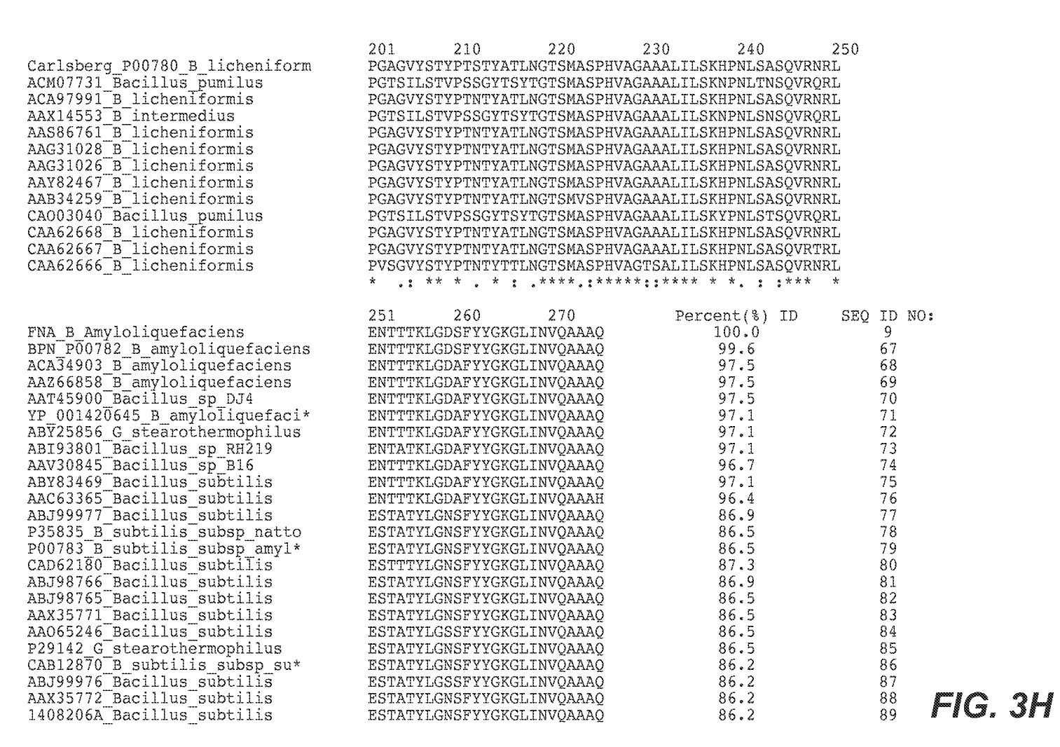

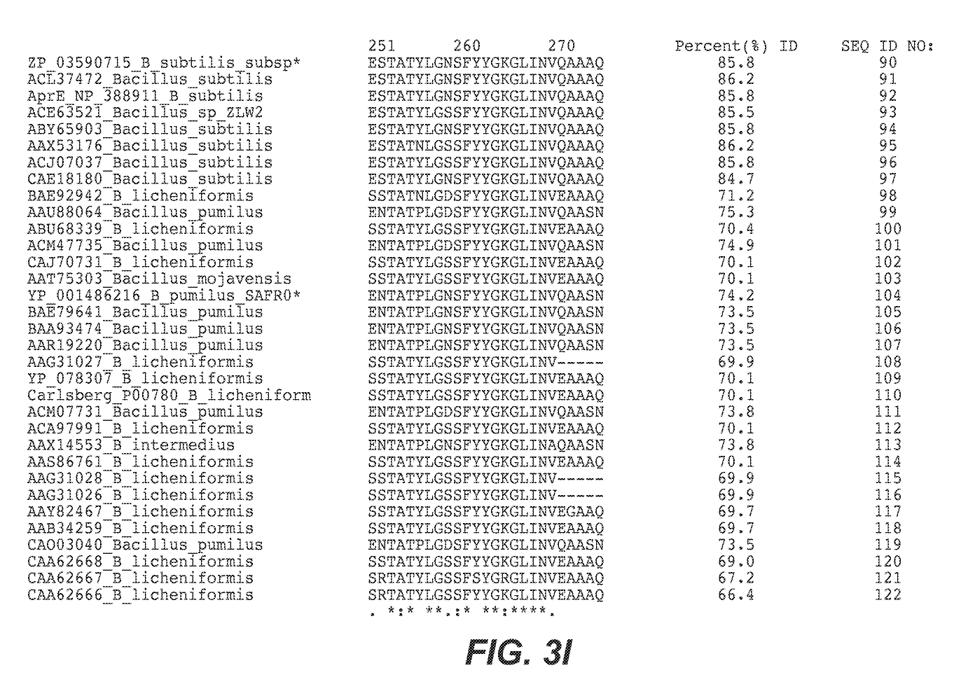

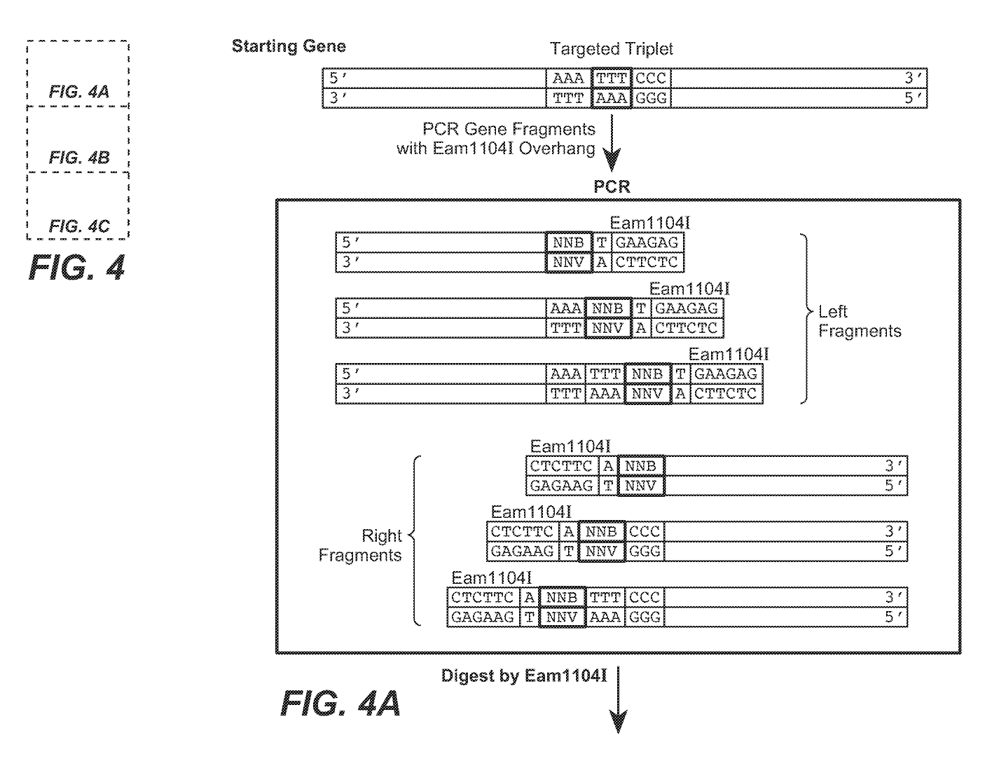

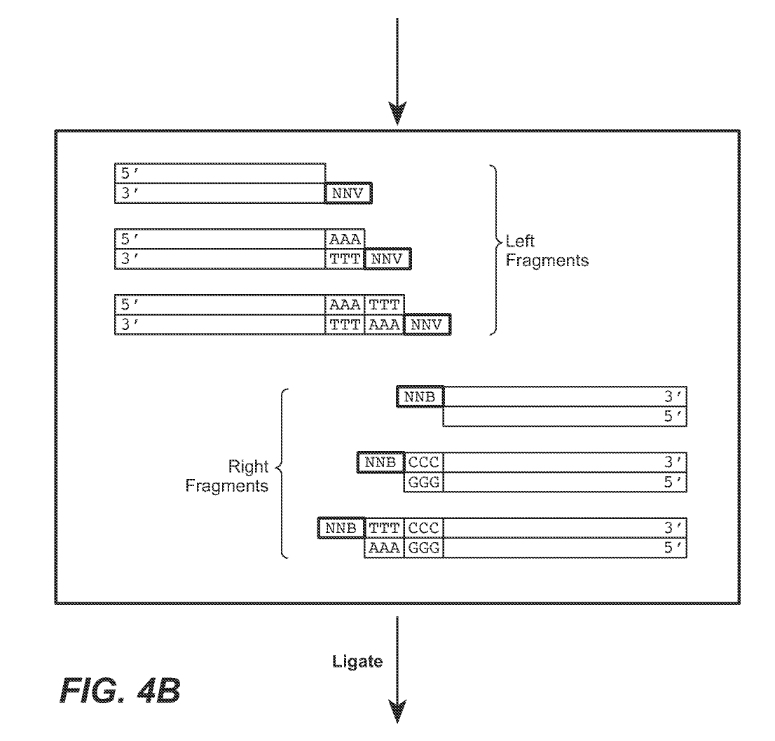

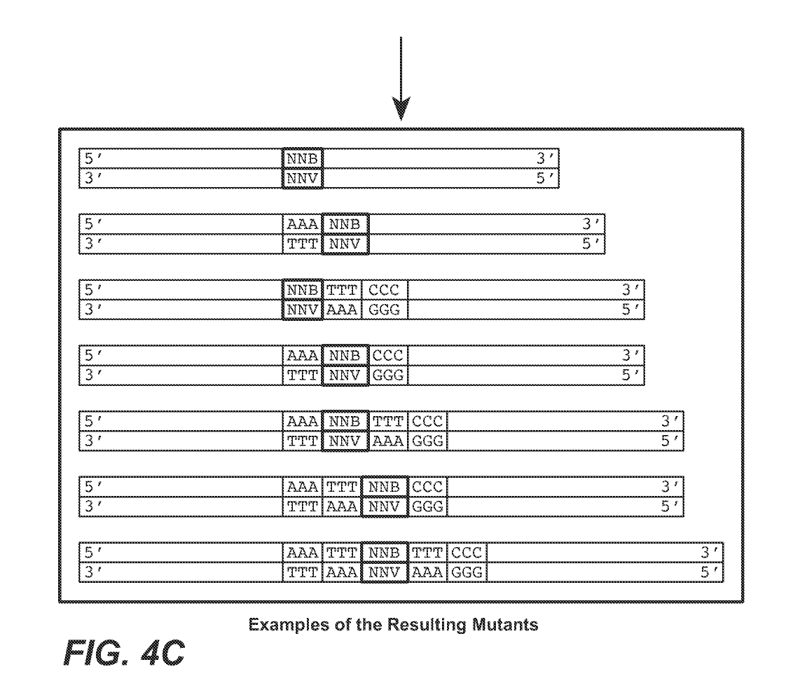

As used herein, “variant” refers to a protein which differs from its corresponding wild-type protein by the addition of one or more amino acids to either or both the C- and N-terminal end, substitution of one or more amino acids at one or a number of different sites in the amino acid sequence, deletion of one or more amino acids at either or both ends of the protein or at one or more sites in the amino acid sequence, and/or insertion of one or more amino acids at one or more sites in the amino acid sequence. A variant protein in the context of the present invention is exemplified by the B. amyloliquifaciens protease FNA (SEQ ID NO:9), which is a variant of the naturally-occurring protein BPN′, from which it differs by a single amino acid substitution Y217L in the mature region. Variant proteases include naturally-occurring homologs. For example, variants of the mature protease of SEQ ID NO:9 include the homologs shown in FIG. 3.

The terms “derived from” and “obtained from” refer to not only a protease produced or producible by a strain of the organism in question, but also a protease encoded by a DNA sequence isolated from such strain and produced in a host organism containing such DNA sequence. Additionally, the term refers to a protease which is encoded by a DNA sequence of synthetic and/or cDNA origin and which has the identifying characteristics of the protease in question. To exemplify, “proteases derived from Bacillus” refers to those enzymes having proteolytic activity which are naturally-produced by Bacillus, as well as to serine proteases like those produced by Bacillus sources but which through the use of genetic engineering techniques are produced by non-Bacillus organisms transformed with a nucleic acid encoding said serine proteases.

A “modified full-length protease” or a “modified protease” are interchangeably used to refer to a full-length protease that comprises a mature region and a pre-pro region that are derived from a parent protease, wherein the pre-pro region is mutated to contain at least one mutation. In some embodiments, the pre-pro region and the mature region are derived from the same parent protease. In other embodiments, the pre-pro region and the mature region are derived from different parent proteases. The modified protease comprises a pre-pro region that is modified to contain at least one mutation, and it is encoded by a modified polynucleotide. The amino acid sequence of the modified protease is said to be “generated” from the precursor protease amino acid sequence by the substitution, deletion or insertion of one or more amino acids of the pre-pro region of the precursor amino acid sequence. In some embodiments, one or more amino acids of the pre-pro region of the precursor protease are substituted to generate the modified full-length protease. Such modification is of the “precursor” or the “parent” DNA sequence which encodes the amino acid sequence of the “precursor” or the “parent” protease rather than manipulation of the precursor protease per se.

The term “enhances” is used herein in reference to the effect of a mutation on the production of a mature protease from a modified precursor being greater than the production of the same mature protease when processed from an unmodified precursor.

The term “full-length protein” herein refers to a primary gene product of a gene and comprising a signal peptide, a pro sequence and a mature sequence. For example, the full-length protease of SEQ ID NO:1 comprises the signal peptide (pre region) (VRSKKLWISL LFALALIFTM AFGSTSSAQA; SEQ ID NO:3, encoded for example by the pre polynucleotide of SEQ ID NO:4), the pro region (AGKSNGEKKY IVGFKQTMST MSAAKKKDVI SEKGGKVQKQ FKYVDAASAT LNEKAVKELK KDPSVAYVEE DHVAHAY; SEQ ID NO:5, encoded for example by the pre polynucleotide

GCAGGGAAATCAAACGGGGAAAAGAAATATATTGTCGGGTTTAAACAGAC
AATGAGCACGATGAGCGCCGCTAAGAAGAAAGATGTCATTTCTGAAAAAG
GCGGGAAAGTGCAAAAGCAATTCAAATATGTAGACGCAGCTTCAGCTACA
TTAAACGAAAAAGCTGTAAAAGAATTGAAAAAAGACCCGAGCGTCGCTT
ACGTTGAAGAAGATCACGTAGCACACGCGTAC: SEQ ID NO: 6),
and the mature region (SEQ ID NO: 9).

The term “signal sequence”, “signal peptide” or “pre region” refers to any sequence of nucleotides and/or amino acids which may participate in the secretion of the mature or precursor forms of the protein. This definition of signal sequence is a functional one, meant to include all those amino acid sequences encoded by the N-terminal portion of the protein gene, which participate in the effectuation of the secretion of protein. To exemplify, a pre peptide of a protease of the present invention at least includes the amino acid sequence identical to residues 1-30 of SEQ ID NO:1.

The term “pro sequence” or “pro region” is an amino acid sequence between the signal sequence and mature protease that is necessary for the secretion/production of the protease. Cleavage of the pro sequence will result in a mature active protease. To exemplify, a pro region of a protease of the present invention at least includes the amino acid sequence identical to residues 31-107 of SEQ ID NO:1.

The term “pre-pro region” or “pre-pro polypeptide” herein refer to the N-terminal region of a protease that encompasses the pre region and the pro region of the full-length protease. To exemplify, a pre-pro region is set forth in SEQ ID NO:7, and it comprises the pro region of SEQ ID NO:5 and the signal peptide (pre region) of SEQ ID NO:3).

The terms “mature form” or “mature region” refer to the final functional portion of the protein. To exemplify, a mature form of the protease of the present invention includes the amino acid sequence identical to residues 108-382 of SEQ ID NO:1. In this context, the “mature form” is “processed from” a full-length protease, wherein the processing of the full-length protease encompasses the removal of the signal peptide and the removal of the pro region.

As used herein, “homologous protein” refers to a protein or polypeptide native or naturally occurring in a cell. Similarly, a “homologous polynucleotide” refers to a polynucleotide that is native or naturally occurring in a cell.

As used herein, the term “heterologous protein” refers to a protein or polypeptide that does not naturally occur in the host cell. Similarly, a “heterologous polynucleotide” refers to a polynucleotide that does not naturally occur in the host cell. Heterologous polypeptides and/or heterologous polynucleotides include chimeric polypeptides and/or polynucleotides.

As used herein, “substituted” and “substitutions” refer to replacement(s) of an amino acid residue or nucleic acid base in a parent sequence. In some embodiments, the substitution involves the replacement of a naturally occurring residue or base. The modified proteases herein encompass the substitution of any of the nineteen naturally occurring amino acids at any one of the amino acid residues of the pre-pro region of the precursor protease. In some embodiments, two or more amino acids are substituted to generate a modified protease that comprises a combination of amino acid substitutions. In some embodiments, combinations of substitutions are denoted by the amino acid position at which the substitution is made. For example, a combination denoted by X49A-X93S means that whichever is the amino acid (X) at position 49 in a parent protein is replaced with an alanine (A), and whichever the amino acid (X) at position 93 in a parent protein is replaced with a serine (S). Amino acid positions are given as corresponding to the numbered position in the full-length parent protein.

As used herein, “deletion” refers to loss of genetic material in which part of a sequence of DNA is missing. While any number of nucleotides can be deleted, deletion of a number of nucleotides that is not evenly divisible by three will lead to a frameshift mutation, causing all of the codons occurring after the deletion to be read incorrectly during translation, producing a severely altered and potentially nonfunctional protein. A deletion can be terminal—a deletion that occurs towards the end of a chromosome, or a deletion can be intercalary deletion—a deletion that occurs from the interior of a gene. Deletions are denoted herein by the amino acid(s) and the position(s) of the amino acid(s) that is/are deleted. For example, p.I18del denotes that isoleucine (I) at position 18 is deleted; and p.I18_T19del denotes that both amino acids isoleucine (I) and threonine (T) at positions 18 and 19, respectively, are deleted.

Deletions of one or more amino acids can be made alone or in combination with one or more substitutions and/or insertions.

As used herein “insertion” refers to the addition of multiples of three nucleotides acids into the DNA to encode the addition of one or more amino acids in the encoded protein. Insertions are denoted herein by the amino acid(s) and the position(s) of the amino acid(s) that is/are inserted. For example, pR2_S3insT denotes that a threonine (T) is inserted between the arginine (R) at position 2 and the serine (S) at position 3. Insertions of one or more amino acids can be made alone or in combination with one or more substitutions and/or deletions.

The term “production” with reference to a protease, encompasses the two processing steps of a full-length protease including: 1. the removal of the signal peptide, which is known to occur during protein secretion; and 2. the removal of the pro region, which creates the active mature form of the enzyme and which is known to occur during the maturation process (Wang et al., Biochemistry 37:3165-3171 (1998); Power et al., Proc Natl Acad Sci USA 83:3096-3100 (1986)).

As used herein, “corresponding to,” and “by correspondence” refer to a residue at the enumerated position in a protein or peptide that is equivalent to an enumerated residue in a reference protein or peptide.

The term “processed” with reference to a mature protease refers to the maturation process that a full-length protein e.g. a protease, undergoes to become an active mature enzyme. The term “enhanced production” herein refers to the production of a mature protease that is processed from a modified full-length protease, that occurs at a level that is greater than the level of production of the same mature protease when processed from an unmodified full-length protease.

“Activity” with respect to enzymes means “catalytic activity” and encompasses any acceptable measure of enzyme activity, such as the rate of activity, the amount of activity, or the specific activity. Catalytic activity refers to the ability to catalyze a specific chemical reaction, such as the hydrolysis of a specific chemical bond. As the skilled artisan will appreciate, the catalytic activity of an enzyme only accelerates the rate of an otherwise slow chemical reaction. Because the enzyme only acts as a catalyst, it is neither produced nor consumed by the reaction itself. The skilled artisan will also appreciate that not all polypeptides have a catalytic activity. “Specific activity” is a measure of activity of an enzyme per unit of total protein or enzyme. Thus, specific activity may be expressed by unit weight (e.g. per gram, or per milligram) or unit volume (e.g. per ml) of enzyme. Further, specific activity may include a measure of purity of the enzyme, or can provide an indication of purity, for example, where a standard of activity is known, or available for comparison. The amount of activity reflects to the amount of enzyme that is produced by the host cell that expresses the enzyme being measured.

The term “relative activity” or “ratio of production” are used herein interchangeably to refer to the ratio of the enzymatic activity of a mature protease that was processed from a modified protease to the enzymatic activity of a mature protease that was processed from an unmodified protease. The ratio of production is determined by dividing the value of the activity of the protease processed from a modified precursor by the value of the activity of the same protease when processed from an unmodified precursor. The relative activity is the ratio of production expressed as a percentage.

As used herein, the term “expression” refers to the process by which a polypeptide is generated based on the nucleic acid sequence of a gene. The process includes both transcription and translation.

The term “chimeric” or “fusion” when used in reference to a protein, herein refer to a protein created through the joining of two or more polynucleotides which originally coded for separate proteins. Translation of this fusion polynucleotide results in a single chimeric polynucleotide with functional properties derived from each of the original proteins. Recombinant fusion proteins are created artificially by recombinant DNA technology. A “chimeric polypeptide,” or “chimera” means a protein containing sequences from more than one polypeptide. A modified protease can be chimeric in the sense that it contains a portion, region, or domain from one protease fused to one or more portions, regions, or domains from one or more other protease. By way of example, a chimeric protease might comprise a sequence for a mature protease linked to the sequence for the pre-pro peptide of another protease. The skilled artisan will appreciate that chimeric polypeptides and proteases need not consist of actual fusions of the protein sequences, but rather, polynucleotides with the corresponding encoding sequences can also be used to express chimeric polypeptides or proteases.

The term “percent (%) identity” is defined as the percentage of amino acid/nucleotide residues in a candidate sequence that are identical with the amino acid residues/nucleotide residues of the precursor sequence (i.e., the parent sequence). A % amino acid sequence identity value is determined by the number of matching identical residues divided by the total number of residues of the “longer” sequence in the aligned region. Amino acid sequences may be similar, but are not “identical” where an amino acid is substituted, deleted, or inserted in the subject sequence relative to the reference sequence. For proteins, the percent sequence identity is preferably measured between sequences that are in a similar state with respect to posttranslational modification. Typically, the “mature sequence” of the subject protease, i.e. the sequence that remains after processing to remove the signal sequence and the pro region, is compared to a mature sequence of the reference protein. In other instances, a precursor sequence of a subject polypeptide sequence may be compared to the precursor of the reference sequence.

As used herein, the term “promoter” refers to a nucleic acid sequence that functions to direct transcription of a downstream gene. In some embodiments, the promoter is appropriate to the host cell in which the target gene is being expressed. The promoter, together with other transcriptional and translational regulatory nucleic acid sequences (also termed “control sequences”) is necessary to express a given gene. In general, the transcriptional and translational regulatory sequences include, but are not limited to, promoter sequences, ribosomal binding sites, transcriptional start and stop sequences, translational start and stop sequences, and enhancer or activator sequences.

A nucleic acid or a polypeptide is “operably linked” when it is placed into a functional relationship with another nucleic acid or polypeptide sequence, respectively. For example, a promoter or enhancer is operably linked to a coding sequence if it affects the transcription of the sequence; a ribosome binding site is operably linked to a coding sequence if it is positioned so as to facilitate translation; or a modified pre-pro region is operably linked to a mature region of a protease if it enables the processing of the full-length protease to produce the mature active form of the enzyme. Generally, “operably linked” means that the DNA or polypeptide sequences being linked are contiguous.

A “host cell” refers to a suitable cell that serves as a host for an expression vector comprising DNA according to the present invention. A suitable host cell may be a naturally occurring or wild-type host cell, or it may be an altered host cell. In one embodiment, the host cell is a Gram positive microorganism. In some embodiments, the term refers to cells in the genus Bacillus.

As used herein, “Bacillus sp.” includes all species within the genus “Bacillus,” as known to those of skill in the art, including but not limited to B. subtilis, B. licheniformis, B. lentus, B. brevis, B. pumilis, B. stearothermophilus, B. alkalophilus, B. amyloliquefaciens, B. clausii, B. halodurans, B. megaterium, B. coagulans, B. circulans, B. lautus, and B. thuringiensis. It is recognized that the genus Bacillus continues to undergo taxonomical reorganization. Thus, it is intended that the genus include species that have been reclassified, including but not limited to such organisms as B. stearothermophilus, which is now named “Geobacillus stearothermophilus.” The production of resistant endospores in the presence of oxygen is considered the defining feature of the genus Bacillus, although this characteristic also applies to the recently named Alicyclobacillus, Amphibacillus, Aneurinibacillus, Anoxybacillus, Brevibacillus, Filobacillus, Gracilibacillus, Halobacillus, Paenibacillus, Salibacillus, Thermobacillus, Ureibacillus, and Virgibacillus.

The terms “polynucleotide” and “nucleic acid”, used interchangeably herein, refer to a polymeric form of nucleotides of any length. These terms include, but are not limited to, a single-, double-stranded DNA, genomic DNA, cDNA, or a polymer comprising purine and pyrimidine bases, or other natural, chemically, biochemically modified, non-natural or derivatized nucleotide bases. Non-limiting examples of polynucleotides include genes, gene fragments, chromosomal fragments, ESTs, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers.

As used herein, the terms “DNA construct” and “transforming DNA” are used interchangeably to refer to DNA used to introduce sequences into a host cell or organism. The DNA construct may be generated in vitro by PCR or any other suitable technique(s) known to those in the art. In some embodiments, the DNA construct comprises a sequence of interest (e.g., a modified sequence). In some embodiments, the sequence is operably linked to additional elements such as control elements (e.g., promoters, etc.). The DNA construct may further comprise a selectable marker. In some embodiments, the DNA construct comprises sequences homologous to the host cell chromosome. In other embodiments, the DNA construct comprises non-homologous sequences. Once the DNA construct is assembled in vitro it may be used to mutagenize a region of the host cell chromosome (i.e., replace an endogenous sequence with a heterologous sequence).

As used herein, the term “expression cassette” refers to a nucleic acid construct generated recombinantly or synthetically, with a series of specified nucleic acid elements that permit transcription of a particular nucleic acid in a target cell. The recombinant expression cassette can be incorporated into a vector such as a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In some embodiments, expression vectors have the ability to incorporate and express heterologous DNA fragments in a host cell. Many prokaryotic and eukaryotic expression vectors are commercially available. Selection of appropriate expression vectors is within the knowledge of those of skill in the art. The term “expression cassette” is used interchangeably herein with “DNA construct,” and their grammatical equivalents. Selection of appropriate expression vectors is within the knowledge of those of skill in the art.

As used herein, the term “heterologous DNA sequence” refers to a DNA sequence that does not naturally occur in a host cell. In some embodiments, a heterologous DNA sequence is a chimeric DNA sequence that is comprised of parts of different genes, including regulatory elements.

As used herein, the term “vector” refers to a polynucleotide construct designed to introduce nucleic acids into one or more cell types. Vectors include cloning vectors, expression vectors, shuttle vectors, and plasmids. In some embodiments, the polynucleotide construct comprises a DNA sequence encoding the full-length protease (e.g., modified protease or unmodified precursor protease). As used herein, the term “plasmid” refers to a circular double-stranded (ds) DNA construct used as a cloning vector, and which forms an extrachromosomal self-replicating genetic element in some eukaryotes or prokaryotes, or integrates into the host chromosome.

As used herein in the context of introducing a nucleic acid sequence into a cell, the term “introduced” refers to any method suitable for transferring the nucleic acid sequence into the cell. Such methods for introduction include but are not limited to protoplast fusion, transfection, transformation, conjugation, and transduction (See e.g., Ferrari et al., “Genetics,” in Hardwood et al, (eds.), Bacillus, Plenum Publishing Corp., pages 57-72, [1989]).

As used herein, the terms “transformed” and “stably transformed” refers to a cell that has a non-native (heterologous) polynucleotide sequence integrated into its genome or as an episomal plasmid that is maintained for at least two generations.

As used herein, the term “expression” refers to the process by which a polypeptide is produced based on the nucleic acid sequence of a gene. The process includes both transcription and translation.

### Modified Proteases

The present invention provides methods and compositions for the production of mature proteases in bacterial host cells. In particular, the invention provides compositions and methods for enhancing the production of mature serine proteases in bacterial cells. The compositions of the invention include modified polynucleotides that encode modified proteases, which have at least one mutation in the pre-pro region, the modified serine proteases encoded by the modified polynucleotides, expression cassettes, DNA constructs, and vectors comprising the modified polynucleotides that encode the modified serine proteases, and the bacterial host cells transformed with the vectors of the invention. The methods of the invention include methods for enhancing the production of mature proteases in bacterial host cells. The produced proteases find use in the industrial production of enzymes, suitable for use in various industries, including but not limited to the cleaning, animal feed and textile processing industry.

In some embodiments, the invention provides a modified full-length polynucleotide encoding a modified full-length protease that is generated by introducing at least one mutation in the pre-pro polynucleotide derived from that encoding a wild-type or full-length variant precursor protease of animal, vegetable or microbial origin. In some embodiments, the precursor protease is of bacterial origin. In some embodiments, the precursor protease is a protease of the subtilisin type (subtilases, subtilopeptidases, EC 3.4.21.62), which comprise catalytically active amino acids, also referred to as serine proteases. In some embodiments, the precursor protease is a Bacillus sp. protease. Preferably, the precursor protease is a serine protease derived from Bacillus subtilis, Bacillus amyloliquifaciens, Bacillus licheniformis and Bacillus pumilis.

Examples of precursor proteases include Subtilisin BPN′ (SEQ ID NO:67), which derives from Bacillus amyloliquefaciens, and is known from the work of Vasantha et al. (1984) in J. Bacteriol., Volume 159, pp. 811-819, and of J. A. Wells et al. (1983) in Nucleic Acids Research, Volume 11, pp. 7911-7925; subtilisin Carlsberg, which is described in the publications of E. L. Smith et al. (1968) in J. Biol. Chem., Volume 243, pp. 2184-2191, and of Jacobs et al. (1985) in Nucl. Acids Res., Volume 13, pp. 8913-8926, and is formed naturally by Bacillus licheniformis, Protease PB92, which is produced naturally by the alkalophilic bacterium Bacillus nov. spec. 92, and AprE which is produced naturally by Bacillus subtilis. In some embodiments, the precursor protease is FNA (SEQ ID NO:1), which is a variant of the naturally occurring BPN′ from which it differs in the mature region by a single amino acid substitution at position 217 of the mature region, wherein the Tyr (Y) at position 217 of BPN′ is substituted to a Leu (L) i.e. the 217^{th} amino acid of the mature region of FNA is L (SEQ ID NO:9). In other embodiments, the precursor protease comprises a pre-pro region that is at least about 30% identical to that of SEQ ID NO:7 (VRSKKLWISL LFALALIFTM AFGSTSSAQA AGKSNGEKKY IVGFKQTMST MSAAKKKDVI SEKGGKVQKQ FKYVDAASAT LNEKAVKELK KDPSVAYVEE DHVAHAY; SEQ ID NO:7) operably linked to the mature region of SEQ ID NO:9

(AQSVPYGVSQIKAPALHSQGYTGSNVKVAVIDSGIDSSHPDLKVAGGA
SMVPSETNPFQDNNSHGTHVAGTVAALNNSIGVLGVAPSASLYAVKVL
GADGSGQYSWIINGIEWAIANNMDVINMSLGGPSGSAALKAAVDKAVAS
GVVVVAAAGNEGTSGSSSTVGYPGKYPSVIAVGAVDSSNQRASFSSVG
PELDVMAPGVSIQSTLPGNKYGALNGTSMASPHVAGAAALILSKHPNWT
NTQVRSSLENTTTKLGDSFYYGKGLINVQAAAQ; SEQ ID NO: 9).

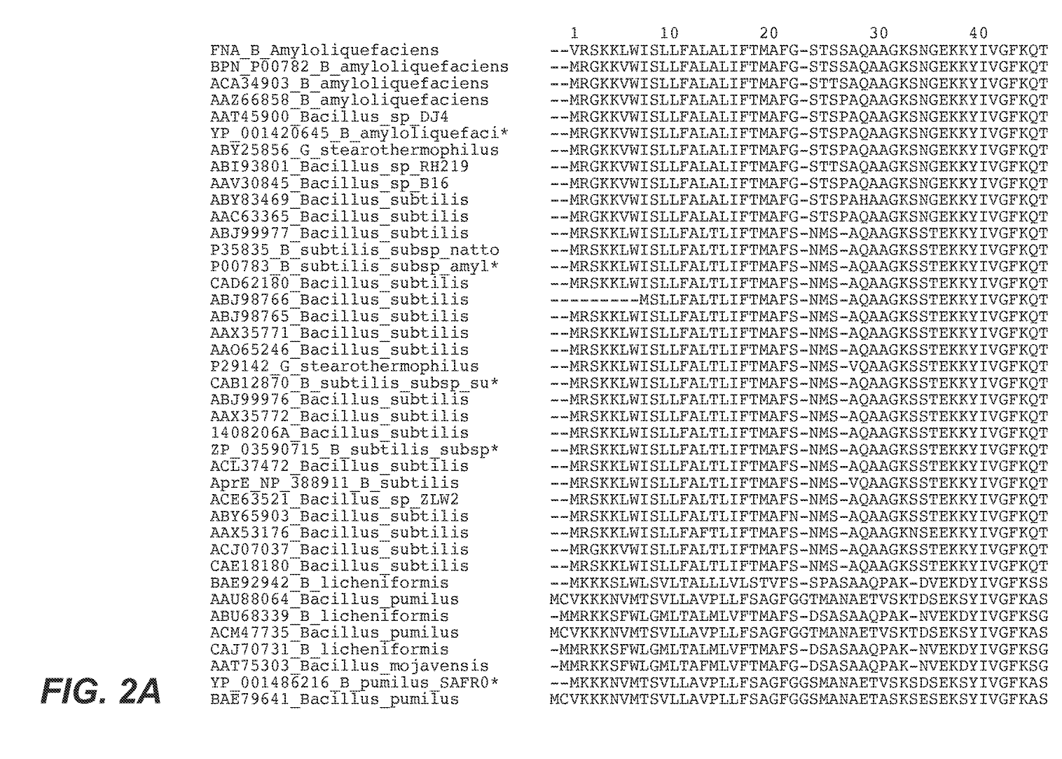

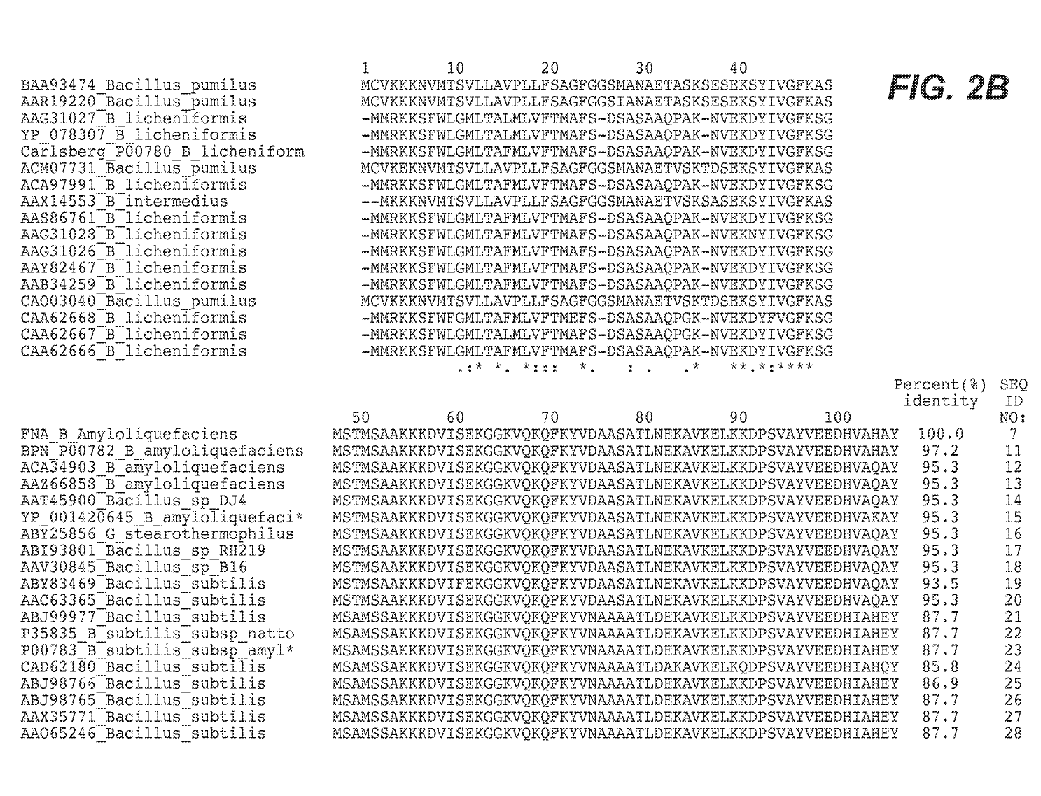

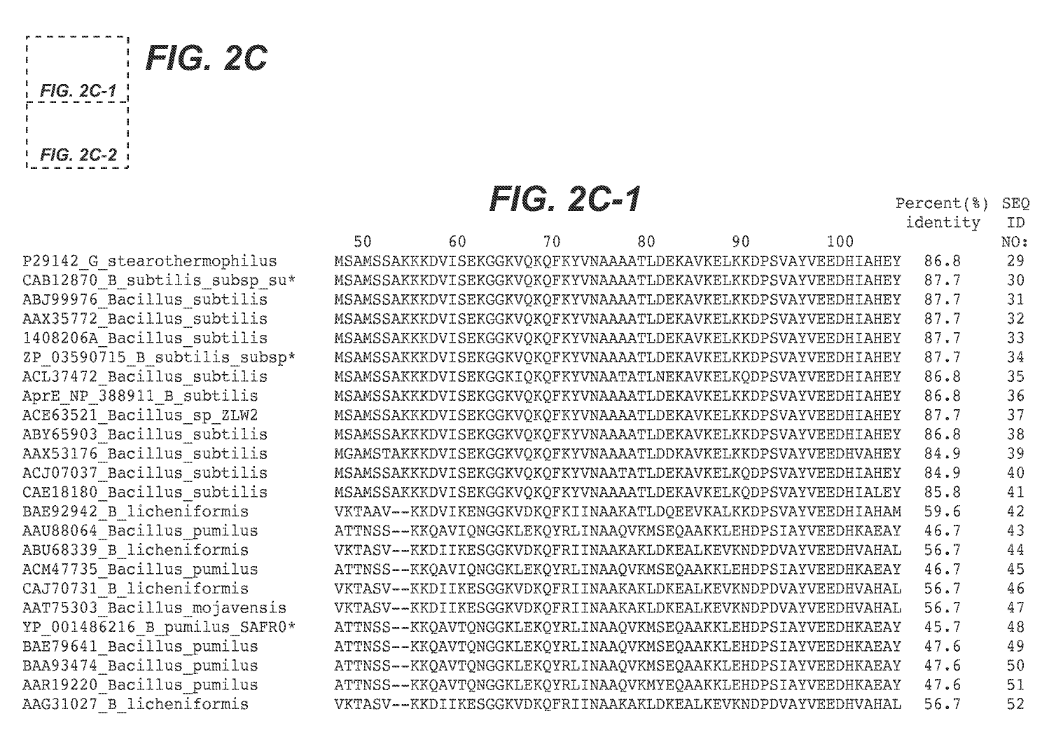

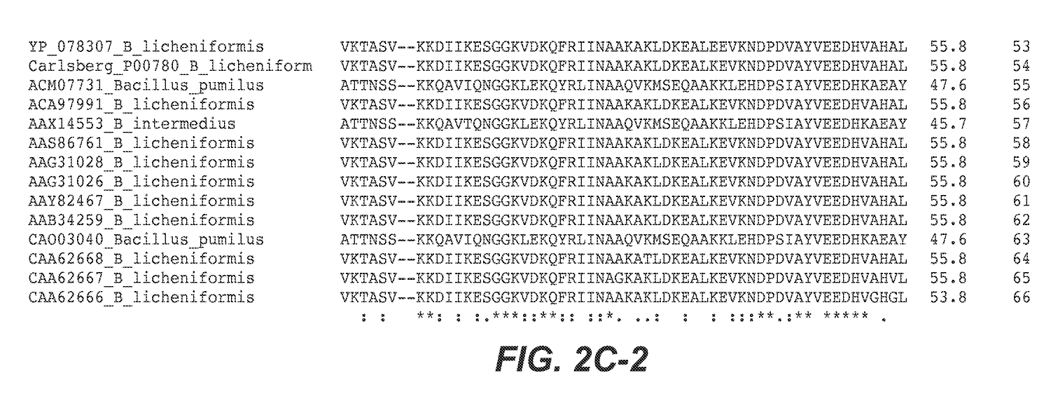

In other embodiments, the precursor protease comprises a pre-pro region that is at least about 30% identical to that of SEQ ID NO:7 operably linked a mature region that is at least about 65% of SEQ ID NO:9. In yet other embodiments, the precursor protease comprises the pre-pro region of SEQ ID NO:7 operably linked to a mature region that is at least about 65% identical to that of SEQ ID NO:9. Examples of pre-pro regions of serine proteases that are at least about 30% identical to the pre-pro region of SEQ ID NO:7 include SEQ ID NOS:11-66 as shown in FIG. 2. Examples of mature regions that are at least about 65% identical to that of SEQ ID NO:9 include SEQ ID NOS:67-122 as shown in FIG. 3.

The percent identity shared by polynucleotide sequences is determined by direct comparison of the sequence information between the molecules by aligning the sequences and determining the identity by methods known in the art. An example of an algorithm that is suitable for determining sequence similarity is the BLAST algorithm, which is described in Altschul, et al., J. Mol. Biol., 215:403-410 (1990). Software for performing BLAST analyses is publicly available through the National Center for Biotechnology Information. This algorithm involves first identifying high scoring sequence pairs (HSPs) by identifying short words of length W in the query sequence that either match or satisfy some positive-valued threshold score T when aligned with a word of the same length in a database sequence. These initial neighborhood word hits act as starting points to find longer HSPs containing them. The word hits are expanded in both directions along each of the two sequences being compared for as far as the cumulative alignment score can be increased. Extension of the word hits is stopped when: the cumulative alignment score falls off by the quantity X from a maximum achieved value; the cumulative score goes to zero or below; or the end of either sequence is reached. The BLAST algorithm parameters W, T, and X determine the sensitivity and speed of the alignment. The BLAST program uses as defaults a wordlength (W) of 11, the BLOSUM62 scoring matrix (See, Henikoff & Henikoff, Proc. Natl. Acad. Sci. USA 89:10915 (1989)) alignments (B) of 50, expectation (E) of 10, M′5, N′-4, and a comparison of both strands.

The BLAST algorithm then performs a statistical analysis of the similarity between two sequences (See e.g., Karlin and Altschul, Proc. Nat'l. Acad. Sci. USA 90:5873-5787 [1993]). One measure of similarity provided by the BLAST algorithm is the smallest sum probability (P(N)), which provides an indication of the probability by which a match between two nucleotide or amino acid sequences would occur by chance. For example, a nucleic acid is considered similar to a serine protease nucleic acid of this invention if the smallest sum probability in a comparison of the test nucleic acid to a serine protease nucleic acid is less than about 0.1, more preferably less than about 0.01, and most preferably less than about 0.001. Where the test nucleic acid encodes a serine protease polypeptide, it is considered similar to a specified serine protease nucleic acid if the comparison results in a smallest sum probability of less than about 0.5, and more preferably less than about 0.2.

The alignments of the amino acid sequences of the pre-pro region (FIG. 2) and the mature region (FIG. 3) of various serine proteases to the pre-pro region and mature region of FNA were obtained using the BLAST program as follows. The pre-pro region of FNA or the mature protein region was used to search the NCBI non-redundant protein database (version Feb. 9, 2009). The command line BLAST program (version 2.2.17) was used with default parameters except for −v 5000 and −b 5000. Only sequences that have the desired eventual percent identity were chosen. The alignment was done using the program clustalw (version 1.83) with default parameters. The alignment was refined five times using the program MUSCLE (version 3.51) with default parameters. Only the regions corresponding to the mature region or pre-pro region of FNA are chosen in the alignment. The sequences in the alignment are ordered in deceasing order according to the percent identities to that of FNA. The percent identity was calculated as the number of identical residues aligned between the two sequences in question divided by the number of residues aligned in the alignment.

In some embodiments, the modified polynucleotides are generated from precursor polynucleotides that comprise a pre-pro polynucleotide encoding a pre-pro region that shares at least about 30%, least about 35%, least about 40%, least about 45%, least about 50%, least about 55%, least about 60%, least about 65% amino acid sequence identity, preferably at least about 70% amino acid sequence identity, more preferably at least about 75% amino acid sequence identity, still more preferably at least about 80% amino acid sequence identity, more preferably at least about 85% amino acid sequence identity, even more preferably at least about 90% amino acid sequence identity, more preferably at least about 92% amino acid sequence identity, yet more preferably at least about 95% amino acid sequence identity, more preferably at least about 97% amino acid sequence identity, still more preferably at least about 98% amino acid sequence identity, and most preferably at least about 99% amino acid sequence identity with the amino acid sequence of the pre-pro region (SEQ ID NO:7) of the precursor protease of SEQ ID NO:1 (FNA) operably linked to the polynucleotide that encodes the mature region set forth in SEQ ID NO:9. Preferably, the modified polynucleotides are generated from precursor polynucleotides that comprise a pre-pro polynucleotide that encodes the pre-pro region of SEQ ID NO:7 operably linked to the polynucleotide that encodes the mature region set forth in SEQ ID NO:9. In other embodiments, the modified polynucleotides are generated from precursor polynucleotides that encode a pre-pro region of any one of SEQ ID NOS: 11-66 operably linked to the polynucleotide that encodes the mature region set forth in SEQ ID NO:9. An example of a polynucleotide that encodes the mature protease of SEQ ID NO:9 is the polynucleotide of SEQ ID NO:10

(GCGCAGTCCGTGCCTTACGGCGTATCACAAATTAAAGCCCCTGCTCTG
CACTCTCAAGGCTACACTGGATCAAATGTTAAAGTAGCGGTTATCGACA
GCGGTATCGATTCTTCTCATCCTGATTTAAAGGTAGCAGGCGGAGCCAG
CATGGTTCCTTCTGAAACAAATCCTTTCCAAGACAACAACTCTCACGGAA
CTCACGTTGCCGGCACAGTTGCGGCTCTTAATAACTCAATCGGTGTATTA
GGCGTTGCGCCAAGCGCATCACTTTACGCTGTAAAAGTTCTCGGTGCTGA
CGGTTCCGGCCAATACAGCTGGATCATTAACGGAATCGAGTGGGCGATC
GCAAACAATATGGACGTTATTAACATGAGCCTCGGCGGACCTTCTGGTTC
TGCTGCTTTAAAAGCGGCAGTTGATAAAGCCGTTGCATCCGGCGTCGTAG
TCGTTGCGGCAGCCGGTAACGAAGGCACTTCCGGCAGCTCAAGCACAGT
GGGCTACCCTGGTAAATACCCTTCTGTCATTGCAGTAGGCGCTGTTGACA
GCAGCAACCAAAGAGCATCTTTCTCAAGCGTAGGACCTGAGCTTGATGTC
ATGGCACCTGGCGTATCTATCCAAAGCACGCTTCCTGGAAACAAATACGG
CGCGTTGAACGGTACATCAATGGCATCTCCGCACGTTGCCGGAGCGGCTG
CTTTGATTCTTTCTAAGCACCCGAACTGGACAAACACTCAAGTCCGCAG
CAGTTTAGAAAACACCACTACAAAACTTGGTGATTCTTTCTACTATGGAA
AAGGGCTGATCAACGTACAGGCGGCAGCTCAGTAA;
SEQ ID NO: 10).

As described above, the pre-pro region polynucleotides are further modified to introduce at least one mutation in the pre-pro region of the encoded polypeptide to enhance the level of production of the mature form of the protease when compared to the level of production of the same mature protease when processed from an unmodified polynucleotide. The modified pre-pro polynucleotides are operably linked to a mature polynucleotide to encode the modified proteases of the invention.

In some embodiments, the modified polynucleotides are generated from precursor polynucleotides that comprise a pre-pro polynucleotide encoding a pre-pro region that shares at least about 30%, least about 35%, least about 40%, least about 45%, least about 50%, least about 55%, least about 60%, least about 65% amino acid sequence identity, preferably at least about 70% amino acid sequence identity, more preferably at least about 75% amino acid sequence identity, still more preferably at least about 80% amino acid sequence identity, more preferably at least about 85% amino acid sequence identity, even more preferably at least about 90% amino acid sequence identity, more preferably at least about 92% amino acid sequence identity, yet more preferably at least about 95% amino acid sequence identity, more preferably at least about 97% amino acid sequence identity, still more preferably at least about 98% amino acid sequence identity, and most preferably at least about 99% amino acid sequence identity with the amino acid sequence of the pre-pro region (SEQ ID NO:7) of the precursor protease of SEQ ID NO:1 operably linked to the polynucleotide that encodes a mature region of a protease that shares at least about 65% amino acid sequence identity, preferably at least about 70% amino acid sequence identity, more preferably at least about 75% amino acid sequence identity, still more preferably at least about 80% amino acid sequence identity, more preferably at least about 85% amino acid sequence identity, even more preferably at least about 90% amino acid sequence identity, more preferably at least about 92% amino acid sequence identity, yet more preferably at least about 95% amino acid sequence identity, more preferably at least about 97% amino acid sequence identity, still more preferably at least about 98% amino acid sequence identity, and most preferably at least about 99% amino acid sequence identity with the amino acid sequence of the mature region (SEQ ID NO:9) of the precursor protease of SEQ ID NO:1.

In some embodiments, the modified polynucleotides are generated from a precursor polynucleotide that encodes the pro-pro region (SEQ ID NO:7) of the protease of SEQ ID NO:1 operably linked to the mature region of a protease that shares at least about 65% amino acid sequence identity, preferably at least about 70% amino acid sequence identity, more preferably at least about 75% amino acid sequence identity, still more preferably at least about 80% amino acid sequence identity, more preferably at least about 85% amino acid sequence identity, even more preferably at least about 90% amino acid sequence identity, more preferably at least about 92% amino acid sequence identity, yet more preferably at least about 95% amino acid sequence identity, more preferably at least about 97% amino acid sequence identity, still more preferably at least about 98% amino acid sequence identity, and most preferably at least about 99% amino acid sequence identity with the amino acid sequence of the mature form (SEQ ID NO:9) of the precursor protease of SEQ ID NO:1.

In yet other embodiments, the modified polynucleotides are generated from a precursor polynucleotide that encodes the pro-pro region (SEQ ID NO:7) of the protease of SEQ ID NO:1 operably linked to the mature region (SEQ ID NO:9) of the protease of SEQ ID NO:1, i.e. the precursor polynucleotide encodes the protease of SEQ ID NO:1. As described above, the pre-pro region polynucleotides are modified to introduce at least one mutation that enhances the level of production of the mature form of the protease when compared to the level of production of the same mature protease when processed from an unmodified polynucleotide.

The precursor polynucleotides are mutated to generate the modified polynucleotides of the invention. In some embodiments, the portion of a precursor polynucleotide sequence encoding a pre-pro region is mutated to encode at least one mutation at least at one amino acid position selected from positions 1-107, wherein the positions are numbered by correspondence with the amino acid sequence of the pre-pro polypeptide of the FNA protease set forth as SEQ ID NO:7. Thus, in some embodiments, the modified full-length polynucleotides of the invention comprise at least one mutation at least at one amino acid position selected from positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 85, 86, 87, 88, 89, 90, 91, 92, 93, 94, 95, 96, 97, 98, 99, 100, 101, 102, 103, 104, 105, 106, and 107 wherein the positions are numbered by correspondence with the amino acid sequence of the pre-pro polypeptide of the FNA protease set forth as SEQ ID NO:7.

In other embodiments, the modified full-length polynucleotide s comprise at least one mutation at amino acid positions 2, 3, 6, 7, 8, 10, 11, 12, 13, 14, 15, 16, 17, 19, 20, 21, 22, 23, 24, 25, 26, 27, 28, 29, 30, 31, 32, 33, 34, 35, 36, 37, 38, 39, 45, 46, 47, 48, 49, 50, 51, 52, 53, 54, 55, 57, 58, 59, 61, 62, 63, 64, 66, 67, 68, 69, 70, 72, 74, 75, 76, 77, 78, 80, 82, 83, 84, 87, 88, 89, 90, 91, 93, 96, 100, and 102, wherein the positions are numbered by correspondence with the amino acid sequence of the pre-pro polypeptide of the FNA protease set forth as SEQ ID NO:7.

In some embodiments, the at least one mutation is a substitution chosen from the following substitutions: X2F, N, P, and Y; X3A, M, P, and R; X6K, and M; X7E; I8W; X10A, C, G, M, and T; X11A, F, and T; X12C, P, T; X13C, G, and S; X14F; X15G, M, T, and V; X16V; X17S; X19P, and S; X20V; X21S; X22E; X23F, Q, and W; X24G, T and V; X25A, D, and W; X26C, and H; X27A, F, H, P, T, V, and Y; X28V; X29E, I, R, S, and T; X30C; X31H, K, N, S, V, and W; X32C, F, M, N, P, S, and V; X33E, F, M, P, and S; X34D, H, P, and V; X35C, Q, and S; X36C, D, L, N, S, W, and Y; X37C, G, K, and Q; X38F, Q, S, and W; X39A, C, G, I, L, M, P, S, T, and V; X45G and S; X46S; X47E and F; X48G, I, T, W, and Y; X49A, C, E and I; X50D, and Y; X51A and H; X52A, H, I, and M; X53D, E, M, Q, and T; X54F, G, H, I, and S; X55D; X57E, N, and R; X58A, C, E, F, G, K, R, S, T, W; X59E; X61A, F, I, and R; X62A, F, G, H, N, S, T and V; X63A, C, E, F, G, N, Q, R, and T; G64D, M, Q, and S; X66E; X67G and L; X68C, D, and R; X69Y; X70E, G, K, L, M, P, S, and V; X72D and N; X74C and Y; X75G; X76V; X77E, V, and Y; X78M, Q and V; X80D, L, and N; X82C, D, P, Q, S, and T; X83G, and N; X84M; X87R; X88A, D, G, T, and V; X89V; X90D and Q; X91A; X92E and S; X93G, N, and S; X96G, N, and T; X100Q; and X102T, wherein the positions are numbered by correspondence with the amino acid sequence of the pre-pro polypeptide of the FNA protease set forth as SEQ ID NO:7. In other embodiments, the at least one mutation is a combination of substitutions chosen from X49A-X24T, X49A-X72D, X49A-X78M, X49A-X78V, X49A-X93S, X49C-X24T, X49C-X72D, X49C-X78M, X49C-X78V, X490-X91A, X49C-X93S, X91A-x24T, X91A-X49A, X91A-X52H, X91A-X72D, X91A-X78M, X91A-X78V, X93S-X24T, X93S-X49C, X93S-X52H, X93S-X72D, X93S-X78M, and X93S-X78V, wherein the positions are numbered by correspondence with the amino acid sequence of the pre-pro polypeptide of the FNA protease set forth as SEQ ID NO:7.

In some embodiments, the at least one mutation encodes at least one deletion selected from p.X18_X19del, p.X22_{—}23del, pX37del, pX49del, p.X47del, pX55del and p.X57del, wherein the positions are numbered by correspondence with the amino acid sequence of the pre-pro polypeptide of the FNA protease set forth as SEQ ID NO:7.

In some embodiments, the at least one mutation encodes at least one insertion selected from p.X2_X3insT, p.X30_X31insA, p.X19_X20insAT, p.X21_X22insS, p.X32_X33insG, p.X36_X37insG, and p.X58_X59insA, wherein the positions are numbered by correspondence with the amino acid sequence of the pre-pro polypeptide of the FNA protease set forth as SEQ ID NO:7.

In some embodiments, the at least one mutation encodes at least one substitution and at least one deletion selected from X46H-p.X47del, X49A-p.X22_X23del, x49C-p.X22_X23del, X48I-p.X49del, X17W-p.X18_X19del, X78M-p.X22_X23del, X78V-p.X22_X23del, X78V-p.X57del, X91A-p.X22_X23del, X91A-X48I-pX49del, X91A-p.X57del, X93S-p.X22_X23del, and X93S-X48I-p.X49del, and wherein the positions are numbered by correspondence with the amino acid sequence of the pre-pro polypeptide of the FNA protease set forth as SEQ ID NO:7.

In some embodiments, the at least one mutation encodes at least one substitution and at least one insertion selected from X49A-p.X2_X3insT, X49A-p32X_X33insG, X49A-p.X19_X20insAT, X49C-p.X19_X20insAT, X49-p.X32_X33insG, X52H-p.X19_X20insAT, X72D-p.X19_X20insAT, X78M-p.X19_X20insAT, X78V-p.X19_X20insAT, X91A-p.X19_X20insAT, X91A-p.X32_X33insG, X93S-p.X19_X20insAT, and X93S-p.X32_X33insG, and wherein the positions are numbered by correspondence with the amino acid sequence of the pre-pro polypeptide of the FNA protease set forth as SEQ ID NO:7.

In some embodiments, the at least one mutation encodes at least two mutations encoding at least one deletion and at least one insertion selected from p.X57del-p.X19_X20insAT, and p.X 22_X23del-p.X2_X3insT, and wherein the positions are numbered by correspondence with the amino acid sequence of the pre-pro polypeptide of the FNA protease set forth as SEQ ID NO:7.

In some embodiments, the at least one mutation encodes at least three mutations encoding at least one deletion, one insertion and one substitution corresponding to p.S49del-p.T19_M20insAT-M48I, wherein the positions are numbered by correspondence with the amino acid sequence of the pre-pro polypeptide of the FNA protease set forth as SEQ ID NO:7.

In some embodiments, the precursor polynucleotide encodes the full-length FNA protease of SEQ ID NO:1. In some embodiments, the precursor polynucleotide that encodes the encodes the full-length FNA protease of SEQ ID NO:1 is the polynucleotide of SEQ ID NO:2. Modified full-length polynucleotides are generated from the precursor polynucleotide of SEQ ID NO:2 by introducing at least one mutation in the pre-pro region (SEQ ID NO:4) of the precursor polynucleotide (SEQ ID NO:2). In some embodiments, the at least one mutation is at least one substitution chosen from at least one substitution selected from R2F, N, P, and Y; S3A, M, P, and R; L6K, and M; W7E; I8W; L10A, C, G, M, and T; L11A, F, and T; F12C, P, T; A13C, G, and S; L14F; A15G, M, T, and V; L16V; I17S; T19P, and S; M20V; A21S; F22E; G23F, Q, and W; S24G, T and V; T25A, D, and W; S26C, and H; S27A, F, H, P, T, V, and Y; A28V; Q29E, I, R, S, and T; A30C; A31H, K, N, S, V, and W; G32C, F, M, N, P, S, and T; K33E, F, M, P, and S; S34D, H, P, and V; N35C, Q, and S; G36C, D, L, N, S, W, and Y; E37C, G, K, and Q; K38F, Q, S, and W; K39A, C, G, I, L, M, P, S, T, and V; K45G and S; Q46S; T47E and F; M48G, I, T, W, and Y; S49A, C, E and I; T50D, and Y; M51A and H; S52A, H, I, and M; A53D, E, M, Q, and T; A54F, G, H, I, and S; K55D; K57E, N, and R; D58A, C, E, F, G, K, R, S, T, W; V59E; S61A, F, I, and R; E62A, F, G, H, N, S, T and V; K63A, C, E, F, G, N, Q, R, and T; 64D, M, Q, and S; K66E; V67G and L; Q68C, D, and R; K69Y; Q70E, G, K, L, M, P, S, and V; K72D and N; V74C and Y; D75G; A76V; A77E, V, and Y; S78M, Q and V; T80D, L, and N; N82C, D, P, Q, S, and T; E83G, and N; K84M; K87R; E88A, D, G, T, and V; L89V; K90D and Q; K91A; D92E and S; P93G, N, and S; A96G, N, and T; E100Q; and H102T, wherein the positions are numbered by correspondence with the amino acid sequence of the pre-pro polypeptide of the FNA protease set forth as SEQ ID NO:7.

In some embodiments, the precursor FNA polynucleotide is mutated to encode a modified full-length FNA comprising in its pre-pro region least one combination of mutations encoding a combination of substitutions selected from S49A-S24T, S49A-K72D, S49A-S78M, S49A-S78V, S49A-P93S, S49C-S24T, S49C-K72D, S49C-S78M, S49C-S78V, S49C-K91A, S49C-P93S, K91A-S24T, K91A-S49A, K91A-S52H, K91A-K72D, K91A-S78M, K91A-S78V, P93S-S24T, P93S-S49C, P93S-S52H, P93S-K72D, P93S-S78M, and P93S-S78V, wherein the positions are numbered by correspondence with the amino acid sequence of the pre-pro polypeptide of the FNA protease set forth as SEQ ID NO:7.

In some embodiments, the precursor FNA polynucleotide is mutated to encode a modified full-length FNA comprising in its pre-pro region at least one mutation encoding at least one deletion selected from p.I18_T19del, p.F22_G23del, p.E37del, p.T47del 466, p.S49del, p.K55del, and p.K57del, wherein the positions are numbered by correspondence with the amino acid sequence of the pre-pro polypeptide of the FNA protease set forth as SEQ ID NO:7.

In some embodiments, the precursor FNA polynucleotide is mutated to encode a modified full-length FNA comprising in its pre-pro region at least one mutation encoding at least one insertion selected from p.R2_S3insT, p.A30_A31insA, p.T19_M20insAT, p.A21_F22insS, p.G32_K33insG, p.G36_E37insG, and p.D58_V59insA, wherein the positions are numbered by correspondence with the amino acid sequence of the pre-pro polypeptide of the FNA protease set forth as SEQ ID NO:7.

In some embodiments, the precursor FNA polynucleotide is mutated to encode a modified full-length FNA comprising in its pre-pro region at least two mutations encoding at least one substitution and at least one deletion selected from the group consisting of Q46H-p.T47del, S49A-p.F22_G23del, S49C-p.F22_G23del, M48I-p.S49del, I17W-p.I18_T19del, S78M-p.F22_G23del, S78V-p.F22_G23del, K91A-p.F22_G23del, K91A-M48I-pS49del, K91A-p.K57del, P93S-p.F22_G23del, and P93S-M48I-p.S49del, wherein the positions are numbered by correspondence with the amino acid sequence of the pre-pro polypeptide of the FNA protease set forth as SEQ ID NO:7.

In some embodiments, the precursor FNA polynucleotide is mutated to encode a modified full-length FNA comprising in its pre-pro region at least two mutations encoding at least one substitution and at least one insertion selected from S49A-p.R2_S3insT, S49A-p32G_K33insG, S49A-p.T19_M20insAT, S49C-p.T19_M20insAT, S49C-p.G32_K33insG, S49C-p.T19_M20insAT, S52H-p.T19_M20insAT, K72D-p.T19_M20insAT, 578M-p.T19_M20insAT, 578V-p.T19_M20insAT, K91A-p.T19_M20insAT, K91A-p.G32_K33insG, P93S-p.T19_M20insAT, and P93S-p.G32_K33insG, wherein the positions are numbered by correspondence with the amino acid sequence of the pre-pro polypeptide of the FNA protease set forth as SEQ ID NO:7.

In some embodiments, the precursor FNA polynucleotide is mutated to encode a modified full-length FNA comprising in its pre-pro region at least at least two mutations encoding a deletion and an insertion selected from pK57del-p.T19_M20insAT, and p.F22_G23del-p.R2_S3insT, wherein the positions are numbered by correspondence with the amino acid sequence of the pre-pro polypeptide of the FNA protease set forth as SEQ ID NO:7.

In some embodiments, the precursor FNA polynucleotide is mutated to encode a modified full-length FNA comprising in its pre-pro region at least three mutations encoding at least one deletion, one insertion and one substitution corresponding to p.S49del-p.T19_M20insAT-M48I, wherein the positions are numbered by correspondence with the amino acid sequence of the pre-pro polypeptide of the FNA protease set forth as SEQ ID NO:7.

The modification of the pre-pro region of the precursor proteases of the invention includes at least one substitution, at least one deletion, or at least one insertion. In some embodiments, the modification of the pre-pro region includes a combination of mutations. For example, modification of the pre-pro region includes a combination of at least one substitution and at least one deletion. In other embodiments, modification of the pre-pro region includes a combination of at least one substitution and at least one insertion. In other embodiments, modification of the pre-pro region includes a combination of at least one deletion and at least one insertion. In yet other embodiments, modification of the pre-pro region includes a combination of at least one substitution, at least one deletion, and at least one insertion.

Several methods are known in the art that are suitable for generating modified polynucleotide sequences of the present invention, including but not limited to site-saturation mutagenesis, scanning mutagenesis, insertional mutagenesis, deletion mutagenesis, random mutagenesis, site-directed mutagenesis, and directed-evolution, as well as various other recombinatorial approaches. The commonly used methods include DNA shuffling (Stemmer W P, Proc Natl Acad Sci USA. 25; 91(22):10747-51 [1994]), methods based on non-homologous recombination of genes e.g. ITCHY (Ostermeier et al., Bioorg Med. Chem. 7(10):2139-44 [1999]), SCRACHY (Lutz et al. Proc Natl Acad Sci USA. 98(20):11248-53 [2001]), SHIPREC (Sieber et al., Nat. Biotechnol. 19(5):456-60 [2001]), and NRR (Bittker et al., Nat. Biotechnol. 20(10):1024-9 [2001]; Bittker et al., Proc Natl Acad Sci USA. 101(18):7011-6 [2004]), and methods that rely on the use of oligonucleotides to insert random and targeted mutations, deletions and/or insertions (Ness et al., Nat. Biotechnol. 20(12):1251-5 [2002]; Coco et al., Nat. Biotechnol. 20(12):1246-50 [2002]; Zha et al., Chembiochem. 3; 4(1):34-9 [2003], Glaser et al., J. Immunol. 149(12):3903-13 [1992], Sondek and Shortle, Proc Natl Acad Sci USA 89(8):3581-5 [1992], Yáñez et al., Nucleic Acids Res. 32(20):e158 [2004], Osuna et al., Nucleic Acids Res. 32(17):e136 [2004], Gaytán et al., Nucleic Acids Res. 29(3):E9 [2001], and Gaytán et al., Nucleic Acids Res. 30(16):e84 [2002]).

In some embodiments, the full-length parent polynucleotide is ligated into an appropriate expression plasmid, and the following mutagenesis method may be used to facilitate the construction of the modified protease of the present invention, although other methods may be used. The method is based on that described by Pisarchik et al. (Protein engineering, Design and Selection 20:257-265 [2007]) with the added advantage that the restriction enzyme used herein cuts outside its recognition sequence, which allows digestion of practically any nucleotide sequence and precludes formation of a restriction site scar. First, as described herein, a naturally-occurring gene encoding the full-length protease is obtained and sequenced in whole or in part. Subsequently, the pre-pro sequence is scanned for one or more points at which it is desired to make a mutation (deletion, insertion, substitution, or a combination thereof) at one or more amino acids in the encoded pre-pro region. Mutation of the gene in order to change its sequence to conform to the desired sequence is accomplished by primer extension in accord with generally known methods. Fragments to the left and to the right of the desired point(s) of mutation are amplified by PCR and to include the Eam1104I restriction site. The left and right fragments are digested with Eam1104I to generate a plurality of fragments having complimentary three base overhangs, which are then pooled and ligated to generate a library of modified pre-pro sequences containing one or more mutations. The method is diagrammed in FIG. 2. This method avoids the occurrence of frame-shift mutations. In addition, this method simplifies the mutagenesis process because all of the oligonucleotides can be synthesized so as to have the same restriction site, and no synthetic linkers are necessary to create the restriction sites as is required by some other methods.

As indicated above, in some embodiments, the present invention provides vectors comprising the aforementioned polynucleotides. In some embodiments, the vector is an expression vector in which the modified polynucleotide sequence encoding the modified protease of the invention is operably linked to additional segments required for efficient gene expression (e.g., a promoter operably linked to the gene of interest). In some embodiments, these necessary elements are supplied as the gene's own homologous promoter if it is recognized, (i.e., transcribed by the host), and a transcription terminator that is exogenous or is supplied by the endogenous terminator region of the protease gene. In some embodiments, a selection gene such as an antibiotic resistance gene that enables continuous cultural maintenance of plasmid-infected host cells by growth in antimicrobial-containing media is also included.

In some embodiments, the expression vector is derived from plasmid or viral DNA, or in alternative embodiments, contains elements of both. Exemplary vectors include, but are not limited to pXX, pC194, pJH101, pE194, pHP13 (Harwood and Cutting (eds), Molecular Biological Methods for Bacillus, John Wiley & Sons, [1990], in particular, chapter 3; suitable replicating plasmids for B. subtilis include those listed on page 92; Perego, M. (1993) Integrational Vectors for Genetic Manipulations in Bacillus subtilis, p. 615-624; A. L. Sonenshein, J. A. Hoch, and R. Losick (ed.), Bacillus subtilis and other Gram-positive bacteria: biochemistry, physiology and molecular genetics, American Society for Microbiology, Washington, D.C.).

For expression and production of protein(s) of interest e.g. a protease, in a cell, at least one expression vector comprising at least one copy of a polynucleotide encoding the modified protease, and preferably comprising multiple copies, is transformed into the cell under conditions suitable for expression of the protease. In some particularly embodiments, the sequences encoding the proteases (as well as other sequences included in the vector) are integrated into the genome of the host cell, while in other embodiments, the plasmids remain as autonomous extra-chromosomal elements within the cell. Thus, the present invention provides both extrachromosomal elements as well as incoming sequences that are integrated into the host cell genome.

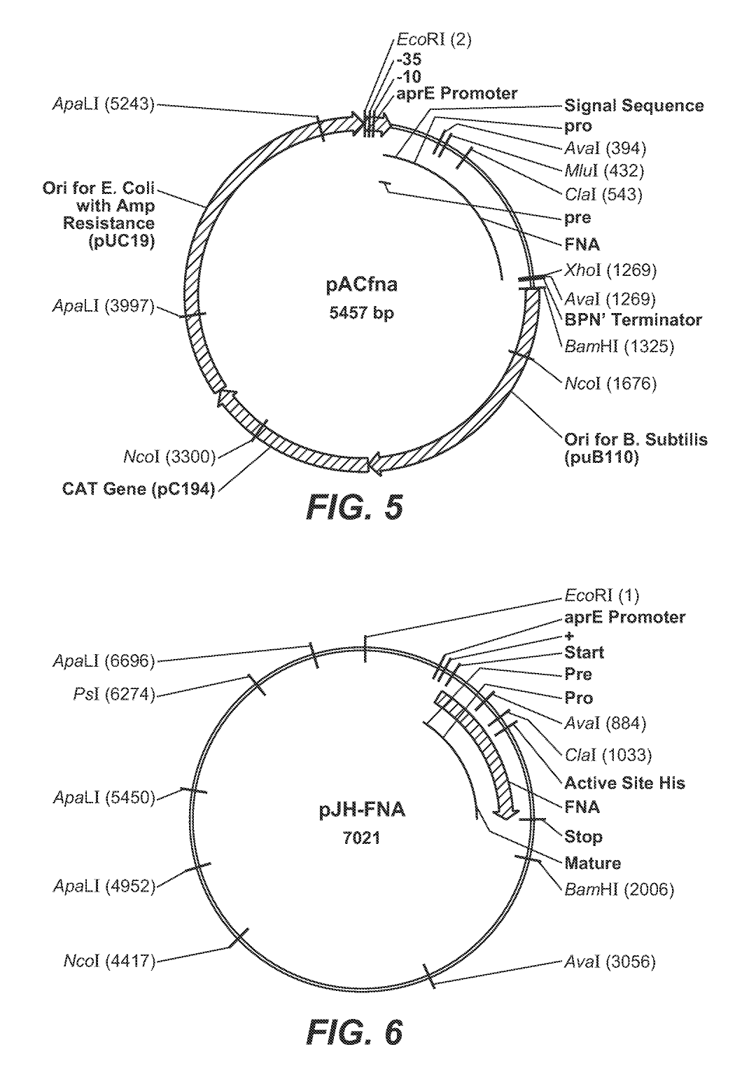

In some embodiments, a replicating vector finds use in the construction of vectors comprising the polynucleotides described herein (e.g., pAC-FNA; See, FIG. 5). It is intended that each of the vectors described herein will find use in the present invention. In some embodiments, the construct is present on an integrating vector (e.g., pJH-FNA; FIG. 6), that enables the integration and optionally the amplification of the modified polynucleotide into the bacterial chromosome. Examples of sites for integration include, but are not limited to the aprE, the amyE, the veg or the pps regions. Indeed, it is contemplated that other sites known to those skilled in the art will find use in the present invention. In some embodiments, the promoter is the wild-type promoter for the selected precursor protease. In some other embodiments, the promoter is heterologous to the precursor protease, but is functional in the host cell. Specifically, examples of suitable promoters for use in bacterial host cells include but are not limited to the pSPAC, pAprE, pAmyE, pVeg, pHpall promoters, the promoter of the B. stearothermophilus maltogenic amylase gene, the B. amyloliquefaciens (BAN) amylase gene, the B. subtilis alkaline protease gene, the B. clausii alkaline protease gene the B. pumilus xylosidase gene, the B. thuringiensis cryIIIA, and the B. licheniformis alpha-amylase gene. In some embodiments, the promoter has a sequence set forth in SEQ ID NO:333. In other embodiments, the promoter has a sequence set forth in SEQ ID NO:445. Additional promoters include, but are not limited to the A4 promoter, as well as phage Lambda P_{R} or P_{L} promoters, and the E. coli lac, trp or tac promoters.

Precursor and modified proteases are produced in host cells of any suitable Gram-positive microorganism, including bacteria and fungi. For example, in some embodiments, the modified protease is produced in host cells of fungal and/or bacterial origin. In some embodiments, the host cells are Bacillus sp., Streptomyces sp., Escherichia sp. or Aspergillus sp. In some embodiments, the modified proteases are produced by Bacillus sp. host cells. Examples of Bacillus sp. host cells that find use in the production of the modified proteins of the present invention include, but are not limited to B. licheniformis, B. lentus, B. subtilis, B. amyloliquefaciens, B. lentus, B. brevis, B. stearothermophilus, B. alkalophilus, B. coagulans, B. circulans, B. pumilus, B. thuringiensis, B. clausii, and B. megaterium, as well as other organisms within the genus Bacillus. In some embodiments, B. subtilis host cells find use. U.S. Pat. Nos. 5,264,366 and 4,760,025 (RE 34,606) describe various Bacillus host strains that find use in the present invention, although other suitable strains find use in the present invention.

Several industrial strains that find use in the present invention include non-recombinant (i.e., wild-type) Bacillus sp. strains, as well as variants of naturally occurring strains and/or recombinant strains. In some embodiments, the host strain is a recombinant strain, wherein a polynucleotide encoding a polypeptide of interest has been introduced into the host. In some embodiments, the host strain is a B. subtilis host strain and particularly a recombinant Bacillus subtilis host strain. Numerous B. subtilis strains are known, including but not limited to 1A6 (ATCC 39085), 168 (1A01), SB19, W23, Ts85, B637, PB1753 through PB1758, PB3360, JH642, 1A243 (ATCC 39,087), ATCC 21332, ATCC 6051, MI113, DE100 (ATCC 39,094), GX4931, PBT 110, and PEP 211strain (See e.g., Hoch et al., Genetics, 73:215-228 [1973]) (See also, U.S. Pat. No. 4,450,235; U.S. Pat. No. 4,302,544; and EP 0134048; each of which is incorporated by reference in its entirety). The use of B. subtilis as an expression host well known in the art (See e.g., See, Palva et al., Gene 19:81-87 [1982]; Fahnestock and Fischer, J. Bacteriol., 165:796-804 [1986]; and Wang et al., Gene 69:39-47 [1988]).

In some embodiments, the Bacillus host is a Bacillus sp. that includes a mutation or deletion in at least one of the following genes, degU, degS, degR and degQ. Preferably the mutation is in a degU gene, and more preferably the mutation is degU(Hy)32. (See e.g., Msadek et al., J. Bacteriol., 172:824-834 [1990]; and Olmos et al., Mol. Gen. Genet., 253:562-567 [1997]). A preferred host strain is a Bacillus subtilis carrying a degU32(Hy) mutation. In some further embodiments, the Bacillus host comprises a mutation or deletion in scoC4, (See, e.g., Caldwell et al., J. Bacteriol., 183:7329-7340 [2001]); spollE (See, Arigoni et al., Mol. Microbiol., 31:1407-1415 [1999]); and/or oppA or other genes of the opp operon (See e.g., Perego et al., Mol. Microbiol., 5:173-185 [1991]). Indeed, it is contemplated that any mutation in the opp operon that causes the same phenotype as a mutation in the oppA gene will find use in some embodiments of the altered Bacillus strain of the present invention. In some embodiments, these mutations occur alone, while in other embodiments, combinations of mutations are present. In some embodiments, an altered Bacillus that can be used to produce the modified proteases of the invention is a Bacillus host strain that already includes a mutation in one or more of the above-mentioned genes. In addition, Bacillus sp. host cells that comprise mutation(s) and/or deletions of endogenous protease genes find use. In some embodiments, the Bacillus host cell comprises a deletion of the aprE and the nprE genes. In other embodiments, the Bacillus sp. host cell comprises a deletion of 5 protease genes (US20050202535), while in other embodiments, the Bacillus sp. host cell comprises a deletion of 9 protease genes (US20050202535).

Host cells are transformed with modified polynucleotides encoding the modified proteases of the present invention using any suitable method known in the art. Whether the modified polynucleotide is incorporated into a vector or is used without the presence of plasmid DNA, it is introduced into a microorganism, in some embodiments, preferably an E. coli cell or a competent Bacillus cell. Methods for introducing DNA into Bacillus cells involving plasmid constructs and transformation of plasmids into E. coli are well known. In some embodiments, the plasmids are subsequently isolated from E. coli and transformed into Bacillus. However, it is not essential to use intervening microorganisms such as E. coli, and in some embodiments, a DNA construct or vector is directly introduced into a Bacillus host.

Those of skill in the art are well aware of suitable methods for introducing polynucleotide sequences into Bacillus cells (See e.g., Ferrari et al., “Genetics,” in Harwood et al. (ed.), Bacillus, Plenum Publishing Corp. [1989], pages 57-72; Saunders et al., J. Bacteriol., 157:718-726 [1984]; Hoch et al., J. Bacteriol., 93:1925-1937 [1967]; Mann et al., Current Microbiol., 13:131-135 [1986]; and Holubova, Folia Microbiol., 30:97 [1985]; Chang et al., Mol. Gen. Genet., 168:11-115 [1979]; Vorobjeva et al., FEMS Microbiol. Lett., 7:261-263 [1980]; Smith et al., Appl. Env. Microbiol., 51:634 [1986]; Fisher et al., Arch. Microbiol., 139:213-217 [1981]; and McDonald, J. Gen. Microbiol., 130:203 [1984]). Indeed, such methods as transformation, including protoplast transformation and congression, transduction, and protoplast fusion are known and suited for use in the present invention. Methods of transformation are used to introduce a DNA construct provided by the present invention into a host cell. Methods known in the art to transform Bacillus, include such methods as plasmid marker rescue transformation, which involves the uptake of a donor plasmid by competent cells carrying a partially homologous resident plasmid (Contente et al., Plasmid 2:555-571 [1979]; Haima et al., Mol. Gen. Genet., 223:185-191 [1990]; Weinrauch et al., J. Bacteriol., 154:1077-1087 [1983]; and Weinrauch et al., J. Bacteriol., 169:1205-1211 [1987]). In this method, the incoming donor plasmid recombines with the homologous region of the resident “helper” plasmid in a process that mimics chromosomal transformation.

In addition to commonly used methods, in some embodiments, host cells are directly transformed (i.e., an intermediate cell is not used to amplify, or otherwise process, the DNA construct prior to introduction into the host cell). Introduction of the DNA construct into the host cell includes those physical and chemical methods known in the art to introduce DNA into a host cell without insertion into a plasmid or vector. Such methods include, but are not limited to calcium chloride precipitation, electroporation, naked DNA, liposomes and the like. In additional embodiments, DNA constructs are co-transformed with a plasmid, without being inserted into the plasmid. In further embodiments, a selective marker is deleted from the altered Bacillus strain by methods known in the art (See, Stahl et al., J. Bacteriol., 158:411-418 [1984]; and Palmeros et al., Gene 247:255-264 [2000]).

In some embodiments, the transformed cells of the present invention are cultured in conventional nutrient media. The suitable specific culture conditions, such as temperature, pH and the like are known to those skilled in the art. In addition, some culture conditions may be found in the scientific literature such as Hopwood (2000) Practical Streptomyces Genetics, John Innes Foundation, Norwich UK; Hardwood et al., (1990) Molecular Biological Methods for Bacillus, John Wiley and from the American Type Culture Collection (ATCC).

In some embodiments, host cells transformed with polynucleotide sequences encoding modified proteases are cultured in a suitable nutrient medium under conditions permitting the expression and production of the present protease, after which the resulting protease is recovered from the culture. The medium used to culture the cells comprises any conventional medium suitable for growing the host cells, such as minimal or complex media containing appropriate supplements. Suitable media are available from commercial suppliers or may be prepared according to published recipes (e.g., in catalogues of the American Type Culture Collection). In some embodiments, the protease produced by the cells is recovered from the culture medium by conventional procedures, including, but not limited to separating the host cells from the medium by centrifugation or filtration, precipitating the proteinaceous components of the supernatant or filtrate by means of a salt (e.g., ammonium sulfate), chromatographic purification (e.g., ion exchange, gel filtration, affinity, etc.). Thus, any method suitable for recovering the protease(s) of the present invention finds use in the present invention. Indeed, it is not intended that the present invention be limited to any particular purification method.

The protein produced by a recombinant host cell comprising a modified protease of the present invention is secreted into the culture media. In some embodiments, other recombinant constructions join the heterologous or homologous polynucleotide sequences to nucleotide sequence encoding a protease polypeptide domain which facilitates purification of the soluble proteins (Kroll D J et al (1993) DNA Cell Biol 12:441-53). Such purification facilitating domains include, but are not limited to, metal chelating peptides such as histidine-tryptophan modules that allow purification on immobilized metals (Porath J (1992) Protein Expr Purif 3:263-281), protein A domains that allow purification on immobilized immunoglobulin, and the domain utilized in the FLAGS extension/affinity purification system (Immunex Corp, Seattle Wash.). The inclusion of a cleavable linker sequence such as Factor XA or enterokinase (Invitrogen, San Diego Calif.) between the purification domain and the heterologous protein also find use to facilitate purification.

As indicated above, the invention provides for modified full-length polynucleotides that encode modified full-length proteases that are processed by a Bacillus host cell to produce the mature form at a level that is greater than that of the same mature protease when processed from an unmodified full-length enzyme by a Bacillus host cell grown under the same conditions. The level of production is determined by the level of activity of the secreted enzyme.

One measure of enhancement of production can be determined as relative activity, which is expressed as a percent of the ratio of the value of the enzymatic activity of the mature form when processed from the modified protease to the value of the enzymatic activity of the mature form when processed from the unmodified precursor protease. A relative activity equal or greater than 100% indicates that the mature form a protease that is processed from a modified precursor is produced at a level that is equal or greater than the level at which the same mature protease is produced but when processed from an unmodified precursor. Thus, in some embodiments, the relative activity of a mature protease processed from the modified protease is at least about 100%, at least about 110%, at least about 120%, at least about 130%, at least about 140%, at least about 150%, at least about 160%, at least about 170%, at least about 180%, at least about 190%, at least about 200%, at least about 225%, at least about 250%, at least about 275%, at least about 300%, at least about 325%, at least about 350%, at least about 375%, at least about 400%, at least about 425%, at least about 450%, at least about 475%, at least about 500%, at least about 525%, at least about 550%, at least about 575%, at least about 600%, at least about 625%, at least about 650%, at least about 675%, at least about 700%, at least about 725%, at least about 750%, at least about 800%, at least about 825%, at least about 850%, at least about 875%, at least about 850%, at least about 875%, at least about 900%, and up to at least about 1000% or more when compared to the corresponding production of the mature form of the protease that was processed from the unmodified precursor protease. Alternatively, the relative activity is expressed as the ratio of production which is determined by dividing the value of the activity of the protease processed from a modified precursor by the value of the activity of the same protease when processed from an unmodified precursor. Thus, in some embodiments, the ratio of production of a mature protease processed from a modified precursor is at least about 1, at least about 1.1, at least about 1.2, at least about 1.3 at least about, 1.4, at least about 1.5, at least about 1.6, at least about 1.7, at least about. 18, at least about 1.9, at least about 2, at least about 2.25, at least about 2.5, at least about 2.75, at least about 3, at least about 3.25, at least about 3.5, at least about 3.75, at least about, at least about 4.25, at least about 4.5, at least about 4.75, at least about 5, at least about 5.25, at least about 5.5, at least about 5.75, at least about 6, at least about 6.25, at least about 6.5, at least about 6.75, at least about 7, at least about 7.25, at least about 7.5, at least about 8, at least about 8.25, at least about 8.5, at least about 8.75, at least about 9, and up to at least about 10.

There are various assays known to those of ordinary skill in the art for detecting and measuring activity of proteases. In particular, assays are available for measuring protease activity that are based on the release of acid-soluble peptides from casein or hemoglobin, measured as absorbance at 280 nm or colorimetrically using the Folin method (See e.g., Bergmeyer et al., “Methods of Enzymatic Analysis” vol. 5, Peptidases, Proteinases and their Inhibitors, Verlag Chemie, Weinheim [1984]). Some other assays involve the solubilization of chromogenic substrates (See e.g., Ward, “Proteinases,” in Fogarty (ed.)., Microbial Enzymes and Biotechnology, Applied Science, London, [1983], pp 251-317). Other exemplary assays include, but are not limited to succinyl-Ala-Ala-Pro-Phe-para nitroanilide assay (SAAPFpNA) and the 2,4,6-trinitrobenzene sulfonate sodium salt assay (TNBS assay). Numerous additional references known to those in the art provide suitable methods (See e.g., Wells et al., Nucleic Acids Res. 11:7911-7925 [1983]; Christianson et al., Anal. Biochem., 223:119-129 [1994]; and Hsia et al., Anal Biochem., 242:221-227 [1999]). It is not intended that the present invention be limited to any particular assay method(s).

Other means for determining the levels of production of a mature protease in a host cell include, but are not limited to methods that use either polyclonal or monoclonal antibodies specific for the protein. Examples include, but are not limited to enzyme-linked immunosorbent assays (ELISA), radioimmunoassays (RIA), fluorescent immunoassays (FIA), and fluorescent activated cell sorting (FACS). These and other assays are well known in the art (See e.g., Maddox et al., J. Exp. Med., 158:1211 [1983]).

All publications and patents mentioned herein are herein incorporated by reference. Various modifications and variations of the described method and system of the invention will be apparent to those skilled in the art without departing from the scope and spirit of the invention. Although the invention has been described in connection with specific embodiments, it should be understood that the invention as should not be unduly limited to such specific embodiments. Indeed, various modifications of the described modes for carrying out the invention that are obvious to those skilled in the art and/or related fields are intended to be within the scope of the present invention.

### EXPERIMENTAL

The following examples are provided in order to demonstrate and further illustrate certain embodiments and aspects of the present invention and are not to be construed as limiting the scope thereof.

In the experimental disclosure which follows, the following abbreviations apply: ppm (parts per million); M (molar); mM (millimolar); μM (micromolar); nM (nanomolar); mol (moles); mmol (millimoles); μmol (micromoles); nmol (nanomoles); gm (grams); mg (milligrams); μg (micrograms); pg (picograms); L (liters); ml and mL (milliliters); μl and μL (microliters); cm (centimeters); mm (millimeters); μm (micrometers); nm (nanometers); U (units); V (volts); MW (molecular weight); sec (seconds); min(s) (minute/minutes); h(s) and hr(s) (hour/hours); ° C. (degrees Centigrade); QS (quantity sufficient); ND (not done); NA (not applicable); rpm (revolutions per minute); w/v (weight to volume); v/v (volume to volume); g (gravity); OD (optical density); aa (amino acid); bp (base pair); kb (kilobase pair); kD (kilodaltons); suc-AAPF-pNA (succinyl-L-alanyl-L-alanyl-L-prolyl-L-phenyl-alanyl-para-nitroanilide); FNA (variant of BPN′); BPN′ (Bacillus amyloliquefaciens subtilisin); DMSO (dimethyl sulfoxide); cDNA (copy or complementary DNA); DNA (deoxyribonucleic acid); ssDNA (single stranded DNA); dsDNA (double stranded DNA); dNTP (deoxyribonucleotide triphosphate); DTT (1,4-dithio-DL-threitol); H2O (water); dH2O (deionized water); HCl (hydrochloric acid); MgCl₂ (magnesium chloride); MOPS (3-[N-morpholino]propanesulfonic acid); NaCl (sodium chloride); PAGE (polyacrylamide gel electrophoresis); PBS (phosphate buffered saline [150 mM NaCl, 10 mM sodium phosphate buffer, pH 7.2]); PEG (polyethylene glycol); PCR (polymerase chain reaction); PMSF (phenylmethylsulfonyl fluoride); RNA (ribonucleic acid); SDS (sodium dodecyl sulfate); Tris (tris(hydroxymethyl) aminomethane); SOC (2% Bacto-Tryptone, 0.5% Bacto Yeast Extract, 10 mM NaCl, 2.5 mM KCl); Terrific Broth (TB; 12 g/l Bacto Tryptone, 24 g/l glycerol, 2.31 g/l KH₂PO₄, and 12.54 g/l K₂HPO₄); OD280 (optical density at 280 nm); OD600 (optical density at 600 nm); A405 (absorbance at 405 nm); Vmax (the maximum initial velocity of an enzyme catalyzed reaction); HEPES (N-[2-Hydroxyethyl]piperazine-N-[2-ethanesulfonic acid]); Tris-HCl (tris[Hydroxymethyl]aminomethane-hydrochloride); TCA (trichloroacetic acid); HPLC (high pressure liquid chromatography); RP-HPLC (reverse phase high pressure liquid chromatography); TLC (thin layer chromatography); EDTA (ethylenediaminetetracetic acid); EtOH (ethanol); SDS (sodium dodecyl sulfate); Tris (tris(hydroxymethyl)aminomethane); TAED (N,N,N′N′-tetraacetylethylenediamine);

### Example 1

### Targeted ISD (Insertion Substitution Deletion) Library Construction

The method used to create a library of modified FNA polynucleotides is outlined in FIG. 2 (ISD method). Two sets of oligonucleotides that evenly covered the FNA gene sequence coding for the pre-pro region (SEQ ID NO:7) of a full-length protein of 392 amino acids (SEQ ID NO:1), in both forward and reverse direction were used to amplify the left and right segments of the portion of the FNA gene that encodes the pre-pro region of FNA. Two PCR reactions (left and right segments) contained either the 5′ forward or the 3′ reverse gene sequence flanking oligonucleotides each in combination with the corresponding opposite priming oligonucleotides. The left fragments were amplified using a single forward primer containing an EcoRI site (P3233, TTATTGTCTCATGAGCGGATAC; SEQ ID NO:123) and reverse primers P3301r-P3404r each containing Eam104I site (SEQ ID NOS:124-227; TABLE 1). The right fragments were amplified using a single reverse primer containing an MluI restriction site (P3237, TGTCGATAACCGCTACTTTAAC; SEQ ID NO:228) and forward primers P3301f-P3401f each containing an Eam104I restriction site (SEQ ID NOS: 229-332; TABLE 2).

TABLE 1
Sequences of reverse primers used to
amplify left fragments

| PRIMER | | SEQ |
|---|---|---|
| NAME | PRIMER SEQUENCE | ID NO: |
| P3301r | AACTCTTCAVNNTCTTTACCCTCTCCTTTTAAAAAA | 124 |
| P3302r | AACTCTTCAVNNCACTCTTTACCCTCTCCTTTTAAA | 125 |
| P3303r | AACTCTTCAVNNTCTCACTCTTTACCCTCTCCTTTT | 126 |
| P3304r | AACTCTTCAVNNGCTTCTCACTCTTTACCCTCTCCT | 127 |
| P3305r | AACTCTTCAVNNTTTGCTTCTCACTCTTTACCCTCT | 128 |
| P3306r | AACTCTTCAVNNTTTTTTGCTTCTCACTCTTTACCCT | 129 |
| P3307r | AACTCTTCAVNNCAATTTTTTGCTTCTCACTCTTTA | 130 |
| P3308r | AACTCTTCAVNNCCACAATTTTTTGCTTCTCACTCT | 131 |
| P3309r | AACTCTTCAVNNGATCCACAATTTTTTGCTTCTCAC | 132 |
| P3310r | AACTCTTCAVNNACTGATCCACAATTTTTTGCTTCT | 133 |
| P3311r | AACTCTTCAVNNCAAACTGATCCACAATTTTTTGCT | 134 |
| P3312r | AACTCTTCAVNNCAGCAAACTGATCCACAATTTTTT | 135 |
| P3313r | AACTCTTCAVNNAAACAGCAAACTGATCCACAATTT | 136 |
| P3314r | AACTCTTCAVNNAGCAAACAGCAAACTGATCCACAA | 137 |
| P3315r | AACTCTTCAVNNTAAAGCAAACAGCAAACTGATCCA | 138 |
| P3316r | AACTCTTCAVNNCGCTAAAGCAAACAGCAAACTGAT | 139 |
| P3317r | AACTCTTCAVNNTAACGCTAAAGCAAACAGCAAACT | 140 |
| P3318r | AACTCTTCAVNNGATTAACGCTAAAGCAAACAGCAA | 141 |
| P3319r | AACTCTTCAVNNAAAGATTAACGCTAAAGCAAACAG | 142 |
| P3320r | AACTCTTCAVNNCGTAAAGATTAACGCTAAAGCAAA | 143 |
| P3321r | AACTCTTCAVNNCATCGTAAAGATTAACGCTAAAG | 144 |
| P3322r | AACTCTTCAVNNCGCCATCGTAAAGATTAACGCTAA | 145 |
| P3323r | AACTCTTCAVNNGAACGCCATCGTAAAGATTAAC | 146 |
| P3324r | AACTCTTCAVNNGCCGAACGCCATCGTAAAGATTAA | 147 |
| P3325r | AACTCTTCAVNNGCTGCCGAACGCCATCGTAAAGAT | 148 |
| P3326r | AACTCTTCAVNNTGTGCTGCCGAACGCCATCGTAAA | 149 |
| P3327r | AACTCTTCAVNNGGATGTGCTGCCGAACGCCATCGT | 150 |
| P3328r | AACTCTTCAVNNGCTGGATGTGCTGCCGAACGCCAT | 151 |
| P3329r | AACTCTTCAVNNCGCGCTGGATGTGCTGCCGAAC | 152 |
| P3330r | AACTCTTCAVNNCTGCGCGCTGGATGTGCTGCCGAA | 153 |
| P3331r | AACTCTTCAVNNCGCCTGCGCGCTGGATGTGCTG | 154 |
| P3332r | AACTCTTCAVNNTGCCGCCTGCGCGCTGGATGTGCT | 155 |
| P3333r | AACTCTTCAVNNCCCTGCCGCCTGCGCGCTGGATGT | 156 |
| P3334r | AACTCTTCAVNNTTTCCCTGCCGCCTGCGCGCTGGA | 157 |
| P3335r | AACTCTTCAVNNTGATTTCCCTGCCGCCTGCGCGCT | 158 |
| P3336r | AACTCTTCAVNNGTTTGATTTCCCTGCCGCCTG | 159 |
| P3337r | AACTCTTCAVNNCCCGTTTGATTTCCCTGCCGCCTG | 160 |
| P3338r | AACTCTTCAVNNTTCCCCGTTTGATTTCCCTG | 161 |
| P3339r | AACTCTTCAVNNCTTTTCCCCGTTTGATTTCCCTG | 162 |
| P3340r | AACTCTTCAVNNTTTCTTTTCCCCGTTTGATTTC | 163 |
| P3341r | AACTCTTCAVNNATATTTCTTTTCCCCGTTTGATTT | 164 |
| P3342r | AACTCTTCAVNNAATATATTTCTTTTCCCCGTTTGA | 165 |
| P3343r | AACTCTTCAVNNGACAATATATTTCTTTTCCCCGTT | 166 |
| P3344r | AACTCTTCAVNNCCCGACAATATATTTCTTTTC | 167 |
| P3345r | AACTCTTCAVNNAAACCCGACAATATATTTCTTTTC | 168 |
| P3346r | AACTCTTCAVNNTTTAAACCCGACAATATATTTCTT | 169 |
| P3347r | AACTCTTCAVNNCTGTTTAAACCCGACAATATATTT | 170 |
| P3348r | AACTCTTCAVNNTGTCTGTTTAAACCCGACAATATA | 171 |
| P3349r | AACTCTTCAVNNCATTGTCTGTTTAAACCCGACAAT | 172 |
| P3350r | AACTCTTCAVNNGCTCATTGTCTGTTTAAACCCGAC | 173 |
| P3351r | AACTCTTCAVNNCGTGCTCATTGTCTGTTTAAAC | 174 |
| P3352r | AACTCTTCAVNNCATCGTGCTCATTGTCTGTTTAAA | 175 |
| P3353r | AACTCTTCAVNNGCTCATCGTGCTCATTGTCTGTTT | 176 |
| P3354r | AACTCTTCAVNNGGCGCTCATCGTGCTCATTGTCTG | 177 |
| P3355r | AACTCTTCAVNNAGCGGCGCTCATCGTGCTCATTGT | 178 |
| P3356r | AACTCTTCAVNNCTTAGCGGCGCTCATCGTGCTCAT | 179 |
| P3357r | AACTCTTCAVNNCTTCTTAGCGGCGCTCATCGTGCT | 180 |
| P3358r | AACTCTTCAVNNTTTCTTCTTAGCGGCGCTCATCGT | 181 |
| P3359r | AACTCTTCAVNNATCTTTCTTCTTAGCGGCGCTCAT | 182 |
| P3360r | AACTCTTCAVNNGACATCTTTCTTCTTAGCGGCGCT | 183 |
| P3361r | AACTCTTCAVNNAATGACATCTTTCTTCTTAGC | 184 |
| P3362r | AACTCTTCAVNNAGAAATGACATCTTTCTTCTTAGC | 185 |
| P3363r | AACTCTTCAVNNTTCAGAAATGACATCTTTCTTCTT | 186 |
| P3364r | AACTCTTCAVNNTTTTTCAGAAATGACATCTTTCTT | 187 |
| P3365r | AACTCTTCAVNNGCCTTTTTCAGAAATGACATCTTT | 188 |
| P3366r | AACTCTTCAVNNCCCGCCTTTTTCAGAAATGACATC | 189 |
| P3367r | AACTCTTCAVNNTTTCCCGCCTTTTTCAGAAATGAC | 190 |
| P3368r | AACTCTTCAVNNCACTTTCCCGCCTTTTTCAGAAAT | 191 |
| P3369r | AACTCTTCAVNNTTGCACTTTCCCGCCTTTTTCAGA | 192 |
| P3370r | AACTCTTCAVNNCTTTTGCACTTTCCCGCCTTTTTC | 193 |
| P3371r | AACTCTTCAVNNTTGCTTTTGCACTTTCCCGCCTTT | 194 |
| P3372r | AACTCTTCAVNNGAATTGCTTTTGCACTTTCC | 195 |
| P3373r | AACTCTTCAVNNTTTGAATTGCTTTTGCACTTTC | 196 |
| P3374r | AACTCTTCAVNNATATTTGAATTGCTTTTGCACTTT | 197 |
| P3375r | AACTCTTCAVNNTACATATTTGAATTGCTTTTGCAC | 198 |
| P3376r | AACTCTTCAVNNGTCTACATATTTGAATTGCTTTTG | 199 |
| P3377r | AACTCTTCAVNNTGCGTCTACATATTTGAATTGCTT | 200 |
| P3378r | AACTCTTCAVNNAGCTGCGTCTACATATTTGAATTG | 201 |
| P3379r | AACTCTTCAVNNTGAAGCTGCGTCTACATATTTGAA | 202 |
| P3380r | AACTCTTCAVNNAGCTGAAGCTGCGTCTACATATTT | 203 |
| P3381r | AACTCTTCAVNNTGTAGCTGAAGCTGCGTCTACATA | 204 |
| P3382r | AACTCTTCAVNNTAATGTAGCTGAAGCTGCGTCTAC | 205 |
| P3383r | AACTCTTCAVNNGTTTAATGTAGCTGAAGCTGCGTC | 206 |
| P3384r | AACTCTTCAVNNTTCGTTTAATGTAGCTGAAGCTGC | 207 |
| P3385r | AACTCTTCAVNNTTTTTCGTTTAATGTAGCTGAAG | 208 |
| P3386r | AACTCTTCAVNNAGCTTTTTCGTTTAATGTAGCTGA | 209 |
| P3387r | AACTCTTCAVNNTACAGCTTTTTCGTTTAATGTAG | 210 |
| P3388r | AACTCTTCAVNNTTTTACAGCTTTTTCGTTTAATGT | 211 |
| P3389r | AACTCTTCAVNNTTCTTTTACAGCTTTTTCGTTTAA | 212 |
| P3390r | AACTCTTCAVNNCAATTCTTTTACAGCTTTTTCGTT | 213 |
| P3391r | AACTCTTCAVNNTTTCAATTCTTTTACAGCTTTTTC | 214 |
| P3392r | AACTCTTCAVNNTTTTTTCAATTCTTTTACAGCTTT | 215 |
| P3393r | AACTCTTCAVNNGTCTTTTTTCAATTCTTTTACAG | 216 |
| P3394r | AACTCTTCAVNNCGGGTCTTTTTTCAATTCTTTTAC | 217 |
| P3395r | AACTCTTCAVNNGCTCGGGTCTTTTTTCAATTCTTT | 218 |
| P3396r | AACTCTTCAVNNGACGCTCGGGTCTTTTTTCAATTC | 219 |
| P3397r | AACTCTTCAVNNAGCGACGCTCGGGTCTTTTTTCAA | 220 |
| P3398r | AACTCTTCAVNNGTAAGCGACGCTCGGGTCTTTTTT | 221 |
| P3399r | AACTCTTCAVNNAACGTAAGCGACGCTCGGGTCTTT | 222 |
| P3400r | AACTCTTCAVNNTTCAACGTAAGCGACGCTCGGGTC | 223 |
| P3401r | AACTCTTCAVNNTTCTTCAACGTAAGCGACGCTC | 224 |
| P3402r | AACTCTTCAVNNATCTTCTTCAACGTAAGCGACGCT | 225 |
| P3403r | AACTCTTCAVNNGTGATCTTCTTCAACGTAAGCGAC | 226 |
| P3404r | AACTCTTCAVNNTACGTGATCTTCTTCAACGTAAG | 227 |

TABLE 2
Sequences of forward primers used to
amplify right fragments

| PRIMER | | SEQ |
|---|---|---|
| NAME | PRIMER SEQUENCE | ID NO: |
| P3301f | AACTCTTCANNBAGAAGCAAAAAATTGTGGATCAGT | 229 |
| P3302f | AACTCTTCANNBAGCAAAAAATTGTGGATCAGTTTG | 230 |
| P3303f | AACTCTTCANNBAAAAAATTGTGGATCAGTTTGCTG | 231 |
| P3304f | AACTCTTCANNBAAATTGTGGATCAGTTTGCTGTTT | 232 |
| P3305f | AACTCTTCANNBTTGTGGATCAGTTTGCTGTTTGCT | 233 |
| P3306f | AACTCTTCANNBTGGATCAGTTTGCTGTTTGCTTTA | 234 |
| P3307f | AACTCTTCANNBATCAGTTTGCTGTTTGCTTTAG | 235 |
| P3308f | AACTCTTCANNBAGTTTGCTGTTTGCTTTAGCGTTA | 236 |
| P3309f | AACTCTTCANNBTTGCTGTTTGCTTTAGCGTTAATC | 237 |
| P3310f | AACTCTTCANNBCTGTTTGCTTTAGCGTTAATCTTT | 238 |
| P3311f | AACTCTTCANNBTTTGCTTTAGCGTTAATCTTTAC | 239 |
| P3312f | AACTCTTCANNBGCTTTAGCGTTAATCTTTACGATG | 240 |
| P3313f | AACTCTTCANNBTTAGCGTTAATCTTTACGATGG | 241 |
| P3314f | AACTCTTCANNBGCGTTAATCTTTACGATGGCGTTC | 242 |
| P3315f | AACTCTTCANNBTTAATCTTTACGATGGCGTTCG | 243 |
| P3316f | AACTCTTCANNBATCTTTACGATGGCGTTCGGCAG | 244 |
| P3317f | AACTCTTCANNBTTTACGATGGCGTTCGGCAGCACA | 245 |
| P3318f | AACTCTTCANNBACGATGGCGTTCGGCAGCACATC | 246 |
| P3319f | AACTCTTCANNBATGGCGTTCGGCAGCACATCCAG | 247 |
| P3320f | AACTCTTCANNBGCGTTCGGCAGCACATCCAGC | 248 |
| P3321f | AACTCTTCANNBTTCGGCAGCACATCCAGCGCGCAG | 249 |
| P3322f | AACTCTTCANNBGGCAGCACATCCAGCGCGCAG | 250 |
| P3323f | AACTCTTCANNBAGCACATCCAGCGCGCAGGCGGCA | 251 |
| P3324f | AACTCTTCANNBACATCCAGCGCGCAGGCGGCAG | 252 |
| P3325f | AACTCTTCANNBTCCAGCGCGCAGGCGGCAGGGAAA | 253 |
| P3326f | AACTCTTCANNBAGCGCGCAGGCGGCAGGGAAATCA | 254 |
| P3327f | AACTCTTCANNBGCGCAGGCGGCAGGGAAATCAAAC | 255 |
| P3328f | AACTCTTCANNBCAGGCGGCAGGGAAATCAAAC | 256 |
| P3329f | AACTCTTCANNBGCGGCAGGGAAATCAAACGGGGAA | 257 |
| P3330f | AACTCTTCANNBGCAGGGAAATCAAACGGGGAAAAG | 258 |
| P3331f | AACTCTTCANNBGGGAAATCAAACGGGGAAAAGAAA | 259 |
| P3332f | AACTCTTCANNBAAATCAAACGGGGAAAAGAAATAT | 260 |
| P3333f | AACTCTTCANNBTCAAACGGGGAAAAGAAATATATT | 261 |
| P3334f | AACTCTTCANNBAACGGGGAAAAGAAATATATTGTC | 262 |
| P3335f | AACTCTTCANNBGGGGAAAAGAAATATATTGTC | 263 |
| P3336f | AACTCTTCANNBGAAAAGAAATATATTGTCGGGTTT | 264 |
| P3337f | AACTCTTCANNBAAGAAATATATTGTCGGGTTTAAA | 265 |
| P3338f | AACTCTTCANNBAAATATATTGTCGGGTTTAAACAG | 266 |
| P3339f | AACTCTTCANNBTATATTGTCGGGTTTAAACAGACA | 267 |
| P3340f | AACTCTTCANNBATTGTCGGGTTTAAACAGACAATG | 268 |
| P3341f | AACTCTTCANNBGTCGGGTTTAAACAGACAATGAG | 269 |
| P3342f | AACTCTTCANNBGGGTTTAAACAGACAATGAGCAC | 270 |
| P3343f | AACTCTTCANNBTTTAAACAGACAATGAGCACGATG | 271 |
| P3344f | AACTCTTCANNBAAACAGACAATGAGCACGATGAG | 272 |
| P3345f | AACTCTTCANNBCAGACAATGAGCACGATGAG | 273 |
| P3346f | AACTCTTCANNBACAATGAGCACGATGAGCGCCGCT | 274 |
| P3347f | AACTCTTCANNBATGAGCACGATGAGCGCCGCTAAG | 275 |
| P3348f | AACTCTTCANNBAGCACGATGAGCGCCGCTAAGAAG | 276 |
| P3349f | AACTCTTCANNBACGATGAGCGCCGCTAAGAAGAAA | 277 |
| P3350f | AACTCTTCANNBATGAGCGCCGCTAAGAAGAAAGAT | 278 |
| P3351f | AACTCTTCANNBAGCGCCGCTAAGAAGAAAGATGTC | 279 |
| P3352f | AACTCTTCANNBGCCGCTAAGAAGAAAGATGTCATT | 280 |
| P3353f | AACTCTTCANNBGCTAAGAAGAAAGATGTCATTTCT | 281 |
| P3354f | AACTCTTCANNBAAGAAGAAAGATGTCATTTCTGAA | 282 |
| P3355f | AACTCTTCANNBAAGAAAGATGTCATTTCTGAAAAA | 283 |
| P3356f | AACTCTTCANNBAAAGATGTCATTTCTGAAAAAG | 284 |
| P3357f | AACTCTTCANNBGATGTCATTTCTGAAAAAGG | 285 |
| P3358f | AACTCTTCANNBGTCATTTCTGAAAAAGGCGGGAAA | 286 |
| P3359f | AACTCTTCANNBATTTCTGAAAAAGGCGGGAAAGTG | 287 |
| P3360f | AACTCTTCANNBTCTGAAAAAGGCGGGAAAGTGCAA | 288 |
| P3361f | AACTCTTCANNBGAAAAAGGCGGGAAAGTGCAAAAG | 289 |
| P3362f | AACTCTTCANNBAAAGGCGGGAAAGTGCAAAAGCAA | 290 |
| P3363f | AACTCTTCANNBGGCGGGAAAGTGCAAAAGCAATTC | 291 |
| P3364f | AACTCTTCANNBGGGAAAGTGCAAAAGCAATTCAAA | 292 |
| P3365f | AACTCTTCANNBAAAGTGCAAAAGCAATTCAAATAT | 293 |
| P3366f | AACTCTTCANNBGTGCAAAAGCAATTCAAATATGTA | 294 |
| P3367f | AACTCTTCANNBCAAAAGCAATTCAAATATGTAGAC | 295 |
| P3368f | AACTCTTCANNBAAGCAATTCAAATATGTAGACGCA | 296 |
| P3369f | AACTCTTCANNBCAATTCAAATATGTAGACGCAGCT | 297 |
| P3370f | AACTCTTCANNBTTCAAATATGTAGACGCAGCTTCA | 298 |
| P3371f | AACTCTTCANNBAAATATGTAGACGCAGCTTCAGCT | 299 |
| P3372f | AACTCTTCANNBTATGTAGACGCAGCTTCAGCTACA | 300 |
| P3373f | AACTCTTCANNBGTAGACGCAGCTTCAGCTACATTA | 301 |
| P3374f | AACTCTTCANNBGACGCAGCTTCAGCTACATTAAAC | 302 |
| P3375f | AACTCTTCANNBGCAGCTTCAGCTACATTAAACGAA | 303 |
| P3376f | AACTCTTCANNBGCTTCAGCTACATTAAACGAAAAA | 304 |
| P3377f | AACTCTTCANNBTCAGCTACATTAAACGAAAAAGCT | 305 |
| P3378f | AACTCTTCANNBGCTACATTAAACGAAAAAGCTGTA | 306 |
| P3379f | AACTCTTCANNBACATTAAACGAAAAAGCTGTAAAA | 307 |
| P3380f | AACTCTTCANNBTTAAACGAAAAAGCTGTAAAAGAA | 308 |
| P3381f | AACTCTTCANNBAACGAAAAAGCTGTAAAAGAATTG | 309 |
| P3382f | AACTCTTCANNBGAAAAAGCTGTAAAAGAATTGAAA | 310 |
| P3383f | AACTCTTCANNBAAAGCTGTAAAAGAATTGAAAAAA | 311 |
| P3384f | AACTCTTCANNBGCTGTAAAAGAATTGAAAAAAGAC | 312 |
| P3385f | AACTCTTCANNBGTAAAAGAATTGAAAAAAGACCCG | 313 |
| P3386f | AACTCTTCANNBAAAGAATTGAAAAAAGACCCGAG | 314 |
| P3387f | AACTCTTCANNBGAATTGAAAAAAGACCCGAGCGTC | 315 |
| P3388f | AACTCTTCANNBTTGAAAAAAGACCCGAGCGTCGCT | 316 |
| P3389f | AACTCTTCANNBAAAAAAGACCCGAGCGTCGCTTAC | 317 |
| P3390f | AACTCTTCANNBAAAGACCCGAGCGTCGCTTACGTT | 318 |
| P3391f | AACTCTTCANNBGACCCGAGCGTCGCTTACGTTGAA | 319 |
| P3392f | AACTCTTCANNBCCGAGCGTCGCTTACGTTGAAGAA | 320 |
| P3393f | AACTCTTCANNBAGCGTCGCTTACGTTGAAGAAGAT | 321 |
| P3394f | AACTCTTCANNBGTCGCTTACGTTGAAGAAGATCAC | 322 |
| P3395f | AACTCTTCANNBGCTTACGTTGAAGAAGATCACGTA | 323 |
| P3396f | AACTCTTCANNBTACGTTGAAGAAGATCACGTAGCA | 324 |
| P3397f | AACTCTTCANNBGTTGAAGAAGATCACGTAGCACAC | 325 |
| P3398f | AACTCTTCANNBGAAGAAGATCACGTAGCACAC | 326 |
| P3399f | AACTCTTCANNBGAAGATCACGTAGCACACGCGTAC | 327 |
| P3400f | AACTCTTCANNBGATCACGTAGCACACGCGTAC | 328 |
| P3401f | AACTCTTCANNBCACGTAGCACACGCGTACGCGCAG | 329 |
| P3402f | AACTCTTCANNBGTAGCACACGCGTACGCGCAGTC | 330 |
| P3403f | AACTCTTCANNBGCACACGCGTACGCGCAGTCCGT | 331 |
| P3404f | AACTCTTCANNBCACGCGTACGCGCAGTCCGTG | 332 |

Each amplification reaction contained 30 pmol of each oligonucleotide and 100 ng of pAC-FNa10 template. Amplifications were carried out using Vent DNA polymerase (New England Biolabs). The PCR mix (20 μl) was initially heated at 95° C. for 2.5 min followed by 30 cycles of denaturation at 94° C. for 15 s, annealing at 55° C. for 15 s and extension at 72° C. for 40 s. Following amplification, left and right fragments generated by the PCR reactions were gel-purified, mixed (200 ng of each fragment), digested with Eam104I, ligated with T4 DNA ligase and amplified by flanking primers (P3233 and P3237). The resulting fragments were digested with EcoRI and MluI, and cloned into the EcoRI/MluI sites in the pAC-FNA10 plasmid (FIG. 5). pAC-FNA10 was engineered to contain an MluI restriction site between the pre-pro region and the mature region of FNA. Transcription of DNA encoding precursor and modified proteases from the pAC-FNA10 plasmid was driven by the aprE short promoter

(SEQ ID NO: 333)

GAATTCATCTCAAAAAAATGGGTCTACTAAAATATTATTCCATCTATTAC
AATAAATTCACAGAATAGTCTTTTAAGTAAGTCTACTCTGAATTTTTTTA
AAAGGAGAGGGTAAAGA.

Thus, the expression cassette (1307 bp) that was contained in the had the polynucleotide sequence shown below (SEQ ID NO:334)

(SEQ ID NO: 334)
GAATTCATCTCAAAAAAATGGGTCTACTAAAATATTATTCCATCTATTACAATAAATTCACAGAATA
GTCTTTTAAGTAAGTCTACTCTGAATTTTTTTAAAAGGAGAGGGTAAAGAGTGAGAAGCAAAAAAT
TGTGGATCAGTTTGCTGTTTGCTTTAGCGTTAATCTTTACGATGGCGTTCGGCAGCACATCCAGC
GCGCAGGCGGCAGGGAAATCAAACGGGGAAAAGAAATATATTGTCGGGTTTAAACAGACAATGA
GCACGATGAGCGCCGCTAAGAAGAAAGATGTCATTTCTGAAAAAGGCGGGAAAGTGCAAAAGCA
ATTCAAATATGTAGACGCAGCTTCAGCTACATTAAACGAAAAAGCTGTAAAAGAATTGAAAAAAGA
CCCGAGCGTCGCTTACGTTGAAGAAGATCACGTAGCACACGCGTACGCGCAGTCCGTGCCTTAC
GGCGTATCACAAATTAAAGCCCCTGCTCTGCACTCTCAAGGCTACACTGGATCAAATGTTAAAGT
AGCGGTTATCGACAGCGGTATCGATTCTTCTCATCCTGATTTAAAGGTAGCAGGCGGAGCCAGC
ATGGTTCCTTCTGAAACAAATCCTTTCCAAGACAACAACTCTCACGGAACTCACGTTGCCGGCAC
AGTTGCGGCTCTTAATAACTCAATCGGTGTATTAGGCGTTGCGCCAAGCGCATCACTTTACGCTG
TAAAAGTTCTCGGTGCTGACGGTTCCGGCCAATACAGCTGGATCATTAACGGAATCGAGTGGGC
GATCGCAAACAATATGGACGTTATTAACATGAGCCTCGGCGGACCTTCTGGTTCTGCTGCTTTAA
AAGCGGCAGTTGATAAAGCCGTTGCATCCGGCGTCGTAGTCGTTGCGGCAGCCGGTAACGAAG
GCACTTCCGGCAGCTCAAGCACAGTGGGCTACCCTGGTAAATACCCTTCTGTCATTGCAGTAGG
CGCTGTTGACAGCAGCAACCAAAGAGCATCTTTCTCAAGCGTAGGACCTGAGCTTGATGTCATG
GCACCTGGCGTATCTATCCAAAGCACGCTTCCTGGAAACAAATACGGCGCGTTGAACGGTACAT
CAATGGCATCTCCGCACGTTGCCGGAGCGGCTGCTTTGATTCTTTCTAAGCACCCGAACTGGAC
AAACACTCAAGTCCGCAGCAGTTTAGAAAACACCACTACAAAACTTGGTGATTCTTTCTACTATGG
AAAAGGGCTGATCAACGTACAGGCGGCAGCTCAGTAAACTCGAGATAAAAAACCGGCCTTGGCC
CCGCCGGTTTTTTATTATTTTTCTTCCTCCGGATCC.

The cassette contains the AprE promoter (underlined), the PRE, PRO and mature regions of FNA, and the transcription terminator.

Ligation mixtures were amplified using rolling circle amplification according to the manufacturer's recommended method (Epicentre Biotech).

One hundred and three libraries containing DNA sequences encoding FNA protease with mutated pre-pro regions were transformed into a competent Bacillus subtilis strain (genotype: ΔaprE, ΔnprE, spollE, amyE::xylRPxylAcomK-phleo) and recovered in 1 ml of Luria Broth (LB) at 37° C. for 1 hour. The bacteria were made competent by the induction of the comKgene under control of a xylose inducible promoter (See e.g., Hahn et al., Mol Microbiol, 21:763-775, 1996). The preparations were plated on LB agar plates containing 1.6% skim milk and 5 mg/l chloramphenicol, and were incubated overnight at 37° C.

One thousand clones from each of the 103 libraries that produced the largest halos were picked, precultured by incubating the individual colonies in a 16-ml tube with 3 ml of LB containing chloramphenicol at a final concentration of 5 mg/L, and incubated 4 h at 37° C. with shaking at 250 rpm. One milliliter of the precultured cells was added to a 250 ml shake-flask containing 25 ml of modified FNII media (7 g/L Cargill Soy Flour #4, 0.275 mM MgSO4, 220 mg/L K2HPO4, 21.32 g/L Na2HPO4 7H2O, 6.1 g/L NaH2PO4.H₂O, 3.6 g/L Urea, 0.5 ml/L Mazu, 35 g/L Maltrin M150 and 23.1 g/L Glucose.H2O). Shake-flasks were incubated at 37° C. with shaking at 250 rpm. Aliquots of the culture (200 ul) were removed every 12 h, spinned down in the bench top centrifuge for 2 min at 8000 rpm and the supernatant was frozen at −20° C. Each isolate was screened for AAPF activity using a 96-well plate assay described below.

### AAPF Protease Assay in 96-Well Microtiter Plates

Clones producing the largest halos were further screened for AAPF activity using a 96-well plate assay. The chosen colonies were picked and precultured by incubating the individual colonies in a 96-well flat bottom microtiter plate (MTP) with 150 ul of LB containing chloramphenicol at a final concentration of 5 mg/L, and incubated at 37° C. with shaking at 220 rpm. One hundred and forty microliters of Grant's II medium (10 g/L soytone, 75 g/L glucose, 3.6 g/L urea, 83.72 g/L MOPS, 7.17 g/L tricine, 3 mM K2HPO4, 0.276 mM K2SO4, 0.528 mM MgCl2, 2.9 g/L NaCl, 1.47 mg/L Trisodium Citrate Dihydrate, 0.4 mg/L FeSO₄.7H₂O, mg/L, 0.1 mg/L MnSO₄.H₂O, 0.1 mg/L ZnSO₄.H₂O, 0.05 mg/L CuCl₂.2H₂O, 0.1 mg/L CoCl₂.6H₂O, 0.1 mg/L Na₂MoO4.2H2O) was placed in each well of a fresh 96-well MTP. Then 10 ul of each preculture from the first MTP was added to the corresponding well in the second MTP containing the Grant's II medium. The cultures were incubated for 40 hours in a humidified chamber at 37° C. with shaking at 220 rpm. Following incubation, cultures were diluted from 10 to 100 times in 100 ul of Tris dilution buffer, and the AAPF activity was measured as follows.

The AAPF activity of a sample was measured as the rate of hydrolysis of N-succinyl-L-alanyl-L-alanyl-L-prolyl-L-phenyl-p-nitroanilide (suc-AAPF-pNA). The reagent solutions used were: 100 mM Tris/HCl, pH 8.6, containing 0.005% TWEEN®-80 (Tris dilution buffer and 160 mM suc-AAPF-pNA in DMSO (suc-AAPF-pNA stock solution) (Sigma: S-7388). To prepare a suc-AAPF-pNA working solution, 1 ml suc-AAPF-pNA stock solution was added to 100 ml Tris/HCl buffer and mixed well for at least 10 seconds. The assay was performed by adding 10 μl of diluted culture to each well, immediately followed by the addition of 190 μl 1 mg/ml suc-AAPF-pNA working solution. The solutions were mixed for 5 sec., and the absorbance change in kinetic mode (20 readings in 5 minutes) was read at 410 nm in an MTP reader, at 25° C. The protease activity was expressed as AU (activity=ΔOD·min⁻¹ ml⁻¹). Relative production was calculated as the ratio of the rate of AAPF conversion for any one experimental sample divided by the rate of AAPF conversion for the control sample (wild-type pAC-FNA10).

The results of the AAPF activity of the clones identified from the ISD Library screen and having the highest AAPF activity are given in Table 3. Clones 1001 and 515 contained two mutations: a deletion and a substitution. While the deletion was intentionally introduced into the pre-pro sequence, the substitution is likely to have resulted from mis-reading errors by the DNA polymerase.

TABLE 3

Production of mature FNA (SEQ ID NO: 9) processed from modified full-length
FNA relative to the production of mature FNA processed from unmodified
full-length FNA comprising at least one mutation in the pre-pro region

| | | | | |
|---|---|---|---|---|
| | | Relative | | |
| | | production | Pre-pro Polypeptide | |
| Clone # | Mutations | (%) | Sequence | Pre-pro Nucleotide sequence |

| | | | | |
|---|---|---|---|---|
| UNMODIFIED | NONE | 100 | VRSKKLWISLLFALA | GTGAGAAGCAAAAAATTGTGGATCAGTT |
| FNA | | | LIFTMAFGSTSSAQA | TGCTGTTTGCTTTAGCGTTAATCTTTACG |
| | | | AGKSNGEKKYIVGF | ATGGCGTTCGGCAGCACATCCAGCGCG |
| | | | KQTMSTMSAAKKK | CAGGCGGCAGGGAAATCAAACGGGGAA |
| | | | DVISEKGGKVQKQF | AAGAAATATATTGTCGGGTTTAAACAGA |
| | | | KYVDAASATLNEKA | CAATGAGCACGATGAGCGCCGCTAAGA |
| | | | VKELKKDPSVAYVE | AGAAAGATGTCATTTCTGAAAAAGGCGG |
| | | | EDHVAHAY (SEQ ID | GAAAGTGCAAAAGCAATTCAAATATGTA |
| | | | NO: 7) | GACGCAGCTTCAGCTACATTAAACGAAA |
| | | | | AAGCTGTAAAAGAATTGAAAAAAGACCC |
| | | | | GAGCGTCGCTTACGTTGAAGAAGATCAC |
| | | | | GTAGCACACGCGTAC (SEQ ID NO: 8) |
| 340 | Q46H, | 364.00 ± 13.40 | VRSKKLWISLLFALA | GTGAGAAGCAAAAAATTGTGGATCAGTT |
| | p.T47del | | LIFTMAFGSTSSAQA | TGCTGTTTGCTTTAGCGTTAATCTTTACG |
| | | | AGKSNGEKKYIVGF | ATGGCGTTCGGCAGCACATCCAGCGCG |
| | | | KHMSTMSAAKKKD | CAGGCGGCAGGGAAATCAAACGGGGAA |
| | | | VISEKGGKVQKQFK | AAGAAATATATTGTCGGGTTTAAACATAT |
| | | | YVDAASATLNEKAV | GAGCACGATGAGCGCCGCTAAGAAGAA |
| | | | KELKKDPSVAYVEE | AGATGTCATTTCTGAAAAAGGCGGGAAA |
| | | | DHVAHAY (SEQ ID | GTGCAAAAGCAATTCAAATATGTAGACG |
| | | | NO: 335) | CAGCTTCAGCTACATTAAACGAAAAAGC |
| | | | | TGTAAAAGAATTGAAAAAAGACCCGAGC |
| | | | | GTCGCTTACGTTGAAGAAGATCACGTAG |
| | | | | CACACGCGTAC (SEQ ID NO: 336) |
| 353 | S49C | 393.00 ± 27.48 | VRSKKLWISLLFALA | GTGAGAAGCAAAAAATTGTGGATCAGTT |
| | | | LIFTMAFGSTSSAQA | TGCTGTTTGCTTTAGCGTTAATCTTTACG |
| | | | AGKSNGEKKYIVGF | ATGGCGTTCGGCAGCACATCCAGCGCG |
| | | | KQTMCTMSAAKKK | CAGGCGGCAGGGAAATCAAACGGGGAA |
| | | | DVISEKGGKVQKQF | AAGAAATATATTGTCGGGTTTAAACAGA |
| | | | KYVDAASATLNEKA | CAATGTGCACGATGAGCGCCGCTAAGA |
| | | | VKELKKDPSVAYVE | AGAAAGATGTCATTTCTGAAAAAGGCGG |
| | | | EDHVAHAY (SEQ ID | GAAAGTGCAAAAGCAATTCAAATATGTA |
| | | | NO: 337) | GACGCAGCTTCAGCTACATTAAACGAAA |
| | | | | AAGCTGTAAAAGAATTGAAAAAAGACCC |
| | | | | GAGCGTCGCTTACGTTGAAGAAGATCAC |
| | | | | GTAGCACACGCGTAC (SEQ ID NO: 338) |
| 369 | Q70G | 166.10 ± 85.80 | VRSKKLWISLLFALA | GTGAGAAGCAAAAAATTGTGGATCAGTT |
| | | | LIFTMAFGSTSSAQA | TGCTGTTTGCTTTAGCGTTAATCTTTACG |
| | | | AGKSNGEKKYIVGF | ATGGCGTTCGGCAGCACATCCAGCGCG |
| | | | KQTMSTMSAAKKK | CAGGCGGCAGGGAAATCAAACGGGGAA |
| | | | DVISEKGGKVQKGF | AAGAAATATATTGTCGGGTTTAAACAGA |
| | | | KYVDAASATLNEKA | CAATGAGCACGATGAGCGCCGCTAAGA |
| | | | VKELKKDPSVAYVE | AGAAAGATGTCATTTCTGAAAAAGGCGG |
| | | | EDHVAHAY (SEQ ID | GAAAGTGCAAAAGGGATTCAAATATGTA |
| | | | NO: 339) | GACGCAGCTTCAGCTACATTAAACGAAA |
| | | | | AAGCTGTAAAAGAATTGAAAAAAGACCC |
| | | | | GAGCGTCGCTTACGTTGAAGAAGATCAC |
| | | | | GTAGCACACGCGTAC (SEQ ID NO: 340) |
| 371 | Q70L | 295.10 ± 44.50 | VRSKKLWISLLFALA | GTGAGAAGCAAAAAATTGTGGATCAGTT |
| | | | LIFTMAFGSTSSAQA | TGCTGTTTGCTTTAGCGTTAATCTTTACG |
| | | | AGKSNGEKKYIVGF | ATGGCGTTCGGCAGCACATCCAGCGCG |
| | | | KQTMSTMSAAKKK | CAGGCGGCAGGGAAATCAAACGGGGAA |
| | | | DVISEKGGKVQKLF | AAGAAATATATTGTCGGGTTTAAACAGA |
| | | | KYVDAASATLNEKA | CAATGAGCACGATGAGCGCCGCTAAGA |
| | | | VKELKKDPSVAYVE | AGAAAGATGTCATTTCTGAAAAAGGCGG |
| | | | EDHVAHAY (SEQ ID | GAAAGTGCAAAAGTTGTTCAAATATGTA |
| | | | NO: 341) | GACGCAGCTTCAGCTACATTAAACGAAA |
| | | | | AAGCTGTAAAAGAATTGAAAAAAGACCC |
| | | | | GAGCGTCGCTTACGTTGAAGAAGATCAC |
| | | | | GTAGCACACGCGTAC (SEQ ID NO: 342) |
| 381 | S52H | 20 | VRSKKLWISLLFALA | GTGAGAAGCAAAAAATTGTGGATCAGTT |
| | | | LIFTMAFGSTSSAQA | TGCTGTTTGCTTTAGCGTTAATCTTTACG |
| | | | AGKSNGEKKYIVGF | ATGGCGTTCGGCAGCACATCCAGCGCG |
| | | | KQTMSTMHAAKKK | CAGGCGGCAGGGAAATCAAACGGGGAA |
| | | | DVISEKGGKVQKQF | AAGAAATATATTGTCGGGTTTAAACAGA |
| | | | KYVDAASATLNEKA | CAATGAGCACGATGCATGCCGCTAAGAA |
| | | | VKELKKDPSVAYVE | GAAAGATGTCATTTCTGAAAAAGGCGGG |
| | | | EDHVAHAY (SEQ ID | AAAGTGCAAAAGCAATTCAAATATGTAG |
| | | | NO: 343) | ACGCAGCTTCAGCTACATTAAACGAAAA |
| | | | | AGCTGTAAAAGAATTGAAAAAAGACCCG |
| | | | | AGCGTCGCTTACGTTGAAGAAGATCACG |
| | | | | TAGCACACGCGTAC (SEQ ID NO: 344) |
| 390 | p.K55del | 154.50 ± 30.60 | VRSKKLWISLLFALA | GTGAGAAGCAAAAAATTGTGGATCAGTT |
| | | | LIFTMAFGSTSSAQA | TGCTGTTTGCTTTAGCGTTAATCTTTACG |
| | | | AGKSNGEKKYIVGF | ATGGCGTTCGGCAGCACATCCAGCGCG |
| | | | KQTMSTMSAAKKD | CAGGCGGCAGGGAAATCAAACGGGGAA |
| | | | VISEKGGKVQKQFK | AAGAAATATATTGTCGGGTTTAAACAGA |
| | | | YVDAASATLNEKAV | CAATGAGCACGATGAGCGCCGCGAAGA |
| | | | KELKKDPSVAYVEE | AAGATGTCATTTCTGAAAAAGGCGGGAA |
| | | | DHVAHAY (SEQ ID | AGTGCAAAAGCAATTCAAATATGTAGAC |
| | | | NO: 345) | GCAGCTTCAGCTACATTAAACGAAAAAG |
| | | | | CTGTAAAAGAATTGAAAAAAGACCCGAG |
| | | | | CGTCGCTTACGTTGAAGAAGATCACGTA |
| | | | | GCACACGCGTAC (SEQ ID NO: 346) |
| 416 | p.E37del | 75.00 | VRSKKLWISLLFALA | GTGAGAAGCAAAAAATTGTGGATCAGTT |
| | | | LIFTMAFGSTSSAQA | TGCTGTTTGCTTTAGCGTTAATCTTTACG |
| | | | AGKSNGKKYIVGFK | ATGGCGTTCGGCAGCACATCCAGCGCG |
| | | | QTMSTMSAAKKKD | CAGGCGGCAGGGAAATCAAACGGGAAG |
| | | | VISEKGGKVQKQFK | AAATATATTGTCGGGTTTAAACAGACAAT |
| | | | YVDAASATLNEKAV | GAGCACGATGAGCGCCGCTAAGAAGAA |
| | | | KELKKDPSVAYVEE | AGATGTCATTTCTGAAAAAGGCGGGAAA |
| | | | DHVAHAY (SEQ ID | GTGCAAAAGCAATTCAAATATGTAGACG |
| | | | NO: 347) | CAGCTTCAGCTACATTAAACGAAAAAGC |
| | | | | TGTAAAAGAATTGAAAAAAGACCCGAGC |
| | | | | GTCGCTTACGTTGAAGAAGATCACGTAG |
| | | | | CACACGCGTAC (SEQ ID NO: 348) |
| 420 | Q70M | 61.00 ± 15.3 | VRSKKLWISLLFALA | GTGAGAAGCAAAAAATTGTGGATCAGTT |
| | | | LIFTMAFGSTSSAQA | TGCTGTTTGCTTTAGCGTTAATCTTTACG |
| | | | AGKSNGEKKYIVGF | ATGGCGTTCGGCAGCACATCCAGCGCG |
| | | | KQTMSTMSAAKKK | CAGGCGGCAGGGAAATCAAACGGGGAA |
| | | | DVISEKGGKVQKMF | AAGAAATATATTGTCGGGTTTAAACAGA |
| | | | KYVDAASATLNEKA | CAATGAGCACGATGAGCGCCGCTAAGA |
| | | | VKELKKDPSVAYVE | AGAAAGATGTCATTTCTGAAAAAGGCGG |
| | | | EDHVAHAY (SEQ ID | GAAAGTGCAAAAGATGTTCAAATATGTA |
| | | | NO: 349) | GACGCAGCTTCAGCTACATTAAACGAAA |
| | | | | AAGCTGTAAAAGAATTGAAAAAAGACCC |
| | | | | GAGCGTCGCTTACGTTGAAGAAGATCAC |
| | | | | GTAGCACACGCGTAC (SEQ ID NO: 350) |
| 422 | p.G36_E37insG | 29.00 | VRSKKLWISLLFALA | GTGAGAAGCAAAAAATTGTGGATCAGTT |
| | | | LIFTMAFGSTSSAQA | TGCTGTTTGCTTTAGCGTTAATCTTTACG |
| | | | AGKSNGGEKKYIVG | ATGGCGTTCGGCAGCACATCCAGCGCG |
| | | | FKQTMSTMSAAKKK | CAGGCGGCAGGGAAATCAAACGGGGG |
| | | | DVISEKGGKVQKQF | GGAAAAGAAATATATTGTCGGGTTTAAA |
| | | | KYVDAASATLNEKA | CAGACAATGAGCACGATGAGCGCCGCT |
| | | | VKELKKDPSVAYVE | AAGAAGAAAGATGTCATTTCTGAAAAAG |
| | | | EDHVAHAY (SEQ ID | GCGGGAAAGTGCAAAAGCAATTCAAATA |
| | | | NO: 351) | TGTAGACGCAGCTTCAGCTACATTAAAC |
| | | | | GAAAAAGCTGTAAAGGAATTGAAAAAAG |
| | | | | ACCCGAGCGTCGCTTACGTTGAAGAAG |
| | | | | ATCACGTAGCACACGCGTAC (SEQ ID |
| | | | | NO: 352) |
| 425 | S61F | 69.00 | VRSKKLWISLLFALA | GTGAGAAGCAAAAAATTGTGGATCAGTT |
| | | | LIFTMAFGSTSSAQA | TGCTGTTTGCTTTAGCGTTAATCTTTACG |
| | | | AGKSNGEKKYIVGF | ATGGCGTTCGGCAGCACATCCAGCGCG |
| | | | KQTMSTMSAAKKK | CAGGCGGCAGGGAAATCAAACGGGGAA |
| | | | DVIFEKGGKVQKQF | AAGAAATATATTGTCGGGTTTAAACAGA |
| | | | KYVDAASATLNEKA | CAATGAGCACGATGAGCGCCGCTAAGA |
| | | | VKELKKDPSVAYVE | AGAAAGATGTCATTTTCGAAAAAGGCGG |
| | | | EDHVAHAY (SEQ ID | GAAAGTGCAAAAGCAATTCAAATATGTA |
| | | | NO: 353) | GACGCAGCTTCAGCTACATTAAACGAAA |
| | | | | AAGCTGTAAAAGAATTGAAAAAAGACCC |
| | | | | GAGCGTCGCTTACGTTGAAGAAGATCAC |
| | | | | GTAGCACACGCGTAC (SEQ ID NO: 354) |
| 426 | Q70G | 62.60 ± 13.40 | VRSKKLWISLLFALA | GTGAGAAGCAAAAAATTGTGGATCAGTT |
| | | | LIFTMAFGSTSSAQA | TGCTGTTTGCTTTAGCGTTAATCTTTACG |
| | | | AGKSNGEKKYIVGF | ATGGCGTTCGGCAGCACATCCAGCGCC |
| | | | KQTMSTMSAAKKK | CAGGCGGCAGGGAAATCAAACGGGGAA |
| | | | DVISEKGGKVQKGF | AAGAAATATATTGTCGGGTTTAAACAGA |
| | | | KYVDAASATLNEKA | CAATGAGCACGATGAGCGCCGCTAAGA |
| | | | VKELKKDPSVAYVE | AGAAAGATGTCATTTCTGAAAAAGGCGG |
| | | | EDHVAHAY (SEQ ID | GAAAGTGCAAAAGGGGTTCAAATATGTA |
| | | | NO: 355) | GACGCAGCTTCAGCTACATTAAACGAAA |
| | | | | AAGCTGTAAAAGAATTGAAAAAAGACCC |
| | | | | GAGCGTCGCTTACGTTGAAGAAGATCAC |
| | | | | GTAGCACACGCGTAC (SEQ ID NO: 356) |
| 429 | E37G | 53.00 | VRSKKLWISLLFALA | GTGAGAAGCAAAAAATTGTGGATCAGTT |
| | | | LIFTMAFGSTSSAQA | TGCTGTTTGCTTTAGCGTTAATCTTTACG |
| | | | AGKSNGGKKYIVGF | ATGGCGTTCGGCAGCACATCCAGCGCG |
| | | | KQTMSTMSAAKKK | CAGGCGGCAGGGAAATCAAACGGGGGT |
| | | | DVISEKGGKVQKQF | AAGAAATATATTGTCGGGTTTAAACAGA |
| | | | KYVDAASATLNEKA | CAATGAGCACGATGAGCGCCGCTAAGA |
| | | | VKELKKDPSVAYVE | AGAAAGATGTCATTTCTGAAAAAGGCGG |
| | | | EDHVAHAY (SEQ ID | GAAAGTGCAAAAGCAATTCAAATATGTA |
| | | | NO: 357) | GACGCAGCTTCAGCTACATTAAACGAAA |
| | | | | AAGCTGTAAAAGAATTGAAAAAAGACCC |
| | | | | GAGCGTCGCTTACGTTGAAGAAGATCAC |
| | | | | GTAGCACACGCGTAC (SEQ ID NO: 358) |
| 441 | E62V | 58.00 | VRSKKLWISLLFALA | GTGAGAAGCAAAAAATTGTGGATCAGTT |
| | | | LIFTMAFGSTSSAQA | TGCTGTTTGCTTTAGCGTTAATCTTTACG |
| | | | AGKSNGEKKYIVGF | ATGGCGTTCGGCAGCACATCCAGCGCG |
| | | | KQTMSTMSAAKKK | CAGGCGGCAGGGAAATCAAACGGGGAA |
| | | | DVISVKGGKVQKQF | AAGAAATATATTGTCGGGTTTAAACAGA |
| | | | KYVDAASATLNEKA | CAATGAGCACGATGAGCGCCGCTAAGA |
| | | | VKELKKDPSVAYVE | AGAAAGATGTCATTTCTGTCAAAGGCGG |
| | | | EDHVAHAY (SEQ ID | GAAAGTGCAAAAGCAATTCAAATATGTA |
| | | | NO: 359) | GACGCAGCTTCAGCTACATTAAACGAAA |
| | | | | AAGCTGTAAAAGAATTGAAAAAAGACCC |
| | | | | GAGCGTCGCTTACGTTGAAGAAGATCAC |
| | | | | GTAGCACACGCGTAC (SEQ ID NO: 360) |
| 462 | p.R2_S3insT | 134.20 ± 68.40 | VRTSKKLWISLLFAL | GTGAGAACGAGCAAAAAATTGTGGATCA |
| | | | ALIFTMAFGSTSSAQ | GTTTGCTGTTTGCTTTAGCGTTAATCTTT |
| | | | AAGKSNGEKKYIVG | ACGATGGCGTTCGGCAGCACATCCAGC |
| | | | FKQTMSTMSAAKKK | GCGCAGGCGGCAGGGAAATCAAACGGG |
| | | | DVISEKGGKVQKQF | GAAAAGAAATATATTGTCGGGTTTAAAC |
| | | | KYVDAASATLNEKA | AGACAATGAGCACGATGAGCGCCGCTA |
| | | | VKELKKDPSVAYVE | AGAAGAAAGATGTCATTTCTGAAAAAGG |
| | | | EDHVAHAY (SEQ ID | CGGGAAAGTGCAAAAGCAATTCAAATAT |
| | | | NO: 361) | GTAGACGCAGCTTCAGCTACATTAAACG |
| | | | | AAAAAGCTGTAAAAGAATTGAAAAAAGA |
| | | | | CCCGAGCGTCGCTTACGTTGAAGAAGAT |
| | | | | CACGTAGCACACGCGTAC (SEQ ID |
| | | | | NO: 362) |
| 464 | pD58_V59insA | 46.60 ± 22.70 | VRSKKLWISLLFALA | GTGAGAAGCAAAAAATTGTGGATCAGTT |
| | | | LIFTMAFGSTSSAQA | TGCTGTTTGCTTTAGCGTTAATCTTTACG |
| | | | AGKSNGEKKYIVGF | ATGGCGTTCGGCAGCACATCCAGCGCG |
| | | | KQTMSTMSAAKKK | CAGGCGGCAGGGAAATCAAACGGGGAA |
| | | | DAVISEKGGKVQKQ | AAGAAATATATTGTCGGGTTTAAACAGA |
| | | | FKYVDAASATLNEK | CAATGAGCACGATGAGCGCCGCTAAGA |
| | | | AVKELKKDPSVAYV | AGAAAGATGCCGTCATTTCTGAAAAAGG |
| | | | EEDHVAHAY (SEQ | CGGGAAAGTGCAAAAGCAATTCAAATAT |
| | | | ID NO: 363) | GTAGACGCAGCTTCAGCTACATTAAACG |
| | | | | AAAAAGCTGTAAAAGAATTGAAAAAAGA |
| | | | | CCCGAGCGTCGCTTACGTTGAAGAAGAT |
| | | | | CACGTAGCACACGCGTAC (SEQ ID |
| | | | | NO: 364) |
| 466 | S78V | 35.04 ± 21.20 | VRSKKLWISLLFALA | GTGAGAAGCAAAAAATTGTGGATCAGTT |
| | | | LIFTMAFGSTSSAQA | TGCTGTTTGCTTTAGCGTTAATCTTTACG |
| | | | AGKSNGEKKYIVGF | ATGGCGTTCGGCAGCACATCCAGCGCG |
| | | | KQTMSTMSAAKKK | CAGGCGGCAGGGAAATCAAACGGGGAA |
| | | | DVISEKGGKVQKQF | AAGAAATATATTGTCGGGTTTAAACAGA |
| | | | KYVDAAVATLNEKA | CAATGAGCACGATGAGCGCCGCTAAGA |
| | | | VKELKKDPSVAYVE | AGAAAGATGTCATTTCTGAAAAAGGCGG |
| | | | EDHVAHAY (SEQ ID | GAAAGTGCAAAAGCAATTCAAATATGTA |
| | | | NO: 365) | GACGCAGCTGTCGCTACATTAAACGAAA |
| | | | | AAGCTGTAAAAGAATTGAAAAAAGACCC |
| | | | | GAGCGTCGCTTACGTTGAAGAAGATCAC |
| | | | | GTAGCACACGCGTAC (SEQ ID NO: 366) |
| 469 | p.K55del | 7.70 ± 2.50 | VRSKKLWISLLFALA | GTGAGAAGCAAAAAATTGTGGATCAGTT |
| | | | LIFTMAFGSTSSAQA | TGCTGTTTGCTTTAGCGTTAATCTTTACG |
| | | | AGKSNGEKKYIVGF | ATGGCGTTCGGCAGCACATCCAGCGCG |
| | | | KQTMSTMSAAKKD | CAGGCGGCAGGGAAATCAAACGGGGAA |
| | | | VISEKGGKVQKQFK | AAGAAATATATTGTCGGGTTTAAACAGA |
| | | | YVDAASATLNEKAV | CAATGAGCACGATGAGCGCCGCGAAGA |
| | | | KELKKDPSVAYVEE | AAGATGTCATTTCTGAAAAAGGCGGGAA |
| | | | DHVAHA (SEQ ID | AGTGCAAAAGCAATTCAAATATGTAGAC |
| | | | NO: 367) | GCAGCTTCAGCTACATTAAACGAAAAAG |
| | | | | CTGTAAAAGAATTGAAAAAAGACCCGAG |
| | | | | CGTCGCTTACGTTGAAGAAGATCACGTA |
| | | | | GCACACGCG (SEQ ID NO: 368) |
| 470 | K91A | 43.61 ± 27.77 | VRSKKLWISLLFALA | GTGAGAAGCAAAAAATTGTGGATCAGTT |
| | | | LIFTMAFGSTSSAQA | TGCTGTTTGCTTTAGCGTTAATCTTTACG |
| | | | AGKSNGEKKYIVGF | ATGGCGTTCGGCAGCACATCCAGCGCG |
| | | | KQTMSTMSAAKKK | CAGGCGGCAGGGAAATCAAACGGGGAA |
| | | | DVISEKGGKVQKQF | AAGAAATATATTGTCGGGTTTAAACAGA |
| | | | KYVDAASATLNEKA | CAATGAGCACGATGAGCGCCGCTAAGA |
| | | | VKELKADPSVAYVE | AGAAAGATGTCATTTCTGAAAAAGGCGG |
| | | | EDHVAHAY (SEQ ID | GAAAGTGCAAAAGCAATTCAAATATGTA |
| | | | NO: 369) | GACGCAGCTTCAGCTACATTAAACGAAA |
| | | | | AAGCTGTAAAAGAATTGAAAGCGGACCC |
| | | | | GAGCGTCGCTTACGTTGAAGAAGATCAC |
| | | | | GTAGCACACGCGTAC(SEQ ID NO: 370) |
| 472 | Q70E | 75.4 ± 30.5 | VRSKKLWISLLFALA | GTGAGAAGCAAAAAATTGTGGATCAGTT |
| | | | LIFTMAFGSTSSAQA | TGCTGTTTGCTTTAGCGTTAATCTTTACG |
| | | | AGKSNGEKKYIVGF | ATGGCGTTCGGCAGCACATCCAGCGCG |
| | | | KQTMSTMSAAKKK | CAGGCGGCAGGGAAATCAAACGGGGAA |
| | | | DVISEKGGKVQKEF | AAGAAATATATTGTCGGGTTTAAACAGA |
| | | | KYVDAASATLNEKA | CAATGAGCACGATGAGCGCCGCTAAGA |
| | | | VKELKKDPSVAYVE | AGAAAGATGTCATTTCTGAAAAAGGCGG |
| | | | EDHVAHAY (SEQ ID | GAAAGTGCAAAAGGAGTTCAAATATGTA |
| | | | NO: 371) | GACGCAGCTTCAGCTACATTAAACGAAA |
| | | | | AAGCTGTAAAAGAATTGAAAAAAGACCC |
| | | | | GAGCGTCGCTTACGTTGAAGAAGATCAC |
| | | | | GTAGCACACGCGTAC (SEQ ID NO: 372) |
| 475 | S49A | 33.23 ± 24.00 | VRSKKLWISLLFALA | GTGAGAAGCAAAAAATTGTGGATCAGTT |
| | | | LIFTMAFGSTSSAQA | TGCTGTTTGCTTTAGCGTTAATCTTTACG |
| | | | AGKSNGEKKYIVGF | ATGGCGTTCGGCAGCACATCCAGCGCG |
| | | | KQTMATMSAAKKK | CAGGCGGCAGGGAAATCAAACGGGGAA |
| | | | DVISEKGGKVQKQF | AAGAAATATATTGTCGGGTTTAAACAGA |
| | | | KYVDAASATLNEKA | CAATGGCCACGATGAGCGCCGCTAAGA |
| | | | VKELKKDPSVAYVE | AGAAAGATGTCATTTCTGAAAAAGGCGG |
| | | | EDHVAHAY (SEQ ID | GAAAGTGCAAAAGCAATTCAAATATGTA |
| | | | NO: 373) | GACGCAGCTTCAGCTACATTAAACGAAA |
| | | | | AAGCTGTAAAAGAATTGAAAAAAGACCC |
| | | | | GAGCGTCGCTTACGTTGAAGAAGATCAC |
| | | | | GTAGCACACGCGTAC (SEQ ID NO: 374) |
| 480 | S24T | 75.76 ± 35.24 | VRSKKLWISLLFALA | GTGAGAAGCAAAAAATTGTGGATCAGTT |
| | | | LIFTMAFGTTSSAQA | TGCTGTTTGCTTTAGCGTTAATCTTTACG |
| | | | AGKSNGEKKYIVGF | ATGGCGTTCGGCACCACATCCAGCGCG |
| | | | KQTMSTMSAAKKK | CAGGCGGCAGGGAAATCAAACGGGGAA |
| | | | DVISEKGGKVQKQF | AAGAAATATATTGTCGGGTTTAAACAGA |
| | | | KYVDAASATLNEKA | CAATGAGCACGATGAGCGCCGCTAAGA |
| | | | VKELKKDPSVAYVE | AGAAAGATGTCATTTCTGAAAAAGGCGG |
| | | | EDHVAHAY (SEQ ID | GAAAGTGCAAAAGCAATTCAAATATGTA |
| | | | NO: 375) | GACGCAGCTTCAGCTACATTAAACGAAA |
| | | | | AAGCTGTAAAAGAATTGAAAAAAGACCC |
| | | | | GAGCGTCGCTTACGTTGAAGAAGATCAC |
| | | | | GTAGCACACGCGTAC (SEQ ID NO: 376) |
| 484 | S78M | 90.30 ± 74.44 | VRSKKLWISLLFALA | GTGAGAAGCAAAAAATTGTGGATCAGTT |
| | | | LIFTMAFGSTSSAQA | TGCTGTTTGCTTTAGCGTTAATCTTTACG |
| | | | AGKSNGEKKYIVGF | ATGGCGTTCGGCAGCACATCCAGCGCG |
| | | | KQTMSTMSAAKKK | CAGGCGGCAGGGAAATCAAACGGGGAA |
| | | | DVISEKGGKVQKQF | AAGAAATATATTGTCGGGTTTAAACAGA |
| | | | KYVDAAMATLNEKA | CAATGAGCACGATGAGCGCCGCTAAGA |
| | | | VKELKKDPSVAYVE | AGAAAGATGTCATTTCTGAAAAAGGCGG |
| | | | EDHVAHAY (SEQ ID | GAAAGTGCAAAAGCAATTCAAATATGTA |
| | | | NO: 377) | GACGCAGCTATGGCTACATTAAACGAAA |
| | | | | AAGCTGTAAAAGAATTGAAAAAAGACCC |
| | | | | GAGCGTCGCTTACGTTGAAGAAGATCAC |
| | | | | GTAGCACACGCGTAC (SEQ ID NO: 378) |
| 486 | P93S | 118.72 ± 14.45 | VRSKKLWISLLFALA | GTGAGAAGCAAAAAATTGTGGATCAGTT |
| | | | LIFTMAFGSTSSAQA | TGCTGTTTGCTTTAGCGTTAATCTTTACG |
| | | | AGKSNGEKKYIVGF | ATGGCGTTCGGCAGCACATCCAGCGCG |
| | | | KQTMSTMSAAKKK | CAGGCGGCAGGGAAATCAAACGGGGAA |
| | | | DVISEKGGKVQKQF | AAGAAATATATTGTCGGGTTTAAACAGA |
| | | | KYVDAASATLNEKA | CAATGAGCACGATGAGCGCCGCTAAGA |
| | | | VKELKKDSSVAYVE | AGAAAGATGTCATTTCTGAAAAAGGCGG |
| | | | EDHVAHAY (SEQ ID | GAAAGTGCAAAAGCAATTCAAATATGTA |
| | | | NO: 379) | GACGCAGCTTCAGCTACATTAAACGAAA |
| | | | | AAGCTGTAAAAGAATTGAAAAAAGACTC |
| | | | | GAGCGTCGCTTACGTTGAAGAAGATCAC |
| | | | | GTAGCACACGCGTAC (SEQ ID NO: 380) |
| 488 | p.T19_M20insAT | 9.13 ± 5.39 | VRSKKLWISLLFALA | GTGAGAAGCAAAAAATTGTGGATCAGTT |
| | | | LIFTATMAFGSTSSA | TGCTGTTTGCTTTAGCGTTAATCTTTACG |
| | | | QAAGKSNGEKKYIV | GCCACGATGGCGTTCGGCAGCACATCC |
| | | | GFKQTMSTMSAAK | AGCGCGCAGGCGGCAGGGAAATCAAAC |
| | | | KKDVISEKGGKVQK | GGGGAAAAGAAATATATTGTCGGGTTTA |
| | | | QFKYVDAASATLNE | AACAGACAATGAGCACGATGAGCGCCG |
| | | | KAVKELKKDPSVAY | CTAAGAAGAAAGATGTCATTTCTGAAAA |
| | | | VEEDHVAHAY (SEQ | AGGCGGGAAAGTGCAAAAGCAATTCAAA |
| | | | ID NO: 381) | TATGTAGACGCAGCTTCAGCTACATTAA |
| | | | | ACGAAAAAGCTGTAAAAGAATTGAAAAA |
| | | | | AGACCCGAGCGTCGCTTACGTTGAAGA |
| | | | | AGATCACGTAGCACACGCGTAC (SEQ ID |
| | | | | NO: 382) |
| 504 | p.T47del | 56.20 ± 12.40 | VRSKKLWISLLFALA | GTGAGAAGCAAAAAATTGTGGATCAGTT |
| | | | LIFTMAFGSTSSAQA | TGCTGTTTGCTTTAGCGTTAATCTTTACG |
| | | | AGKSNGEKKYIVGF | ATGGCGTTCGGCAGCACATCCAGCGCG |
| | | | KQMSTMSAAKKKD | CAGGCGGCAGGGAAATCAAACGGGGAA |
| | | | VISEKGGKVQKQFK | AAGAAATATATTGTCGGGTTTAAACAGAT |
| | | | YVDAASATLNEKAV | GAGCACGATGAGCGCCGCTAAGAAGAA |
| | | | KELKKDPSVAYVEE | AGATGTCATTTCTGAAAAAGGCGGGAAA |
| | | | DHVAHAY (SEQ ID | GTGCAAAAGCAATTCAAATATGTAGACG |
| | | | NO: 383) | CAGCTTCAGCTACATTAAACGAAAAAGC |
| | | | | TGTAAAAGAATTGAAAAAAGACCCGAGC |
| | | | | GTCGCTTACGTTGAAGAAGATCACGTAG |
| | | | | CACACGCGTAC (SEQ ID NO: 384) |
| 506 | Q70G | 71.50 ± 65.30 | VRSKKLWISLLFALA | GTGAGAAGCAAAAAATTGTGGATCAGTT |
| | | | LIFTMAFGSTSSAQA | TGCTGTTTGCTTTAGCGTTAATCTTTACG |
| | | | AGKSNGEKKYIVGF | ATGGCGTTCGGCAGCACATCCAGCGCG |
| | | | KQTMSTMSAAKKK | CAGGCGGCAGGGAAATCAAACGGGGAA |
| | | | DVISEKGGKVQKGF | AAGAAATATATTGTCGGGTTTAAACAGA |
| | | | KYVDAASATLNEKA | CAATGAGCACGATGAGCGCCGCTAAGA |
| | | | VKELKKDPSVAYVE | AGAAAGATGTCATTTCTGAAAAAGGCGG |
| | | | EDHVAHAY (SEQ ID | GAAAGTGCAAAAGGGGTTCAAATATGTA |
| | | | NO: 385) | GACGCAGCTTCAGCTACATTAAACGAAA |
| | | | | AAGCTGTAAAAGAATTGAAAAAAGACCC |
| | | | | GAGCGTCGCTTACGTTGAAGAAGATCAC |
| | | | | GTAGCACACGCGTAC (SEQ ID NO: 386) |
| 515 | M48I, p.S49del | 229.68 ± 29.83 | VRSKKLWISLLFALA | GTGAGAAGCAAAAAATTGTGGATCAGTT |
| | | | LIFTMAFGSTSSAQA | TGCTGTTTGCTTTAGCGTTAATCTTTACG |
| | | | AGKSNGEKKYIVGF | ATGGCGTTCGGCAGCACATCCAGCGCG |
| | | | KQTITMSAAKKKDVI | CAGGCGGCAGGGAAATCAAACGGGGAA |
| | | | SEKGGKVQKQFKY | AAGAAATATATTGTCGGGTTTAAACAGA |
| | | | VDAASATLNEKAVK | CAATCACGATGAGCGCCGCTAAGAAGA |
| | | | ELKKDPSVAYVEED | AAGATGTCATTTCTGAAAAAGGCGGGAA |
| | | | HVAHAY (SEQ ID | AGTGCAAAAGCAATTCAAATATGTAGAC |
| | | | NO: 387) | GCAGCTTCAGCTACATTAAACGAAAAAG |
| | | | | CTGTAAAAGAATTGAAAAAAGACCCGAG |
| | | | | CGTCGCTTACGTTGAAGAAGATCACGTA |
| | | | | GCACACGCGTAC (SEQ ID NO: 388) |
| 521 | S52H | 69.06 ± 33.01 | VRSKKLWISLLFALA | GTGAGAAGCAAAAAATTGTGGATCAGTT |
| | | | LIFTMAFGSTSSAQA | TGCTGTTTGCTTTAGCGTTAATCTTTACG |
| | | | AGKSNGEKKYIVGF | ATGGCGTTCGGCAGCACATCCAGCGCG |
| | | | KQTMSTMHAAKKK | CAGGCGGCAGGGAAATCAAACGGGGAA |
| | | | DVISEKGGKVQKQF | AAGAAATATATTGTCGGGTTTAAACAGA |
| | | | KYVDAASATLNEKA | CAATGAGCACGATGCATGCCGCTAAGAA |
| | | | VKELKKDPSVAYVE | GAAAGATGTCATTTCTGAAAAAGGCGGG |
| | | | EDHVAHAY (SEQ ID | AAAGTGCAAAAGCAATTCAAATATGTAG |
| | | | NO: 389) | ACGCAGCTTCAGCTACATTAAACGAAAA |
| | | | | AGCTGTAAAAGAATTGAAAAAAGACCCG |
| | | | | AGCGTCGCTTACGTTGAAGAAGATCACG |
| | | | | TAGCACACGCGTAC (SEQ ID NO: 390) |
| 524 | p.F22_G23del | 40.00 ± 10.88 | VRSKKLWISLLFALA | GTGAGAAGCAAAAAATTGTGGATCAGTT |
| | | | LIFTMASTSSAQAA | TGCTGTTTGCTTTAGCGTTAATCTTTACG |
| | | | GKSNGEKKYIVGFK | ATGGCGAGCACATCCAGCGCGCAGGCG |
| | | | QTMSTMSAAKKKD | GCAGGGAAATCAAACGGGGAAAAGAAA |
| | | | VISEKGGKVQKQFK | TATATTGTCGGGTTTAAACAGACAATGA |
| | | | YVDAASATLNEKAV | GCACGATGAGCGCCGCTAAGAAGAAAG |
| | | | KELKKDPSVAYVEE | ATGTCATTTCTGAAAAAGGCGGGAAAGT |
| | | | DHVAHAY (SEQ ID | GCAAAAGCAATTCAAATATGTAGACGCA |
| | | | NO: 391) | GCTTCAGCTACATTAAACGAAAAAGCTG |
| | | | | TAAAAGAATTGAAAAAAGACCCGAGCGT |
| | | | | CGCTTACGTTGAAGAAGATCACGTAGCA |
| | | | | CACGCGTAC (SEQ ID NO: 392) |
| 531 | S49A | 91.80 ± 25.10 | VRSKKLWISLLFALA | GTGAGAAGCAAAAAATTGTGGATCAGTT |
| | | | LIFTMAFGSTSSAQA | TGCTGTTTGCTTTAGCGTTAATCTTTACG |
| | | | AGKSNGEKKYIVGF | ATGGCGTTCGGCAGCACATCCAGCGCG |
| | | | KQTMATMSAAKKK | CAGGCGGCAGGGAAATCAAACGGGGAA |
| | | | DVISEKGGKVQKQF | AAGAAATATATTGTCGGGTTTAAACAGA |
| | | | KYVDAASATLNEKA | CAATGGCCACGATGAGCGCCGCTAAGA |
| | | | VKELKKDPSVAYVE | AGAAAGATGTCATTTCTGAAAAAGGCGG |
| | | | EDHVAHAY (SEQ ID | GAAAGTGCAAAAGCAATTCAAATATGTA |
| | | | NO: 393) | GACGCAGCTTCAGCTACATTAAACGAAA |
| | | | | AAGCTGTAAAAGAATTGAAAAAAGACCC |
| | | | | GAGCGTCGCTTACGTTGAAGAAGATCAC |
| | | | | GTAGCACACGCGTAC (SEQ ID NO: 394) |
| 532 | p.K57del | 31.30 ± 8.60 | VRSKKLWISLLFALA | GTGAGAAGCAAAAAATTGTGGATCAGTT |
| | | | LIFTMAFGSTSSAQA | TGCTGTTTGCTTTAGCGTTAATCTTTACG |
| | | | AGKSNGEKKYIVGF | ATGGCGTTCGGCAGCACATCCAGCGCG |
| | | | KQTMSTMSAAKKD | CAGGCGGCAGGGAAATCAAACGGGGAA |
| | | | VISEKGGKVQKQFK | AAGAAATATATTGTCGGGTTTAAACAGA |
| | | | YVDAASATLNEKAV | CAATGAGCACGATGAGCGCCGCTAAGA |
| | | | KELKKDPSVAYVEE | AGGATGTCATTTCTGAAAAAGGCGGGAA |
| | | | DHVAHAY (SEQ ID | AGTGCAAAAGCAATTCAAATATGTAGAC |
| | | | NO: 395) | GCAGCTTCAGCTACATTAAACGAAAAAG |
| | | | | CTGTAAAAGAATTGAAAAAAGACCCGAG |
| | | | | CGTCGCTTACGTTGAAGAAGATCACGTA |
| | | | | GCACACGCGTAC (SEQ ID NO: 396) |
| 541 | p.G32_K33insG | 50.01 ± 13.55 | VRSKKLWISLLFALA | GTGAGAAGCAAAAAATTGTGGATCAGTT |
| | | | LIFTMAFGSTSSAQA | TGCTGTTTGCTTTAGCGTTAATCTTTACG |
| | | | AGGKSNGEKKYIVG | ATGGCGTTCGGCAGCACATCCAGCGCG |
| | | | FKQTMSTMSAAKKK | CAGGCGGCAGGTGGGAAATCAAACGGG |
| | | | DVISEKGGKVQKQF | GAAAAGAAATATATTGTCGGGTTTAAAC |
| | | | KYVDAASATLNEKA | AGACAATGAGCACGATGAGCGCCGCTA |
| | | | VKELKKDPSVAYVE | AGAAGAAAGATGTCATTTCTGAAAAAGG |
| | | | EDHVAHAY (SEQ ID | CGGGAAAGTGCAAAAGCAATTCAAATAT |
| | | | NO: 397) | GTAGACGCAGCTTCAGCTACATTAAACG |
| | | | | AAAAAGCTGTAAAAGAATTGAAAAAAGA |
| | | | | CCCGAGCGTCGCTTACGTTGAAGAAGAT |
| | | | | CACGTAGCACACGCGTAC (SEQ ID |
| | | | | NO: 398) |
| 734 | K72N | 89.42 ± 67.68 | VRSKKLWISLLFALA | GTGAGAAGCAAAAAATTGTGGATCAGTT |
| | | | LIFTMAFGSTSSAQA | TGCTGTTTGCTTTAGCGTTAATCTTTACG |
| | | | AGKSNGEKKYIVGF | ATGGCGTTCGGCAGCACATCCAGCGCG |
| | | | KQTMSTMSAAKKK | CAGGCGGCAGGGAAATCAAACGGGGAA |
| | | | DVISEKGGKVQKQF | AAGAAATATATTGTCGGGTTTAAACAGA |
| | | | DYVDAASATLNEKA | CAATGAGCACGATGAGCGCCGCTAAGA |
| | | | VKELKKDPSVAYVE | AGAAAGATGTCATTTCTGAAAAAGGCGG |
| | | | EDHVAHAY (SEQ ID | GAAAGTGCAAAAGCAATTCGATTATGTA |
| | | | NO: 399) | GACGCAGCTTCAGCTACATTAAACGAAA |
| | | | | AAGCTGTAAAAGAATTGAAAAAAGACCC |
| | | | | GAGCGTCGCTTACGTTGAAGAAGATCAC |
| | | | | GTAGCACACGCGTAC (SEQ ID NO: 400) |
| 767 | p.A21_F22insS | 41.60 ± 17.80 | VRSKKLWISLLFALA | GTGAGAAGCAAAAAATTGTGGATCAGTT |
| | | | LIFTMASFGSTSSAQ | TGCTGTTTGCTTTAGCGTTAATCTTTACG |
| | | | AAGKSNGEKKYIVG | ATGGCGAGTTTCGGCAGCACATCCAGC |
| | | | FKQTMSTMSAAKKK | GCGCAGGCGGCAGGGAAATCAAACGGG |
| | | | DVISEKGGKVQKQF | GAAAAGAAATATATTGTCGGGTTTAAAC |
| | | | KYVDAASATLNEKA | AGACAATGAGCACGATGAGCGCCGCTA |
| | | | VKELKKDPSVAYVE | AGAAGAAAGATGTCATTTCTGAAAAAGG |
| | | | EDHVAHAY (SEQ ID | CGGGAAAGTGCAAAAGCAATTCAAATAT |
| | | | NO: 401) | GTAGACGCAGCTTCAGCTACATTAAACG |
| | | | | AAAAAGCTGTAAAAGAATTGAAAAAAGA |
| | | | | CCCGAGCGTCGCTTACGTTGAAGAAGAT |
| | | | | CACGTAGCACACGCGTAC (SEQ ID |
| | | | | NO: 402) |
| 771 | K57L | 47.40 ± 6.90 | VRSKKLWISLLFALA | GTGAGAAGCAAAAAATTGTGGATCAGTT |
| | | | LIFTMAFGSTSSAQA | TGCTGTTTGCTTTAGCGTTAATCTTTACG |
| | | | AGKSNGEKKYIVGF | ATGGCGTTCGGCAGCACATCCAGCGCG |
| | | | KQTMSTMSAAKKLD | CAGGCGGCAGGGAAATCAAACGGGGAA |
| | | | VISEKGGKVQKQFK | AAGAAATATATTGTCGGGTTTAAACAGA |
| | | | YVDAASATLNEKAV | CAATGAGCACGATGAGCGCCGCTAAGA |
| | | | KELKKDPSVAYVEE | AGTTGGATGTCATTTCTGAAAAAGGCGG |
| | | | DHVAHAY (SEQ ID | GAAAGTGCAAAAGCAATTCAAATATGTA |
| | | | NO: 403) | GACGCAGCTTCAGCTACATTAAACGAAA |
| | | | | AAGCTGTAAAAGAATTGAAAAAAGACCC |
| | | | | GAGCGTCGCTTACGTTGAAGAAGATCAC |
| | | | | GTAGCACACGCGTAC (SEQ ID NO: 404) |
| 773 | p.A30_A31insA | 51.00 ± 37.70 | VRSKKLWISLLFALA | GTGAGAAGCAAAAAATTGTGGATCAGTT |
| | | | LIFTMAFGSTSSAQA | TGCTGTTTGCTTTAGCGTTAATCTTTACG |
| | | | AAGKSNGEKKYIVG | ATGGCGTTCGGCAGCACATCCAGCGCG |
| | | | FKQTMSTMSAAKKK | CAGGCGGCCGCAGGGAAATCAAACGGG |
| | | | DVISEKGGKVQKQF | GAAAAGAAATATATTGTCGGGTTTAAAC |
| | | | KYVDAASATLNEKA | AGACAATGAGCACGATGAGCGCCGCTA |
| | | | VKELKKDPSVAYVE | AGAAGAAAGATGTCATTTCTGAAAAAGG |
| | | | EDHVAHAY (SEQ ID | CGGGAAAGTGCAAAAGCAATTCAAATAT |
| | | | NO: 405) | GTAGACGCAGCTTCAGCTACATTAAACG |
| | | | | AAAAAGCTGTAAAAGAATTGAAAAAAGA |
| | | | | CCCGAGCGTCGCTTACGTTGAAGAAGAT |
| | | | | CACGTAGCACACGCGTAC (SEQ ID |
| | | | | NO: 406) |
| 777 | S24G | 129.60 ± 72.30 | VRSKKLWISLLFALA | GTGAGAAGCAAAAAATTGTGGATCAGTT |
| | | | LIFTMAFGGTSSAQ | TGCTGTTTGCTTTAGCGTTAATCTTTACG |
| | | | AAGKSNGEKKYIVG | ATGGCGTTCGGCGGCACATCCAGCGCG |
| | | | FKQTMSTMSAAKKK | CAGGCGGCAGGGAAATCAAACGGGGAA |
| | | | DVISEKGGKVQKQF | AAGAAATATATTGTCGGGTTTAAACAGA |
| | | | KYVDAASATLNEKA | CAATGAGCACGATGAGCGCCGCTAAGA |
| | | | VKELKKDPSVAYVE | AGAAAGATGTCATTTCTGAAAAAGGCGG |
| | | | EDHVAHAY (SEQ ID | GAAAGTGCAAAAGCAATTCAAATATGTA |
| | | | NO: 407) | GACGCAGCTTCAGCTACATTAAACGAAA |
| | | | | AAGCTGTAAAAGAATTGAAAAAAGACCC |
| | | | | GAGCGTCGCTTACGTTGAAGAAGATCAC |
| | | | | GTAGCACACGCGTAC (SEQ ID NO: 408) |
| 1001 | I17W, | 1.28 ± 0.07 | VRSKKLWISLLFALA | GTGAGAAGCAAAAAATTGTGGATCAGTT |
| | p.I18_T19del | | LWMAFGSTSSAQA | TGCTGTTTGCTTTAGCGTTATGGATGGC |
| | | | AGKSNGEKKYIVGF | GTTCGGCAGCACATCCTCTGCCCAGGC |
| | | | KQTMSTMSAAKKK | GGCAGGGAAATCAAACGGGGAAAAGAA |
| | | | DVISEKGGKVQKQF | ATATATTGTCGGGTTTAAACAGACAATG |
| | | | KYVDAASATLNEKA | AGCACGATGAGCGCCGCTAAGAAGAAA |
| | | | VKELKKDPSVAYVE | GATGTCATTTCTGAAAAAGGCGGGAAAG |
| | | | EDHVAHAY (SEQ ID | TGCAAAAGCAATTCAAATATGTAGACGC |
| | | | NO: 409) | AGCTTCAGCTACATTAAACGAAAAAGCT |
| | | | | GTAAAAGAATTGAAAAAAGACCCGAGCG |
| | | | | TCGCTTACGTTGAAGAAGATCACGTAGC |
| | | | | ACACGCGTAC (SEQ ID NO: 410) |

### Example 2

### Generation of Mutated Pre-Pro Polypeptides Comprising a Combination of Mutations Generated by ISD

To determine the effect of combining at least two mutations in the pre-pro FNA sequence, combinations of the mutations given in Table 3 were made as follows.

The pAC-FNA10 plasmid DNAs comprising a mutant from Table 3 was used as a template for extension PCR to add another mutation also selected from mutations described in Table 3. Two PCR reactions (left and right segments) contained either the 5′ forward or the 3′ reverse gene sequence flanking oligonucleotides each in combination with the corresponding oppositely priming oligonucleotides. The left fragments were amplified using a single forward primer (P3234, ACCCAACTGATCTTCAGCATC; SEQ ID NO:411) and reverse primers for the particular mutation shown in Table D. The right fragments were amplified using a single reverse primer (P3242, ACCGTCAGCACCGAGAACTT; SEQ ID NO:412) and forward primers for that particular mutation shown in Table 4. Two amplified fragments (left and right) were mixed together and amplified by the forward primer containing EcoRI site (P3201, ATAGGAATTCATCTCAAAAAAATG; SEQ ID NO:413) and reverse primer containing MluI restriction site (P3237, TGTCGATAACCGCTACTTTAAC; SEQ ID NO:414).

TABLE 4

Sequences of forward and reverse primers used to amplify the
left and right fragments

| | | | | |
|---|---|---|---|---|
| Mutation | Primer | Primer | | |
| introduced | orientation | name | Primer sequence | SEQ ID NO: |

| | | | | |
|---|---|---|---|---|
| Clone 541 | Forward | P3468 | AGGCGGCAGGTGGGAAATCAAACGGGGA | 415 |
| | | | AAAGAAATA | |
| Clone 541 | Reverse | P3469 | TTTCCCCGTTTGATTTCCCACCTGCCGCC | 416 |
| | | | TGCGCGCTGGA | |
| Clone 462 | Forward | P3408 | TTCCATCTATTACAATAAATTCACAGAATA | 417 |
| | | | GTCTTTTAAGTAAGTCTACTCT | |
| Clone 462 | Reverse | P3409 | CTGTGAATTTATTGTAATAGATGGAA | 418 |
| Clone 515 | Forward | P3446 | TTTAAACAGACAATCACGATGAGCGCCGC | 419 |
| | | | TAAGAA | |
| Clone 515 | Reverse | P3447 | AGCGGCGCTCATCGTGATTGTCTGTTTAA | 420 |
| | | | ACCCGACAATA | |
| Clone 466 | Forward | P3478 | TGTAGACGCAGCTGTCGCTACATTAAACG | 421 |
| | | | AAAAAGCTGTA | |
| Clone 466 | Reverse | P3479 | TCGTTTAATGTAGCGACAGCTGCGTCTAC | 422 |
| | | | ATATTTGAATT | |
| Clone 469 | Forward | P3480 | CGATGAGCGCCGCGAAGAAAGATGTCATT | 423 |
| | | | TCTGAAAAA | |
| Clone 469 | Reverse | P3481 | GAAATGACATCTTTCTTCGCGGCGCTCAT | 424 |
| | | | CGTGCTCA | |
| Clone 470 | Forward | P3482 | TGTAAAAGAATTGAAAGCGGACCCGAGCG | 425 |
| | | | TCGCTTACGT | |
| Clone 470 | Reverse | P3483 | GACGCTCGGGTCCGCTTTCAATTCTTTTA | 426 |
| | | | CAGCTTTTTCG | |
| Clone 521 | Forward | P3454 | AATGAGCACGATGCATGCCGCTAAGAAGA | 427 |
| | | | AAGATGTCA | |
| Clone 521 | Reverse | P3455 | TTCTTCTTAGCGGCATGCATCGTGCTCATT | 428 |
| | | | GTCTGTTTAA | |
| Clone 524 | Forward | P3458 | AATCTTTACGATGGCGAGCACATCCAGCG | 429 |
| | | | CGCAGG | |
| Clone 524 | Reverse | P3459 | CGCGCTGGATGTGCTCGCCATCGTAAAGA | 430 |
| | | | TTAACGCT | |
| Clone 475 | Forward | P3484 | GGTTTAAACAGACAATGGCCACGATGAGC | 431 |
| | | | GCCGCTAAGA | |
| Clone 475 | Reverse | P3485 | GCGGCGCTCATCGTGGCCATTGTCTGTTT | 432 |
| | | | AAACCCGACAA | |
| Clone 480 | Forward | P3486 | ATGGCGTTCGGCACCACATCCAGCGCGC | 433 |
| | | | AGGCGGCA | |
| Clone 480 | Reverse | P3487 | CTGCGCGCTGGATGTGGTGCCGAACGCC | 434 |
| | | | ATCGTAAAGA | |
| Clone 448 | Forward | P3488 | GAGAAGCAAAAAATTATGGATCAGTTTGCT | 435 |
| | | | GTTTGCTTT | |
| Clone 448 | Reverse | P3489 | CAGCAAACTGATCCATAATTTTTTGCTTCT | 436 |
| | | | CACTCTTTAC | |
| Clone 484 | Forward | P3490 | TGTAGACGCAGCTATGGCTACATTAAACG | 437 |
| | | | AAAAAGCTGTA | |
| Clone 484 | Reverse | P3491 | TCGTTTAATGTAGCCATAGCTGCGTCTACA | 438 |
| | | | TATTTGAATT | |
| Clone 486 | Forward | P3492 | AAGAATTGAAAAAAGACTCGAGCGTCGCT | 439 |
| | | | TACGTTGAAG | |
| Clone 486 | Reverse | P3493 | AAGCGACGCTCGAGTCTTTTTTCAATTCTT | 440 |
| | | | TTACAGCT | |
| Clone 488 | Forward | P3494 | GCGTTAATCTTTACGGCCACGATGGCGTT | 441 |
| | | | CGGCAGCACAT | |
| Clone 488 | Reverse | P3495 | GAACGCCATCGTGGCCGTAAAGATTAACG | 442 |
| | | | CTAAAGCAAAC | |
| Clone 734 | Forward | P3456 | GTGCAAAAGCAATTCGATTATGTAGACGC | 443 |
| | | | AGCTTCAGCTA | |
| Clone 734 | Reverse | P3457 | TGCGTCTACATAATCGAATTGCTTTTGCAC | 444 |
| | | | TTTCCCGCCT | |

Amplification, ligation and transformation were performed as described in Example 1. Three clones for each combination of mutations were screened for AAPF activity using a 96-well plate assay as described in Example 1. Results for relative production of FNA (SEQ ID NO:9) processed from full-length FNA protein comprising a combination of mutations in pre-pro polypeptide relative to the production of FNA processed from wild-type full-length FNA are shown in Tables 5-10.

TABLE 5
Effect of combining the S49C substitution with a second mutation in
the pre-pro region of FNA on the production of the mature protein

| | | Relative activity | | Relative activity of | Relative Activity |
|---|---|---|---|---|---|
| | First | of First mutation | | the Second mutation | of both mutations |
| Clone | mutation | to unmodified | | to unmodified | to unmodified |
| No. | (clone 353) | (% mean ± S.D.) | Second mutation | (% mean ± S.D.) | (% mean ± S.D.) |
| 832 | S49C | 393.59 ± 27.48 | 488(p.T19_M20insAT | 9.13 ± 5.39 | 100.97 ± 24.1 |
| 687 | S49C | 393.59 ± 27.48 | 524(p.F22_G23del) | 40 ± 10.88 | 105.02 ± 38.1 |
| 713 | S49C | 393.59 ± 27.48 | 480(S24T) | 75.76 ± 35.24 | 475.29 ± 64 |
| 736 | S49C | 393.59 ± 27.48 | 541(p.G32_K33insG) | 50.01 ± 13.55 | 78.57 ± 31.4 |
| 818 | S49C | 393.59 ± 27.48 | 734(K72D) | 89.42 ± 67.68 | 211.71 ± 62.1 |
| 814 | S49C | 393.59 ± 27.48 | 484(S78M) | 90.3 ± 74.44 | 43.56 ± 23.4 |
| 634 | S49C | 393.59 ± 27.48 | 466(S78V) | 35.04 ± 21.2 | 60.2 ± 37.2 |
| 659 | S49C | 393.59 ± 27.48 | 470(K91A) | 43.61 ± 27.77 | 66.37 ± 7.57 |
| 731 | S49C | 393.59 ± 27.48 | 486(P93S) | 118.72 ± 14.45 | 227.34 ± 45.3 |

TABLE 6
Effect of combining the K91C substitution with a second mutation in
the pre-pro region of FNA on the production of the mature protein

| | | Relative activity | | Relative activity of | Relative activity of |
|---|---|---|---|---|---|
| | First | of First mutation | | the Second mutation | both mutations to |
| Clone | mutation | to unmodified | | to unmodified | unmodified |
| No. | (clone 470) | (% mean ± S.D.) | Second mutation | (% mean ± S.D.) | (% mean ± S.D.) |
| 656 | K91A | 43.61 ± 27.77 | 488(p.T19_M20insAT | 9.13 ± 5.39 | 92.47 ± 46.66 |
| 688 | K91A | 43.61 ± 27.77 | 524(p.F22_G23del) | 40.00 ± 10.88 | 157.25 ± 63.06 |
| 650 | K91A | 43.61 ± 27.77 | 480(S24T) | 75.76 ± 35.24 | 118.35 ± 64.56 |
| 783 | K91A | 43.61 ± 27.77 | 541(p.G32_K33insG) | 50.01 ± 13.55 | 41.77 ± 11.24 |
| 591 | K91A | 43.61 ± 27.77 | 515(M48I, p.S49del) | 229.68 ± 29.83 | 101.97 ± 39.49 |
| 659 | K91A | 43.61 ± 27.77 | 353(S49C) | 393.59 ± 27.48 | 66.37 ± 7.57 |
| 648 | K91A | 43.61 ± 27.77 | 475(S49A) | 33.23 ± 24.00 | 117.68 ± 53.42 |
| 606 | K91A | 43.61 ± 27.77 | 521(S52H) | 69.06 ± 33.01 | 78.91 ± 53.90 |
| 636 | K91A | 43.61 ± 27.77 | 469(p.K57del) | 7.70 ± 2.50 | 132.49 ± 9.07 |
| 672 | K91A | 43.61 ± 27.77 | 734(K72D) | 89.42 ± 67.68 | 125.26 ± 9.14 |
| 654 | K91A | 43.61 ± 27.77 | 484(S78M) | 90.30 ± 74.44 | 68.11 ± 6.26 |
| 752 | K91A | 43.61 ± 27.77 | 466(S78V) | 35.04 ± 21.20 | 96.52 ± 33.49 |

TABLE 7
Effect of combining the S49A substitution with a second mutation in
the pre-pro region of FNA on the production of the mature protein

| | | Relative activity | | Relative activity of | Relative activity |
|---|---|---|---|---|---|
| | First | of First mutation | | the Second mutation | of both mutations |
| Clone | mutation | to unmodified FNA | | to unmodified FNA | to unmodified FNA |
| No. | (clone 475) | (% mean ± S.D.) | Second mutation | (% mean ± S.D.) | (% mean ± S.D.) |
| 698 | S49A | 33.23 ± 24.00 | 462(p.R2_S3insT) | 134.20 ± 68.40 | 100.86 ± 30.28 |
| 803 | S49A | 33.23 ± 24.00 | 488(p.T19_M20insAT | 9.13 ± 5.39 | 108.62 ± 42.45 |
| 802 | S49A | 33.23 ± 24.00 | 524(p.F22_G23del) | 40.00 ± 10.88 | 41.69 ± 19.56 |
| 826 | S49A | 33.23 ± 24.00 | 480(S24T) | 75.00 ± 19.10 | 77.91 ± 19.13 |
| 785 | S49A | 33.23 ± 24.00 | 541(p.G32_K33insG) | 50.01 ± 13.55 | 140.11 ± 20.88 |
| 660 | S49A | 33.23 ± 24.00 | 734(K72D) | 89.42 ± 67.68 | 93.72 ± 18.89 |
| 827 | S49A | 33.23 ± 24.00 | 484(S78M) | 90.30 ± 74.44 | 102.74 ± 43.80 |
| 624 | S49A | 33.23 ± 24.00 | 466(S78V) | 35.04 ± 21.20 | 105.01 ± 34.43 |
| 648 | S49A | 33.23 ± 24.00 | 470(K91A) | 43.61 ± 27.77 | 117.68 ± 53.42 |
| 703 | S49A | 33.23 ± 24.00 | 486(P93S) | 118.72 ± 14.45 | 272.32 ± 45.15 |

TABLE 8
Effect of combining the p.T19_M20insAT insertion with a second mutation
in the pre-pro region of FNA on the production of the mature protein

| | | Relative activity | | Relative activity of | Relative activity |
|---|---|---|---|---|---|
| | | of First mutation | | the Second mutation | of both mutations |
| Clone | First mutation | to unmodified FNA | | to unmodified FNA | to unmodified FNA |
| No. | (clone 488) | (% mean ± S.D.) | Second mutation | (% mean ± S.D.) | (% mean ± S.D.) |
| 811 | p.T19_M20insAT | 9.13 ± 5.39 | 448(wt) | 134.20 ± 68.40 | 55.77 ± 20.57 |
| 567 | p.T19_M20insAT | 9.13 ± 5.39 | 541(p.G32_K33insG) | 50.01 ± 13.55 | 70.06 ± 35.51 |
| 601 | p.T19_M20insAT | 9.13 ± 5.39 | 515(M48I, p.S49del) | 229.68 ± 29.83 | 183.98 ± 9.97 |
| 832 | p.T19_M20insAT | 9.13 ± 5.39 | 353(S49C) | 393.59 ± 27.48 | 100.97 ± 24.08 |
| 803 | p.T19_M20insAT | 9.13 ± 5.39 | 475(S49A) | 33.23 ± 24.00 | 108.62 ± 42.45 |
| 616 | p.T19_M20insAT | 9.13 ± 5.39 | 521(S52H) | 69.06 ± 33.01 | 91.57 ± 56.34 |
| 647 | p.T19_M20insAT | 9.13 ± 5.39 | 469(p.K57del) | 7.70 ± 2.50 | 93.14 ± 41.92 |
| 669 | p.T19_M20insAT | 9.13 ± 5.39 | 734(K72D) | 89.42 ± 67.68 | 110.65 ± 33.54 |
| 725 | p.T19_M20insAT | 9.13 ± 5.39 | 484(S78M) | 90.30 ± 74.44 | 280.25 ± 69.52 |
| 632 | p.T19_M20insAT | 9.13 ± 5.39 | 466(S78V) | 35.04 ± 21.20 | 42.16 ± 20.03 |
| 656 | p.T19_M20insAT | 9.13 ± 5.39 | 470(K91A) | 43.61 ± 27.77 | 92.47 ± 46.66 |
| 829 | p.T19_M20insAT | 9.13 ± 5.39 | 486(P93S) | 118.72 ± 14.45 | 157.29 ± 68.38 |

TABLE 9
Effect of combining the p.F22_G23del deletion with a second mutation
in the pre-pro region of FNA on the production of the mature protein

| | | Relative activity | | Relative activity of | Relative activity |
|---|---|---|---|---|---|
| | | of First mutation | | the Second mutation | of both mutations |
| Clone | First mutation | to unmodified FNA | | to unmodified FNA | to unmodified FNA |
| No. | (clone 524) | (% mean ± S.D.) | Second mutation | (% mean ± S.D.) | (% mean ± S.D.) |
| 823 | p.F22_G23del | 40.00 ± 10.88 | 462(p.R2_S3insT) | 44.30 ± 23.62 | 114.90 ± 17.24 |
| 821 | p.F22_G23del | 40.00 ± 10.88 | 448(wt) | 134.20 ± 68.40 | 52.87 ± 11.04 |
| 687 | p.F22_G23del | 40.00 ± 10.88 | 353(S49C) | 393.59 ± 27.48 | 105.02 ± 38.09 |
| 802 | p.F22_G23del | 40.00 ± 10.88 | 475(S49A) | 33.23 ± 24.00 | 41.69 ± 19.56 |
| 759 | p.F22_G23del | 40.00 ± 10.88 | 484(S78M) | 90.30 ± 74.44 | 58.79 ± 15.06 |
| 692 | p.F22_G23del | 40.00 ± 10.88 | 466(S78V) | 35.04 ± 21.20 | 121.46 ± 44.94 |
| 688 | p.F22_G23del | 40.00 ± 10.88 | 470(K91A) | 43.61 ± 27.77 | 157.25 ± 63.06 |
| 684 | p.F22_G23del | 40.00 ± 10.88 | 486(P93S) | 118.72 ± 14.45 | 812.67 ± 46.20 |

TABLE 10
Effect of combining the P93S substitution with a second mutation in
the pre-pro region of FNA on the production of the mature protein

| | | Relative activity | | Relative activity of | Relative activity |
|---|---|---|---|---|---|
| | First | of First mutation | | the Second mutation | of both mutations |
| Clone | mutation | to unmodified FNA | Second | to unmodified FNA | to unmodified FNA |
| No. | (clone 486) | (% mean ± S.D.) | mutation | (% mean ± S.D.) | (% mean ± S.D.) |
| 829 | P93S | 118.70 ± 14.50 | p.T19_M20insAT | 9.10 ± 5.40 | 157.30 ± 68.40 |
| 684 | P93S | 118.70 ± 14.50 | p.F22_G23del | 40.00 ± 10.90 | 812.20 ± 46.20 |
| 710 | P93S | 118.70 ± 14.50 | S24T | 75.80 ± 35.20 | 299.00 ± 76.00 |
| 564 | P93S | 118.70 ± 14.50 | p.G32_K33insG | 50.00 ± 13.60 | 163.30 ± 53.40 |
| 599 | P93S | 118.70 ± 14.50 | M48I, p.S49del | 229.70 ± 29.80 | 258.20 ± 48.50 |
| 731 | P93S | 118.70 ± 14.50 | S49C | 393.60 ± 27.50 | 227.30 ± 45.30 |
| 703 | P93S | 118.70 ± 14.50 | S49A | 33.20 ± 24.00 | 272.30 ± 45.20 |
| 615 | P93S | 118.70 ± 14.50 | S52H | 69.10 ± 33.00 | 157.40 ± 68.70 |
| 644 | P93S | 118.70 ± 14.50 | pK57del | 7.70 ± 2.50 | 167.00 ± 43.30 |
| 666 | P93S | 118.70 ± 14.50 | K72D | 89.40 ± 67.70 | 187.10 ± 28.30 |
| 722 | P93S | 118.70 ± 14.50 | S78M | 90.30 ± 74.40 | 217.00 ± 39.50 |
| 631 | P93S | 118.70 ± 14.50 | S78V | 35.00 ± 21.20 | 161.00 ± 38.30 |

The data show that the majority of combinations resulted in a relative AAPF activity that was greater than that obtained as a result of individual mutations i.e. most combinations of mutations had a synergistic effect on the AAPF activity.

All B. subtilis cells expressing a full-length FNA comprising a pre-pro polypeptide having a combination of mutations had a level of production of the mature FNA that was greater than that of the B. subtilis cells that expressed the wild-type pre-pro-FNA.

The majority of B. subtilis clones expressing a full-length FNA comprising a pre-pro polypeptide having a combination of mutations had a greater level of production of the mature FNA than clones expressing produced a full-length FNA comprising a pre-pro polypeptide having a single mutation.

### Example 3

Site Evaluation Libraries (SELs) were constructed to generate positional libraries at each of the first 103 amino acid positions that comprise the pre-pro region of FNA. Site saturation mutagenesis of the pre-pro sequence of the full-length FNA protease was performed to identify amino acid substitutions that increase the production of FNA by a bacterial host cell.

### SEL Library Construction

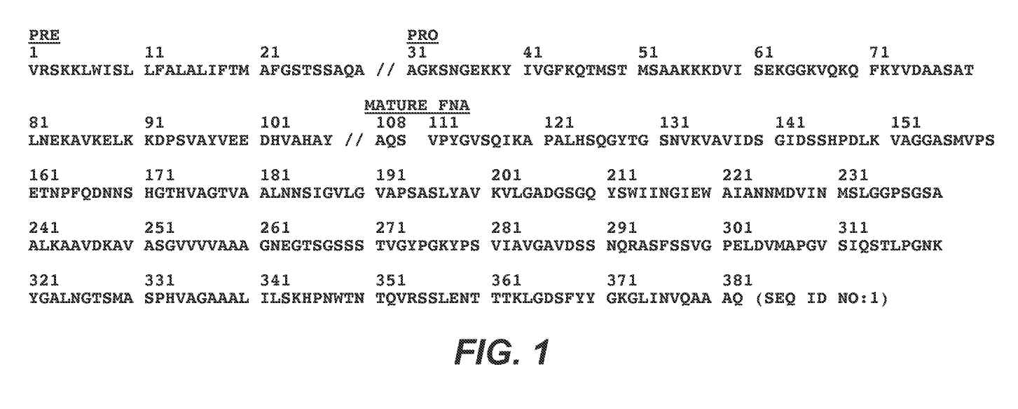

Pre-Pro-FNA SEL production was performed by DNA 2.0 (Menlo Park, Calif.) using their technology platform for gene optimization, gene synthesis and library generation under proprietary DNA 2.0 know how and/or intellectual property. The pAC-FNA10 plasmid containing the full-length FNA polynucleotide (GTGAGAAGCAAAAAATTGTGGATCAGTTTGCTGTTTGCTTTAGCGTTAATCTTTACGATGGCGTT CGGCAGCACATCCAGCGCGCAGGCGGCAGGGAAATCAAACGGGGAAAAGAAATATATTGTCGG GTTTAAACAGACAATGAGCACGATGAGCGCCGCTAAGAAGAAAGATGTCATTTCTGAAAAAGGC GGGAAAGTGCAAAAGCAATTCAAATATGTAGACGCAGCTTCAGCTACATTAAACGAAAAAGCTGT AAAAGAATTGAAAAAAGACCCGAGCGTCGCTTACGTTGAAGAAGATCACGTAGCACACGCGTAC GCGCAGTCCGTGCCTTACGGCGTATCACAAATTAAAGCCCCTGCTCTGCACTCTCAAGGCTACA CTGGATCAAATGTTAAAGTAGCGGTTATCGACAGCGGTATCGATTCTTCTCATCCTGATTTAAAG GTAGCAGGCGGAGCCAGCATGGTTCCTTCTGAAACAAATCCTTTCCAAGACAACAACTCTCACG GAACTCACGTTGCCGGCACAGTTGCGGCTCTTAATAACTCAATCGGTGTATTAGGCGTTGCGCC AAGCGCATCACTTTACGCTGTAAAAGTTCTCGGTGCTGACGGTTCCGGCCAATACAGCTGGATC ATTAACGGAATCGAGTGGGCGATCGCAAACAATATGGACGTTATTAACATGAGCCTCGGCGGAC CTTCTGGTTCTGCTGCTTTAAAAGCGGCAGTTGATAAAGCCGTTGCATCCGGCGTCGTAGTCGTT GCGGCAGCCGGTAACGAAGGCACTTCCGGCAGCTCAAGCACAGTGGGCTACCCTGGTAAATAC CCTTCTGTCATTGCAGTAGGCGCTGTTGACAGCAGCAACCAAAGAGCATCTTTCTCAAGCGTAG GACCTGAGCTTGATGTCATGGCACCTGGCGTATCTATCCAAAGCACGCTTCCTGGAAACAAATAC GGCGCGTTGAACGGTACATCAATGGCATCTCCGCACGTTGCCGGAGCGGCTGCTTTGATTCTTT CTAAGCACCCGAACTGGACAAACACTCAAGTCCGCAGCAGTTTAGAAAACACCACTACAAAACTT GGTGATTCTTTCTACTATGGAAAAGGGCTGATCAACGTACAGGCGGCAGCTCAGTAA; SEQ ID NO:2) was sent to DNA 2.0 for the generation of the SELs. A request was made to DNA 2.0 to generate positional libraries at each of the 107 amino acids of the pre-pro region of FNA (FIG. 1). For each of the 107 sites shown enumerated in FIG. 1, DNA 2.0 provided no less than 15 substitution variants at each of the positions. These gene constructs were obtained in 96 well plates each containing 4 single position libraries per plate. The libraries consisted of transformed B. subtilis host cells (genotype: ΔaprE, ΔnprE, ΔspollE, amyE::xylRPxylAcomK-phleo) that had been transformed with expression plasmids encoding the FNA variant sequences. These cells were received as glycerol stocks plated in 96 well plates, and the polynucleotide encoding each variant was sequenced, and the activity of the encoded variant protein was determined as described above. Individual clones were cultured as described in Example 1 in order to obtain the different FNA protein variants for functional characterization. FNA production is reported in Table 11 as the ratio of production of FNA processed from full-length FNA protein comprising mutated pre-pro polypeptides relative to the production of FNA processed from wild-type full-length FNA at a given position.

TABLE 11
Effect of mutations in the pre-pro region of FNA on the production
of the mature protein

| | Original | Variant amino acids | | | | | | | | | |
|---|---|---|---|---|---|---|---|---|---|---|---|
| Position | residue | A | C | D | E | F | G | H | I | K | L |
| 1 | V | | | | | | | | | | |
| 2 | R | | 0.57 | 0.93 | 0.27 | 1.19 | | 0.23 | 0.64 | 0.46 | 0.25 |
| 3 | S | 1.00 | 0.78 | 0.81 | 0.97 | 0.32 | 0.33 | 0.27 | | | 0.56 |
| 4 | K | 0.02 | 0.60 | 0.49 | −0.04 | 0.27 | 0.32 | | 0.51 | 0.60 | 0.57 |
| 5 | K | 0.00 | 0.00 | | 0.20 | 0.39 | 0.40 | | | 1.26 | 0.25 |
| 6 | L | 0.38 | 0.88 | | 0.80 | 0.37 | 0.83 | 0.43 | 0.44 | 1.17 | 0.82 |
| 7 | W | 0.46 | 0.37 | 0.38 | 1.05 | 0.32 | 0.26 | | 0.47 | | 0.28 |
| 8 | I | 0.48 | 0.02 | 0.19 | 0.41 | 0.46 | 0.80 | | 1.04 | 0.03 | 0.70 |
| 9 | S | 0.98 | 0.58 | 0.44 | 0.12 | 0.58 | 0.22 | | 0.47 | 0.59 | 0.24 |
| 10 | L | 1.10 | 1.24 | 0.00 | 0.01 | 0.03 | 1.15 | 0.43 | −0.01 | 0.25 | 0.86 |
| 11 | L | 1.04 | 0.00 | | 0.44 | 1.26 | 0.75 | 0.73 | 0.68 | 0.66 | 1.16 |
| 12 | F | 0.67 | 1.07 | 0.11 | −0.13 | 0.90 | 0.39 | 0.44 | | 0.16 | 0.77 |
| 13 | A | 0.95 | 1.20 | 0.42 | | 0.77 | 1.47 | | | 0.80 | 0.70 |
| 14 | L | | 0.30 | 0.12 | 0.00 | 1.49 | | 0.62 | 0.95 | 0.15 | −0.01 |
| 15 | A | 0.38 | 0.56 | | 0.36 | 0.38 | 1.05 | | 0.61 | 0.14 | 0.45 |
| 16 | L | | 0.57 | | 0.17 | 0.91 | 0.53 | 0.37 | 0.85 | | 0.41 |
| 17 | I | 0.46 | 0.52 | 0.24 | 0.31 | 0.45 | 0.67 | 0.34 | 0.34 | | 0.64 |
| 18 | F | 0.56 | 0.84 | 0.06 | 0.27 | 0.37 | 0.63 | | 0.72 | 0.04 | 0.75 |
| 19 | T | 0.54 | 0.49 | 0.42 | | 0.55 | 0.73 | | 0.68 | | 0.46 |
| 20 | M | 0.57 | 0.72 | 0.38 | 0.65 | 0.78 | 0.53 | 0.60 | 0.93 | | 0.48 |
| 21 | A | 0.92 | 0.53 | 0.48 | 0.52 | 0.62 | 0.25 | | | −0.02 | 0.48 |
| 22 | F | 0.43 | 0.43 | | 1.23 | 0.37 | 0.41 | 0.66 | 0.55 | 0.60 | 0.73 |
| 23 | G | 0.55 | 0.78 | | | 1.33 | 1.09 | 0.41 | 0.47 | | 0.47 |
| 24 | S | | 0.67 | | 0.61 | 0.61 | 0.82 | 0.29 | | | 0.55 |
| 25 | T | 1.12 | 0.58 | 1.32 | 0.61 | 0.52 | 0.59 | 0.49 | 0.79 | | 0.64 |
| 26 | S | 0.81 | 1.35 | 0.79 | 0.69 | 0.01 | 0.81 | 1.36 | 0.64 | 0.37 | 0.41 |
| 27 | S | 1.06 | 0.63 | | 0.89 | 1.76 | 0.31 | 1.86 | 0.96 | | 0.90 |
| 28 | A | 0.98 | | | 0.57 | 0.80 | 0.68 | | 0.81 | 0.38 | 0.83 |
| 29 | Q | 0.61 | 0.51 | | 1.22 | 0.93 | 0.86 | | 1.15 | | 0.91 |
| 30 | A | 0.81 | 1.13 | 0.97 | 0.61 | 0.98 | 0.47 | 0.97 | | 0.35 | 0.54 |
| 31 | A | 1.06 | 0.49 | 0.29 | 0.56 | 0.27 | 0.63 | 1.39 | 0.49 | 1.45 | 0.49 |
| 32 | G | 0.94 | 1.41 | 0.61 | 0.92 | 1.30 | 0.56 | | 0.52 | | 0.73 |
| 33 | K | 0.41 | 0.51 | 0.42 | 1.07 | 1.33 | 0.76 | | 0.77 | 0.23 | −0.02 |
| 34 | S | 0.64 | 0.98 | 1.18 | 0.83 | 0.50 | 0.89 | 1.08 | | 0.57 | 0.38 |
| 35 | N | 0.75 | 1.47 | | 0.43 | 0.63 | 0.71 | | 0.72 | | 0.14 |
| 36 | G | 0.68 | 1.20 | 1.68 | 0.50 | 0.73 | 1.40 | | 0.49 | | 0.78 |
| 37 | E | 0.95 | 1.20 | 0.64 | 0.54 | 0.66 | 1.29 | | 0.85 | 1.39 | 0.44 |
| 38 | K | 0.25 | 0.60 | 0.03 | 1.17 | 1.30 | 0.60 | 0.57 | 0.51 | 0.99 | 0.57 |
| 39 | K | 1.21 | 1.03 | | 0.84 | | 1.11 | 0.87 | 1.25 | | 2.64 |
| 40 | Y | 0.41 | 0.82 | | 0.22 | 0.04 | 0.16 | 0.14 | 0.39 | | 0.22 |
| 41 | I | 0.03 | 0.15 | −0.03 | 0.03 | 0.64 | 0.06 | | 0.71 | | 0.54 |
| 42 | V | −0.03 | 0.31 | −0.02 | | 0.03 | −0.03 | −0.02 | | | 0.22 |
| 43 | G | 0.00 | | 0.01 | 0.00 | | 0.01 | | 0.00 | 0.00 | 0.00 |
| 44 | F | 0.46 | 0.06 | 0.22 | | 0.65 | 0.25 | | 0.27 | | 0.49 |
| 45 | K | 0.62 | 0.40 | | 0.65 | 0.70 | 1.56 | | | | 0.48 |
| 46 | Q | 0.48 | 0.59 | 0.37 | | 0.63 | 0.46 | | 0.64 | | 0.54 |
| 47 | T | 0.13 | | 0.56 | 1.31 | 1.43 | 0.52 | −0.03 | | 0.58 | 0.37 |
| 48 | M | −0.02 | 0.60 | −0.02 | −0.11 | 0.00 | 1.42 | | 1.46 | 0.45 | 0.76 |
| 49 | S | 0.60 | 0.47 | | 1.08 | 0.55 | 0.60 | | 0.04 | 0.62 | −0.06 |
| 50 | T | 0.98 | 0.97 | 1.15 | 0.70 | 0.83 | 0.45 | 0.68 | 0.43 | | 0.96 |
| 51 | M | 1.37 | 0.74 | 0.76 | 0.73 | 0.46 | 0.81 | 1.07 | 0.75 | 0.91 | 0.72 |
| 52 | S | 2.67 | | | 0.85 | | 0.97 | | 1.31 | 0.89 | 0.41 |
| 53 | A | 0.91 | 0.56 | 1.11 | 1.64 | 0.68 | 0.88 | | 0.55 | 0.57 | 0.59 |
| 54 | A | 0.55 | 0.46 | 0.98 | 0.54 | 1.26 | 1.08 | 0.04 | 1.19 | 0.08 | 0.57 |
| 55 | K | 0.86 | | 1.01 | | 0.60 | 0.90 | | 0.47 | 0.73 | 0.54 |
| 56 | K | | | | 0.98 | 0.50 | 0.83 | | 0.86 | 0.43 | 0.51 |
| 57 | K | | 0.69 | 0.54 | 1.55 | 0.50 | 0.84 | | 0.42 | 0.19 | 0.75 |
| 58 | D | 1.21 | 1.02 | 0.66 | 1.30 | 1.04 | 1.35 | | 0.92 | 2.25 | 0.82 |
| 59 | V | 0.43 | 0.64 | 0.46 | 1.12 | 0.63 | 0.43 | | 0.71 | 0.51 | 0.44 |
| 60 | I | 0.13 | | 0.05 | 0.05 | 0.19 | 0.13 | | 0.32 | 0.00 | 0.31 |
| 61 | S | 1.07 | 0.41 | 0.97 | 0.57 | | 0.65 | | 1.13 | 0.26 | 0.51 |
| 62 | E | 1.07 | 0.81 | 0.76 | 0.71 | 0.53 | 1.21 | 1.07 | 0.52 | 0.54 | 0.40 |
| 63 | K | 1.13 | 1.19 | −0.08 | 1.45 | 1.60 | 1.36 | | 0.83 | 0.72 | 0.91 |
| 64 | G | 0.32 | | 1.22 | | 0.54 | 1.13 | | 0.24 | 0.71 | 0.03 |
| 65 | G | 0.05 | | 0.06 | 0.06 | 0.25 | 0.55 | 0.13 | 0.49 | 0.02 | 0.44 |
| 66 | K | | | 0.62 | 1.03 | | 0.33 | 0.67 | 0.17 | 0.18 | 0.15 |
| 67 | V | | 0.60 | 0.96 | 0.57 | 0.85 | 1.44 | 0.30 | 0.61 | 0.45 | 1.17 |
| 68 | Q | 0.52 | 1.55 | 1.05 | | 0.53 | 0.67 | 0.56 | 0.47 | 0.31 | 0.74 |
| 69 | K | 0.74 | 0.44 | 0.30 | 0.69 | 0.66 | 0.42 | 0.57 | 0.93 | 0.42 | 0.49 |
| 70 | Q | 0.98 | 0.49 | | 1.01 | | 0.60 | 0.43 | 0.54 | 1.11 | 0.80 |
| 71 | F | 0.11 | 0.15 | 0.03 | 0.03 | 0.15 | 0.08 | 0.11 | 0.00 | 0.02 | 0.41 |
| 72 | K | 0.50 | 0.70 | 0.50 | 0.28 | | 0.98 | 0.09 | 0.81 | 0.66 | 0.71 |
| 73 | Y | 0.45 | 0.74 | 0.42 | 0.65 | 0.60 | 0.28 | 0.50 | 0.63 | 0.25 | 0.53 |
| 74 | V | 0.53 | 1.82 | 0.22 | 0.65 | 0.56 | 0.22 | 0.12 | | −0.05 | 0.58 |
| 75 | D | 0.58 | | 0.33 | 0.73 | | 1.22 | 0.55 | 0.43 | 0.50 | 0.92 |
| 76 | A | 0.66 | 0.36 | | 0.18 | | 0.62 | 0.08 | | 0.06 | 0.21 |
| 77 | A | 1.15 | 0.74 | 0.66 | 1.26 | 0.63 | 0.38 | 0.48 | | 0.47 | 0.02 |
| 78 | S | | 0.68 | 0.52 | | 0.92 | 0.78 | 0.53 | | 0.99 | 0.95 |
| 79 | A | 0.89 | 0.94 | 0.03 | 0.07 | 0.38 | 0.50 | 0.03 | 0.61 | 0.02 | 0.48 |
| 80 | T | | 0.90 | 1.09 | 0.72 | | 0.57 | 0.79 | 0.83 | 0.48 | 1.22 |
| 81 | L | 0.56 | 0.79 | −0.09 | 0.04 | | 0.11 | 0.02 | | −0.03 | 0.81 |
| 82 | N | 0.62 | 1.09 | 1.05 | 0.68 | | 0.97 | | | 0.86 | 0.33 |
| 83 | E | 0.60 | | 0.09 | 0.44 | | 1.49 | 0.56 | 0.94 | 0.52 | |
| 84 | K | 0.97 | 0.44 | 0.44 | 0.51 | | 0.54 | 0.85 | | 0.47 | |
| 85 | A | 0.13 | 0.57 | | 0.60 | 0.51 | 0.62 | | 0.48 | 0.41 | 0.40 |
| 86 | V | 0.59 | | 0.25 | 0.95 | 0.57 | 0.37 | | 0.97 | 0.81 | 0.84 |
| 87 | K | 0.54 | | 0.98 | 0.09 | 0.40 | 0.52 | | | 2.22 | 0.20 |
| 88 | E | 1.02 | 0.49 | 1.09 | | | 1.98 | 0.64 | 0.43 | 0.53 | 0.49 |
| 89 | L | 0.21 | 0.47 | 0.03 | 0.09 | 0.18 | 0.10 | | −0.02 | −0.20 | −0.02 |
| 90 | K | 0.90 | | 1.01 | 0.60 | 0.57 | 0.51 | 0.68 | 0.80 | 0.55 | 0.10 |
| 91 | K | 0.52 | | 0.53 | 0.05 | 0.67 | 0.23 | 0.90 | | 0.55 | 0.41 |
| 92 | D | 0.47 | | 3.51 | 1.13 | 0.44 | 0.28 | 0.57 | 0.61 | 0.16 | 0.67 |
| 93 | P | 0.78 | 0.77 | 0.76 | 0.80 | | 1.10 | | | 0.44 | 0.46 |
| 94 | S | 0.57 | | 0.64 | 0.71 | | 0.60 | | 0.89 | 0.84 | 0.82 |
| 95 | V | 0.19 | | −0.03 | 0.03 | | −0.04 | −0.03 | 0.55 | −0.03 | 0.35 |
| 96 | A | 0.82 | | 0.49 | 0.09 | 0.36 | 1.11 | | 0.57 | 0.77 | 0.89 |
| 97 | Y | 0.17 | 0.16 | 0.12 | 0.15 | | 0.06 | 0.15 | | −0.11 | |
| 98 | V | | 0.53 | 0.02 | 0.07 | 0.11 | 0.02 | 0.02 | 0.38 | 0.02 | 0.93 |
| 99 | E | 0.32 | 0.23 | 0.38 | 0.57 | | 0.16 | 0.21 | | 0.05 | 0.11 |
| 100 | E | 0.69 | | 0.73 | 0.78 | 0.42 | 0.75 | 0.39 | | 0.46 | 0.67 |
| 101 | D | −0.10 | | 0.28 | 0.14 | | −0.03 | −0.14 | | 0.03 | 0.03 |
| 102 | H | 0.57 | | 0.83 | 0.62 | | 0.42 | 0.98 | | 0.96 | 0.24 |
| 103 | V | 0.03 | | 0.90 | 0.02 | 0.90 | 0.01 | 0.54 | 0.55 | 0.53 | 0.07 |
| | Original | Variant amino acids | | | | | | | | | |
| Position | residue | M | N | P | Q | R | S | T | V | W | Y |
| 1 | V | | | | | | | | | | |
| 2 | R | 0.47 | 1.02 | 1.03 | 0.15 | 0.40 | 0.44 | 0.71 | | | 1.67 |
| 3 | S | 1.04 | | 1.06 | 0.67 | 1.49 | 1.13 | | 0.68 | 0.87 | 0.85 |
| 4 | K | −0.01 | 0.47 | 0.58 | | −0.02 | 0.41 | | −0.04 | −0.02 | 0.37 |
| 5 | K | 0.34 | 0.71 | 0.10 | | 0.53 | 0.75 | 0.88 | 0.89 | 0.38 | |
| 6 | L | 1.03 | 0.46 | 0.34 | | 0.34 | 0.83 | 0.59 | 0.80 | | 0.69 |
| 7 | W | 0.72 | | 0.54 | | 0.35 | 0.86 | 0.71 | 0.76 | 0.92 | |
| 8 | I | | 0.01 | 0.53 | −0.02 | 0.01 | 0.39 | −0.06 | 0.43 | 1.05 | 0.29 |
| 9 | S | 0.44 | | 0.54 | | 0.60 | 0.57 | 0.38 | 0.72 | 0.33 | |
| 10 | L | 1.14 | 0.00 | 0.83 | 0.61 | 0.31 | 0.80 | 1.73 | 0.87 | 0.73 | 0.00 |
| 11 | L | 0.61 | 0.67 | 0.60 | 0.67 | 0.60 | 0.95 | 1.24 | 0.86 | 0.00 | 0.68 |
| 12 | F | | | 1.05 | 0.51 | 0.12 | 0.79 | 1.00 | 0.86 | 0.73 | 0.38 |
| 13 | A | 0.86 | 0.42 | 0.36 | 0.79 | 0.35 | 1.22 | 0.42 | 0.94 | 0.37 | 0.16 |
| 14 | L | | 0.55 | 0.55 | 0.60 | 0.04 | 0.41 | 0.47 | 0.50 | 0.61 | 0.22 |
| 15 | A | 1.23 | 0.53 | 0.42 | 0.43 | −0.02 | 0.44 | 1.03 | 1.28 | 0.29 | |
| 16 | L | 0.45 | 0.24 | 0.32 | 0.54 | −0.04 | 0.48 | | 1.21 | 0.37 | |
| 17 | I | | 0.28 | 0.30 | 0.42 | −0.04 | 1.25 | | 0.56 | 0.29 | |
| 18 | F | 0.47 | 0.22 | 0.44 | 0.44 | 0.09 | 0.42 | 0.47 | 0.51 | 0.31 | 0.38 |
| 19 | T | 0.48 | | 1.01 | 0.63 | 0.14 | 1.36 | 0.22 | 0.71 | 0.40 | |
| 20 | M | 0.83 | | 0.40 | | 0.34 | 0.51 | 0.84 | 1.06 | 0.53 | 0.88 |
| 21 | A | 0.55 | 0.13 | 0.60 | 0.12 | 0.17 | 1.07 | 0.51 | 0.56 | 0.33 | |
| 22 | F | 0.41 | 0.72 | 0.19 | 0.43 | 0.42 | 0.48 | 0.47 | 0.51 | 0.39 | 0.50 |
| 23 | G | | | 0.56 | 1.21 | 0.67 | 0.58 | 0.50 | 0.66 | 1.50 | 0.45 |
| 24 | S | 0.59 | 0.71 | 0.82 | 0.89 | 0.34 | 0.92 | | 1.61 | 0.67 | 0.48 |
| 25 | T | 0.55 | | 0.40 | | 0.31 | 0.84 | 0.60 | 0.76 | 1.15 | 0.43 |
| 26 | S | 0.73 | 0.65 | | 0.75 | 0.47 | 0.25 | 0.63 | 0.75 | 0.71 | 0.75 |
| 27 | S | 0.64 | | 3.23 | | 0.72 | 0.80 | 1.07 | 2.04 | 0.66 | 1.03 |
| 28 | A | 0.66 | 0.97 | 0.49 | 0.56 | 0.35 | 0.88 | 0.87 | 1.14 | 0.50 | |
| 29 | Q | 0.54 | | 0.40 | 0.49 | 1.18 | 1.45 | 1.47 | 0.62 | 0.64 | |
| 30 | A | 0.66 | 0.51 | | 0.49 | 0.93 | 0.29 | 0.72 | 0.88 | 0.81 | 0.62 |
| 31 | A | 0.95 | 2.60 | 0.37 | 0.19 | 0.49 | 1.80 | −0.01 | 1.17 | 1.12 | |
| 32 | G | 1.05 | 1.68 | 1.11 | | 0.90 | 1.14 | | 1.19 | −0.02 | 0.85 |
| 33 | K | 1.04 | | 1.17 | | 0.55 | 1.23 | | 0.12 | 0.30 | 0.21 |
| 34 | S | 0.84 | 0.56 | 1.02 | 0.53 | 0.76 | 0.65 | 0.54 | 1.41 | 0.55 | 0.72 |
| 35 | N | 0.37 | 0.50 | 0.98 | 1.18 | 0.91 | 1.39 | 0.03 | 0.57 | 0.19 | 0.79 |
| 36 | G | 0.58 | | 0.51 | 0.59 | 0.47 | 1.25 | 0.60 | 0.59 | 1.17 | 1.97 |
| 37 | E | 0.52 | 0.16 | 0.00 | 1.09 | 0.28 | 0.59 | 0.35 | 0.98 | 0.87 | 0.39 |
| 38 | K | 0.20 | 0.97 | | 1.13 | 0.48 | 1.03 | 0.86 | 0.67 | 1.14 | 0.99 |
| 39 | K | 1.00 | | 1.35 | 0.94 | 0.82 | 1.17 | 1.17 | 1.27 | 0.77 | 0.49 |
| 40 | Y | 0.75 | | −0.05 | 0.13 | 0.07 | 0.10 | | 0.59 | 0.82 | 0.36 |
| 41 | I | 0.68 | | −0.03 | 0.05 | −0.04 | 0.06 | | 0.55 | | −0.03 |
| 42 | V | 0.06 | | 0.02 | −0.02 | −0.03 | −0.03 | 0.06 | 0.49 | 0.00 | 0.02 |
| 43 | G | 0.26 | | 0.00 | | 0.00 | 0.19 | 0.00 | −0.01 | 0.00 | 0.00 |
| 44 | F | 0.05 | | −0.07 | 0.06 | 0.20 | 0.58 | 0.49 | 0.42 | 0.58 | |
| 45 | K | 0.74 | | 0.34 | 0.53 | 0.96 | 1.14 | 0.51 | 0.59 | 0.25 | 0.83 |
| 46 | Q | 0.48 | | | 0.60 | 0.49 | 1.27 | 0.56 | 0.48 | 0.42 | 0.46 |
| 47 | T | 0.43 | | 0.66 | 0.51 | 0.94 | 0.53 | 1.16 | 0.48 | | |
| 48 | M | 0.53 | | | | 0.43 | 0.42 | 1.54 | 0.48 | 1.38 | 2.55 |
| 49 | S | 0.31 | 0.47 | 0.03 | 0.03 | 0.81 | 0.68 | −0.02 | 0.58 | | −0.04 |
| 50 | T | 0.94 | | | 0.65 | 0.15 | 0.68 | 0.74 | 0.91 | | 1.09 |
| 51 | M | 0.52 | 0.79 | 0.78 | 0.34 | 0.79 | 0.61 | 0.73 | 0.59 | 0.58 | 0.55 |
| 52 | S | 1.06 | | 0.72 | 0.67 | 0.95 | 0.55 | 0.95 | 0.45 | 0.95 | 0.85 |
| 53 | A | 1.02 | 0.72 | 0.68 | 1.50 | 0.64 | 0.67 | 1.26 | 0.80 | 0.50 | 0.46 |
| 54 | A | | | 0.96 | 0.99 | 0.80 | 1.04 | 0.71 | 0.89 | 0.43 | |
| 55 | K | 0.73 | 0.75 | | 0.11 | 0.50 | 0.75 | 0.75 | | 0.42 | 0.64 |
| 56 | K | 0.81 | | 0.39 | 0.46 | 0.82 | 0.60 | 0.80 | 0.27 | 0.54 | 0.72 |
| 57 | K | 0.48 | 1.06 | | 0.62 | 1.37 | 0.10 | 0.42 | 0.52 | 0.44 | 0.15 |
| 58 | D | 0.94 | 0.46 | | | 1.45 | 1.13 | 1.06 | 0.79 | 1.05 | 0.44 |
| 59 | V | | 0.73 | | 0.54 | 0.80 | 0.57 | 0.52 | 0.60 | 0.36 | 0.50 |
| 60 | I | 0.65 | 0.05 | | 0.07 | 0.08 | 0.08 | 0.09 | 0.43 | 0.08 | 0.07 |
| 61 | S | | | 0.46 | | 1.41 | 1.25 | 0.51 | 0.62 | 0.47 | 0.80 |
| 62 | E | | 1.38 | 0.15 | 0.66 | 0.81 | 1.92 | 1.40 | 0.50 | 0.77 | |
| 63 | K | 0.04 | 1.12 | | 1.27 | 1.73 | 0.73 | 1.05 | 0.86 | 0.44 | 0.41 |
| 64 | G | 1.07 | | 0.26 | 1.20 | 0.56 | 1.31 | | 0.42 | 0.17 | 0.50 |
| 65 | G | 0.14 | | 0.04 | 0.16 | 0.09 | 0.17 | 0.11 | 0.59 | 0.25 | 0.25 |
| 66 | K | 0.79 | | 0.14 | 0.57 | 0.79 | 0.60 | 0.44 | 0.56 | | 0.45 |
| 67 | V | 0.56 | 0.65 | 0.79 | 0.93 | 0.50 | 0.78 | 0.65 | 0.97 | 0.27 | 0.47 |
| 68 | Q | 0.52 | 0.58 | 0.51 | 0.53 | 1.24 | 0.97 | 0.87 | | | 0.70 |
| 69 | K | 0.48 | 0.60 | 0.18 | | 0.70 | 0.74 | 0.48 | 0.49 | | 1.40 |
| 70 | Q | 0.18 | 0.67 | 1.29 | 0.67 | 0.28 | 1.48 | 0.63 | 1.37 | 0.57 | 0.46 |
| 71 | F | 0.72 | 0.13 | 0.03 | −0.07 | 0.04 | 0.03 | 0.07 | 0.07 | 0.26 | 0.76 |
| 72 | K | 0.47 | 0.58 | 0.60 | 0.00 | 0.32 | 0.54 | 0.65 | 0.10 | 0.79 | 0.22 |
| 73 | Y | 0.52 | 0.09 | −0.11 | 0.90 | 0.35 | 0.49 | 0.25 | 0.55 | 0.74 | 0.82 |
| 74 | V | | 0.18 | | 0.68 | 0.02 | 0.55 | 0.50 | 0.66 | 0.15 | 0.14 |
| 75 | D | 0.62 | 0.67 | 0.76 | 0.40 | 0.63 | 0.62 | 0.61 | 0.80 | 0.54 | |
| 76 | A | 0.96 | | −0.01 | 0.69 | 0.04 | 0.44 | 0.06 | 2.62 | −0.02 | 0.00 |
| 77 | A | 0.79 | 0.44 | 0.02 | 0.67 | 0.37 | 0.36 | 0.70 | 2.43 | | 1.54 |
| 78 | S | 0.39 | 0.47 | 0.98 | 1.19 | 0.68 | 0.88 | | 0.56 | 0.40 | 0.60 |
| 79 | A | 0.45 | 0.01 | −0.02 | 0.02 | 0.04 | 0.58 | 0.62 | 0.66 | 0.08 | 0.07 |
| 80 | T | | 1.01 | 0.09 | 0.93 | 0.80 | 0.78 | 0.89 | 0.85 | 0.71 | 0.79 |
| 81 | L | | 0.02 | −0.14 | 0.04 | 0.02 | 0.14 | 0.07 | 0.35 | 0.07 | |
| 82 | N | 0.85 | 0.42 | 1.00 | 1.22 | 0.99 | 1.06 | 1.25 | 0.62 | 0.34 | 0.31 |
| 83 | E | | 1.02 | 0.57 | 0.79 | 0.49 | 0.46 | 0.59 | 0.59 | 0.30 | |
| 84 | K | 1.33 | | 0.23 | 0.61 | 0.72 | 0.56 | 0.50 | 0.47 | | 0.71 |
| 85 | A | | 0.49 | 0.15 | 0.19 | 0.13 | 0.52 | 0.63 | 0.50 | | 0.33 |
| 86 | V | 0.63 | 0.33 | 0.05 | | 0.41 | 0.53 | 0.58 | | 0.63 | 0.55 |
| 87 | K | 0.47 | 0.29 | 0.67 | | 1.08 | 0.48 | 0.51 | 0.95 | 0.43 | 0.50 |
| 88 | E | | 0.96 | 0.16 | | 0.72 | 0.14 | 1.13 | 1.74 | 0.42 | |
| 89 | L | | −0.01 | −0.02 | 0.03 | 0.02 | | 0.41 | 1.11 | −0.07 | −0.09 |
| 90 | K | | | 0.36 | 1.66 | 0.28 | 0.56 | 0.88 | | | 0.56 |
| 91 | K | 0.43 | 0.45 | −0.02 | 0.53 | 0.55 | | 0.98 | 0.66 | | 0.33 |
| 92 | D | 0.49 | 0.52 | | 0.71 | 0.22 | 1.22 | 0.74 | 0.90 | 0.36 | −0.05 |
| 93 | P | 0.49 | 1.33 | 0.64 | 0.69 | 0.80 | | 0.83 | 0.70 | 0.78 | |
| 94 | S | 0.78 | 0.29 | 0.44 | 0.68 | 0.47 | 0.62 | 0.62 | 0.67 | | |
| 95 | V | 0.15 | 0.03 | 0.02 | 0.03 | −0.02 | 0.04 | 0.40 | 0.14 | | |
| 96 | A | 0.46 | 1.25 | 0.04 | 0.58 | 0.56 | 0.93 | 1.23 | 0.46 | | 0.36 |
| 97 | Y | 0.17 | 0.05 | −0.11 | 0.19 | 0.27 | 0.25 | 0.25 | 0.18 | 0.93 | |
| 98 | V | | −0.01 | −0.01 | 0.05 | 0.02 | 0.02 | 0.28 | 0.61 | 0.02 | |
| 99 | E | 0.09 | 0.16 | −0.03 | 0.53 | 0.39 | | 0.19 | 0.12 | 0.03 | |
| 100 | E | 0.41 | 0.11 | 0.70 | 0.63 | 0.20 | 0.43 | 0.75 | 0.43 | 0.69 | |
| 101 | D | 0.06 | 0.23 | | 0.02 | −0.02 | 0.03 | 0.08 | 0.06 | −0.03 | −0.02 |
| 102 | H | 0.39 | | 0.06 | 0.63 | 0.73 | 0.90 | 1.13 | 0.97 | 0.96 | |
| 103 | V | 0.04 | 0.74 | 0.04 | 0.07 | 0.03 | | 0.03 | 0.05 | | |

### Example 4

### Production of Protease from Bacillus subtilis Having Stably Integrated Constructs Encoding Modified Proteases

Enhanced production of protease in Bacillus subtilis when expressed from a replicating vector pAC-FNA10 was confirmed when the vector was integrated into the chromosome of Bacillus subtilis using the pJH integrating vector (Ferrari et al. J. Bacteriol. 154:1513-1515 [1983]).

For vector integration, the upstream region of AprE promoter was added to the short promoter present in pAC-FNA10 by extension PCR. For this purpose, two fragments were amplified-one using the pJH-FNA plasmid (FIG. 6) as the template and the other using the pAC-FNA10 plasmid with a chosen mutation in the pre-pro region of FNA as template. The first fragment, containing the missing upstream region of the AprE promoter, was amplified from the pJH-FNA plasmid using primers P3249 and P3439 (Table 12). The second fragment, spanning the short aprE promoter, modified pre-pro and mature FNA region as well as transcription terminator was amplified by primers P3438 and P3435 (Table 12) using the pAC-FNA10 with the chosen modified pre-pro as template. These two fragments contained an overlap, which allowed to recreate the full-length aprE promoter (with FNA and terminator) by mixing both fragments together and amplifying with the flanking primers containing EcoRI and BamHI restriction sites (P3255 and P3246; Table 12). The resulting fragment containing the full-length aprE promoter, modified pre-pro region, mature FNA region and the transcription terminator was digested by EcoRI and BamHI and ligated with pJH-FNA vector, which was also digested by the same restriction enzymes. Similarly, a control fragment containing the full-length aprE promoter, the unmodified sequence encoding the unmodified parent pre-pro region and mature FNA region, and the transcription terminator was created (SEQ ID NO:452). The pJH-FNA construct containing DNA encoding the control unmodified or a modified protease was transformed into Bacillus subtilis strain (genotype ΔaprE, ΔnprE, spollE, amyE::xylRPxylAcomK-phleo) and cultured as described in Example 1. AAPF activity of the mature FNA proteases produced when processed from a modified full-length FNA was determined and quantified as described in Example 1, and its production was compared to that of the mature FNA processed from the unmodified full-length FNA.

The sequence of the long aprE promoter is set forth as SEQ ID NO:445

(SEQ ID NO: 445)
AATTCTCCATTTTCTTCTGCTATCAAAATAACAGACTCGTGATTTTCCAAACGAGCTTTCAAAA
AAGCCTCTGCCCCTTGCAAATCGGATGCCTGTCTATAAAATTCCCGATATTGGTTAAACAGC
GGCGCAATGGCGGCCGCATCTGATGTCTTTGCTTGGCGAATGTTCATCTTATTTCTTCCTCC
CTCTCAATAATTTTTTCATTCTATCCCTTTTCTGTAAAGTTTATTTTTCAGAATACTTTTATCATC
ATGCTTTGAAAAAATATCACGATAATATCCATTGTTCTCACGGAAGCACACGCAGGTCATTTG
AACGAATTTTTTCGACAGGAATTTGCCGGGACTCAGGAGCATTTAACCTAAAAAAGCATGAC
ATTTCAGCATAATGAACATTTACTCATGTCTATTTTCGTTCTTTTCTGTATGAAAATAGTTATTT
CGAGTCTCTACGGAAATAGCGAGAGATGATATACCTAAATAGAGATAAAATCATCTCAAAAAA
ATGGGTCTACTAAAATATTATTCCATCTATTACAATAAATTCACAGAATAGTCTTTTAAGTAAG
TCTACTCTGAATTTTTTTAAAAGGAGAGGGTAAAGA

TABLE 12
Primers used for production of stably
integrated constructs

| PRIMER | | SEQ ID |
|---|---|---|
| NAME | PRIMER SEQUENCE | NO: |
| P3249 | GCGCGCGTAATACGACTCAC | 446 |
| P3439 | ATTTTTTTGAGATGATTTTATCTCTATTTAGGTATAT | 447 |
| | CATCTC | |
| P3438 | TAAATAGAGATAAAATCATCTCAAAAAAATGGGTCTA | 448 |
| | CTAAA | |
| P3435 | ATGTATCAAGATAAGAAAGAACAAG | 449 |
| P3255 | GCAGGAATTCTCCATTTTCTTC | 450 |
| P3246 | TTTATTTTATAAACTCATTCCCTGAT | 451 |

The nucleotide sequence of the expression cassette comprising the unmodified parent FNA polynucleotide in the pJH-FNA vector is set forth as SEQ ID NO:452

(SEQ ID NO: 452)
AATTCTCCATTTTCTTCTGCTATCAAAATAACAGACTCGTGATTTTCCAAACGAGCTTTCAAAA
AAGCCTCTGCCCCTTGCAAATCGGATGCCTGTCTATAAAATTCCCGATATTGGTTAAACAGC
GGCGCAATGGCGGCCGCATCTGATGTCTTTGCTTGGCGAATGTTCATCTTATTTCTTCCTCC
CTCTCAATAATTTTTTCATTCTATCCCTTTTCTGTAAAGTTTATTTTTCAGAATACTTTTATCATC
ATGCTTTGAAAAAATATCACGATAATATCCATTGTTCTCACGGAAGCACACGCAGGTCATTTG
AACGAATTTTTTCGACAGGAATTTGCCGGGACTCAGGAGCATTTAACCTAAAAAAGCATGAC
ATTTCAGCATAATGAACATTTACTCATGTCTATTTTCGTTCTTTTCTGTATGAAAATAGTTATTT
CGAGTCTCTACGGAAATAGCGAGAGATGATATACCTAAATAGAGATAAAATCATCTCAAAAAA
ATGGGTCTACTAAAATATTATTCCATCTATTACAATAAATTCACAGAATAGTCTTTTAAGTAAG
TCTACTCTGAATTTTTTTAAAAGGAGAGGGTAAAGAGTGAGAAGCAAAAAATTGTGGATCAGT
TTGCTGTTTGCTTTAGCGTTAATCTTTACGATGGCGTTCGGCAGCACATCCTCTGCCCAGGC
GGCAGGGAAATCAAACGGGGAAAAGAAATATATTGTCGGGTTTAAACAGACAATGAGCACG
ATGAGCGCCGCTAAGAAGAAAGATGTCATTTCTGAAAAAGGCGGGAAAGTGCAAAAGCAATT
CAAATATGTAGACGCAGCTTCAGCTACATTAAACGAAAAAGCTGTAAAAGAATTGAAAAAAGA
CCCGAGCGTCGCTTACGTTGAAGAAGATCACGTAGCACATGCGTACGCGCAGTCCGTGCCT
TACGGCGTATCACAAATTAAAGCCCCTGCTCTGCACTCTCAAGGCTACACTGGATCAAATGT
TAAAGTAGCGGTTATCGACAGCGGTATCGATTCTTCTCATCCTGATTTAAAGGTAGCAGGCG
GAGCCAGCATGGTTCCTTCTGAAACAAATCCTTTCCAAGACAACAACTCTCACGGAACTCAC
GTTGCCGGCACAGTTGCGGCTCTTAATAACTCAATCGGTGTATTAGGCGTTGCGCCAAGCG
CATCACTTTACGCTGTAAAAGTTCTCGGTGCTGACGGTTCCGGCCAATACAGCTGGATCATT
AACGGAATCGAGTGGGCGATCGCAAACAATATGGACGTTATTAACATGAGCCTCGGCGGAC
CTTCTGGTTCTGCTGCTTTAAAAGCGGCAGTTGATAAAGCCGTTGCATCCGGCGTCGTAGTC
GTTGCGGCAGCCGGTAACGAAGGCACTTCCGGCAGCTCAAGCACAGTGGGCTACCCTGGT
AAATACCCTTCTGTCATTGCAGTAGGCGCTGTTGACAGCAGCAACCAAAGAGCATCTTTCTC
AAGCGTAGGACCTGAGCTTGATGTCATGGCACCTGGCGTATCTATCCAAAGCACGCTTCCT
GGAAACAAATACGGCGCGTTGAACGGTACATCAATGGCATCTCCGCACGTTGCCGGAGCGG
CTGCTTTGATTCTTTCTAAGCACCCGAACTGGACAAACACTCAAGTCCGCAGCAGTTTAGAA
AACACCACTACAAAACTTGGTGATTCTTTCTACTATGGAAAAGGGCTGATCAACGTACAGGC
GGCAGCTCAGTAAAACATAAAAAACCGGCCTTGGCCCCGCCGGTTTTTTATTATTTTTCTTCC
TCCGCATGTTCAATCCGCTCCATAATCGACGGATGGCTCCCTCTGAAAATTTTAACGAGAAA
CGGCGGGTTGACCCGGCTCAGTCCCGTAACGGCCAAGTCCTGAAACGTCTCAATCGCCGCT
TCCCGGTTTCCGGTCAGCTCAATGCCGTAACGGTCGGCGGCGTTTTCCTGATACCGGGAGA
CGGCATTCGTAATCGGATCC.

The cassette contains the sequence of the long AprE promoter (underlined, SEQ ID NO:445), the pre-pro region (SEQ ID NO:7) and mature regions of FNA (SEQ ID NO:(9), and a transcription terminator.

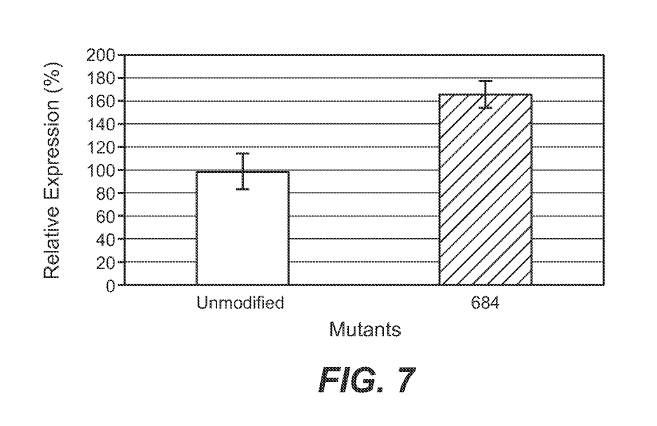

Results of FNA production processed from one of the mutants (clone 684; Table 9) are shown in FIG. 7 relative to the production of FNA production processed from the unmodified full-length FNA. These data confirmed that production of protease encoded from the integrated construct containing the modified pre-pro region was enhanced compared to that produced from the unmodified pre-pro region.

We claim:

1. An isolated modified polynucleotide encoding a modified full-length protease, said isolated modified polynucleotide comprising a first polynucleotide encoding the pre-pro region of said full-length protease operably linked to a second polynucleotide encoding the mature region of said full-length protease, wherein said first polynucleotide encodes the pre-pro region of SEQ ID NO:7 and is further mutated to comprise at least one mutation, wherein said at least one mutation enhances the production of said protease by a host cell.

2. The isolated modified polynucleotide of claim 1, wherein said modified full-length protease is an alkaline serine protease derived from a wild-type or variant precursor alkaline serine protease.

3. The isolated modified polynucleotide of claim 2, wherein said precursor alkaline serine protease is a Bacillus subtilis, a Bacillus amyloliquefaciens, a Bacillus pumilis or a Bacillus licheniformis serine protease.

4. The isolated polynucleotide of claim 1, wherein said host cell is a Bacillus sp. host cell.

5. The isolated polynucleotide of claim 4, wherein said Bacillus sp. host cell is a Bacillus subtilis host cell.

6. The isolated modified polynucleotide of claim 1, wherein said second polynucleotide encodes a protease having at least about 65% identity to the protease of SEQ ID NO:9.

7. The isolated modified polynucleotide of claim 1, wherein said second polynucleotide encodes the protease of SEQ ID NO:9.

8. The isolated modified polynucleotide of claim 1, wherein said first polynucleotide comprises at least one mutation encoding at least one substitution at one or more positions selected from positions 2, 3, 6, 7, 8, 10, 11, 12, 13, 14, 15, 16, 17, 19, 20, 21, 22, 23, 24, 25, 26, 27, 28, 29, 30, 31, 32, 33, 34, 35, 36, 37, 38, 39, 45, 46, 47, 48, 49, 50, 51, 52, 53, 54, 55, 57, 58, 59, 61, 62, 63, 64, 66, 67, 68, 69, 70, 72, 74, 75, 76, 77, 78, 80, 82, 83, 84, 87, 88, 89, 90, 91, 93, 96, 100, and 102, wherein the positions are numbered by correspondence with the amino acid sequence of the pre-pro polypeptide of SEQ ID NO:7.

9. The isolated modified polynucleotide of claim 1, wherein said first polynucleotide comprises at least one mutation encoding at least one substitution selected from X2F, N, P, and Y; X3A, M, P, and R; X6K, and M; X7E; I8W; X10A, C, G, M, and T; X11A, F, and T; X12C, P, T; X13C, G, and S; X14F; X15G, M, T, and V; X16V; X17S; X19P, and S; X20V; X21S; X22E; X23F, Q, and W; X24G, T and V; X25A, D, and W; X26C, and H; X27A, F, H, P, T, V, and Y; X28V; X29E, I, R, S, and T; X30C; X31H, K, N, S, V, and W; X32C, F, M, N, P, S, and V; X33E, F, M, P, and S; X34D, H, P, and V; X35C, Q, and S; X36C, D, L, N, S, W, and Y; X37C, G, K, and Q; X38F, Q, S, and W; X39A, C, G, I, L, M, P, S, T, and V; X45G and S; X46S; X47E and F; X48G, I, T, W, and Y; X49A, C, E and I; X50D, and Y; X51A and H; X52A, H, I, and M; X53D, E, M, Q, and T; X54F, G, H, I, and S; X55D; X57E, N, and R; X58A, C, E, F, G, K, R, S, T, W; X59E; X61A, F, I, and R; X62A, F, G, H, N, S, T and V; X63A, C, E, F, G, N, Q, R, and T; G64D, M, Q, and S; X66E; X67G and L; X68C, D, and R; X69Y; X70E, G, K, L, M, P, S, and V; X72D and N; X74C and Y; X75G; X76V; X77E, V, and Y; X78M, Q and V; X80D, L, and N; X82C, D, P, Q, S, and T; X83G, and N; X84M; X87R; X88A, D, G, T, and V; X89V; X90D and Q; X91A; X92E and S; X93G, N, and S; X96G, N, and T; X100Q; and X102T, wherein the positions are numbered by correspondence with the amino acid sequence of the pre-pro polypeptide of the FNA protease set forth as SEQ ID NO:7.

10. The isolated modified polynucleotide of claim 9, wherein said first polynucleotide comprises at least one mutation encoding at least one substitution selected from R2F, N, P, and Y; S3A, M, P, and R; L6K, and M; W7E; I8W; L10A, C, G, M, and T; L11A, F, and T; F12C, P, T; A13C, G, and S; L14F; A15G, M, T, and V; L16V; I17S; T19P, and S; M20V; A21S; F22E; G23F, Q, and W; S24G, T and V; T25A, D, and W; S26C, and H; S27A, F, H, P, T, V, and Y; A28V; Q29E, I, R, S, and T; A30C; A31H, K, N, S, V, and W; G32C, F, M, N, P, S, and T; K33E, F, M, P, and S; S34D, H, P, and V; N35C, Q, and S; G36C, D, L, N, S, W, and Y; E37C, G, K, and Q; K38F, Q, S, and W; K39A, C, G, I, L, M, P, S, T, and V; K45G and S; Q46S; T47E and F; M48G, I, T, W, and Y; S49A, C, E and I; T50D, and Y; M51A and H; S52A, H, I, and M; A53D, E, M, Q, and T; A54F, G, H, I, and S; K55D; K57E, N, and R; D58A, C, E, F, G, K, R, S, T, W; V59E; S61A, F, I, and R; E62A, F, G, H, N, S, T and V; K63A, C, E, F, G, N, Q, R, and T; 64D, M, Q, and S; K66E; V67G and L; Q68C, D, and R; K69Y; Q70E, G, K, L, M, P, S, and V; K72D and N; V74C and Y; D75G; A76V; A77E, V, and Y; S78M, Q and V; T80D, L, and N; N82C, D, P, Q, S, and T; E83G, and N; K84M; K87R; E88A, D, G, T, and V; L89V; K90D and Q; K91A; D92E and S; P93G, N, and S; A96G, N, and T; E100Q; and H102T, wherein the positions are numbered by correspondence with the amino acid sequence of the pre-pro polypeptide of the FNA protease set forth as SEQ ID NO:7.

11. The isolated modified polynucleotide of claim 1, wherein said first polynucleotide comprises at least one combination of mutations encoding a combination of substitutions selected from X49A-X24T, X49A-X72D, X49A-X78M, X49A-X78V, X49A-X93S, X49C-X24T, X49C-X72D, X49C-X78M, X49C-X78V, X49C-X91A, X49C-X93S, X91A-x24T, X91A-X49A, X91A-X52H, X91A-X72D, X91A-X78M, X91A-X78V, X93S-X24T, X93S-X49C, X93S-X52H, X93S-X72D, X93S-X78M, and X93S-X78V, wherein the positions are numbered by correspondence with the amino acid sequence of the pre-pro polypeptide of the FNA protease set forth as SEQ ID NO:7.

12. The isolated modified polynucleotide of claim 11, wherein said first polynucleotide comprises at least one combination of mutations encoding a combination of substitutions selected from S49A-S24T, S49A-K72D, S49A-S78M, S49A-S78V, S49A-P93S, S49C-S24T, S49C-K72D, S49C-S78M, S49C-S78V, S49C-K91A, S49C-P93S, K91A-S24T, K91A-S49A, K91A-S52H, K91A-K72D, K91A-S78M, K91A-S78V, P93S-S24T, P93S-S49C, P93S-S52H, P93S-K72D, P93S-S78M, and P93S-S78V, wherein the positions are numbered by correspondence with the amino acid sequence of the pre-pro polypeptide of the FNA protease set forth as SEQ ID NO:7.

13. The isolated modified polynucleotide of claim 1, wherein said first polynucleotide comprises at least one mutation encoding at least one deletion selected from p.X18_X19del, p.X22_{—}23del, pX37del, pX49del, p.X47del, pX55del and p.X57del, wherein the positions are numbered by correspondence with the amino acid sequence of the pre-pro polypeptide of the FNA protease set forth as SEQ ID NO:7.

14. The isolated modified polynucleotide of claim 13, wherein said first polynucleotide comprises at least one mutation encoding at least one deletion selected from p. I18_T19del, p.F22_G23del, p.E37del, p.T47del, p.S49del, p.K55del, and p.K57del, wherein the positions are numbered by correspondence with the amino acid sequence of the pre-pro polypeptide of the FNA protease set forth as SEQ ID NO:7.

15. The isolated polynucleotide of claim 1, wherein said first polynucleotide comprises at least one mutation encoding at least one insertion selected from p.X2_X3insT, p.X30_X31insA, p.X19_X20insAT, p.X21_X22insS, p.X32_X33insG, p.X36_X37insG, and p.X58_X59insA, wherein the positions are numbered by correspondence with the amino acid sequence of the pre-pro polypeptide of the FNA protease set forth as SEQ ID NO:7.

16. The isolated modified polynucleotide of claim 15, wherein said first polynucleotide comprises at least one mutation encoding an insertion selected from p.R2_S3insT, p.A30_A31insA, p.T19_M20insAT, p.A21_F22insS, p.G32_K33insG, p.G36_E37insG, and p.D58_V59insA, wherein the positions are numbered by correspondence with the amino acid sequence of the pre-pro polypeptide of the FNA protease set forth as SEQ ID NO:7.

17. The isolated polynucleotide of claim 1, wherein said first polynucleotide comprises at least two mutations encoding at least one substitution and at least one deletion selected from X46H-p.X47del, X49A-p.X22_X23del, x49C-p.X22_X23del, X48I-p.X49del, X17W-p.X18_X19del, X78M-p.X22_X23del, X78V-p.X22_X23del, X78V-p.X57del, X91A-p.X22_X23del, X91A-X48I-pX49del, X91A-p.X57del, X93S-p.X22_X23del, and X93S-X48I-p.X49del, and wherein the positions are numbered by correspondence with the amino acid sequence of the pre-pro polypeptide of the FNA protease set forth as SEQ ID NO:7.

18. The isolated modified polynucleotide of claim 17, wherein said first polynucleotide comprises at least two mutations encoding at least one substitution and at least one deletion selected from the group consisting of Q46H-p.T47del, S49A-p.F22_G23del, S49C-p.F22_G23del, M48I-p.S49de, I17W-p.I18_T19del, S78M-p.F22_G23del, S78V-p.F22_G23del, K91A-p.F22_G23del, K91A-M48I-pS49del, K91A-p.K57del, P93S-p.F22_G23del, and P93S-M48I-p.S49del, wherein the positions are numbered by correspondence with the amino acid sequence of the pre-pro polypeptide of the FNA protease set forth as SEQ ID NO:7.

19. The isolated modified polynucleotide of claim 1, wherein said first polynucleotide comprises at least two mutations encoding at least one substitution and at least one insertion selected from X49A-p.X2_X3insT, X49A-p32X_X33insG, X49A-p.X19_X20insAT, X49C-p.X19_X20insAT, X49C-p.X32_X33insG, X52H-p.X19_X20insAT, X72 D-p.X19_X20insAT, X78M-p.X19_X20insAT, X78V-p.X19_X20insAT, X91A-p.X19_X20insAT, X91A-p.X32_X33insG, X93S-p.X19_X20insAT, and X93S-p.X32_X33insG, and wherein the positions are numbered by correspondence with the amino acid sequence of the pre-pro polypeptide of the FNA protease set forth as SEQ ID NO:7.

20. The isolated modified polynucleotide of claim 19, wherein said first polynucleotide comprises at least two mutations encoding at least one substitution and at least one insertion selected from S49A-p.R2_S3insT, S49A-p32G_K33insG, S49A-p.T19_M20insAT, S49C-p.T19_M20insAT, S49C-p.G32 K33insG, S49C-p.T19_M20insAT, S52H-p.T19_M20insAT, K72D-p.T19_M20insAT, S78M-p.T19_M20insAT, S78V-p.T19_M20insAT, K91A-p.T19_M20insAT, K91A-p.G32_K33insG, P93S-p.T19_M20insAT, and P93S-p.G32_K33insG, wherein the positions are numbered by correspondence with the amino acid sequence of the pre-pro polypeptide of the FNA protease set forth as SEQ ID NO:7.

21. The isolated modified polynucleotide of claim 1, wherein said first polynucleotide comprises at least two mutations encoding at least one deletion and at least one insertion selected from p.X57del-p.X19_X20insAT, and p.X 22_X23del-p.X2_X3insT, and wherein the positions are numbered by correspondence with the amino acid sequence of the pre-pro polypeptide of the FNA protease set forth as SEQ ID NO:7.

22. The isolated modified polynucleotide of claim 21, wherein said first polynucleotide comprises at least two mutations encoding a deletion and an insertion selected from pK57del-p.T19_M20insAT, and p.F22_G23del-p.R2_S3insT.

23. The isolated polynucleotide of claim 1, wherein said first polynucleotide comprises at least three mutations encoding at least one deletion, one insertion and one substitution corresponding to p.X49del-p.X19_X20insAT-X48I, and wherein the positions are numbered by correspondence with the amino acid sequence of the pre-pro polypeptide of the FNA protease set forth as SEQ ID NO:7.

24. The isolated polynucleotide of claim 23, wherein said first polynucleotide comprises at least three mutations encoding at least one deletion, one insertion and one substitution corresponding to p.S49del-p.T19_M20insAT-M48I, wherein the positions are numbered by correspondence with the amino acid sequence of the pre-pro polypeptide of the FNA protease set forth as SEQ ID NO:7.

25. An isolated polypeptide encoded by the modified full-length polynucleotide of claim 1.

26. An expression vector comprising the isolated modified polynucleotide of claim 1.

27. The expression vector of claim 26, further comprising an AprE promoter.

28. A host cell comprising the expression vector of claim 26.

29. The host cell of claim 28, wherein the host cell is a Bacillus sp. host cell.

30. The host cell of claim 29, wherein said Bacillus sp. host cell is selected from B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, and B. thuringiensis.

31. The host cell of claim 28, wherein said host cell is a B. subtilis host cell.

32. A method of producing a mature protease in a Bacillus sp. host cell, said method comprising:
(a) providing the expression vector of claim 26;
(b) transforming a host cell with said expression vector;
(c) culturing said host cell under suitable conditions such that said protease is produced by said host cell.

33. The method of claim 32, wherein said Bacillus sp. host cell is a Bacillus subtilis host cell.

34. The method of claim 32, wherein said protease is an alkaline serine protease.

35. The method of claim 32, wherein said modified polynucleotide encodes a protease comprising a mature region that is at least 65% identical to SEQ ID NO:9.

36. The method of claim 32, wherein said first polynucleotide encodes the pre-pro region of SEQ ID NO:7, wherein said first polynucleotide comprises at least one mutation to increase the production of said mature region of said protease, and wherein said second polynucleotide encodes the mature region of SEQ ID NO:9.
